(19) United States
(12) Patent Application Publication
(10) Pub. No.: US 2023/0045557 A1
(43) Pub. Date: Feb. 9, 2023

(54) ANTISENSE NUCLEIC ACID ENABLING EXON SKIPPING

(71) Applicants: NIPPON SHINYAKU CO., LTD., Kyoto-shi, Kyoto (JP); NATIONAL CENTER OF NEUROLOGY AND PSYCHIATRY, Kodaira-shi, Tokyo (JP)

(72) Inventors: Naoki WATANABE, Ibaraki (JP); Yuichiro TONE, Ibaraki (JP); Shin'ichi TAKEDA, Tokyo (JP); Yoshitsugu AOKI, Tokyo (JP)

(73) Assignees: NIPPON SHINYAKU CO., LTD., Kyoto-shi, Kyoto (JP); NATIONAL CENTER OF NEUROLOGY AND PSYCHIATRY, Kodaira-shi, Tokyo (JP)

(21) Appl. No.: 17/785,565

(22) PCT Filed: Dec. 18, 2020
(86) PCT No.: PCT/JP2020/047340

§ 371 Date: Jun. 15, 2022

(30) Foreign Application Priority Data
Dec. 19, 2019 (JP) 2019-229763

(51) Int. Cl. C12N 15/113

(52) U.S. Cl. CPC C12N 15/113; C12N 2310/11; C12N 2310/3233; C12N 2310/314; C12N 2320/33

(57) ABSTRACT

The present specification provides an antisense oligomer capable of causing simultaneous skipping of a plurality of exons in pre-mRNA of interest, and a pharmaceutical composition comprising the oligomer. The present specification also provides an antisense oligomer or a pharmaceutically acceptable salt thereof, or hydrate thereof which causes simultaneous skipping of two or more numerically consecutive exons from pre-mRNA of interest, the antisense oligomer comprising a base sequence complementary to a base sequence of a region including the vicinity of a donor of any intron in the pre-mRNA of interest, or a region including the vicinity of an acceptor of any intron in the pre-mRNA of interest, or a partial base sequence thereof.

### TECHNICAL FIELD

The present invention relates to an antisense oligomer capable of causing simultaneous skipping of a plurality of exons in a target gene, and a pharmaceutical composition comprising the oligomer.

### BACKGROUND ART

In recent years, exon skipping therapy has received attention which involves causing exon skipping of a gene having a mutation that causes a disease so that a protein having partial functions arises, thereby treating the disease. Examples of the disease that may be treated by such exon skipping therapy include Duchenne muscular dystrophy (DMD).

DMD is the most frequent form of hereditary progressive muscular disease that affects one in about 3,500 newborn boys. Although DMD patients exhibit motor functions rarely different from healthy humans in their infancy and childhood, muscle weakness is observed in children from around 4 to 5 years old. Then, muscle weakness in DMD patients progresses with age to the loss of ambulation by about 12 years old and death due to cardiac or respiratory insufficiency in their twenties. Therefore, it has been strongly desired to develop an effective therapeutic agent.

DMD is known to be caused by a mutation in the dystrophin gene. The dystrophin gene is located on X chromosome and is a huge gene consisting of 2.2 million DNA base pairs. DNA is transcribed into pre-mRNA, and introns are removed by splicing to form mRNA of 13,993 bases in which 79 exons are joined together. This mRNA is translated into 3,685 amino acids to produce the dystrophin protein. The dystrophin protein is associated with the maintenance of membrane stability in muscle cells and necessary to make muscle cells less fragile. Patients with DMD have a mutation in the dystrophin gene and hence, the functional dystrophin protein is rarely expressed in muscle cells of the patients. Therefore, the structure of muscle cells cannot be maintained at the time of muscle contraction in the body of the patients with DMD, leading to a large influx of calcium ions into muscle cells. Consequently, muscle cell necrosis and fibrosis progress so that muscle cells become progressively more difficult to regenerate.

Becker muscular dystrophy (BMD) is also caused by a mutation in the dystrophin gene. The symptoms involve muscle weakness but are typically mild and slow in the progress of muscle weakness, when compared to DMD. In many cases, its onset is in adulthood. Differences in clinical symptoms between DMD and BMD are considered to reside in whether the reading frame for amino acids on the translation of dystrophin mRNA into the dystrophin protein is disrupted by the mutation or not (Non Patent Literature 1). More specifically, in DMD, the presence of mutation shifts the amino acid reading frame so that the expression of functional dystrophin protein is abolished, whereas in BMD the dystrophin protein that is capable of functioning, though imperfectly, is produced because the amino acid reading frame is preserved, while part of the exons are deleted by the mutation.

Exon skipping is expected to serve as a method for treating DMD. This method involves modifying splicing to restore the amino acid reading frame of dystrophin mRNA and induce expression of the dystrophin protein having the function partially restored (Non Patent Literature 2). The amino acid sequence part to be translated from an exon, which is a target for exon skipping, will be lost. For this reason, the dystrophin protein expressed by this treatment becomes shorter than normal one but since the amino acid reading frame is maintained, the function to stabilize muscle cells is partially retained. Consequently, it is expected that exon skipping will lead DMD to the similar symptoms to that of BMD which is milder. The exon skipping approach has passed the animal tests using mice or dogs and now is currently assessed in clinical trials on human DMD patients.

The skipping of an exon can be induced by binding of antisense nucleic acids targeting site(s) surrounding either 5′ or 3′ splice site or both sites, or exon-internal sites. An exon will only be included in the mRNA when both splice sites thereof are recognized by the spliceosome complex. Thus, exon skipping can be induced by targeting the sites surrounding the splice sites with antisense nucleic acids. Furthermore, the binding of an SR protein rich in serine and arginine to an exonic splicing enhancer (ESE) is considered necessary for an exon to be recognized by the splicing mechanism. Accordingly, exon skipping can also be induced by targeting ESE.

Since a mutation of the dystrophin gene may vary depending on DMD patients, antisense nucleic acids need to be designed based on the site or type of respective genetic mutation. There is a plurality of reports on an antisense nucleic acid that induces exon skipping targeting one sequence of consecutive bases for a single exon in the dystrophin gene (Patent Literatures 1 to 6 and Non Patent Literatures 1 and 2). It has also been reported that when two types of antisense nucleic acids that target the same exon in the dystrophin gene are mixed and allowed to act (dual targeting), skipping activity may be enhanced as compared to use of each antisense nucleic acid alone (Patent Literature 7).

A method called multi-exon skipping has received attention which involves causing skipping of a plurality of exons (exon group), not one exon as described above. This method enables a wide range of mutations in the dystrophin gene to be treated by exon skipping. For example, exons 45 to 55 in the dystrophin gene are known as hot spots of genetic mutation, and it has been reported that skipping of these 11 exons enables about 60% of DMD patients having a deletion mutation to be treated (Non Patent Literature 3). Most of patients congenitally lacking exons 45 to 55 are known to manifest no or mild symptoms, though developing BMD (Non Patent Literature 4). Thus, it is expected that drugs capable of inducing exon 45 to 55 skipping are promising as therapeutic agents for DMD.

For example, a method using antisense nucleic acids respectively targeting all exons in a region which is the target of exon skipping (Non Patent Literatures 5, 7, 8, and 10), and a method using antisense nucleic acids respectively targeting two different exons on the 3′ side and 5′ side of a region which is the target of exon skipping (Non Patent Literatures 6 and 9 and Patent Literature 8) have been reported as methods for inducing multi-exon skipping.

However, there is still no report stating that an antisense nucleic acid targeting a region including the vicinity of a donor of an intron or the vicinity of an acceptor induces multi-exon skipping.

### CITATION LIST

### Patent Literature

Patent Literature 1: International Publication WO2004/048570
Patent Literature 2: International Publication WO2009/139630
Patent Literature 3: International Publication WO2010/048586
Patent Literature 4: U.S. Patent Publication Nos. 2010/0168212
Patent Literature 5: International Publication WO2011/057350
Patent Literature 6: International Publication WO2006/000057
Patent Literature 7: International Publication WO2007/135105
Patent Literature 8: International Publication WO2004/083446

### Non Patent Literature

Non Patent Literature 1: Annemieke Aartsma-Rus et al., (2002) Neuromuscular Disorders 12: S71-S77
Non Patent Literature 2: Wilton S. D., e t al., Molecular Therapy 2007: 15: p. 1288-96
Non Patent Literature 3: Christophe Beroud et al., Human Mutation, 28(2), 2007, 196-202
Non Patent Literature 4: Yusuke Echigoya et al., Molecular Therapy-Nucleic Acids, 4(2), 2015, e225
Non Patent Literature 5: Yoshitsugu Aoki et al., PNAS, 109(34), 2012, 13763-13768
Non Patent Literature 6: Laura van Vliet et al., BMC Medical Genetics, 9, 105, 2008
Non Patent Literature 7: Joshua Lee et al., PLoS ONE, 13(5), e0197084, 2018
Non Patent Literature 8: Joshua Lee et al., Methods in Molecular Biology, 1828, 141-150, 2018
Non Patent Literature 9: Annemieke Aartsma-Rus et al, Am. J. Hum. Genet. 74(1), 83-92, 2004
Non Patent Literature 10: Yusuke Echigoya et al., Molecular Therapy, 27(11), 1-13, 2019

### SUMMARY OF INVENTION

### Technical Problem

Under the foregoing circumstances, medicaments for treating patients having various mutations by causing simultaneous skipping of a plurality of exons (exon group) in pre-mRNA of interest have been desired.

### Solution to Problem

As a result of detailed studies of the technical contents of the above documents and the structure of the dystrophin gene, the present inventors have found that an antisense nucleic acid targeting a specific region in human dystrophin pre-mRNA is capable of causing simultaneous skipping of a plurality of exons among exons 45 to 55. Based on this finding, the present inventors have accomplished the present invention.

Specifically, the present invention is as follows.

[1]

An antisense oligomer or a pharmaceutically acceptable salt thereof, or hydrate thereof which causes simultaneous skipping of any two or more numerically consecutive exons selected from the group consisting of the 45th exon to the 55th exon in human dystrophin pre-mRNA,

the antisense oligomer comprising a base sequence complementary to a base sequence of at least one region selected from the group consisting of regions R1 to R24 represented by

region Rn (wherein n is an odd number of 1 to 23) which consists of a base sequence of NX bases in the upstream direction from the 3′ end of the NAth exon and a base sequence of NY bases in the downstream direction from the 5′ end of the NBth intron in the human dystrophin pre-mRNA, and

region Rn (wherein n is an even number of 2 to 24) which consists of a base sequence of NX bases in the upstream direction from the 3′ end of the NAth intron and a base sequence of NY bases in the downstream direction from the 5′ end of the NBth exon in the human dystrophin pre-mRNA,

or a partial base sequence thereof, wherein

when n is 1, NA=44, NB=44, NX=20, and NY=400,

when n is 2, NA=44, NB=45, NX=600, and NY=50,

when n is 3, NA=45, NB=45, NX=20, and NY=400,

when n is 4, NA=45, NB=46, NX=400, and NY=50,

when n is 5, NA=46, NB=46, NX=20, and NY=400,

when n is 6, NA=46, NB=47, NX=400, and NY=50,

when n is 7, NA=47, NB=47, NX=20, and NY=400,

when n is 8, NA=47, NB=48, NX=400, and NY=50,

when n is 9, NA=48, NB=48, NX=20, and NY=400,

when n is 10, NA=48, NB=49, NX=400, and NY=50,

when n is 11, NA=49, NB=49, NX=20, and NY=400,

when n is 12, NA=49, NB=50, NX=400, and NY=50,

when n is 13, NA=50, NB=50, NX=20, and NY=400,

when n is 14, NA=50, NB=51, NX=400, and NY=50,

when n is 15, NA=51, NB=51, NX=20, and NY=400,

when n is 16, NA=51, NB=52, NX=400, and NY=50,

when n is 17, NA=52, NB=52, NX=20, and NY=400,

when n is 18, NA=52, NB=53, NX=400, and NY=50,

when n is 19, NA=53, NB=53, NX=20, and NY=400,

when n is 20, NA=53, NB=54, NX=400, and NY=50,

when n is 21, NA=54, NB=54, NX=20, and NY=400,

when n is 22, NA=54, NB=55, NX=400, and NY=50,

when n is 23, NA=55, NB=55, NX=20, and NY=400, or

when n is 24, NA=55, NB=56, NX=400, and NY=50.

[2]

The antisense oligomer or the pharmaceutically acceptable salt thereof, or hydrate thereof according to [1], wherein

the region R1 is a region that consists of a base sequence of 20 bases in the upstream direction from the 3′ end of the 44th exon and a base sequence of 400 bases in the downstream direction from the 5′ end of the 44th intron in the human dystrophin pre-mRNA,

the region R2 is a region that consists of a base sequence of 600 bases in the upstream direction from the 3′ end of the 44th intron and a base sequence of 50 bases in the downstream direction from the 5′ end of the 45th exon in the human dystrophin pre-mRNA,

the region R3 is a region that consists of a base sequence of 20 bases in the upstream direction from the 3′ end of the 45th exon and a base sequence of 400 bases in the downstream direction from the 5′ end of the 45th intron in the human dystrophin pre-mRNA,

the region R4 is a region that consists of a base sequence of 400 bases in the upstream direction from the 3′ end of the 45th intron and a base sequence of 50 bases in the downstream direction from the 5′ end of the 46th exon in the human dystrophin pre-mRNA,

the region R5 is a region that consists of a base sequence of 20 bases in the upstream direction from the 3′ end of the 46th exon and a base sequence of 400 bases in the downstream direction from the 5′ end of the 46th intron in the human dystrophin pre-mRNA,

the region R6 is a region that consists of a base sequence of 400 bases in the upstream direction from the 3′ end of the 46th intron and a base sequence of 50 bases in the downstream direction from the 5′ end of the 47th exon in the human dystrophin pre-mRNA,

the region R7 is a region that consists of a base sequence of 20 bases in the upstream direction from the 3′ end of the 47th exon and a base sequence of 400 bases in the downstream direction from the 5′ end of the 47th intron in the human dystrophin pre-mRNA,

the region R8 is a region that consists of a base sequence of 400 bases in the upstream direction from the 3′ end of the 47th intron and a base sequence of 50 bases in the downstream direction from the 5′ end of the 48th exon in the human dystrophin pre-mRNA,

the region R9 is a region that consists of a base sequence of 20 bases in the upstream direction from the 3′ end of the 48th exon and a base sequence of 400 bases in the downstream direction from the 5′ end of the 48th intron in the human dystrophin pre-mRNA,

the region R10 is a region that consists of a base sequence of 400 bases in the upstream direction from the 3′ end of the 48th intron and a base sequence of 50 bases in the downstream direction from the 5′ end of the 49th exon in the human dystrophin pre-mRNA,

the region R11 is a region that consists of a base sequence of 20 bases in the upstream direction from the 3′ end of the 49th exon and a base sequence of 400 bases in the downstream direction from the 5′ end of the 49th intron in the human dystrophin pre-mRNA,

the region R12 is a region that consists of a base sequence of 400 bases in the upstream direction from the 3′ end of the 49th intron and a base sequence of 50 bases in the downstream direction from the 5′ end of the 50th exon in the human dystrophin pre-mRNA,

the region R13 is a region that consists of a base sequence of 20 bases in the upstream direction from the 3′ end of the 50th exon and a base sequence of 400 bases in the downstream direction from the 5′ end of the 50th intron in the human dystrophin pre-mRNA,

the region R14 is a region that consists of a base sequence of 400 bases in the upstream direction from the 3′ end of the 50th intron and a base sequence of 50 bases in the downstream direction from the 5′ end of the 51st exon in the human dystrophin pre-mRNA,

the region R15 is a region that consists of a base sequence of 20 bases in the upstream direction from the 3′ end of the 51st exon and a base sequence of 400 bases in the downstream direction from the 5′ end of the 51st intron in the human dystrophin pre-mRNA,

the region R16 is a region that consists of a base sequence of 400 bases in the upstream direction from the 3′ end of the 51st intron and a base sequence of 50 bases in the downstream direction from the 5′ end of the 52nd exon in the human dystrophin pre-mRNA,

the region R17 is a region that consists of a base sequence of 20 bases in the upstream direction from the 3′ end of the 52nd exon and a base sequence of 400 bases in the downstream direction from the 5′ end of the 52nd intron in the human dystrophin pre-mRNA,

the region R18 is a region that consists of a base sequence of 400 bases in the upstream direction from the 3′ end of the 52nd intron and a base sequence of 50 bases in the downstream direction from the 5′ end of the 53rd exon in the human dystrophin pre-mRNA,

the region R19 is a region that consists of a base sequence of 20 bases in the upstream direction from the 3′ end of the 53rd exon and a base sequence of 400 bases in the downstream direction from the 5′ end of the 53rd intron in the human dystrophin pre-mRNA,

the region R20 is a region that consists of a base sequence of 400 bases in the upstream direction from the 3′ end of the 53rd intron and a base sequence of 50 bases in the downstream direction from the 5′ end of the 54th exon in the human dystrophin pre-mRNA,

the region R21 is a region that consists of a base sequence of 20 bases in the upstream direction from the 3′ end of the 54th exon and a base sequence of 400 bases in the downstream direction from the 5′ end of the 54th intron in the human dystrophin pre-mRNA,

the region R22 is a region that consists of a base sequence of 400 bases in the upstream direction from the 3′ end of the 54th intron and a base sequence of 50 bases in the downstream direction from the 5′ end of the 55th exon in the human dystrophin pre-mRNA,

the region R23 is a region that consists of a base sequence of 20 bases in the upstream direction from the 3′ end of the 55th exon and a base sequence of 400 bases in the downstream direction from the 5′ end of the 55th intron in the human dystrophin pre-mRNA, or

the region R24 is a region that consists of a base sequence of 400 bases in the upstream direction from the 3′ end of the 55th intron and a base sequence of 50 bases in the downstream direction from the 5′ end of the 56th exon in the human dystrophin pre-mRNA.

[3]

The antisense oligomer or the pharmaceutically acceptable salt thereof, or hydrate thereof according to [1] or [2], wherein

the antisense oligomer comprises a base sequence complementary to

(a) any one base sequence selected from the group consisting of SEQ ID NOs: 233 to 256, 341 to 369, and 385 to 389,
(b) a base sequence that hybridizes under stringent conditions to a base sequence complementary to any one base sequence selected from the group consisting of SEQ ID NOs: 233 to 256, 341 to 369, and 385 to 389,
(c) a base sequence that has at least 85% identity with any one base sequence selected from the group consisting of SEQ ID NOs: 233 to 256, 341 to 369, and 385 to 389, and has a length within ±15% of the length of the any one base sequence selected, or
(d) a partial base sequence of any one base sequence selected from the group consisting of the base sequences (a), (b), and (c).
[4]

The antisense oligomer or the pharmaceutically acceptable salt thereof, or hydrate thereof according to any one of [1] to [3], wherein

the antisense oligomer is an antisense oligomer comprising two or more unit oligomers linked to each other, wherein

each of the unit oligomers comprises a base sequence complementary to a base sequence of any one region selected from the group consisting of the regions R1 to R24, or a partial base sequence thereof, and the respective base sequences of the unit oligomers are neither consecutive nor overlapped with each other.

[5]

The antisense oligomer or the pharmaceutically acceptable salt thereof, or hydrate thereof according to any one of [1] to [3], wherein

the antisense oligomer is an antisense oligomer comprising two or more unit oligomers linked to each other, wherein

each of the unit oligomers comprises a base sequence complementary to

(a) any one base sequence selected from the group consisting of SEQ ID NOs: 233 to 256, 341 to 369, and 385 to 389,
(b) a base sequence that hybridizes under stringent conditions to a base sequence complementary to any one base sequence selected from the group consisting of SEQ ID NOs: 233 to 256, 341 to 369, and 385 to 389,
(c) a base sequence that has at least 85% identity with any one base sequence selected from the group consisting of SEQ ID NOs: 233 to 256, 341 to 369, and 385 to 389, and has a length within ±15% of the length of the any one base sequence selected, or
(d) a partial base sequence of any one base sequence selected from the group consisting of the base sequences (a), (b), and (c),
and the respective base sequences of the unit oligomers are neither consecutive nor overlapped with each other.
[6]

The antisense oligomer or the pharmaceutically acceptable salt thereof, or hydrate thereof according to [4] or [5], wherein each of the unit oligomers comprises a base sequence complementary to a consecutive base sequence of 5- to 20-base length in the region.

[7]

The antisense oligomer or the pharmaceutically acceptable salt thereof, or hydrate thereof according to any one of [1] to [3], wherein the antisense oligomer consists of

(1) any one base sequence selected from the group consisting of SEQ ID NOs: 1 to 111, 113 to 177, 179, 180, 182, 183, 185 to 193, 195 to 198, 200 to 223, and 225 to 232, or
(2) a base sequence that has at least 85% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±15% of the length of the any one base sequence selected.
[8]

The antisense oligomer or the pharmaceutically acceptable salt thereof, or hydrate thereof according to any one of [1] to [3] and [7], wherein the antisense oligomer consists of any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232.

[9]

The antisense oligomer or the pharmaceutically acceptable salt thereof, or hydrate thereof according to any one of [1] to [3], [7] and [8], wherein the antisense oligomer consists of any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228.

[10]

The antisense oligomer or the pharmaceutically acceptable salt thereof, or hydrate thereof according to any one of [1] to [9], wherein the antisense oligomer is an oligonucleotide.

[11]

The antisense oligomer or the pharmaceutically acceptable salt thereof, or hydrate thereof according to [10], wherein the sugar moiety and/or the phosphate bond moiety of at least one nucleotide constituting the oligonucleotide is modified.

[12]

The antisense oligomer or the pharmaceutically acceptable salt thereof, or hydrate thereof according to [10] or [11], wherein the sugar moiety of at least one nucleotide constituting the oligonucleotide is a ribose in which the 2′-OH group is replaced by any one selected from the group consisting of —OR, —R, —R′OR, —SH, —SR, —NH₂, —NHR, —NR₂, —N₃, —CN, —F, —Cl, —Br, and —I (wherein R is an alkyl or an aryl and R′ is an alkylene).

[13]

The antisense oligomer or the pharmaceutically acceptable salt thereof, or hydrate thereof according to any one of [10] to [12], wherein the phosphate bond moiety of at least one nucleotide constituting the oligonucleotide is any one selected from the group consisting of a phosphorothioate bond, a phosphorodithioate bond, an alkylphosphonate bond, a phosphoramidate bond and a boranophosphate bond.

[14]

The antisense oligomer or the pharmaceutically acceptable salt thereof, or hydrate thereof according to any one of [1] to [9], wherein the antisense oligomer is a morpholino oligomer.

[15]

The antisense oligomer or the pharmaceutically acceptable salt thereof, or hydrate thereof according to [14], wherein the antisense oligomer is a phosphorodiamidate morpholino oligomer.

[16]

The antisense oligomer or the pharmaceutically acceptable salt thereof, or hydrate thereof according to [14] or [15], wherein the 5′ end is any one of chemical formulae (1) to (3) below:

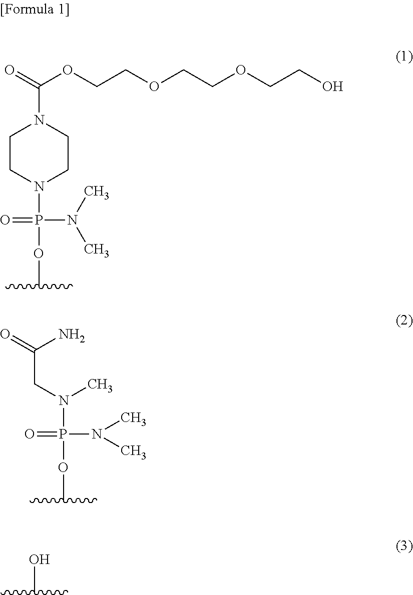

[17]

A suppressor antisense oligomer or a pharmaceutically acceptable salt thereof, or hydrate thereof which suppresses single skipping of any one exon selected from the group consisting of the 45th exon to the 55th exon in human dystrophin pre-mRNA,

the suppressor antisense oligomer comprising a base sequence complementary to

(a) any one base sequence of a base sequence selected from the group consisting of SEQ ID NOs: 370 to 384,
(b) a base sequence that has at least 85% identity with any one base sequence of a base sequence selected from the group consisting of SEQ ID NOs: 370 to 384, and has a length within ±15% of the length of the any one base sequence selected, or
(c) a partial base sequence of the base sequence (a) or (b).
[18]

The suppressor antisense oligomer or the pharmaceutically acceptable salt thereof, or hydrate thereof according to [17], wherein the suppressor antisense oligomer consists of

(1) any one base sequence selected from the group consisting of SEQ ID NOs: 257 to 275, or (2) a base sequence that has at least 85% identity with any one base sequence selected from the group consisting of SEQ ID NOs: 257 to 275, and has a length within ±15% of the length of the any one base sequence selected.
[19]

The suppressor antisense oligomer or the pharmaceutically acceptable salt thereof, or hydrate thereof according to [17] or [18], wherein the suppressor antisense oligomer consists of any one base sequence selected from the group consisting of SEQ ID NOs: 260, 261, and 263.

[20]

The suppressor antisense oligomer or the pharmaceutically acceptable salt thereof, or hydrate thereof according to any one of [17] to [19], wherein the suppressor antisense oligomer is an oligonucleotide.

[21]

The suppressor antisense oligomer or the pharmaceutically acceptable salt thereof, or hydrate thereof according to [20], wherein the sugar moiety and/or the phosphate bond moiety of at least one nucleotide constituting the oligonucleotide is modified.

[22]

The suppressor antisense oligomer or the pharmaceutically acceptable salt thereof, or hydrate thereof according to [20] or [21], wherein the sugar moiety of at least one nucleotide constituting the oligonucleotide is a ribose in which the 2′-OH group is replaced by any one selected from the group consisting of —OR, —R, —R′OR, —SH, —SR, —NH₂, —NHR, —NR₂, —N₃, —CN, —F, —Cl, —Br, and —I

(wherein R is an alkyl or an aryl and R′ is an alkylene).
[23]

The suppressor antisense oligomer or the pharmaceutically acceptable salt thereof, or hydrate thereof according to any one of [20] to [22], wherein the phosphate bond moiety of at least one nucleotide constituting the oligonucleotide is any one selected from the group consisting of a phosphorothioate bond, a phosphorodithioate bond, an alkylphosphonate bond, a phosphoramidate bond and a boranophosphate bond.

[24]

The suppressor antisense oligomer or the pharmaceutically acceptable salt thereof, or hydrate thereof according to any one of [17] to [19], wherein the suppressor antisense oligomer is a morpholino oligomer.

[25]

The suppressor antisense oligomer or the pharmaceutically acceptable salt thereof, or hydrate thereof according to [24], wherein the suppressor antisense oligomer is a phosphorodiamidate morpholino oligomer.

[26]

The suppressor antisense oligomer or the pharmaceutically acceptable salt thereof, or hydrate thereof according to [24] or [25], wherein the 5′ end is any one of chemical formulae (1) to (3) below:

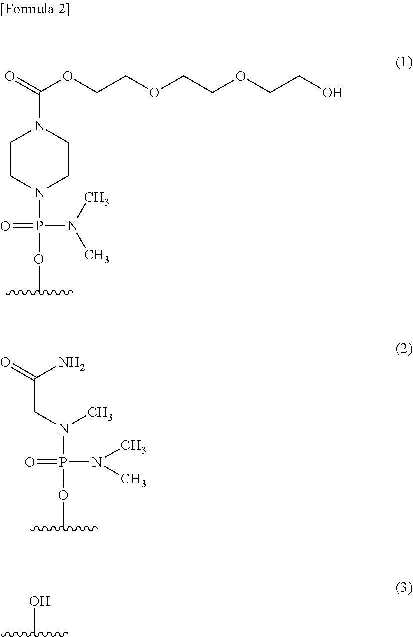

[27]

A pharmaceutical composition comprising the antisense oligomer or the pharmaceutically acceptable salt thereof, or hydrate thereof according to any one of [1] to [16].

[28]

The pharmaceutical composition according to [27], further comprising the suppressor antisense oligomer or the pharmaceutically acceptable salt thereof, or hydrate thereof according to any one of [17] to [26].

[29]

A pharmaceutical composition comprising the antisense oligomer or the pharmaceutically acceptable salt thereof, or hydrate thereof according to any one of [1] to [16], and the suppressor antisense oligomer or the pharmaceutically acceptable salt thereof, or hydrate thereof according to any one of [17] to [26].

[30]

The pharmaceutical composition according to [28] or [29], wherein

(1) the antisense oligomer is an oligomer consisting of SEQ ID NO: 75, and the suppressor antisense oligomer is an oligomer consisting of SEQ ID NO: 260,
(2) the antisense oligomer is an oligomer consisting of SEQ ID NO: 75, and the suppressor antisense oligomer is an oligomer consisting of SEQ ID NO: 261, or
(3) the antisense oligomer is an oligomer consisting of SEQ ID NO: 75, and the suppressor antisense oligomer is an oligomer consisting of SEQ ID NO: 263.
[31]

The pharmaceutical composition according to any one of [27] to [30], further comprising a pharmaceutically acceptable carrier.

[32]

The pharmaceutical composition according to any one of [27] to [31] for treatment of muscular dystrophy.

[33]

The pharmaceutical composition according to any one of [27] to [32] for being administered to a human patient.

[34]

A method for treatment of muscular dystrophy, which comprises administering to a patient with muscular dystrophy the antisense oligomer or the pharmaceutically acceptable salt thereof, or hydrate thereof according to any one of [1] to [16], or the pharmaceutical composition according to any one of [27] to [33].

[35]

The method for treatment according to [34], wherein the patient with muscular dystrophy is a patient with a mutation that is amenable to exon 45 to 55 skipping in the dystrophin gene.

[36]

The method for treatment according to [34] or [35], wherein the patient is a human.

[37]

Use of the antisense oligomer or the pharmaceutically acceptable salt thereof, or hydrate thereof according to any one of [1] to [16], or the pharmaceutical composition according to any one of [27] to [33] in manufacturing of a medicament for the treatment of muscular dystrophy.

[38]

The antisense oligomer or the pharmaceutically acceptable salt thereof, or hydrate thereof according to any one of [1] to [16], or the pharmaceutical composition according to any one of [27] to [33] for use in the treatment of muscular dystrophy.

[39]

The antisense oligomer or the pharmaceutically acceptable salt thereof, or hydrate thereof, or the pharmaceutical composition according to [38], wherein the treatment involves performing skipping of any two or more numerically consecutive exons selected from the group consisting of the 45th exon to the 55th exon in human dystrophin pre-mRNA.

[40]

The antisense oligomer or the pharmaceutically acceptable salt thereof, or hydrate thereof, or the pharmaceutical composition according to [38] or [39], wherein in the treatment, a patient with muscular dystrophy is a human.

[41]

A method for enhancing the efficiency of skipping of two or more numerically consecutive exons, which comprises

inhibiting a splicing silencer sequence, a splice site sequence, or a branch site sequence of pre-mRNA of interest when the two or more numerically consecutive exons are skipped from the pre-mRNA of interest.

[42]

The method according to [41], wherein the splicing silencer sequence is a recognition sequence of heterogeneous nuclear ribonucleoprotein A1 (hnRNPA1).

[43]

The method according to [41] or [42], wherein the pre-mRNA of interest is human dystrophin pre-mRNA.

[44]

The method according to any one of [41] to [43], wherein the two or more numerically consecutive exons are selected from the group consisting of the 45th exon to the 55th exon in human dystrophin pre-mRNA.

[45]

The method according to [44], wherein the skipping of the two or more numerically consecutive exons of the pre-mRNA of interest is performed using the antisense oligomer or the pharmaceutically acceptable salt thereof, or hydrate thereof according to any one of [1] to [16].

[46]

The method according to [44] or [45], wherein specific inhibition of the splicing silencer sequence, the splice site sequence, or the branch site sequence is performed using the suppressor antisense oligomer or the pharmaceutically acceptable salt thereof, or hydrate thereof according to any one of [17] to [26].

The present invention may also include the following embodiments.

[47]

An antisense oligomer or a pharmaceutically acceptable salt thereof, or hydrate thereof which causes simultaneous skipping of two or more numerically consecutive exons from pre-mRNA of interest,

the antisense oligomer comprising a base sequence complementary to a base sequence of any one region selected from the group consisting of

(1) a region that is constituted by a base sequence of 20 bases in the upstream direction from the 3′ end of any exon and a base sequence of 400 bases in the downstream direction from the 5′ end of an intron which is adjacent to the 3′ end of the exon in the pre-mRNA of interest, or

(2) a region that is constituted by a base sequence of 400 bases or 600 bases in the upstream direction from the 3′ end of any intron and a base sequence of 50 bases in the downstream direction from the 5′ end of an exon which is adjacent to the 3′ end of the intron in the pre-mRNA of interest,

or a partial base sequence thereof.
[48]

The antisense oligomer or the pharmaceutically acceptable salt thereof, or hydrate thereof according to [47], wherein the pre-mRNA of interest is human dystrophin pre-mRNA.

### Advantageous Effects of Invention

The present invention provides an antisense oligomer that causes simultaneous skipping of a plurality of exons in a target. Another embodiment of the present invention provides a pharmaceutical composition for treating patients having various mutations by causing simultaneous skipping of a plurality of exons in pre-mRNA of interest. An alternative embodiment of the present invention provides a suppressor antisense oligomer that suppresses single skipping of an exon in pre-mRNA of interest or a pharmaceutical composition comprising the oligomer. An alternative embodiment of the present invention enables simultaneous skipping of exons 45 to 55 in human dystrophin pre-mRNA to be caused with high efficiency.

### BRIEF DESCRIPTION OF DRAWINGS

FIG. 1 is a diagram showing results of studying exon 45 to 55 skipping in human dystrophin pre-mRNA in RD cells (human rhabdomyosarcoma cells) by RT-PCR.

FIG. 2 is a diagram showing results of studying exon 45 to 55 skipping in human dystrophin pre-mRNA in RD cells by RT-PCR.

FIG. 3 is a diagram showing results of studying exon 45 to 55 skipping in human dystrophin pre-mRNA in RD cells by RT-PCR.

FIG. 4 is a diagram showing results of studying exon 45 to 55 skipping in human dystrophin pre-mRNA in RD cells by RT-PCR.

FIG. 5 is a diagram showing results of studying exon 45 to 55 skipping in human dystrophin pre-mRNA in RD cells by RT-PCR.

FIG. 6 is a diagram showing results of studying exon 45 to 55 skipping in human dystrophin pre-mRNA in RD cells by RT-PCR.

FIG. 7 is a diagram showing results of studying exon 45 to 55 skipping in human dystrophin pre-mRNA in RD cells by RT-PCR.

FIG. 8 is a diagram showing results of studying exon 45 to 55 skipping in human dystrophin pre-mRNA in RD cells by RT-PCR.

FIG. 9 is a diagram showing results of studying exon 45 to 55 skipping in human dystrophin pre-mRNA in RD cells by RT-PCR.

FIG. 10 is a diagram showing results of studying exon 45 to 55 skipping in human dystrophin pre-mRNA in RD cells by RT-PCR.

FIG. 11 is a diagram showing results of studying exon 45 to 55 skipping in human dystrophin pre-mRNA in RD cells by RT-PCR.

FIG. 12 is a diagram showing results of studying exon 45 to 55 skipping in human dystrophin pre-mRNA in RD cells by RT-PCR.

FIG. 13 is a diagram showing results of studying exon 45 to 55 skipping in human dystrophin pre-mRNA in RD cells by RT-PCR.

FIG. 14 is a diagram showing results of studying exon 45 to 55 skipping in human dystrophin pre-mRNA in RD cells by RT-PCR.

FIG. 15 is a diagram showing results of studying exon 45 to 55 skipping in human dystrophin pre-mRNA in RD cells by RT-PCR.

FIG. 16 is a diagram showing results of studying exon 45 to 55 skipping in human dystrophin pre-mRNA in RD cells by RT-PCR.

FIG. 17 is a diagram showing results of studying exon 45 to 55 multi-skipping and exon 45 single skipping in human dystrophin pre-mRNA in RD cells by RT-PCR.

FIG. 18 is a diagram showing results of studying exon 45 to 55 multi-skipping and exon 45 single skipping in human dystrophin pre-mRNA in RD cells by RT-PCR.

FIG. 19 is a diagram showing results of studying multi-skipping of exons selected from exons 45 to 49 and exon 45 single skipping in human dystrophin pre-mRNA in RD cells by RT-PCR.

FIG. 20 is a diagram showing results of studying multi-skipping of exons selected from exons 45 to 49 in human dystrophin pre-mRNA in RD cells by RT-PCR, and calculating the total amounts of the respective skips of exons 45 and 46, exons 45 to 47, exons 45 to 48, and exons 45 to 49 (total multi-skipping products). Each of these skips is considered to have therapeutic effects.

FIG. 21 is a diagram showing results of studying multi-skipping of exons selected from exons 45 to 52 and exon 45 single skipping in human dystrophin pre-mRNA in RD cells by RT-PCR.

FIG. 22 is a diagram showing results of studying multi-skipping of exons selected from exons 45 to 52 in human dystrophin pre-mRNA in RD cells by RT-PCR, and calculating the total amounts of the respective skips of exons 45 to 47, exons 45 to 48, exons 45 to 49, exons 45 to 50, exons 45 to 51, and exons 45 to 52 (left) or the total amounts of the respective skips of exons 45 to 47, exons 45 to 48, exons 45 to 49, and exons 45 to 51 (right). Each of the skips of exons 45 to 47, exons 45 to 48, exons 45 to 49, and exons 45 to 51 has therapeutic effects.

### DESCRIPTION OF EMBODIMENTS

Hereinafter, the present invention is described in detail. The embodiments described below are intended to be presented by way of example merely to describe the invention but not limited only to the following embodiments. The present invention may be implemented in various ways without departing from the gist of the invention.

### 1. Antisense Oligomer

The present invention provides an antisense oligomer or a pharmaceutically acceptable salt thereof, or hydrate thereof which causes simultaneous skipping of two or more numerically consecutive exons from pre-mRNA of interest,

the antisense oligomer comprising a base sequence complementary to a base sequence of any one region selected from the group consisting of

(1) a region that is constituted by a base sequence of 20 bases in the upstream direction from the 3′ end of any exon and a base sequence of 400 bases in the downstream direction from the 5′ end of an intron which is adjacent to the 3′ end of the exon in the pre-mRNA of interest, or

(2) a region that is constituted by a base sequence of 400 bases or 600 bases in the upstream direction from the 3′ end of any intron and a base sequence of 50 bases in the downstream direction from the 5′ end of an exon which is adjacent to the 3′ end of the intron in the pre-mRNA of interest,

or a partial base sequence thereof.

As used herein, the term “cause simultaneous skipping” of two or more numerically consecutive exons includes not only removal of the respective exons from pre-mRNA at completely the same timings but also sequential removal of the respective exons within a period from pre-mRNA to mature mRNA. Specifically, the term “cause simultaneous skipping” of two or more numerically consecutive exons refers to removal of a plurality of (two or more) numerically consecutive exons from pre-mRNA.

As used herein, the term “two or more numerically consecutive exons” means a plurality of exons that increase one by one in exon number among exons (the total number of exons is referred to as Texon) included in pre-mRNA of interest. The exon number means a number assigned to exons in order from the 5′ end to the 3′ end with an exon at the most upstream position of pre-mRNA defined as the first exon, followed by the second, the third, . . . . In the case of skipping of two or more numerically consecutive exons in a certain gene, its exon numbers a₁, . . . , aⱼ can be represented by the sequence {aⱼ}. The general term aⱼ in the sequence {aⱼ} is represented by the expression below:

aⱼ=m+(j−1) [Expression 1]

wherein m is a given natural number that satisfies 1≤m≤(Texon−1), and j is a natural number that satisfies 2≤(m+j)≤Texon+1.

When the pre-mRNA of interest is, for example, human dystrophin pre-mRNA, Texon is 79.

In a certain embodiment, j is a given natural number selected from 1 to 11. In another embodiment, j is 11, j is 10, j is 9, j is 8, j is 7, j is 6, j is 5, j is 4, j is 3, j is 2, or j is 1.

As used herein, the term “gene” is intended to mean a genomic gene and also include cDNA, pre-mRNA and mRNA. Preferably, the gene is pre-mRNA. As used herein, the term “pre-mRNA” is an RNA molecule comprising an exon and an intron transcribed from a target gene on the genome and is a mRNA precursor.

In a certain embodiment, the present invention provides an antisense oligomer or a pharmaceutically acceptable salt thereof, or hydrate thereof which causes simultaneous skipping of any two or more numerically consecutive exons selected from the group consisting of the 45th exon to the 55th exon in human dystrophin pre-mRNA (hereinafter, the antisense oligomer and the pharmaceutically acceptable salt thereof and the hydrate of the antisense oligomer or the salt are also collectively referred to as the “antisense oligomer of the present invention”; the antisense oligomer of the present invention may refer to any of the antisense oligomer or the pharmaceutically acceptable salt thereof, or hydrate thereof).

The human dystrophin pre-mRNA is an RNA molecule comprising an exon and an intron transcribed from the human dystrophin gene on the genome and is a mRNA precursor. Those skilled in the art can obtain information on the base sequence of the human dystrophin pre-mRNA by analogy from the genomic sequence of the human dystrophin gene (GenBank Accession Nos. NG_012232.1).

In the human genome, the human dystrophin gene locates at locus Xp21.2. The human dystrophin gene has a size of about 3.0 Mbp and is the largest gene among known human genes. However, the coding regions of the human dystrophin gene are only about 14 kb, distributed as 79 exons throughout the human dystrophin gene (Roberts, R G, et al., Genomics, 16: 536-538 (1993)). The pre-mRNA, which is the transcript of the human dystrophin gene, undergoes splicing to form mature mRNA of about 14 kb. The base sequence of mature mRNA of human wild-type dystrophin gene is known (GenBank Accession No. NM_004006).

The antisense oligomer of the present invention according to the foregoing embodiment targets at least any one region selected from the group consisting of regions R1 to R24 represented by

region Rn (wherein n is an odd number of 1 to 23) which consists of a base sequence of NX bases in the upstream direction from the 3′ end of the NAth exon and a base sequence of NY bases in the downstream direction from the 5′ end of the NBth intron in the human dystrophin pre-mRNA, and

region Rn (wherein n is an even number of 2 to 24) which consists of a base sequence of NX bases in the upstream direction from the 3′ end of the NAth intron and a base sequence of NY bases in the downstream direction from the 5′ end of the NBth exon in the human dystrophin pre-mRNA.

Hereinafter, the regions R1 to R24 are each or collectively referred to as a “target region of the antisense oligomer of the present invention” or interchangeably the “target region of the present invention”.

As used herein, the term “targeting” means that an intended base sequence is a base sequence complementary to the base sequence of a target region or a partial base sequence of the target sequence.

The antisense oligomer of the present invention according to the foregoing embodiment comprises a base sequence complementary to a base sequence of at least any one region selected from the group consisting of the target regions R1 to R24 of the present invention, or a partial base sequence thereof.

Examples of the target region Rn of the present invention according to the foregoing embodiment include regions wherein

when n is 1, NA=44, NB=44, NX=20, and NY=400,

when n is 2, NA=44, NB=45, NX=600, and NY=50,

when n is 3, NA=45, NB=45, NX=20, and NY=400,

when n is 4, NA=45, NB=46, NX=400, and NY=50,

when n is 5, NA=46, NB=46, NX=20, and NY=400,

when n is 6, NA=46, NB=47, NX=400, and NY=50,

when n is 7, NA=47, NB=47, NX=20, and NY=400,

when n is 8, NA=47, NB=48, NX=400, and NY=50,

when n is 9, NA=48, NB=48, NX=20, and NY=400,

when n is 10, NA=48, NB=49, NX=400, and NY=50,

when n is 11, NA=49, NB=49, NX=20, and NY=400,

when n is 12, NA=49, NB=50, NX=400, and NY=50,

when n is 13, NA=50, NB=50, NX=20, and NY=400,

when n is 14, NA=50, NB=51, NX=400, and NY=50,

when n is 15, NA=51, NB=51, NX=20, and NY=400,

when n is 16, NA=51, NB=52, NX=400, and NY=50,

when n is 17, NA=52, NB=52, NX=20, and NY=400,

when n is 18, NA=52, NB=53, NX=400, and NY=50,

when n is 19, NA=53, NB=53, NX=20, and NY=400,

when n is 20, NA=53, NB=54, NX=400, and NY=50,

when n is 21, NA=54, NB=54, NX=20, and NY=400,

when n is 22, NA=54, NB=55, NX=400, and NY=50,

when n is 23, NA=55, NB=55, NX=20, and NY=400, or

when n is 24, NA=55, NB=56, NX=400, and NY=50.

In another embodiment, examples of the target region Rn of the present invention include, but not limited to, regions wherein

when n is 1, NA=44, NB=44, NX=20, and NY=400,

when n is 2, NA=44, NB=45, NX=600, and NY=19,

when n is 3, NA=45, NB=45, NX=20, and NY=400,

when n is 4, NA=45, NB=46, NX=400, and NY=50,

when n is 5, NA=46, NB=46, NX=20, and NY=400,

when n is 6, NA=46, NB=47, NX=400, and NY=37,

when n is 7, NA=47, NB=47, NX=20, and NY=400,

when n is 8, NA=47, NB=48, NX=400, and NY=19,

when n is 9, NA=48, NB=48, NX=20, and NY=400,

when n is 10, NA=48, NB=49, NX=400, and NY=42,

when n is 11, NA=49, NB=49, NX=20, and NY=400,

when n is 12, NA=49, NB=50, NX=400, and NY=44,

when n is 13, NA=50, NB=50, NX=20, and NY=400,

when n is 14, NA=50, NB=51, NX=400, and NY=25,

when n is 15, NA=51, NB=51, NX=20, and NY=400,

when n is 16, NA=51, NB=52, NX=400, and NY=24,

when n is 17, NA=52, NB=52, NX=20, and NY=400,

when n is 18, NA=52, NB=53, NX=400, and NY=34,

when n is 19, NA=53, NB=53, NX=20, and NY=400,

when n is 20, NA=53, NB=54, NX=400, and NY=43,

when n is 21, NA=54, NB=54, NX=20, and NY=400,

when n is 22, NA=54, NB=55, NX=400, and NY=25,

when n is 23, NA=55, NB=55, NX=20, and NY=400, or

when n is 24, NA=55, NB=56, NX=400, and NY=50.

In another embodiment, R1 to R24 which are examples of the target region Rn of the present invention are, but not limited to, as follows:

for example, in a certain embodiment,

the region R1 is a region that consists of a base sequence of 20 bases in the upstream direction from the 3′ end of the 44th exon and a base sequence of 400 bases in the downstream direction from the 5′ end of the 44th intron in the human dystrophin pre-mRNA,

the region R2 is a region that consists of a base sequence of 600 bases in the upstream direction from the 3′ end of the 44th intron and a base sequence of 50 bases in the downstream direction from the 5′ end of the 45th exon in the human dystrophin pre-mRNA,

the region R3 is a region that consists of a base sequence of 20 bases in the upstream direction from the 3′ end of the 45th exon and a base sequence of 400 bases in the downstream direction from the 5′ end of the 45th intron in the human dystrophin pre-mRNA,

the region R4 is a region that consists of a base sequence of 400 bases in the upstream direction from the 3′ end of the 45th intron and a base sequence of 50 bases in the downstream direction from the 5′ end of the 46th exon in the human dystrophin pre-mRNA,

the region R5 is a region that consists of a base sequence of 20 bases in the upstream direction from the 3′ end of the 46th exon and a base sequence of 400 bases in the downstream direction from the 5′ end of the 46th intron in the human dystrophin pre-mRNA,

the region R6 is a region that consists of a base sequence of 400 bases in the upstream direction from the 3′ end of the 46th intron and a base sequence of 50 bases in the downstream direction from the 5′ end of the 47th exon in the human dystrophin pre-mRNA,

the region R7 is a region that consists of a base sequence of 20 bases in the upstream direction from the 3′ end of the 47th exon and a base sequence of 400 bases in the downstream direction from the 5′ end of the 47th intron in the human dystrophin pre-mRNA,

the region R8 is a region that consists of a base sequence of 400 bases in the upstream direction from the 3′ end of the 47th intron and a base sequence of 50 bases in the downstream direction from the 5′ end of the 48th exon in the human dystrophin pre-mRNA,

the region R9 is a region that consists of a base sequence of 20 bases in the upstream direction from the 3′ end of the 48th exon and a base sequence of 400 bases in the downstream direction from the 5′ end of the 48th intron in the human dystrophin pre-mRNA,

the region R10 is a region that consists of a base sequence of 400 bases in the upstream direction from the 3′ end of the 48th intron and a base sequence of 50 bases in the downstream direction from the 5′ end of the 49th exon in the human dystrophin pre-mRNA,

the region R11 is a region that consists of a base sequence of 20 bases in the upstream direction from the 3′ end of the 49th exon and a base sequence of 400 bases in the downstream direction from the 5′ end of the 49th intron in the human dystrophin pre-mRNA,

the region R12 is a region that consists of a base sequence of 400 bases in the upstream direction from the 3′ end of the 49th intron and a base sequence of 50 bases in the downstream direction from the 5′ end of the 50th exon in the human dystrophin pre-mRNA,

the region R13 is a region that consists of a base sequence of 20 bases in the upstream direction from the 3′ end of the 50th exon and a base sequence of 400 bases in the downstream direction from the 5′ end of the 50th intron in the human dystrophin pre-mRNA,

the region R14 is a region that consists of a base sequence of 400 bases in the upstream direction from the 3′ end of the 50th intron and a base sequence of 50 bases in the downstream direction from the 5′ end of the 51st exon in the human dystrophin pre-mRNA,

the region R15 is a region that consists of a base sequence of 20 bases in the upstream direction from the 3′ end of the 51st exon and a base sequence of 400 bases in the downstream direction from the 5′ end of the 51st intron in the human dystrophin pre-mRNA,

the region R16 is a region that consists of a base sequence of 400 bases in the upstream direction from the 3′ end of the 51st intron and a base sequence of 50 bases in the downstream direction from the 5′ end of the 52nd exon in the human dystrophin pre-mRNA,

the region R17 is a region that consists of a base sequence of 20 bases in the upstream direction from the 3′ end of the 52nd exon and a base sequence of 400 bases in the downstream direction from the 5′ end of the 52nd intron in the human dystrophin pre-mRNA,

the region R18 is a region that consists of a base sequence of 400 bases in the upstream direction from the 3′ end of the 52nd intron and a base sequence of 50 bases in the downstream direction from the 5′ end of the 53rd exon in the human dystrophin pre-mRNA,

the region R19 is a region that consists of a base sequence of 20 bases in the upstream direction from the 3′ end of the 53rd exon and a base sequence of 400 bases in the downstream direction from the 5′ end of the 53rd intron in the human dystrophin pre-mRNA,

the region R20 is a region that consists of a base sequence of 400 bases in the upstream direction from the 3′ end of the 53rd intron and a base sequence of 50 bases in the downstream direction from the 5′ end of the 54th exon in the human dystrophin pre-mRNA,

the region R21 is a region that consists of a base sequence of 20 bases in the upstream direction from the 3′ end of the 54th exon and a base sequence of 400 bases in the downstream direction from the 5′ end of the 54th intron in the human dystrophin pre-mRNA,

the region R22 is a region that consists of a base sequence of 400 bases in the upstream direction from the 3′ end of the 54th intron and a base sequence of 50 bases in the downstream direction from the 5′ end of the 55th exon in the human dystrophin pre-mRNA,

the region R23 is a region that consists of a base sequence of 20 bases in the upstream direction from the 3′ end of the 55th exon and a base sequence of 400 bases in the downstream direction from the 5′ end of the 55th intron in the human dystrophin pre-mRNA, or

the region R24 is a region that consists of a base sequence of 400 bases in the upstream direction from the 3′ end of the 55th intron and a base sequence of 50 bases in the downstream direction from the 5′ end of the 56th exon in the human dystrophin pre-mRNA.

In an alternative embodiment, R1 to R24 which are examples of the target region Rn of the present invention are, but not limited to, as follows:

for example, in a certain embodiment,

the region R1 is a region that consists of a base sequence of 20 bases in the upstream direction from the 3′ end of the 44th exon and a base sequence of 400 bases in the downstream direction from the 5′ end of the 44th intron in the human dystrophin pre-mRNA,

the region R2 is a region that consists of a base sequence of 600 bases in the upstream direction from the 3′ end of the 44th intron and a base sequence of 19 bases in the downstream direction from the 5′ end of the 45th exon in the human dystrophin pre-mRNA,

the region R3 is a region that consists of a base sequence of 20 bases in the upstream direction from the 3′ end of the 45th exon and a base sequence of 400 bases in the downstream direction from the 5′ end of the 45th intron in the human dystrophin pre-mRNA,

the region R4 is a region that consists of a base sequence of 400 bases in the upstream direction from the 3′ end of the 45th intron and a base sequence of 50 bases in the downstream direction from the 5′ end of the 46th exon in the human dystrophin pre-mRNA,

the region R5 is a region that consists of a base sequence of 20 bases in the upstream direction from the 3′ end of the 46th exon and a base sequence of 400 bases in the downstream direction from the 5′ end of the 46th intron in the human dystrophin pre-mRNA,

the region R6 is a region that consists of a base sequence of 400 bases in the upstream direction from the 3′ end of the 46th intron and a base sequence of 37 bases in the downstream direction from the 5′ end of the 47th exon in the human dystrophin pre-mRNA,

the region R7 is a region that consists of a base sequence of 20 bases in the upstream direction from the 3′ end of the 47th exon and a base sequence of 400 bases in the downstream direction from the 5′ end of the 47th intron in the human dystrophin pre-mRNA,

the region R8 is a region that consists of a base sequence of 400 bases in the upstream direction from the 3′ end of the 47th intron and a base sequence of 19 bases in the downstream direction from the 5′ end of the 48th exon in the human dystrophin pre-mRNA,

the region R9 is a region that consists of a base sequence of 20 bases in the upstream direction from the 3′ end of the 48th exon and a base sequence of 400 bases in the downstream direction from the 5′ end of the 48th intron in the human dystrophin pre-mRNA,

the region R10 is a region that consists of a base sequence of 400 bases in the upstream direction from the 3′ end of the 48th intron and a base sequence of 42 bases in the downstream direction from the 5′ end of the 49th exon in the human dystrophin pre-mRNA,

the region R11 is a region that consists of a base sequence of 20 bases in the upstream direction from the 3′ end of the 49th exon and a base sequence of 400 bases in the downstream direction from the 5′ end of the 49th intron in the human dystrophin pre-mRNA,

the region R12 is a region that consists of a base sequence of 400 bases in the upstream direction from the 3′ end of the 49th intron and a base sequence of 44 bases in the downstream direction from the 5′ end of the 50th exon in the human dystrophin pre-mRNA,

the region R13 is a region that consists of a base sequence of 20 bases in the upstream direction from the 3′ end of the 50th exon and a base sequence of 400 bases in the downstream direction from the 5′ end of the 50th intron in the human dystrophin pre-mRNA,

the region R14 is a region that consists of a base sequence of 400 bases in the upstream direction from the 3′ end of the 50th intron and a base sequence of 25 bases in the downstream direction from the 5′ end of the 51st exon in the human dystrophin pre-mRNA,

the region R15 is a region that consists of a base sequence of 20 bases in the upstream direction from the 3′ end of the 51st exon and a base sequence of 400 bases in the downstream direction from the 5′ end of the 51st intron in the human dystrophin pre-mRNA,

the region R16 is a region that consists of a base sequence of 400 bases in the upstream direction from the 3′ end of the 51st intron and a base sequence of 24 bases in the downstream direction from the 5′ end of the 52nd exon in the human dystrophin pre-mRNA,

the region R17 is a region that consists of a base sequence of 20 bases in the upstream direction from the 3′ end of the 52nd exon and a base sequence of 400 bases in the downstream direction from the 5′ end of the 52nd intron in the human dystrophin pre-mRNA,

the region R18 is a region that consists of a base sequence of 400 bases in the upstream direction from the 3′ end of the 52nd intron and a base sequence of 34 bases in the downstream direction from the 5′ end of the 53rd exon in the human dystrophin pre-mRNA,

the region R19 is a region that consists of a base sequence of 20 bases in the upstream direction from the 3′ end of the 53rd exon and a base sequence of 400 bases in the downstream direction from the 5′ end of the 53rd intron in the human dystrophin pre-mRNA,

the region R20 is a region that consists of a base sequence of 400 bases in the upstream direction from the 3′ end of the 53rd intron and a base sequence of 43 bases in the downstream direction from the 5′ end of the 54th exon in the human dystrophin pre-mRNA,

the region R21 is a region that consists of a base sequence of 20 bases in the upstream direction from the 3′ end of the 54th exon and a base sequence of 400 bases in the downstream direction from the 5′ end of the 54th intron in the human dystrophin pre-mRNA,

the region R22 is a region that consists of a base sequence of 400 bases in the upstream direction from the 3′ end of the 54th intron and a base sequence of 25 bases in the downstream direction from the 5′ end of the 55th exon in the human dystrophin pre-mRNA,

the region R23 is a region that consists of a base sequence of 20 bases in the upstream direction from the 3′ end of the 55th exon and a base sequence of 400 bases in the downstream direction from the 5′ end of the 55th intron in the human dystrophin pre-mRNA, or

the region R24 is a region that consists of a base sequence of 400 bases in the upstream direction from the 3′ end of the 55th intron and a base sequence of 50 bases in the downstream direction from the 5′ end of the 56th exon in the human dystrophin pre-mRNA.

In an alternative embodiment, R1 to R24 which are examples of the target region Rn of the present invention are, but not limited to, as follows:

### (A1) Region R1

The region R1 is a region indicated by the range of −20 bases to +400 bases when the boundary between the 3′ end of exon 44 and the 5′ end of intron 44 is defined as basing point 0, a base sequence region on the 5′ side (upstream) from the basing point is indicated by “+”, and a base sequence region on the 3′ side (downstream) therefrom is indicated by “−” (minus). In this respect, the region indicated by the range of −20 bases to −1 base belongs to exon 44, and the region indicated by the range of +1 base to +400 bases belongs to intron 44.

### (A2) Region R2

The region R2 is a region indicated by the range of −600 bases to +50 bases when the boundary between the 3′ end of intron 44 and the 5′ end of exon 45 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −600 bases to −1 base belongs to intron 44, and the region indicated by the range of +1 base to +50 bases belongs to exon 45.

### (A3) Region R3

The region R3 is a region indicated by the range of −20 bases to +400 bases when the boundary between the 3′ end of exon 45 and the 5′ end of intron 45 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −20 bases to −1 base belongs to exon 45, and the region indicated by the range of +1 base to +400 bases belongs to intron 45.

### (A4) Region R4

The region R4 is a region indicated by the range of −400 bases to +50 bases when the boundary between the 3′ end of intron 45 and the 5′ end of exon 46 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 45, and the region indicated by the range of +1 base to +50 bases belongs to exon 46.

### (A5) Region R5

The region R5 is a region indicated by the range of −20 bases to +400 bases when the boundary between the 3′ end of exon 46 and the 5′ end of intron 46 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −20 bases to −1 base belongs to exon 46, and the region indicated by the range of +1 base to +400 bases belongs to intron 46.

### (A6) Region R6

The region R6 is a region indicated by the range of −400 bases to +50 bases when the boundary between the 3′ end of intron 46 and the 5′ end of exon 47 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 46, and the region indicated by the range of +1 base to +50 bases belongs to exon 47.

### (A7) Region R7

The region R7 is a region indicated by the range of −20 bases to +400 bases when the boundary between the 3′ end of exon 47 and the 5′ end of intron 47 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −20 bases to −1 base belongs to exon 47, and the region indicated by the range of +1 base to +400 bases belongs to intron 47.

### (A8) Region R8

The region R8 is a region indicated by the range of −400 bases to +50 bases when the boundary between the 3′ end of intron 47 and the 5′ end of exon 48 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 47, and the region indicated by the range of +1 base to +50 bases belongs to exon 48.

### (A9) Region R9

The region R9 is a region indicated by the range of −20 bases to +400 bases when the boundary between the 3′ end of exon 48 and the 5′ end of intron 48 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −20 bases to −1 base belongs to exon 48, and the region indicated by the range of +1 base to +400 bases belongs to intron 48.

### (A10) Region R10

The region R10 is a region indicated by the range of −400 bases to +50 bases when the boundary between the 3′ end of intron 48 and the 5′ end of exon 49 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 48, and the region indicated by the range of +1 base to +50 bases belongs to exon 49.

### (A11) Region R11

The region R11 is a region indicated by the range of −20 bases to +400 bases when the boundary between the 3′ end of exon 49 and the 5′ end of intron 49 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −20 bases to −1 base belongs to exon 49, and the region indicated by the range of +1 base to +400 bases belongs to intron 49.

### (A12) Region R12

The region R12 is a region indicated by the range of −400 bases to +50 bases when the boundary between the 3′ end of intron 49 and the 5′ end of exon 50 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 49, and the region indicated by the range of +1 base to +50 bases belongs to exon 50.

### (A13) Region R13

The region R13 is a region indicated by the range of −20 bases to +400 bases when the boundary between the 3′ end of exon 50 and the 5′ end of intron 50 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −20 bases to −1 base belongs to exon 50, and the region indicated by the range of +1 base to +400 bases belongs to intron 50.

### (A14) Region R14

The region R14 is a region indicated by the range of −400 bases to +50 bases when the boundary between the 3′ end of intron 50 and the 5′ end of exon 51 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 50, and the region indicated by the range of +1 base to +50 bases belongs to exon 51.

### (A15) Region R15

The region R15 is a region indicated by the range of −20 bases to +400 bases when the boundary between the 3′ end of exon 51 and the 5′ end of intron 51 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −20 bases to −1 base belongs to exon 51, and the region indicated by the range of +1 base to +400 bases belongs to intron 51.

### (A16) Region R16

The region R16 is a region indicated by the range of −400 bases to +50 bases when the boundary between the 3′ end of intron 51 and the 5′ end of exon 52 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 51, and the region indicated by the range of +1 base to +50 bases belongs to exon 52.

### (A17) Region R17

The region R17 is a region indicated by the range of −20 bases to +400 bases when the boundary between the 3′ end of exon 52 and the 5′ end of intron 52 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −20 bases to −1 base belongs to exon 52, and the region indicated by the range of +1 base to +400 bases belongs to intron 52.

### (A18) Region R18

The region R18 is a region indicated by the range of −400 bases to +50 bases when the boundary between the 3′ end of intron 52 and the 5′ end of exon 53 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 52, and the region indicated by the range of +1 base to +50 bases belongs to exon 53.

### (A19) Region R19

The region R19 is a region indicated by the range of −20 bases to +400 bases when the boundary between the 3′ end of exon 53 and the 5′ end of intron 53 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −20 bases to −1 base belongs to exon 53, and the region indicated by the range of +1 base to +400 bases belongs to intron 53.

### (A20) Region R20

The region R20 is a region indicated by the range of −400 bases to +50 bases when the boundary between the 3′ end of intron 53 and the 5′ end of exon 54 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 53, and the region indicated by the range of +1 base to +50 bases belongs to exon 54.

### (A21) Region R21

The region R21 is a region indicated by the range of −20 bases to +400 bases when the boundary between the 3′ end of exon 54 and the 5′ end of intron 54 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −20 bases to −1 base belongs to exon 54, and the region indicated by the range of +1 base to +400 bases belongs to intron 54.

### (A22) Region R22

The region R22 is a region indicated by the range of −400 bases to +50 bases when the boundary between the 3′ end of intron 54 and the 5′ end of exon 55 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 54, and the region indicated by the range of +1 base to +50 bases belongs to exon 55.

### (A23) Region R23

The region R23 is a region indicated by the range of −20 bases to +400 bases when the boundary between the 3′ end of exon 55 and the 5′ end of intron 55 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −20 bases to −1 base belongs to exon 55, and the region indicated by the range of +1 base to +400 bases belongs to intron 55.

### (A24) Region R24

The region R24 is a region indicated by the range of −400 bases to +50 bases when the boundary between the 3′ end of intron 55 and the 5′ end of exon 56 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 55, and the region indicated by the range of +1 base to +50 bases belongs to exon 56.

In an alternative aspect, R2, R6, R8, R10, R12, R14, R16, R18, R20, and R22 which are examples of the target region Rn of the present invention are, but not limited to, as follows:

### (A2) Region R2

The region R2 is a region indicated by the range of −600 bases to +19 bases when the boundary between the 3′ end of intron 44 and the 5′ end of exon 45 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −600 bases to −1 base belongs to intron 44, and the region indicated by the range of +1 base to +19 bases belongs to exon 45.

### (A6) Region R6

The region R6 is a region indicated by the range of −400 bases to +37 bases when the boundary between the 3′ end of intron 46 and the 5′ end of exon 47 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 46, and the region indicated by the range of +1 base to +37 bases belongs to exon 47.

### (A8) Region R8

The region R8 is a region indicated by the range of −400 bases to +19 bases when the boundary between the 3′ end of intron 47 and the 5′ end of exon 48 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 47, and the region indicated by the range of +1 base to +19 bases belongs to exon 48.

### (A10) Region R10

The region R10 is a region indicated by the range of −400 bases to +42 bases when the boundary between the 3′ end of intron 48 and the 5′ end of exon 49 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 48, and the region indicated by the range of +1 base to +42 bases belongs to exon 49.

### (A12) Region R12

The region R12 is a region indicated by the range of −400 bases to +44 bases when the boundary between the 3′ end of intron 49 and the 5′ end of exon 50 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 49, and the region indicated by the range of +1 base to +44 bases belongs to exon 50.

### (A14) Region R14

The region R14 is a region indicated by the range of −400 bases to +25 bases when the boundary between the 3′ end of intron 50 and the 5′ end of exon 51 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 50, and the region indicated by the range of +1 base to +25 bases belongs to exon 51.

### (A16) Region R16

The region R16 is a region indicated by the range of −400 bases to +24 bases when the boundary between the 3′ end of intron 51 and the 5′ end of exon 52 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 51, and the region indicated by the range of +1 base to +24 bases belongs to exon 52.

### (A18) Region R18

The region R18 is a region indicated by the range of −400 bases to +34 bases when the boundary between the 3′ end of intron 52 and the 5′ end of exon 53 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 52, and the region indicated by the range of +1 base to +34 bases belongs to exon 53.

### (A20) Region R20

The region R20 is a region indicated by the range of −400 bases to +43 bases when the boundary between the 3′ end of intron 53 and the 5′ end of exon 54 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 53, and the region indicated by the range of +1 base to +43 bases belongs to exon 54.

### (A22) Region R22

The region R22 is a region indicated by the range of −400 bases to +25 bases when the boundary between the 3′ end of intron 54 and the 5′ end of exon 55 is defined as basing point 0, a base sequence region on the 5′ side (upstream) from the basing point is indicated by “−” (minus), and a base sequence region on the 3′ side (downstream) therefrom is indicated by “+”. In this respect, the region indicated by the range of −400 bases to −1 base belongs to intron 54, and the region indicated by the range of +1 base to +25 bases belongs to exon 55.

Specific examples of the base sequences of the regions R1 to R24 will be given below. As used herein, thymine “T” and uracil “U” are interchangeable with each other. It does not essentially affect the exon skipping activity of the antisense oligomer of the present invention whether it is “T” or “U”. Therefore, as used herein, identical base sequences, even when “T” are replaced with “U”, are also included and represented by the same SEQ ID NO. In the tables below, “U” may be described as “T” even in the base sequence of pre-mRNA. Those skilled in the art can understand an RNA sequence by appropriately replacing “T” with “U”.

TABLE 1
Table 1

| Region | Base sequence of target region | SEQ ID NO |
|---|---|---|
| R1 | Region in vicinity of donor of intron 44 (in range of −20 to | SEQ No. 233 |
| | +400 bases from 5′ end of intron 44 as basing point) | |
| | AATACAAATGGTATCTTAAGGTAAGTCTTTGATTTGTTTTTTCGAAATTGTATTTATCTTC | |
| | AGCACATCTGGACTCTTTAACTTCTTAAAGATCAGGTTCTGAAGGGTGATGGAAATTACTT | |
| | TTGACTGTTGTTGTCATCATTATATTACTAGAAAGAAAATTATCATAATGATAATATTAGA | |
| | GCACGGTGCTATGGACTTTTTGTGTCAGGATGAGAGAGTTTGCCTGGACGGAGCTGGTTTA | |
| | TCTGATAAACTGCAAAATATAATTGAATCTGTGACAGAGGGAAGCATCGTAACAGCAAGGT | |
| | GTTTTGTGGCTTTGGGGCAGTGTGTATTTCGGCTTTATGTTGGAACCTTTCCAGAAGGAGA | |
| | ACTTGTGGCATACTTAGCTAAAATGAAGTTGCTAGAAATATCCATCATGATAAA | |
| R2 | Region in vicinity of acceptor of intron 44 (in range of −600 | SEQ No. 234 |
| | to +50 bases from 3′ end of intron 44 as basing point) | |
| | TCTTGATGGGATGCTCCTGAAAGCAATTAATTCTCAGTTTTTTGTGGCTTCTAATGCAAAA | |
| | TACATTGACGCAGACAGAATTTGAAATGAATTTTCTTCTAATATAGCAATTAATTTTATTT | |
| | AAATATCTCTAGAGTTTTTTTTTAATACTGTGACTAACCTATGTTTGTTCTTTTTCACCTC | |
| | TCGTATCCACGATCACTAAGAAACCCAAATACTTTGTTCATGTTTAAATTTTACAACATTT | |
| | CATAGACTATTAAACATGGAACATCCTTGTGGGGACAAGAAATCGAATTTGCTCTTGAAAA | |
| | GGTTTCCAACTAATTGATTTGTAGGACATTATAACATCCTCTAGCTGACAAGCTTACAAAA | |
| | ATAAAAACTGGAGCTAACCGAGAGGGTGCTTTTTTCCCTGACACATAAAAGGTGTCTTTCT | |
| | GTCTTGTATCCTTTGGATATGGGCATGTCAGTTTCATAGGGAAATTTTCACATGGAGCTTT | |
| | TGTATTTCTTTCTTTGCCAGTACAACTGCATGTGGTAGCACACTGTTTAATCTTTTCTCAA | |
| | ATAAAAAGACATGGGGCTTCATTTTTGTTTTGCCTTTTTGGTATCTTACAGGAACTCCAGG | |
| | ATGGCATTGGGCAGCGGCAAACTGTTGTCAGAACATTGAA | |
| R3 | Region in vicinity of donor of intron 45 (in range of −20 to | SEQ No. 235 |
| | +400 bases from 5′ end of intron 45 as basing point) | |
| | CTGTCAGACAGAAAAAAGAGGTAGGGCGACAGATCTAATAGGAATGAAAACATTTTAGCAG | |
| | ACTTTTTAAGCTTTCTTTAGAAGAATATTTCATGAGAGATTATAAGCAGGGTGAAAGGCAC | |
| | TAACATTAAAGAACCTATCAACCATTAATCAACAGCAGTAAAGAAATTTTTTATTTCTTTT | |
| | TTTCATATACTAAAATATATACTTGTGGCTAGTTAGTGGTTTTCTGCTATTTTAAACTTGA | |
| | AGTTTGCTTTAAAAATCACCCATGATTGCTTAAAGGTGAATATCTTCAATATATTTTAACT | |
| | TCAACAAGCTGAATCTCAGTTGTTTTTCAAGAAGATTTTAGAAAGCAATTATAAATGATTG | |
| | TTTTGTAGGAAAGACAGATCTTTGCTTAGTTTTAAAAATAGCTATGAATATGAC | |
| R4 | Region in vicinity of acceptor of intron 45 (in range of −400 | SEQ No. 236 |
| | to +50 bases from 3′ end of intron 45 as basing point) | |
| | ATTACAGGCGCCTGCCACCAAACCTGGCAAATTTTTGTATTTTTAGTGTAGACGGGGTTTC | |
| | ACCATATTTGCCAGGCTGGTCGCAAACTCCTGACCTCAAGTGATCCGCCCACATCGGCCTC | |
| | CCTAAGCGCTAGGGTTACAGGCATGAGCCACTGCGCCTGGCCAGGAATTTTTGAATCAGAA | |
| | TTTTTCTTGTTCGATTTTAATCTCTTATCATTTAGAGATTCTTGAAATATTGAAATTACTT | |
| | TGTTCAAAGTGAATGAATTTTCTTAAATTATGTATGGTTAACATCTTTTAAATTGCTTATT | |
| | TTTAAATTGCCATGTTTGTGTCCCAGTTTGCATTAACAAATAGTTTGAGAACTATGTTGGA | |
| | AAAAAAAATAACAATTTTATTCTTCTTTCTCCAGGCTAGAAGAACAAAAGAATATCTTGTC | |
| | AGAATTTCAAAGAGATTTAAATG | |
| R5 | Region in vicinity of donor of intron 46 (in range of −20 to | SEQ No. 237 |
| | +400 bases from 5′ end of intron 46 as basing point) | |
| | AAAAGCTTGAGCAAGTCAAGGTAATTTTATTTTCTCAAATCCCCCAGGGCCTGCTTGCATA | |
| | AAGAAGTATATGAATCTATTTTTTAATTCAATCATTGGTTTTCTGCCCATTAGGTTATTCA | |
| | TAGTTCCTTGCTAAAGTGTTTTTCTCACAACTTTATTTCTTCTTAACCCTGCAGTTCTGAA | |
| | CCAGTGCACATAAGAACATATGTATATATGTGTGTGTGTGTATTTATATATACACACACAC | |
| | ATATTGCATCTATACATCTACACATATAGATGTATAGATTCAATATGTCTAAATGTATATA | |
| | ATTCACAGTTTTTATCTTTGATTTGAAATTAATTTTAGATTTTACTTGAGAACTTCACAAC | |
| | TTCATATAATTTTAAAAACTGAAGACCAGATTGTGGAATCATAAAATCTAAATC | |
| R6 | Region in vicinity of acceptor of intron 46 (in range of −400 | SEQ No. 238 |
| | to +50 bases from 3′ end of intron 46 as basing point) | |
| | ATAATGCAATTTCTCAGCAGCAAGCTACGGTATGCTATGGCATGCTATGATACCCAAGAGG | |
| | CTGATGAATTTGTTCACATTGTTCTATTTCTGATAGAGAGATAGGTTTTCAGACACTAACT | |
| | TTATTTGGAGTGTTGCTTTACCATCTCACATTTTTCTCTTAAAAAATTTATGAGGGATAAT | |
| | ATAATCGTTTATTTTCTACAGAGATTTATCTACTGAGGGGGTGAGTGTTTCAGTCAATCAG | |
| | CTCTGTGCTCAGATAGAAAACTGTTGGTATTTGAGGTACCACTGGGCCCTCGGTCAAGTCG | |
| | CTTCATTTTGATAGACTAATCAATAGAAGCAAAGACAAGGTAGTTGGAATTGTGCTGTAAT | |
| | TCATTTTAAACGTTGTTGCATTTGTCTGTTTCAGTTACTGGTGGAAGAGTTGCCCCTGCGC | |
| | CAGGGAATTCTCAAACAATTAAA | |
| R7 | Region in vicinity of donor of intron 47 (in range of −20 to | SEQ No. 239 |
| | +400 bases from 5′ end of intron 47 as basing point) | |
| | CAAATCTCCAGTGGATAAAGGTTAGACATTAACCATCTCTTCCGTCACATGTGTTAAATGT | |
| | TGCAAGTATTTGTATGTATTTTGTTTCCTGGGTGCTTCATTGGTCGGGGAGGAGGCTGGTA | |
| | TGTGGATTGTTGTTTTGTTTTGTTTTTTTAACCTGACCGTTTGCTTTGGCTATATGTTTTG | |
| | TTGTGGCTAGAAAAAATGATGATGGTGAATGGCTTTACATTAATGACCAAATGCCAAAATT | |
| | TATACCACAATTTTTTGCATAAATTATTCTGAAGAATCAGACTGAAGAAATGGCGAAGTAT | |
| | TTAATTCAGTGGCCAGGCATGTACTGACAGTATTTAAGCTGAAAGGACGTGGTCTGGTTCT | |
| | AGTTAAACAAGTGTCATAAATCAAAATTAATTATTCACACCTGTGGTATGGACT | |
| R8 | Region in vicinity of acceptor of intron 47 (in range of −400 | |
| | to +50 bases from 3′ end of intron 47 as basing point) | SEQ No. 240 |
| | TATTCATTTTTATAACTGCAAAGGAAGCGCGTATGGCATATAATACACAACACACCAGTAT | |
| | ATTTAGTAACTGAGTGAATAAATGAAAGATGTATTTCTTTACTTTATCAGTTGCAGTTGGC | |
| | TATGCCTTTGTGTAAGGTGTGTGTTTTGAAATTCCAAAAAGGTATTAGTTTCTTTAAAGCA | |
| | AAGAATTTTTGTAGCAGGTTAATGAATAATTTTGAATACATTGGTTAAATCCCAACATGTA | |
| | ATATATGTAAATAATCAATATTATGCTGCTAAAATAACACAAATCAGTAAGATTCTGTAAT | |
| | ATTTCATGATAAATAACTTTTGAAAATATATTTTTAAACATTTTGGCTTATGCCTTGAGAA | |
| | TTATTTACCTTTTTAAAATGTATTTTCCTTTCAGGTTTCCAGAGCTTTACCTGAGAAACAA | |
| | GGAGAAATTGAAGCTCAAATAAA | |
| R9 | Region in vicinity of donor of intron 48 (in range of −20 to | SEQ No. 241 |
| | +400 bases from 5′ end of intron 48 as basing point) | |
| | AAGGACCATTTGACGTTCAGGTAGGGAACTTTTTGCTTTAAATATTTTTGTCTTTTTTAAG | |
| | AAAAATGGCAATATCACTGAATTTTCTCATTTGGTATCATTATTAAAGACAAAATATTACT | |
| | TGTTAAAGTGTGGTAAGGAAGACTTTATTCAGGATAACCACAATAGGCACAGGGACCACTG | |
| | CAATGGAGTATTACAGGAGGTTGGATAGAGAGAGATTGGGCTCAACTCTAAATACAGCACA | |
| | GTGGAAGTAGGAATTTATAGCCAAGGAGCAGTGTAGGAGTCAGTAGATGGAAAATTATTAA | |
| | GAGGAAACATCAGGGGTAAGTGGGATTCTGGCTAAACCAACCTCACAGGATTCTTGCTGAA | |
| | GATAGGCCAGGGTTATCTTATCAGACAACCCTTGGGGAATGGTGGAGAATACTG | |
| R10 | Region in vicinity of acceptor of intron 48 (in range of −400 | SEQ No. 242 |
| | to +50 bases from 3′ end of intron 48 as basing point) | |
| | TACTAAACACAGAATTTTGTAAAACAATAAGTGTATAAAGTAAAATGAACATTAGGATTAT | |
| | TGAGATTATTGTAGCTAAAACTAGTGTTTATTCATATAAATTATGTTAATAAATTGTATTG | |
| | TCATTATTGCATTTTACTTTTTTGAAAAGTAGTTAATGCCTGTGTTTCTATATGAGTATTA | |
| | TATAATTCAAGAAGATATTGGATGAATTTTTTTTAAGTTTAATGTGTTTCACATCTCTGTT | |
| | TCTTTTCTCTGCACCAAAAGCTACATTTTTGTGCCCTTATGTACCAGGCAGAAATTGATCT | |
| | GCAATACATGTGGAGTCTCCAAGGGTATATTTAAATTTAGTAATTTTATTGCTAACTGTGA | |
| | AGTTAATCTGCACTATATGGGTTCTTTTCCCCAGGAAACTGAAATAGCAGTTCAAGCTAAA | |
| | CAACCGGATGTGGAAGAGATTTT | |
| R11 | Region in vicinity of donor of intron 49 (in range of −20 to | SEQ No. 243 |
| | +400 bases from 5′ end of intron 49 as basing point) | |
| | CAGCCACTCAGCCAGTGAAGGTAATGAAGCAACCTCTAGCAATATCCATTACCTCATAATG | |
| | GGTTATGCTTCCCCTGTTGTACATTTGCCATTGACGTGGACTATTTATAATCAGTGAAATA | |
| | ACTTGTAAGGAAATACTGGCCATACTGTAATAGCAGAGGCAAAGCTGTCTTTTTGATCAGC | |
| | ATATCCTATTTATATATTGTGATCTTAAGGCTATTAACGAGTCATTGCTTTAAAGGACTCA | |
| | TTTGTGTCCTGGTGTGCTGCCATCAATACAAAAGTAGTCCCACCTTCAAGGTAGATTAAAT | |
| | TCTTTGGGGCTTTATTGCTTTGCTTGCCAGCCTTGATGCTTTTCATATTGTTTGGTTTAAT | |
| | TCAAATCAAGCTACTGCATCATAGTGTCTGTCTCCAACAGCTGTAAAGAATCAC | |
| R12 | Region in vicinity of acceptor of intron 49 (in range of −400 | SEQ No. 244 |
| | to +50 bases from 3′ end of intron 49 as basing point) | |
| | ATATAATTGACTGGGGGTGAGCCAGTACATTAGGATTTTCCTAAAGTTATCTGGATAATTT | |
| | TAGTATGCAACCACAATAGATACTCTTCAAGAATTAAGCTAGTTGCTGAGAGGGAACTGTT | |
| | TTTTGTTGGTTTGTTTTCACTAATGTTTGCACTCTACTTCCTTTAAATAAAATTATGCCTG | |
| | GAGAAAGGGTTTTTGTATGGAGCAATTGATAAATATTTGTAGGGTGGTTGGCTAAAATAAT | |
| | TATAATTCCTTTAAAAGAAATTCTACCCACTAAAGTTAATTTAGAAGTAAAATATAATAGA | |
| | AATCCAATAATATATTCACCAAATGGATTAAGATGTTCATGAATTATCTTCAAAGTGTTAA | |
| | TCGAATAAGTAATGTGTATGCTTTTCTGTTAAAGAGGAAGTTAGAAGATCTGAGCTCTGAG | |
| | TGGAAGGCGGTAAACCGTTTACT | |
| R13 | Region in vicinity of donor of intron 50 (in range of −20 to | SEQ No. 245 |
| | +400 bases from 5′ end of intron 50 as basing point) | |
| | ACTGACCACTATTGGAGCCTGTAAGTATACTGGATCCCATTCTCTTTGGCTCTAGCTATTT | |
| | GTTCAAAAGTGCAACTATGAAGTGATGACTGGGTGAGAGAGAAAATTTGTTTCAATTCTAA | |
| | AGATAGAGATAAACCTTTGTGTTATTGACTGTGCAAAAAGTCTTAGAGTACATTCCTTGGA | |
| | AATTGACTCTGATTCAAAGTGTTGCATGACAACGGGATATGGGGAGTGTTCTCTGGAGATA | |
| | CACCCACAAGGAAGAGAAGAGCACAAGGGAGATTGTGGGAGAGTCTGAAATGTGATTTGTC | |
| | TGCAGCAGAGGCCTAAGCCAGTCTCGCAGGAGCCCTACATCTGGGCTGGCTGTGCAGAGCT | |
| | GTCCTGAATTGCAGGCAGTGGGCCTGGCCCTTGTATTCCTGATCCAGCCAGCCA | |
| R14 | Region in vicinity of acceptor of intron 50 (in range of −400 | SEQ No. 246 |
| | to +50 bases from 3′ end of intron 50 as basing point) | |
| | TCTTGAATAAAAAAAAAATAAGTAAAATTTATTTCCCTGGCAAGGTCTGAAAACTTTTGTT | |
| | TTCTTTACCACTTCCACAATGTATATGATTGTTACTGAGAAGGCTTATTTAACTTAAGTTA | |
| | CTTGTCCAGGCATGAGAATGAGCAAAATCGTTTTTTAAAAAATTGTTAAATGTATATTAAT | |
| | GAAAAGGTTGAATCTTTTCATTTTCTACCATGTATTGCTAAACAAAGTATCCACATTGTTA | |
| | GAAAAAGATATATAATGTCATGAATAAGAGTTTGGCTCAAATTGTTACTCTTCAATTAAAT | |
| | TTGACTTATTGTTATTGAAATTGGCTCTTTAGCTTGTGTTTCTAATTTTTCTTTTTCTTCT | |
| | TTTTTCCTTTTTGCAAAAACCCAAAATATTTTAGCTCCTACTCAGACTGTTACTCTGGTGA | |
| | CACAACCTGTGGTTACTAAGGAA | |
| R15 | Region in vicinity of donor of intron 51 (in range of −20 to | SEQ No. 247 |
| | +400 bases from 5′ end of intron 51 as basing point) | |
| | AGATGATCATCAAGCAGAAGGTATGAGAAAAAATGATAAAAGTTGGCAGAAGTTTTTCTTT | |
| | AAAATGAAGATTTTCCACCAATCACTTTACTCTCCTAGACCATTTCCCACCAGTTCTTAGG | |
| | CAACTGTTTCTCTCTCAGCAAACACATTACTCTCACTATTCAGCCTAAGTATAATCAAGGA | |
| | TATAAATTAATGCAAATAACAAAAGTAGCCATACATTAAAAAGGAAATATACAAAAAAAAA | |
| | AAAAAAAAAAAGCAGAAACCTTACAAGAATAGTTGTCTCAGTTAAATTTACTAAACAACCT | |
| | GGTATTTTAAAAATCTATTTTATACCAAATAAGTCACTCAACTGAGCTATTTACATTTAAA | |
| | CTGTTTGTTTTGGCACTACGCAGCCCAACATATTGCAGAATCAAATATAATAGT | |
| R16 | Region in vicinity of acceptor of intron 51 (in range of −400 | SEQ No. 248 |
| | to +50 bases from 3′ end of intron 51 as basing point) | |
| | TTTAAAATAAATATACCTTAATTTTGACGTCACACAGAATGATATTATAAGTATAAATAGT | |
| | TATCTATCTTTTAAATACATTGTCGTAATTCAGAATAACATTTCTTACTCAAGGCATTCAG | |
| | ACAGTGGTTTAAGTAATCCGAGGTACTCCGGAATGTCTCCATTTGAGCCTTTAAATGAAGA | |
| | AAATCTATAGTCAAGATTTTCATTTGAAATATTTTTGATATCTAAGAATGAAACATATTTC | |
| | CTGTTAAATTGTTTTCTATAAACCCTTATACAGTAACATCTTTTTTATTTCTAAAAGTGTT | |
| | TTGGCTGGTCTCACAATTGTACTTTACTTTGTATTATGTAAAAGGAATACACAACGCTGAA | |
| | GAACCCTGATACTAAGGGATATTTGTTCTTACAGGCAACAATGCAGGATTTGGAACAGAGG | |
| | CGTCCCCAGTTGGAAGAACTCAT | |
| R17 | Region in vicinity of donor of intron 52 (in range of −20 to | SEQ No. 249 |
| | +400 bases from 5′ end of intron 52 as basing point) | |
| | AACAATCATTACGGATCGAAGTAAGTTTTTTAACAAGCATGGGACACACAAAGCAAGATGC | |
| | ATGACAAGTTTCAATAAAAACTTAAGTTCATATATCCCCCTCACATTTATAAAAATAATGT | |
| | GAAATAATTGTAAATGATAACAATTGTGCTGAGATTTTCAGTCCATAATGTTACCTTTTAA | |
| | TAAATGAATGTAATTCCATTGAATAGAAGAAATACATTTTTAAATCAATTCAGGGCTTATA | |
| | TAGTTGCAAAGCATGCATTGATGGGTGTGGTGACCACAGTGTGGCAGAACATTTGTGGCAG | |
| | AACATTTGTTCTTTAGTTGTCATCTGGGCTGGCATCCATGGAGATGCCAGTCTCTCCCTCA | |
| | TATCCTTGGCTGTTGGTCCAAGCAGGCAGTGGCTTCTTCCTGGGCCATCTTTCA | |
| R18 | Region in vicinity of acceptor of intron 52 (in range of −400 | SEQ No. 250 |
| | to +50 bases from 3′ end of intron 52 as basing point) | |
| | CAAACTCCTGTGGCAACAGAAAGCCTTCAGGCAATGAAATGCTGGCACTGGGAAATCAGGC | |
| | TGATGGGTGCTGAAGTGGCAAGGATGAGGGGATATGGATATTCTGCTGTAGTGCTTTTCTA | |
| | ACAGATGATTCATATTTGGTTCTAGGGATCAAGAATTGAGTTAAAATTTTATATATATGTT | |
| | GATGTTCTATGTCACCTTCAGGAAAATAATTTAACAGAAACTAATATTTGCCATCAAAAAA | |
| | GCAAAGAATCCTGTTGTTCATCATCCTAGCCATAACACAATGAATAATTTTTTAAATAAGC | |
| | AACATAAATGTGAGATAACGTTTGGAAGTTACATTTAAAATGTCTCCTCCAGACTAGCATT | |
| | TACTACTATATATTTATTTTTCCTTTTATTCTAGTTGAAAGAATTCAGAATCAGTGGGATG | |
| | AAGTACAAGAACACCTTCAGAAC | |
| R19 | Region in vicinity of donor of intron 53 (in range of −20 to | SEQ No. 251 |
| | +400 bases from 5′ end of intron 53 as basing point) | |
| | AGAAAATCACAGAAACCAAGGTTAGTATCAAAGATACCTTTTTAAAATAAAATACTGGTTA | |
| | CATTTGATAAAATTATACCATAGATTGTAATTTAATGATGTTTAATGTAAAGTTATTAACA | |
| | GAAAATCACGTTAAAGCTGAAATGAACAGTAGACTTTGTATATTTATTTTCTTAGAGACAG | |
| | AGTCTCACTGTCACCCAGGCTAAAGTGCAGTGGCACAATCATAGCTCACTGAGCCTTGAAC | |
| | TCTGGGGCTCAAGCAGTCCTCCTGCCTCAGCCTCCCTAGTAGCTGGGACTACTAGCCAGGC | |
| | GTGTACCACCACGCCTGGCTAATTTTTTAAAAATTTTTGTTTTCTGTAGAGATGGGTTCTT | |
| | GAACTCTTGGCCTCAAGCAATTCTCCTTCCTTGGCCTCCCAAAGCACTAGGATT | |
| R20 | Region in vicinity of acceptor of intron 53 (in range of −400 | SEQ No. 252 |
| | to +50 bases from 3′ end of intron 53 as basing point) | |
| | CAACAAGTTAATGAAGAGGGAAAGAAATGTATGAGGTTTTTTTCGTTCAAATGTTGTTATA | |
| | TGTCACATATTCAACAATTATATATGAGCTTATTTTTGTAGTTTTTTTCTCTTGTGATAAA | |
| | AACAATTAAGCCCACTTTATTGCCAATTAATTGCTACTAAGTTGAAATACTTGATACTGGT | |
| | TATTGCTCAAGATGCTGCATTTGAAAAGTTTGTCCTGAAAGGTGGGTTACCTTATACTGTC | |
| | ATGATTGACTAAATCATATGGTAGGTTAAAAGCAATCTAATATATGTATTCTGACCTGAGG | |
| | ATTCAGAAGCTGTTTACGAAGTATTTTAAGACACTCCAACTAGAGATTTCATAAAAAAAAC | |
| | TGACATTCATTCTCTTTCTCATAAAAATCTATAGCAGTTGGCCAAAGACCTCCGCCAGTGG | |
| | CAGACAAATGTAGATGTGGCAAA | |
| R21 | Region in vicinity of donor of intron 54 (in range of −20 to | SEQ No. 253 |
| | +400 bases from 5′ end of intron 54 as basing point) | |
| | TGGAGAAGCATTCATAAAAGGTATGAATTACATTATTTCTAAAACTACTGTTGGCTGTAAT | |
| | AATGGGGTGGTGAAACTGGATGGACCATGAGGATTTGTTTTTCCAATCCAGCTAAACTGGA | |
| | GCTTGGGAGGGTTCAAGACGATAAATACCAACTAAACTCACGGACTTGGCTCAGACTTCTA | |
| | TTTTAAAAACGAGGAACATAAGATCTCATTTGCCCGCTGTCACAAAAGTAGTGACATAACC | |
| | AAGAGATTAAACAAAAAGCAAAATACTGATTTATAGCTAGAAGAGCCATTTATCAGTCTAC | |
| | TTTGATAACTCTATCCAAAGGAATATCTTTCTATCTCATCATGGCGCACACTGCCTTACCT | |
| | GTTATCTGATAAATAAGTCACTTTGGGATTCATGATAGAGTTATAGCTGTACAT | |
| R22 | Region in vicinity of acceptor of intron 54 (in range of −400 | SEQ No. 254 |
| | to +50 bases from 3′ end of intron 54 as basing point) | |
| | TCTCAAATTTGGCAGTATATTAAAAATAAGCTTTCAAAATTGACCAACAAAAACTACAAAA | |
| | TTGAAAAAAAGGTACTTTGAACTTTCACATGTTCAAATATATGTATATATATTTCACATAT | |
| | ATATATGAAACCTCCTCTGTGGAGAGGGGTTTATAGAAATCTGTAATTGTCATTCTTGCAT | |
| | GCCTTCCCCCATACAAACGCCTTTAAGTTAAATAAAAATGAAAGTAAATAGACTGCACAAT | |
| | ATTATAGTTGTTGCTTAAAGGAAGAGCTGTAGCAACAACTCACCCCATTGTTGGTATATTA | |
| | CAATTTAGTTCCTCCATCTTTCTCTTTTTATGGAGTTCACTAGGTGCACCATTCTGATATT | |
| | TAATAATTGCATCTGAACATTTGGTCCTTTGCAGGGTGAGTGAGCGAGAGGCTGCTTTGGA | |
| | AGAAACTCATAGATTACTGCAAC | |
| R23 | Region in vicinity of donor of intron 55 (in range of −20 to | SEQ No. 255 |
| | +400 bases from 5′ end of intron 55 as basing point) | |
| | AGCTGATGAAACAATGGCAAGTAAGTCAGGCATTTCCGCTTTAGCACTCTTGTGGATCCAA | |
| | TTGAACAATTCTCAGCATTTGTACTTGTAACTGACAAGCCAGGGACAAAACAAAATAGTTG | |
| | CTTTTATACAGCCTGATGTATTTCGGTATTTGGACAAGGAGGAGAGAGGCAGAGGGAGAAG | |
| | GAAACATCATTTATAATTCCACTTAACACCCTCGTCTTAGAAAAAGTACATGCTCTGACCA | |
| | GGAAAACATTTGCATATAAAACCAGAGCTTCGGTCAAGGAGAAACTTTGCTCAGAGAAATA | |
| | ACTTAGGGATTGGTTTATTAAATTTTAAAAGTTGACATTTTTGAGTGTTTATTTAATATTT | |
| | TACAGGGAAAGCATCTGTATGAATTGTCTGTTTTATTTAGCGTTGCTAACTGAA | |
| R24 | Region in vicinity of acceptor of intron 55 (in range of −400 | SEQ No. 256 |
| | to +50 bases from 3′ end of intron 55 as basing point) | |
| | ACTTGATCCATATAGTAATGAAATTATTGGCACTGGGGTACACTTTATCATAGAATTTTAT | |
| | TGCCTATCACTTCCATAAAATAATACATTTTGTCCATAGACTAGAAGATATAACTTGTGAA | |
| | CTTTATAAAGTTATAAATACATTACTTTCCAACTCATAATGGCAAGGAATAAATCTATTAC | |
| | AACTAATAAGATGCCCATTTTAAATCTACATAATAACAGGAGAAGGCAATACGCCAAGAAA | |
| | AGGGATTTGAGATGTATCTTCTTGTTAGTTTAGCCTGATTGAAATGTCTTTTGAACTAATA | |
| | ATTATTTATATTTTGCAATTCTCCAAATTCACATTCATCGCTTGTTTCTTTTGTTTGGTAA | |
| | TTCTGCACATATTCTTCTTCCTGCTGTCCTGTAGGACCTCCAAGGTGAAATTGAAGCTCAC | |
| | ACAGATGTTTATCACAACCTGGA | |

In an alternative aspect, specific examples of the base sequences of the regions R2, R6, R8, R10, R12, R14, R16, R18, R20, and R22 are as follows.

TABLE 2
Table 2

| Region | Base sequence of target region | SEQ ID NO |
|---|---|---|
| R2 | Region in vicinity of acceptor of intron 44 (in range of −600 | SEQ No. 341 |
| | to +19 bases from 3′ end of intron 44 as basing point) | |
| | TCTTGATGGGATGCTCCTGAAAGCAATTAATTCTCAGTTTTTTGTGGCTTCTAATGCAAAA | |
| | TACATTGACGCAGACAGAATTTGAAATGAATTTTCTTCTAATATAGCAATTAATTTTATTT | |
| | AAATATCTCTAGAGTTTTTTTTTAATACTGTGACTAACCTATGTTTGTTCTTTTTCACCTC | |
| | TCGTATCCACGATCACTAAGAAACCCAAATACTTTGTTCATGTTTAAATTTTACAACATTT | |
| | CATAGACTATTAAACATGGAACATCCTTGTGGGGACAAGAAATCGAATTTGCTCTTGAAAA | |
| | GGTTTCCAACTAATTGATTTGTAGGACATTATAACATCCTCTAGCTGACAAGCTTACAAAA | |
| | ATAAAAACTGGAGCTAACCGAGAGGGTGCTTTTTTCCCTGACACATAAAAGGTGTCTTTCT | |
| | GTCTTGTATCCTTTGGATATGGGCATGTCAGTTTCATAGGGAAATTTTCACATGGAGCTTT | |
| | TGTATTTCTTTCTTTGCCAGTACAACTGCATGTGGTAGCACACTGTTTAATCTTTTCTCAA | |
| | ATAAAAAGACATGGGGCTTCATTTTTGTTTTGCCTTTTTGGTATCTTACAGGAACTCCAGG | |
| | ATGGCATTG | |
| R6 | Region in vicinity of acceptor of intron 46 (in range of −400 | SEQ No. 385 |
| | to +37 bases from 3′ end of intron 46 as basing point) | |
| | ATAATGCAATTTCTCAGCAGCAAGCTACGGTATGCTATGGCATGCTATGATACCCAAGAGG | |
| | CTGATGAATTTGTTCACATTGTTCTATTTCTGATAGAGAGATAGGTTTTCAGACACTAACT | |
| | TTATTTGGAGTGTTGCTTTACCATCTCACATTTTTCTCTTAAAAAATTTATGAGGGATAAT | |
| | ATAATCGTTTATTTTCTACAGAGATTTATCTACTGAGGGGGTGAGTGTTTCAGTCAATCAG | |
| | CTCTGTGCTCAGATAGAAAACTGTTGGTATTTGAGGTACCACTGGGCCCTCGGTCAAGTCG | |
| | CTTCATTTTGATAGACTAATCAATAGAAGCAAAGACAAGGTAGTTGGAATTGTGCTGTAAT | |
| | TCATTTTAAACGTTGTTGCATTTGTCTGTTTCAGTTACTGGTGGAAGAGTTGCCCCTGCGC | |
| | CAGGGAATTC | |
| R8 | Region in vicinity of acceptor of intron 47 (in range of −400 | SEQ No. 386 |
| | to +19 bases from 3′ end of intron 47 as basing point) | |
| | TATTCATTTTTATAACTGCAAAGGAAGCGCGTATGGCATATAATACACAACACACCAGTAT | |
| | ATTTAGTAACTGAGTGAATAAATGAAAGATGTATTTCTTTACTTTATCAGTTGCAGTTGGC | |
| | TATGCCTTTGTGTAAGGTGTGTGTTTTGAAATTCCAAAAAGGTATTAGTTTCTTTAAAGCA | |
| | AAGAATTTTTGTAGCAGGTTAATGAATAATTTTGAATACATTGGTTAAATCCCAACATGTA | |
| | ATATATGTAAATAATCAATATTATGCTGCTAAAATAACACAAATCAGTAAGATTCTGTAAT | |
| | ATTTCATGATAAATAACTTTTGAAAATATATTTTTAAACATTTTGGCTTATGCCTTGAGAA | |
| | TTATTTACCTTTTTAAAATGTATTTTCCTTTCAGGTTTCCAGAGCTTTACCTG | |
| R10 | Region in vicinity of acceptor of intron 48 (in range of −400 | SEQ No. 387 |
| | to +42 bases from 3′ end of intron 48 as basing point) | |
| | TACTAAACACAGAATTTTGTAAAACAATAAGTGTATAAAGTAAAATGAACATTAGGATTAT | |
| | TGAGATTATTGTAGCTAAAACTAGTGTTTATTCATATAAATTATGTTAATAAATTGTATTG | |
| | TCATTATTGCATTTTACTTTTTTGAAAAGTAGTTAATGCCTGTGTTTCTATATGAGTATTA | |
| | TATAATTCAAGAAGATATTGGATGAATTTTTTTTAAGTTTAATGTGTTTCACATCTCTGTT | |
| | TCTTTTCTCTGCACCAAAAGCTACATTTTTGTGCCCTTATGTACCAGGCAGAAATTGATCT | |
| | GCAATACATGTGGAGTCTCCAAGGGTATATTTAAATTTAGTAATTTTATTGCTAACTGTGA | |
| | AGTTAATCTGCACTATATGGGTTCTTTTCCCCAGGAAACTGAAATAGCAGTTCAAGCTAAA | |
| | CAACCGGATGTGGAA | |
| R12 | Region in vicinity of acceptor of intron 49 (in range of −400 | SEQ No. 388 |
| | to +44 bases from 3′ end of intron 49 as basing point) | |
| | ATATAATTGACTGGGGGTGAGCCAGTACATTAGGATTTTCCTAAAGTTATCTGGATAATTT | |
| | TAGTATGCAACCACAATAGATACTCTTCAAGAATTAAGCTAGTTGCTGAGAGGGAACTGTT | |
| | TTTTGTTGGTTTGTTTTCACTAATGTTTGCACTCTACTTCCTTTAAATAAAATTATGCCTG | |
| | GAGAAAGGGTTTTTGTATGGAGCAATTGATAAATATTTGTAGGGTGGTTGGCTAAAATAAT | |
| | TATAATTCCTTTAAAAGAAATTCTACCCACTAAAGTTAATTTAGAAGTAAAATATAATAGA | |
| | AATCCAATAATATATTCACCAAATGGATTAAGATGTTCATGAATTATCTTCAAAGTGTTAA | |
| | TCGAATAAGTAATGTGTATGCTTTTCTGTTAAAGAGGAAGTTAGAAGATCTGAGCTCTGAG | |
| | TGGAAGGCGGTAAACCG | |
| R14 | Region in vicinity of acceptor of intron 50 (in range of −400 | SEQ No. 342 |
| | to +25 bases from 3′ end of intron 50 as basing point) | |
| | TCTTGAATAAAAAAAAAATAAGTAAAATTTATTTCCCTGGCAAGGTCTGAAAACTTTTGTT | |
| | TTCTTTACCACTTCCACAATGTATATGATTGTTACTGAGAAGGCTTATTTAACTTAAGTTA | |
| | CTTGTCCAGGCATGAGAATGAGCAAAATCGTTTTTTAAAAAATTGTTAAATGTATATTAAT | |
| | GAAAAGGTTGAATCTTTTCATTTTCTACCATGTATTGCTAAACAAAGTATCCACATTGTTA | |
| | GAAAAAGATATATAATGTCATGAATAAGAGTTTGGCTCAAATTGTTACTCTTCAATTAAAT | |
| | TTGACTTATTGTTATTGAAATTGGCTCTTTAGCTTGTGTTTCTAATTTTTCTTTTTCTTCT | |
| | TTTTTCCTTTTTGCAAAAACCCAAAATATTTTAGCTCCTACTCAGACTGTTACTCTGGTGA | |
| | CAC | |
| R16 | Region in vicinity of acceptor of intron 51 (in range of −400 | SEQ No. 343 |
| | to +24 bases from 3′ end of intron 51 as basing point) | |
| | TTTAAAATAAATATACCTTAATTTTGACGTCACACAGAATGATATTATAAGTATAAATAGT | |
| | TATCTATCTTTTAAATACATTGTCGTAATTCAGAATAACATTTCTTACTCAAGGCATTCAG | |
| | ACAGTGGTTTAAGTAATCCGAGGTACTCCGGAATGTCTCCATTTGAGCCTTTAAATGAAGA | |
| | AAATCTATAGTCAAGATTTTCATTTGAAATATTTTTGATATCTAAGAATGAAACATATTTC | |
| | CTGTTAAATTGTTTTCTATAAACCCTTATACAGTAACATCTTTTTTATTTCTAAAAGTGTT | |
| | TTGGCTGGTCTCACAATTGTACTTTACTTTGTATTATGTAAAAGGAATACACAACGCTGAA | |
| | GAACCCTGATACTAAGGGATATTTGTTCTTACAGGCAACAATGCAGGATTTGGAACAGAGG | |
| | CGTCCCCAGTTGGAAGAACTCA | |
| R18 | Region in vicinity of acceptor of intron 52 (in range of −400 | SEQ No. 344 |
| | to +34 bases from 3′ end of intron 52 as basing point) | |
| | CAAACTCCTGTGGCAACAGAAAGCCTTCAGGCAATGAAATGCTGGCACTGGGAAATCAGGC | |
| | TGATGGGTGCTGAAGTGGCAAGGATGAGGGGATATGGATATTCTGCTGTAGTGCTTTTCTA | |
| | ACAGATGATTCATATTTGGTTCTAGGGATCAAGAATTGAGTTAAAATTTTATATATATGTT | |
| | GATGTTCTATGTCACCTTCAGGAAAATAATTTAACAGAAACTAATATTTGCCATCAAAAAA | |
| | GCAAAGAATCCTGTTGTTCATCATCCTAGCCATAACACAATGAATAATTTTTTAAATAAGC | |
| | AACATAAATGTGAGATAACGTTTGGAAGTTACATTTAAAATGTCTCCTCCAGACTAGCATT | |
| | TACTACTATATATTTATTTTTCCTTTTATTCTAGTTGAAAGAATTCAGAATCAGTGGGATG | |
| | AAGTACA | |
| R20 | Region in vicinity of acceptor of intron 53 (in range of −400 | SEQ No. 389 |
| | to +43 bases from 3′ end of intron 53 as basing point) | |
| | CAACAAGTTAATGAAGAGGGAAAGAAATGTATGAGGTTTTTTTCGTTCAAATGTTGTTATA | |
| | TGTCACATATTCAACAATTATATATGAGCTTATTTTTGTAGTTTTTTTCTCTTGTGATAAA | |
| | AACAATTAAGCCCACTTTATTGCCAATTAATTGCTACTAAGTTGAAATACTTGATACTGGT | |
| | TATTGCTCAAGATGCTGCATTTGAAAAGTTTGTCCTGAAAGGTGGGTTACCTTATACTGTC | |
| | ATGATTGACTAAATCATATGGTAGGTTAAAAGCAATCTAATATATGTATTCTGACCTGAGG | |
| | ATTCAGAAGCTGTTTACGAAGTATTTTAAGACACTCCAACTAGAGATTTCATAAAAAAAAC | |
| | TGACATTCATTCTCTTTCTCATAAAAATCTATAGCAGTTGGCCAAAGACCTCCGCCAGTGG | |
| | CAGACAAATGTAGATG | |
| R22 | Region in vicinity of acceptor of intron 54 (in range of −400 | SEQ No. 345 |
| | to +25 bases from 3′ end of intron 54 as basing point) | |
| | TCTCAAATTTGGCAGTATATTAAAAATAAGCTTTCAAAATTGACCAACAAAAACTACAAAA | |
| | TTGAAAAAAAGGTACTTTGAACTTTCACATGTTCAAATATATGTATATATATTTCACATAT | |
| | ATATATGAAACCTCCTCTGTGGAGAGGGGTTTATAGAAATCTGTAATTGTCATTCTTGCAT | |
| | GCCTTCCCCCATACAAACGCCTTTAAGTTAAATAAAAATGAAAGTAAATAGACTGCACAAT | |
| | ATTATAGTTGTTGCTTAAAGGAAGAGCTGTAGCAACAACTCACCCCATTGTTGGTATATTA | |
| | CAATTTAGTTCCTCCATCTTTCTCTTTTTATGGAGTTCACTAGGTGCACCATTCTGATATT | |
| | TAATAATTGCATCTGAACATTTGGTCCTTTGCAGGGTGAGTGAGCGAGAGGCTGCTTTG | |

In a further alternative aspect, the antisense oligomer of the present invention targets at least any one region selected from the group consisting of regions R1 to R24 represented by

region Rn (wherein n is an odd number of 1 to 23) which consists of a base sequence of NY bases in the downstream direction from the 5′ end of the NBth intron in the human dystrophin pre-mRNA, and

region Rn (wherein n is an even number of 2 to 24) which consists of a base sequence of NX bases in the upstream direction from the 3′ end of the NAth intron in the human dystrophin pre-mRNA.

In the foregoing embodiment, examples of the target region Rn of the present invention include, but not limited to, regions wherein

when n is 1, NB=44 and NY=400,

when n is 2, NA=44 and NX=600,

when n is 3, NB=45 and NY=400,

when n is 4, NA=45 and NX=400,

when n is 5, NB=46 and NY=400,

when n is 6, NA=46 and NX=400,

when n is 7, NB=47 and NY=400,

when n is 8, NA=47 and NX=400,

when n is 9, NB=48 and NY=400,

when n is 10, NA=48 and NX=400,

when n is 11, NB=49 and NY=400,

when n is 12, NA=49 and NX=400,

when n is 13, NB=50 and NY=400,

when n is 14, NA=50 and NX=400,

when n is 15, NB=51 and NY=400,

when n is 16, NA=51 and NX=400,

when n is 17, NB=52 and NY=400,

when n is 18, NA=52 and NX=400,

when n is 19, NB=53 and NY=400,

when n is 20, NA=53 and NX=400,

when n is 21, NB=54 and NY=400,

when n is 22, NA=54 and NX=400,

when n is 23, NB=55 and NY=400, or

when n is 24, NA=55 and NX=400.

R1 to R24 which are examples of the target region Rn of the present invention according to the foregoing embodiment are, but not limited to, as follows:

for example, in a certain embodiment,

the region R1 is a region that consists of a base sequence of 400 bases in the downstream direction from the 5′ end of the 44th intron in the human dystrophin pre-mRNA,

the region R2 is a region that consists of a base sequence of 600 bases in the upstream direction from the 3′ end of the 44th intron in the human dystrophin pre-mRNA,

the region R3 is a region that consists of a base sequence of 400 bases in the downstream direction from the 5′ end of the 45th intron in the human dystrophin pre-mRNA,

the region R4 is a region that consists of a base sequence of 400 bases in the upstream direction from the 3′ end of the 45th intron in the human dystrophin pre-mRNA,

the region R5 is a region that consists of a base sequence of 400 bases in the downstream direction from the 5′ end of the 46th intron in the human dystrophin pre-mRNA,

the region R6 is a region that consists of a base sequence of 400 bases in the upstream direction from the 3′ end of the 46th intron in the human dystrophin pre-mRNA,

the region R7 is a region that consists of a base sequence of 400 bases in the downstream direction from the 5′ end of the 47th intron in the human dystrophin pre-mRNA,

the region R8 is a region that consists of a base sequence of 400 bases in the upstream direction from the 3′ end of the 47th intron in the human dystrophin pre-mRNA,

the region R9 is a region that consists of a base sequence of 400 bases in the downstream direction from the 5′ end of the 48th intron in the human dystrophin pre-mRNA,

the region R10 is a region that consists of a base sequence of 400 bases in the upstream direction from the 3′ end of the 48th intron in the human dystrophin pre-mRNA,

the region R11 is a region that consists of a base sequence of 400 bases in the downstream direction from the 5′ end of the 49th intron in the human dystrophin pre-mRNA,

the region R12 is a region that consists of a base sequence of 400 bases in the upstream direction from the 3′ end of the 49th intron in the human dystrophin pre-mRNA,

the region R13 is a region that consists of a base sequence of 400 bases in the downstream direction from the 5′ end of the 50th intron in the human dystrophin pre-mRNA,

the region R14 is a region that consists of a base sequence of 400 bases in the upstream direction from the 3′ end of the 50th intron in the human dystrophin pre-mRNA,

the region R15 is a region that consists of a base sequence of 400 bases in the downstream direction from the 5′ end of the 51st intron in the human dystrophin pre-mRNA,

the region R16 is a region that consists of a base sequence of 400 bases in the upstream direction from the 3′ end of the 51st intron in the human dystrophin pre-mRNA,

the region R17 is a region that consists of a base sequence of 400 bases in the downstream direction from the 5′ end of the 52nd intron in the human dystrophin pre-mRNA,

the region R18 is a region that consists of a base sequence of 400 bases in the upstream direction from the 3′ end of the 52nd intron in the human dystrophin pre-mRNA,

the region R19 is a region that consists of a base sequence of 400 bases in the downstream direction from the 5′ end of the 53rd intron in the human dystrophin pre-mRNA,

the region R20 is a region that consists of a base sequence of 400 bases in the upstream direction from the 3′ end of the 53rd intron in the human dystrophin pre-mRNA,

the region R21 is a region that consists of a base sequence of 400 bases in the downstream direction from the 5′ end of the 54th intron in the human dystrophin pre-mRNA,

the region R22 is a region that consists of a base sequence of 400 bases in the upstream direction from the 3′ end of the 54th intron in the human dystrophin pre-mRNA,

the region R23 is a region that consists of a base sequence of 400 bases in the downstream direction from the 5′ end of the 55th intron in the human dystrophin pre-mRNA, or

the region R24 is a region that consists of a base sequence of 400 bases in the upstream direction from the 3′ end of the 55th intron in the human dystrophin pre-mRNA.

In another embodiment, R1 to R24 which are examples of the target region Rn of the present invention according to the foregoing embodiment are, but not limited to, as follows:

### (A1) Region R1

The region R1 is a region indicated by the range of +1 base to +400 bases when the boundary between the 3′ end of exon 44 and the 5′ end of intron 44 is defined as basing point 0 and a base sequence region on the 3′ side (downstream) from the basing point is indicated by “+”. In this respect, the region indicated by the range of +1 base to +400 bases belongs to intron 44.

### (A2) Region R2

The region R2 is a region indicated by the range of −600 bases to −1 base when the boundary between the 3′ end of intron 44 and the 5′ end of exon 45 is defined as basing point 0 and a base sequence region on the 5′ side (upstream) from the basing point is indicated by “−” (minus). In this respect, the region indicated by the range of −600 bases to −1 base belongs to intron 44.

### (A3) Region R3

The region R3 is a region indicated by the range of +1 base to +400 bases when the boundary between the 3′ end of exon 45 and the 5′ end of intron 45 is defined as basing point 0 and a base sequence region on the 3′ side (downstream) from the basing point is indicated by “+”. In this respect, the region indicated by the range of +1 base to +400 bases belongs to intron 45.

### (A4) Region R4

The region R4 is a region indicated by the range of −400 bases to −1 base when the boundary between the 3′ end of intron 45 and the 5′ end of exon 46 is defined as basing point 0 and a base sequence region on the 5′ side (upstream) from the basing point is indicated by “−” (minus). In this respect, the region indicated by the range of −400 bases to −1 base belongs to intron 45.

### (A5) Region R5

The region R5 is a region indicated by the range of +1 base to +400 bases when the boundary between the 3′ end of exon 46 and the 5′ end of intron 46 is defined as basing point 0 and a base sequence region on the 3′ side (downstream) from the basing point is indicated by “+”. In this respect, the region indicated by the range of +1 base to +400 bases belongs to intron 46.

### (A6) Region R6

The region R6 is a region indicated by the range of −400 bases to −1 base when the boundary between the 3′ end of intron 46 and the 5′ end of exon 47 is defined as basing point 0 and a base sequence region on the 5′ side (upstream) from the basing point is indicated by “−” (minus). In this respect, the region indicated by the range of −400 bases to −1 base belongs to intron 46.

### (A7) Region R7

The region R7 is a region indicated by the range of +1 base to +400 bases when the boundary between the 3′ end of exon 47 and the 5′ end of intron 47 is defined as basing point 0 and a base sequence region on the 3′ side (downstream) from the basing point is indicated by “+”. In this respect, the region indicated by the range of +1 base to +400 bases belongs to intron 47.

### (A8) Region R8

The region R8 is a region indicated by the range of −400 bases to −1 base when the boundary between the 3′ end of intron 47 and the 5′ end of exon 48 is defined as basing point 0 and a base sequence region on the 5′ side (upstream) from the basing point is indicated by “−” (minus). In this respect, the region indicated by the range of −400 bases to −1 base belongs to intron 47.

### (A9) Region R9

The region R9 is a region indicated by the range of +1 base to +400 bases when the boundary between the 3′ end of exon 48 and the 5′ end of intron 48 is defined as basing point 0 and a base sequence region on the 3′ side (downstream) from the basing point is indicated by “+”. In this respect, the region indicated by the range of +1 base to +400 bases belongs to intron 48.

### (A10) Region R10

The region R10 is a region indicated by the range of −400 bases to −1 base when the boundary between the 3′ end of intron 48 and the 5′ end of exon 49 is defined as basing point 0 and a base sequence region on the 5′ side (upstream) from the basing point is indicated by “−” (minus). In this respect, the region indicated by the range of −400 bases to −1 base belongs to intron 48.

### (A11) Region R11

The region R11 is a region indicated by the range of +1 base to +400 bases when the boundary between the 3′ end of exon 49 and the 5′ end of intron 49 is defined as basing point 0 and a base sequence region on the 3′ side (downstream) from the basing point is indicated by “+”. In this respect, the region indicated by the range of +1 base to +400 bases belongs to intron 49.

### (A12) Region R12

The region R12 is a region indicated by the range of −400 bases to −1 base when the boundary between the 3′ end of intron 49 and the 5′ end of exon 50 is defined as basing point 0 and a base sequence region on the 5′ side (upstream) from the basing point is indicated by “−” (minus). In this respect, the region indicated by the range of −400 bases to −1 base belongs to intron 49.

### (A13) Region R13

The region R13 is a region indicated by the range of +1 base to +400 bases when the boundary between the 3′ end of exon 50 and the 5′ end of intron 50 is defined as basing point 0 and a base sequence region on the 3′ side (downstream) from the basing point is indicated by “+”. In this respect, the region indicated by the range of +1 base to +400 bases belongs to intron 50.

### (A14) Region R14

The region R14 is a region indicated by the range of −400 bases to −1 base when the boundary between the 3′ end of intron 50 and the 5′ end of exon 51 is defined as basing point 0 and a base sequence region on the 5′ side (upstream) from the basing point is indicated by “−” (minus). In this respect, the region indicated by the range of −400 bases to −1 base belongs to intron 50.

### (A15) Region R15

The region R15 is a region indicated by the range of +1 base to +400 bases when the boundary between the 3′ end of exon 51 and the 5′ end of intron 51 is defined as basing point 0 and a base sequence region on the 3′ side (downstream) from the basing point is indicated by “+”. In this respect, the region indicated by the range of +1 base to +400 bases belongs to intron 51.

### (A16) Region R16

The region R16 is a region indicated by the range of −400 bases to −1 base when the boundary between the 3′ end of intron 51 and the 5′ end of exon 52 is defined as basing point 0 and a base sequence region on the 5′ side (upstream) from the basing point is indicated by “−” (minus). In this respect, the region indicated by the range of −400 bases to −1 base belongs to intron 51.

### (A17) Region R17

The region R17 is a region indicated by the range of +1 base to +400 bases when the boundary between the 3′ end of exon 52 and the 5′ end of intron 52 is defined as basing point 0 and a base sequence region on the 3′ side (downstream) from the basing point is indicated by “+”. In this respect, the region indicated by the range of +1 base to +400 bases belongs to intron 52.

### (A18) Region R18

The region R18 is a region indicated by the range of −400 bases to −1 base when the boundary between the 3′ end of intron 52 and the 5′ end of exon 53 is defined as basing point 0 and a base sequence region on the 5′ side (upstream) from the basing point is indicated by “−” (minus). In this respect, the region indicated by the range of −400 bases to −1 base belongs to intron 52.

### (A19) Region R19

The region R19 is a region indicated by the range of +1 base to +400 bases when the boundary between the 3′ end of exon 53 and the 5′ end of intron 53 is defined as basing point 0 and a base sequence region on the 3′ side (downstream) from the basing point is indicated by “+”. In this respect, the region indicated by the range of +1 base to +400 bases belongs to intron 53.

### (A20) Region R20

The region R20 is a region indicated by the range of −400 bases to −1 base when the boundary between the 3′ end of intron 53 and the 5′ end of exon 54 is defined as basing point 0 and a base sequence region on the 5′ side (upstream) from the basing point is indicated by “−” (minus). In this respect, the region indicated by the range of −400 bases to −1 base belongs to intron 53.

### (A21) Region R21

The region R21 is a region indicated by the range of +1 base to +400 bases when the boundary between the 3′ end of exon 54 and the 5′ end of intron 54 is defined as basing point 0 and a base sequence region on the 3′ side (downstream) from the basing point is indicated by “+”. In this respect, the region indicated by the range of +1 base to +400 bases belongs to intron 54.

### (A22) Region R22

The region R22 is a region indicated by the range of −400 bases to −1 base when the boundary between the 3′ end of intron 54 and the 5′ end of exon 55 is defined as basing point 0 and a base sequence region on the 5′ side (upstream) from the basing point is indicated by “−” (minus). In this respect, the region indicated by the range of −400 bases to −1 base belongs to intron 54.

### (A23) Region R23

The region R23 is a region indicated by the range of +1 base to +400 bases when the boundary between the 3′ end of exon 55 and the 5′ end of intron 55 is defined as basing point 0 and a base sequence region on the 3′ side (downstream) from the basing point is indicated by “+”. In this respect, the region indicated by the range of +1 base to +400 bases belongs to intron 55.

### (A24) Region R24

The region R24 is a region indicated by the range of −400 bases to −1 base when the boundary between the 3′ end of intron 55 and the 5′ end of exon 56 is defined as basing point 0 and a base sequence region on the 5′ side (upstream) from the basing point is indicated by “−” (minus). In this respect, the region indicated by the range of −400 bases to −1 base belongs to intron 55.

In the foregoing embodiment, specific examples of the base sequences of the regions R1 to R24 are as follows.

TABLE 3

| | | SEQ |
|---|---|---|
| Region | Base sequence of target region | ID NO |
| R1 | Region in vicinity of donor of intron 44 (in range of +1 | SEQ |
| | to +400 bases from 5′ end of intron 44 as basing point) | No. |
| | GTAAGTCTTTGATTTGTTTTTTCGAAATTGTATTTATCTTCAGCACATCTGGACTCTTTAA | 346 |
| | CTTCTTAAAGATCAGGTTCTGAAGGGTGATGGAAATTACTTTTGACTGTTGTTGTCATCAT | |
| | TATATTACTAGAAAGAAAATTATCATAATGATAATATTAGAGCACGGTGCTATGGACTTTT | |
| | TGTGTCAGGATGAGAGAGTTTGCCTGGACGGAGCTGGTTTATCTGATAAACTGCAAAATAT | |
| | AATTGAATCTGTGACAGAGGGAAGCATCGTAACAGCAAGGTGTTTTGTGGCTTTGGGGCAG | |
| | TGTGTATTTCGGCTTTATGTTGGAACCTTTCCAGAAGGAGAACTTGTGGCATACTTAGCTA | |
| | AAATGAAGTTGCTAGAAATATCCATCATGATAAA | |
| R2 | Region in vicinity of acceptor of intron 44 (in range of | SEQ |
| | −600 to −1 bases from 3′ end of intron 44 as basing point) | No. |
| | TCTTGATGGGATGCTCCTGAAAGCAATTAATTCTCAGTTTTTTGTGGCTTCTAATGCAAAA | 347 |
| | TACATTGACGCAGACAGAATTTGAAATGAATTTTCTTCTAATATAGCAATTAATTTTATTT | |
| | AAATATCTCTAGAGTTTTTTTTTAATACTGTGACTAACCTATGTTTGTTCTTTTTCACCTC | |
| | TCGTATCCACGATCACTAAGAAACCCAAATACTTTGTTCATGTTTAAATTTTACAACATTT | |
| | CATAGACTATTAAACATGGAACATCCTTGTGGGGACAAGAAATCGAATTTGCTCTTGAAAA | |
| | GGTTTCCAACTAATTGATTTGTAGGACATTATAACATCCTCTAGCTGACAAGCTTACAAAA | |
| | ATAAAAACTGGAGCTAACCGAGAGGGTGCTTTTTTCCCTGACACATAAAAGGTGTCTTTCT | |
| | GTCTTGTATCCT7TGGATATGGGCATGTCAGTTTCATAGGGAAATTTTCACATGGAGCTTT | |
| | TGTATTTCTTTCTTTGCCAGTACAACTGCATGTGGTAGCACACTGTTTAATCTTTTCTCAA | |
| | ATAAAAAGACATGGGGCTTCATTTTTGTTTTGCCTTTTTGGTATCTTACAG | |
| R3 | Region in vicinity of donor of intron 45 (in range of +1 to | SEQ |
| | +400 bases from 5′ end of intron 45 as basing point) | No. |
| | GTAGGGCGACAGATCTAATAGGAATGAAAACATTTTAGCAGACTTTTTAAGCTTTCTTTAG | 348 |
| | AAGAATATTTCATGAGAGATTATAAGCAGGGTGAAAGGCACTAACATTAAAGAACCTATCA | |
| | ACCATTAATCAACAGCAGTAAAGAAATTTTTTATTTCTTTTTTTCATATACTAAAATATAT | |
| | ACTTGTGGCTAGTTAGTGGTTTTCTGCTATTTTAAACTTGAAGTTTGCTTTAAAAATCACC | |
| | CATGATTGCTTAAAGGTGAATATCTTCAATATATTTTAACTTCAACAAGCTGAATCTCAGT | |
| | TGTTTTTCAAGAAGATTTTAGAAAGCAATTATAAATGATTGTTTTGTAGGAAAGACAGATC | |
| | TTTGCTTAGTTTTAAAAATAGCTATGAATATGAC | |
| R4 | Region in vicinity of acceptor of intron 45 (in range of | SEQ |
| | −400 to −1 bases from 3′ end of intron 45 as basing point) | No. |
| | ATTACAGGCGCCTGCCACCAAACCTGGCAAATTTTTGTATTTTTAGTGTAGACGGGGTTTC | 349 |
| | ACCATATTTGCCAGGCTGGTCGCAAACTCCTGACCTCAAGTGATCCGCCCACATCGGCCTC | |
| | CCTAAGCGCTAGGGTTACAGGCATGAGCCACTGCGCCTGGCCAGGAATTTTTGAATCAGAA | |
| | TTTTTCTTGTTCGATTTTAATCTCTTATCATTTAGAGATTCTTGAAATATTGAAATTACTT | |
| | TGTTCAAAGTGAATGAATTTTCTTAAATTATGTATGGTTAACATCTTTTAAATTGCTTATT | |
| | TTTAAATTGCCATGTTTGrGTCCCAGTTTGCATTAACAAATAGTTTGAGAACTATGTTGGA | |
| | AAAAAAAATAACAATTTTATTCTTCTTTCTCCAG | |
| R5 | Region in vicinity of donor of intron 46 (in range of +1 to | SEQ |
| | +400 bases from 5′ end of intron 46 as basing point) | No. |
| | GTAATTTTATTTTCTCAAATCCCCCAGGGCCTGCTTGCATAAAGAAGTATATGAATCTATT | 350 |
| | TTTTAATTCAATCATTGGTTTTCTGCCCATTAGGTTATTCATAGTTCCTTGCTAAAGTGTT | |
| | TTTCTCACAACTTTATTTCTTCTTAACCCTGCAGTTCTGAACCAGTGCACATAAGAACATA | |
| | TGTATATATGTGTGTGTGTGTATTTATATATACACACACACATATTGCATCTATACATCTA | |
| | CACATATAGATGTATAGATTCAATATGTCTAAATGTATATAATTCACAGTTTTTATCTTTG | |
| | ATTTGAAATTAATTTTAGATTTTACTTGAGAACTTCACAACTTCATATAATTTTAAAAACT | |
| | GAAGACCAGATTGTGGAATCATAAAATCTAAATC | |
| R6 | Region in vicinity of acceptor of intron 46 (in range of | SEQ |
| | −400 to −1 n bases from 3′ end of intron 46 as basing point) | No. |
| | ATAATGCAATTTCTCAGCAGCAAGCTACGGTATGCTATGGCATGCTATGATACCCAAGAGG | 351 |
| | CTGATGAATTTGTTCACATTGTTCTATTTCTGATAGAGAGATAGGTTTTCAGACACTAACT | |
| | TTATTTGGAGTGTTGCTTTACCATCTCACATTTTTCTCTTAAAAAATTTATGAGGGATAAT | |
| | ATAATCGTTTATTTTCTACAGAGATTTATCTACTGAGGGGGTGAGTGTTTCAGTCAATCAG | |
| | CTCTGTGCTCAGATAGAAAACTGTTGGTATTTGAGGTACCACTGGGCCCTCGGTCAAGTCG | |
| | CTTCATTTTGATAGACTAATCAATAGAAGCAAAGACAAGGTAGTTGGAATTGTGCTGTAAT | |
| | TCATTTTAAACGTTGTTGCATTTGTCTGTTTCAG | |
| R7 | Region in vicinity of donor of intron 47 (in range of +1 to | SEQ |
| | +400 bases from 5′ end of intron 47 as basing point) | No. |
| | GTTAGACATTAACCATCTCTTCCGTCACATGTGTTAAATGTTGCAAGTATTTGTATGTATT | 352 |
| | TTGTTTCCTGGGTGCTTCATTGGTCGGGGAGGAGGCTGGTATGTGGATTGTTGTTTTGTTT | |
| | TGTTTTTTTAACCTGACCGTTTGCTTTGGCTATATGTTTTGTTGTGGCTAGAAAAAATGAT | |
| | GATGGTGAATGGCTTTACATTAATGACCAAATGCCAAAATTTATACCACAATTTTTTGCAT | |
| | AAATTATTCTGAAGAATCAGACTGAAGAAATGGCGAAGTATTTAATTCAGTGGCCAGGCAT | |
| | GTACTGACAGTATTTAAGCTGAAAGGACGTGGTCTGGTTCTAGTTAAACAAGTGTCATAAA | |
| | TCAAAATTAATTATTCACACCTG7GGTATGGACT | |
| R8 | Region in vicinity of acceptor of intron 47 (in range of | SEQ |
| | −400 to −1 bases from 3′ end of intron 47 as basing point) | No. |
| | TATTCATTTTTATAACTGCAAAGGAAGCGCGTATGGCATATAATACACAACACACCAGTAT | 353 |
| | ATTTAGTAACTGAGTGAATAAATGAAAGATGTATTTCTTTACTTTATCAGTTGCAGTTGGC | |
| | TATGCCTTTGTGTAAGGTGTGTGTTTTGAAATTCCAAAAAGGTATTAGTTTCTTTAAAGCA | |
| | AAGAATTTTTGTAGCAGGTTAATGAATAATTTTGAATACATTGGTTAAATCCCAACATGTA | |
| | ATATATGTAAATAATCAATATTATGCTGCTAAAATAACACAAATCAGTAAGATTCTGTAAT | |
| | ATTTCA7GATAAATAACTTTTGAAAATATATTTTTAAACATTTTGGCTTATGCCTTGAGAA | |
| | TTATTTACCTTTTTAAAATGTATTTTCCTTTCAG | |
| R9 | Region in vicinity of donor of intron 48 (in range of +1 to | |
| | +400 bases from 5′ end of intron 48 as basing point) | |
| | GTAGGGAACTTTTTGCTTTAAATATTTTTGTCTTTTTTAAGAAAAATGGCAATATCACTGA | SEQ |
| | ATTTTCTCATTTGGTATCATTATTAAAGACAAAATATTACTTGTTAAAGTGTGGTAAGGAA | No. |
| | GACTTTATTCAGGATAACCACAATAGGCACAGGGACCACTGCAATGGAGTATTACAGGAGG | 354 |
| | TTGGATAGAGAGAGATTGGGCTCAACTCTAAATACAGCACAGTGGAAGTAGGAATTTATAG | |
| | CCAAGGAGCAGTGTAGGAGTCAGTAGATGGAAAATTATTAAGAGGAAACATCAGGGGTAAG | |
| | TGGGATTCTGGCTAAACCAACCTCACAGGATTCTTGCTGAAGATAGGCCAGGGTTATCTTA | |
| | TCAGACAACCCTTGGGGAATGGTGGAGAATACTG | |
| R10 | Region in vicinity of acceptor of intron 48 (in range of | SEQ |
| | −400 to −1 bases from 3′ end of intron 48 as basing point) | No. |
| | TACTAAACACAGAATTTTGTAAAACAATAAGTGTATAAAGTAAAATGAACATTAGGATTAT | 355 |
| | TGAGATTATTGTAGCTAAAACTAGTGTTTATTCATATAAATTATGTTAATAAATTGTATTG | |
| | TCATTATTGCATTTTACTTTTTTGAAAAGTAGTTAATGCCTGTGTTTCTATATGAGTATTA | |
| | TATAATTCAAGAAGATATTGGATGAATTTTTTTTAAGTTTAATGTGTTTCACATCTCTGTT | |
| | TCTTTTCTCTGCACCAAAAGCTACATTTTTGTGCCC7TATGTACCAGGCAGAAATTGATCT | |
| | GCAATACATGTGGAGTCTCCAAGGGTATATTTAAATTTAGTAATTTTATTGCTAACTGTGA | |
| | AGTTAATCTGCACTATATGGGTTCTTTTCCCCAG | |
| R11 | Region in vicinity of donor of intron 49 (in range of +1 to | SEQ |
| | +400 bases from 5′ end of intron 49 as basing point) | No. |
| | GTAATGAAGCAACCTCTAGCAATATCCATTACCTCATAATGGGTTATGCTTCCCCTGTTGT | 356 |
| | ACATTTGCCATTGACGTGGACTATTTATAATCAGTGAAATAACTTGTAAGGAAATACTGGC | |
| | CATACTGTAATAGCAGAGGCAAAGCTGTCTTTTTGATCAGCATATCCTATTTATATATTGT | |
| | GATCT7AAGGCTATTAACGAGTCATTGCTTTAAAGGACTCATTTCTGTCCTGGTGTGCTGC | |
| | CATCAATACAAAAGTAGTCCCACCTTCAAGGTAGATTAAATTCTTTGGGGCTTTATTGCTT | |
| | TGCTTGCCAGCCTTGATGCTTTTCATATTGTTTGGTTTAATTCAAATCAAGCTACTGCATC | |
| | ATAGTGTCTGTCTCCAACAGCTGTAAAGAATCAC | |
| R12 | Region in vicinity of acceptor of intron 49 (in range of | SEQ |
| | −400 to −1 bases from 3′ end of intron 49 as basing point) | No. |
| | ATATAATTGACTGGGGGTGAGCCAGTACATTAGGATTTTCCTAAAGTTATCTGGATAATTT | 357 |
| | TACTATGCAACCACAATAGATACTCTTCAAGAATTAAGCTAGTTGCTGAGAGGGAACTGTT | |
| | TTTTGTTGGTTTGTTTTCACTAATGTTTGCACTCTACTTCCTTTAAATAAAATTATGCCTG | |
| | GAGAAAGGGTTTTTGTATGGAGCAATTGATAAATATTTGTAGGGTGGTTGGCTAAAATAAT | |
| | TATAATTCCTTTAAAAGAAATTCTACCCACTAAAGTTAATTTAGAAGTAAAATATAATAGA | |
| | AATCCAATAATATATTCACCAAATGGATTAAGATGTTCATGAATTATCTTCAAAGTGTTAA | |
| | TCGAATAAGTAATGTGTATGCTTTTCTGTTAAAG | |
| R13 | Region in vicinity of donor of intron 50 (in range of +1 to | SEQ |
| | +400 bases from 5′ end of intron 50 as basing point) | No. |
| | GTAAGTATACTGGATCCCATTCTCTTTGGCTCTAGCTATTTGTTCAAAAGTGCAACTATGA | 358 |
| | AGTGATGACTGGGTGAGAGAGAAAATTTGTTTCAATTCTAAAGATAGAGATAAACCTTTGT | |
| | GTTATTGACTGTGCAAAAAGTCTTAGAGTACATTCCTTGGAAATTGACTCTGATTCAAAGT | |
| | GTTGCATGACAACGGGATATGGGGAGTGTTCTCTGGAGATACACCCACAAGGAAGAGAAGA | |
| | GCACAAGGGAGATTGTGGGAGAGTCTGAAATGTGATTTGTCTGCAGCAGAGGCCTAAGCCA | |
| | GTCTCGCAGGAGCCCTACATCTGGGCTGGCTGTGCAGAGCTGTCCTGAATTGCAGGCAGTG | |
| | GGCCTGGCCCTTGTATTCCTGATCCAGCCAGCCA | |
| R14 | Region in vicinity of acceptor of intron 50 (in range of | SEQ |
| | −400 to −1 bases from 3′ end of intron 50 as basing point) | No. |
| | TCTTGAATAAAAAAAAAATAAGTAAAATTTATTTCCCTGGCAAGGTCTGAAAACTTTTGTT | 359 |
| | TTCTTTACCACTTCCACAATGTATATGATTGTTACTGAGAAGGCTTATTTAACTTAAGTTA | |
| | CTTGTCCAGGCATGAGAATGAGCAAAATCGTTTTTTAAAAAATTGTTAAATGTATATTAAT | |
| | GAAAAGGTTGAATCTTTTCATTTTCTACCATGTATTGCTAAACAAAGTATCCACATTGTTA | |
| | GAAAAAGATATATAATGTCATGAATAAGAGTTTGGCTCAAATTGTTACTCTTCAATTAAAT | |
| | TTGACTTATTGTTATTGAAATTGGCTCTTTAGCTTGTGTTTCTAATTTTTCTTTTTCTTCT | |
| | TTTTTCCTTTTTGCAAAAACCCAAAATATTTTAG | |
| R15 | Region in vicinity of donor of intron 51 (in range of +1 to | SEQ |
| | +400 bases from 5′ end of intron 51 as basing point) | No. |
| | GTATGAGAAAAAATGATAAAAGTTGGCAGAAGTTTTTCTTTAAAATGAAGATTTTCCACCA | 360 |
| | ATCACTTTACTCTCCTAGACCATTTCCCACCAGTTCTTAGGCAACTGTTTCTCTCTCAGCA | |
| | AACACATTACTCTCACTATTCAGCCTAAGTATAATCAAGGATATAAATTAATGCAAATAAC | |
| | AAAAGTAGCCATACATTAAAAAGGAAATATACAAAAAAAAAAAAAAAAAAAAGCAGAAACC | |
| | TTACAAGAATAGTTGTCTCAGTTAAATTTACTAAACAACCTGGTATTTTAAAAATCTATTT | |
| | TATACCAAATAAGTCACTCAACTGAGCTATTTACATTTAAACTGTTTGTTTTGGCACTACG | |
| | CAGCCCAACATATTGCAGAATCAAATATAATAGT | |
| R16 | Region in vicinity of acceptor of intron 51 (in range of | SEQ |
| | −400 to −1 bases from 3′ end of intron 51 as basing point) | No. |
| | TTTAAAATAAATATACCTTAATTTTGACGTCACACAGAATGATATTATAAGTATAAATAGT | 361 |
| | TATCTATCTTTTAAATACATTGTCGTAATTCAGAATAACATTTCTTACTCAAGGCATTCAG | |
| | ACAGTGGTTTAAGTAATCCGAGGTACTCCGGAATGTCTCCATTTGAGCCTTTAAATGAAGA | |
| | AAATCTATAGTCAAGATTTTCATTTGAAATATTTTTGATATCTAAGAATGAAACATATTTC | |
| | CTGTTAAATTGTTTTCTATAAACCCTTATACAGTAACATCTTTTTTATTTCTAAAAGTGTT | |
| | TTGGCTGGTCTCACAATTGTACTTTACTTTGTATTATGTAAAAGGAATACACAACGCTGAA | |
| | GAACCCTGATACTAAGGGATATTTGTTCTTACAG | |
| R17 | Region in vicinity of donor of intron 52 (in range of +1 to | |
| | +400 bases from 5′ end of intron 52 as basing point) | |
| | GTAAGTTTTTTAACAAGCATGGGACACACAAAGCAAGATGCATGACAAGTTTCAATAAAAA | SEQ |
| | CTTAAGTTCATATATCCCCCTCACATTTATAAAAATAATGTGAAATAATTGTAAATGATAA | No. |
| | CAATTGTGCTGAGATTTTCAGTCCATAATGTTACCTTTTAATAAATGAATGTAATTCCATT | 362 |
| | GAATAGAAGAAATACATTTTTAAATCAATTCAGGGCTTATATAGTTGCAAAGCATGCATTG | |
| | ATGGGTGTGGTGACCACAGTGTGGCAGAACATTTGTGGCAGAACATTTGTTCTTTAGTTGT | |
| | CATCTGGGCTGGCATCCATGGAGATGCCAGTCTCTCCCTCATATCCTTGGCTGTTGGTCCA | |
| | AGCAGGCAGTGGCTTCTTCCTGGGCCATCTTTCA | |
| R18 | Region in vicinity of acceptor of intron 52 (in range of | SEQ |
| | −400 to −1 bases from 3′ end of intron 52 as basing point) | No. |
| | CAAACTCCTGTGGCAACAGAAAGCCTTCAGGCAATGAAATGCTGGCACTGGGAAATCAGGC | 363 |
| | TGATGGGTGCTGAAGTGGCAAGGATGAGGGGATATGGATATTCTGCTGTAGTGCTTTTCTA | |
| | ACAGATGATTCATATTTGGTTCTAGGGATCAAGAATTGAGTTAAAATTTTATATATATGTT | |
| | GATGTTCTATGTCACCTTCAGGAAAATAATTTAACAGAAACTAATATTTGCCATCAAAAAA | |
| | GCAAAGAATCCTGTTGTTCATCATCCTAGCCATAACACAATGAATAATTTTTTAAATAAGC | |
| | AACATAAATGTGAGATAACGTTTGGAAGTTACATTTAAAATGTCTCCTCCAGACTAGCATT | |
| | TACTACTATATATTTATTTTTCCTTTTATTCTAG | |
| R19 | Region in vicinity of donor of intron 53 (in range of +1 to | SEQ |
| | +400 bases from 5′ end of intron 53 as basing point) | No. |
| | GTTAGTATCAAAGATACCTTTTTAAAATAAAATACTGGTTACATTTGATAAAATTATACCA | 364 |
| | TAGATTGTAATTTAATGATGTTTAATGTAAAGTTATTAACAGAAAATCACGTTAAAGCTGA | |
| | AATGAACAGTAGACTTTGTATATTTATTTTCTTAGAGACAGAGTCTCACTGTCACCCAGGC | |
| | TAAAGTGCAGTGGCACAATCATAGCTCACTGAGCCTTGAACTCTGGGGCTCAAGCAGTCCT | |
| | CCTGCCTCAGCCTCCCTAGTAGCTGGGACTACTAGCCAGGCGTGTACCACCACGCCTGGCT | |
| | AATTTTTTAAAAATTTTTGTTTTCTGTAGAGATGGGTTCTTGAACTCTTGGCCTCAAGCAA | |
| | TTCTCCTTCCTTGGCCTCCCAAAGCACTAGGATT | |
| R20 | Region in vicinity of acceptor of intron 53 (in range of | SEQ |
| | −400 to −1 bases from 3′ end of intron 53 as basing point) | No. |
| | CAACAAGTTAATGAAGAGGGAAAGAAATGTATGAGGTTTTTTTCGTTCAAATGTTGTTATA | 365 |
| | TGTCACATATTCAACAATTATATATGAGCTTATTTTTGTAGTTTTTTTCTCTTG7GATAAA | |
| | AACAATTAAGCCCACTTTATTGCCAATTAATTGCTACTAAGTTGAAATACTTGATACTGGT | |
| | TATTGCTCAAGATGCTGCATTTGAAAAGTTTGTCCTGAAAGGTGGGTTACCTTATACTGTC | |
| | ATGATTGACTAAATCATATGGTAGGTTAAAAGCAATCTAATATATGTATTCTGACCTGAGG | |
| | ATTCAGAAGCTGTTTACGAAGTATTTTAAGACACTCCAACTAGAGATTTCATAAAAAAAAC | |
| | TGACATTCATTCTCTTTCTCATAAAAATCTATAG | |
| R21 | Region in vicinity of donor of intron 54 (in range of +1 to | SEQ |
| | +400 bases from 5′ end of intron 54 as basing point) | No. |
| | GTATGAATTACATTATTTCTAAAACTACTGTTGGCTGTAATAATGGGGTGGTGAAACTGGA | 366 |
| | TGGACCATGAGGATTTGTTTTTCCAATCCAGCTAAACTGGAGCTTGGGAGGGTTCAAGACG | |
| | ATAAATACCAACTAAACTCACGGACTTGGCTCAGACTTCTATTTTAAAAACGAGGAACATA | |
| | AGATCTCATTTGCCCGCTGTCACAAAAGTAGTGACATAACCAAGAGATTAAACAAAAAGCA | |
| | AAATACTGATTTATAGCTAGAAGAGCCATTTATCAGTCTACTTTGATAACTCTATCCAAAG | |
| | GAATATCTTTCTATCTCATCATGGCGCACACTGCCTTACCTGTTATCTGATAAATAAGTCA | |
| | CTTTGGGATTCATGATAGAGTTATAGCTGTACAT | |
| R22 | Region in vicinity of acceptor of intron 54 (in range of | SEQ |
| | −400 to −1 bases from 3′ end of intron 54 as basing point) | No. |
| | TCTGAAATTTGGCAGTATATTAAAAATAAGCTTTCAAAATTGACCAACAAAAACTACAAAA | 367 |
| | TTGAAAAAAAGGTACTTTGAACTTTCACATGTTCAAATATATGTATATATATTTCACATAT | |
| | ATATATGAAACCTCCTCTGTGGAGAGGGGTTTATAGAAATCTGTAATTGTCATTCTTGCAT | |
| | GCCTTCCCCCATACAAACGCCTTTAAGTTAAATAAAAATGAAAGTAAATAGACTGCACAAT | |
| | ATTATAGTTGTTGCTTAAAGGAAGAGCTGTAGCAACAACTCACCCCATTGTTGGTATATTA | |
| | CAATTTAGTTCCTCCATCTTTCTCTTTTTATGGAGTTCACTAGGTGCACCATTCTGATATT | |
| | TAATAATTGCATCTGAACATTTGGTCCTTTGCAG | |
| R23 | Region in vicinity of donor of intron 55 (in range of +1 to | SEQ |
| | +400 nucleotides from 5′ end of intron 55 as basing point) | No. |
| | GTACTTGTAACTGACAAGCCAGGGACAAAACAAAATAGTTGCTTTTATACAGCCTGATGTA | 368 |
| | TTTCGGTATTTGGACAAGGAGGAGAGAGGCAGAGGGAGAAGGAAACATCATTTATAATTCC | |
| | ACTTAACACCCTCGTCTTAGAAAAAGTACATGCTCTGACCAGGAAAACATTTGCATATAAA | |
| | ACCAGAGCTTCGGTCAAGGAGAAACTTTGCTCAGAGAAATAACTTAGGGATTGGTTTATTA | |
| | AATTTTAAAAGTTGACATTTTTGAGTGTTTATTTAATATTTTACAGGGAAAGCATCTGTAT | |
| | GAATTGTCTGTTTTATTTAGCGTTGCTAACTGAA | |
| R24 | Region in vicinity of acceptor of intron 55 (in range of | SEQ |
| | −400 to −1 bases from 3′ end of intron 55 as basing point) | No. |
| | ACTTGATCCATATAGTAATGAAATTATTGGCACTGGGGTACACTTTATCATAGAATTTTAT | 369 |
| | TGCCTATCACTTCCATAAAATAATACATTTTGTCCATAGACTAGAAGATATAACTTGTGAA | |
| | CTTTATAAAGTTATAAATACATTACTTTCCAACTCATAATGGCAAGGAATAAATCTATTAC | |
| | AACTAATAAGATGCCCATTTTAAATCTACATAATAACAGGAGAAGGCAATACGCCAAGAAA | |
| | AGGGATTTGAGATGTATCTTCTTGTTAGTTTAGCCTGATTGAAATGTCTTTTGAACTAATA | |
| | ATTATTTATATTTTGCAATTCTCCAAATTCACATTCATCGCTTGTTTCTTTTGTTTGGTAA | |
| | TTCTGCACATATTCTTCTTCCTGCTGTCCTGTAG | |

The target regions R1 to R24 of the antisense oligomer of the present invention include both wild (e.g., the regions represented by SEQ ID NOs: 233 to 256, 341 to 369, and 385 to 389) and mutant types in relation to the human dystrophin pre-mRNA. Such a mutant type specifically has any one base sequence selected from the group consisting of base sequences (B0) and (B1) to (B16) below:

(B0) a base sequence that hybridizes under stringent conditions to a base sequence complementary to any one base sequence selected from the group consisting of SEQ ID NOs: 233 to 256, 341 to 369, and 385 to 389;
(B1) a base sequence that has at least 85% identity with any one base sequence selected from the group consisting of SEQ ID NOs: 233 to 256, 341 to 369, and 385 to 389, and has a length within ±15% of the length of the any one base sequence selected;
(B2) a base sequence that has at least 86% identity with any one base sequence selected from the group consisting of SEQ ID NOs: 233 to 256, 341 to 369, and 385 to 389, and has a length within ±14% of the length of the any one base sequence selected;
(B3) a base sequence that has at least 87% identity with any one base sequence selected from the group consisting of SEQ ID NOs: 233 to 256, 341 to 369, and 385 to 389, and has a length within ±13% of the length of the any one base sequence selected;
(B4) a base sequence that has at least 88% identity with any one base sequence selected from the group consisting of SEQ ID NOs: 233 to 256, 341 to 369, and 385 to 389, and has a length within ±12% of the length of the any one base sequence selected;
(B5) a base sequence that has at least 89% identity with any one base sequence selected from the group consisting of SEQ ID NOs: 233 to 256, 341 to 369, and 385 to 389, and has a length within ±11% of the length of the any one base sequence selected;
(B6) a base sequence that has at least 90% identity with any one base sequence selected from the group consisting of SEQ ID NOs: 233 to 256, 341 to 369, and 385 to 389, and has a length within ±10% of the length of the any one base sequence selected;
(B7) a base sequence that has at least 91% identity with any one base sequence selected from the group consisting of SEQ ID NOs: 233 to 256, 341 to 369, and 385 to 389, and has a length within ±9% of the length of the any one base sequence selected;
(B8) a base sequence that has at least 92% identity with any one base sequence selected from the group consisting of SEQ ID NOs: 233 to 256, 341 to 369, and 385 to 389, and has a length within ±8% of the length of the any one base sequence selected;
(B9) a base sequence that has at least 93% identity with any one base sequence selected from the group consisting of SEQ ID NOs: 233 to 256, 341 to 369, and 385 to 389, and has a length within ±7% of the length of the any one base sequence selected;
(B10) a base sequence that has at least 94% identity with any one base sequence selected from the group consisting of SEQ ID NOs: 233 to 256, 341 to 369, and 385 to 389, and has a length within ±6% of the length of the any one base sequence selected;
(B11) a base sequence that has at least 95% identity with any one base sequence selected from the group consisting of SEQ ID NOs: 233 to 256, 341 to 369, and 385 to 389, and has a length within ±5% of the length of the any one base sequence selected;
(B12) a base sequence that has at least 96% identity with any one base sequence selected from the group consisting of SEQ ID NOs: 233 to 256, 341 to 369, and 385 to 389, and has a length within ±4% of the length of the any one base sequence selected;
(B13) a base sequence that has at least 97% identity with any one base sequence selected from the group consisting of SEQ ID NOs: 233 to 256, 341 to 369, and 385 to 389, and has a length within ±3% of the length of the any one base sequence selected;
(B14) a base sequence that has at least 98% identity with any one base sequence selected from the group consisting of SEQ ID NOs: 233 to 256, 341 to 369, and 385 to 389, and has a length within ±2% of the length of the any one base sequence selected;
(B15) a base sequence that has at least 99% identity with any one base sequence selected from the group consisting of SEQ ID NOs: 233 to 256, 341 to 369, and 385 to 389, and has a length within ±1% of the length of the any one base sequence selected;
and
(B16) a base sequence that has at least 99.5% identity with any one base sequence selected from the group consisting of SEQ ID NOs: 233 to 256, 341 to 369, and 385 to 389, and has a length within ±0.5% of the length of the any one base sequence selected.

As used herein, the term “base sequence that hybridizes under stringent conditions” refers to, for example, a base sequence obtained by colony hybridization, plaque hybridization, Southern hybridization or the like, using as a probe all or part of a base sequence complementary to, e.g., any one base sequence selected from the group consisting of SEQ ID NOs: 233 to 256, 341 to 369, and 385 to 389. The hybridization method which may be used includes methods described in, for example, “Sambrook & Russell, Molecular Cloning: A Laboratory Manual Vol. 3, Cold Spring Harbor, Laboratory Press, 2001,” “Ausubel, Current Protocols in Molecular Biology, John Wiley & Sons, 1987-1997,” etc.

As used herein, the term “complementary base sequence” is not limited to a base sequence that forms Watson-Crick pairs with an intended base sequence, and also includes a base sequence that forms wobble base pairs therewith. Herein, the Watson-Crick pair means a base pair that forms a hydrogen bond between adenine and thymine, between adenine and uracil, or between guanine and cytosine, and the wobble base pair means a base pair that forms a hydrogen bond between guanine and uracil, between inosine and uracil, between inosine and adenine, or between inosine and cytosine. The term “complementary base sequence” does not have to have 100% complementarity with the intended base sequence and may contain, for example, 1, 2, 3, 4, or 5 noncomplementary bases to the intended base sequence or may be a base sequence shorter by 1 base, 2 bases, 3 bases, 4 bases, or 5 bases than the intended base sequence.

As used herein, the term “stringent conditions” may be any of low stringent conditions, moderate stringent conditions or high stringent conditions. The term “low stringent condition” is, for example, 5×SSC, 5×Denhardt's solution, 0.5% SDS, 50% formamide at 32° C. The term “moderate stringent condition” is, for example, 5×SSC, 5×Denhardt's solution, 0.5% SDS, 50% formamide at 42° C., or 5×SSC, 1% SDS, 50 mM Tris-HCl (pH 7.5), 50% formamide at 42° C. The term “high stringent condition” is, for example, 5×SSC, 5×Denhardt's solution, 0.5% SDS, 50% formamide at 50° C., or 0.2×SSC, 0.1% SDS at 65° C. Under these conditions, base sequences with higher identity are expected to be obtained efficiently at higher temperatures, although multiple factors are involved in hybridization stringency including temperature, probe concentration, probe length, ionic strength, time, salt concentration and others, and those skilled in the art may appropriately select these factors to achieve similar stringency.

When commercially available kits are used for hybridization, for example, an Alkphos Direct Labelling and Detection System (GE Healthcare) may be used. In this case, according to the attached protocol, after incubation with a labeled probe overnight, the membrane can be washed with a primary wash buffer containing 0.1% (w/v) SDS at 55° C., thereby detecting hybridization. Alternatively, when the probe is labeled with digoxigenin (DIG) using a commercially available reagent (e.g., a PCR Labelling Mix (Roche Diagnostics), etc.) in producing a probe based on all or part of the complementary sequence to any one base sequence selected from the group consisting of SEQ ID NOs: 233 to 256, 341 to 369, and 385 to 389, hybridization can be detected with a DIG Nucleic Acid Detection Kit (Roche Diagnostics) or the like.

The identity between base sequences may be determined using algorithm BLAST (Basic Local Alignment Search Tool) by Karlin and Altschul (Proc. Natl. Acad. Sci. USA 872264-2268, 1990; Proc. Natl. Acad. Sci. USA 90: 5873, 1993). Programs called BLASTN and BLASTX based on the BLAST algorithm have been developed (Altschul S F, et al: J. Mol. Biol. 215: 403, 1990). When a base sequence is analyzed using BLASTN, the parameters are, for example, score=100 and wordlength=12. When BLAST and Gapped BLAST programs are used, the default parameters for each program are employed.

The antisense oligomer of the present invention comprises a base sequence complementary to a base sequence of at least any one region selected from the group consisting of the target regions R1 to R24 of the present invention, or a partial base sequence thereof. The term “partial” means a region, except for the full length, of at least any one region selected from the group consisting of the target regions R1 to R24, i.e., a partial region of at least any one region selected from the group consisting of the target regions R1 to R24. The partial region may be 10 to 60 bases long, 10 to 55 bases long, 10 to 50 bases long, 10 to 45 bases long, 10 to 40 bases long, 10 to 35 bases long, 10 to 30 bases long, 10 to 25 bases long, 15 to 60 bases long, 15 to 55 bases long, 15 to 50 bases long, 15 to 45 bases long, 15 to 40 bases long, 15 to 35 bases long, 15 to 30 bases long, 15 to 25 bases long, 16 to 60 bases long, 16 to 55 bases long, 16 to 50 bases long, 16 to 45 bases long, 16 to 40 bases long, 16 to 35 bases long, 16 to 30 bases long, 16 to 25 bases long, 17 to 60 bases long, 17 to 55 bases long, 17 to 50 bases long, 17 to 45 bases long, 17 to 40 bases long, 17 to 35 bases long, 17 to 30 bases long, 17 to 25 bases long, 18 to 60 bases long, 18 to 55 bases long, 18 to 50 bases long, 18 to 45 bases long, 18 to 40 bases long, 18 to 35 bases long, 18 to 30 bases long, 18 to 25 bases long, 19 to 60 bases long, 19 to 55 bases long, 19 to 50 bases long, 19 to 45 bases long, 19 to 40 bases long, 19 to 35 bases long, 19 to 30 bases long, 19 to 25 bases long, 20 to 60 bases long, 20 to 55 bases long, 20 to 50 bases long, 20 to 45 bases long, 20 to 40 bases long, 20 to 35 bases long, 20 to 30 bases long, 20 to 25 bases long, 15 to 30 bases long, 15 to 29 bases long, 15 to 28 bases long, 15 to 27 bases long, 15 to 26 bases long, 15 to 25 bases long, 15 to 24 bases long, 15 to 23 bases long, 15 to 22 bases long, 15 to 21 bases long, 15 to 20 bases long, 15 to 19 bases long, 15 to 18 bases long, 16 to 30 bases long, 16 to 29 bases long, 16 to 28 bases long, 16 to 27 bases long, 16 to 26 bases long, 16 to 25 bases long, 16 to 24 bases long, 16 to 23 bases long, 16 to 22 bases long, 16 to 21 bases long, 16 to 20 bases long, 16 to 19 bases long, 16 to 18 bases long, 17 to 30 bases long, 17 to 29 bases long, 17 to 28 bases long, 17 to 27 bases long, 17 to 26 bases long, 17 to 25 bases long, 17 to 24 bases long, 17 to 23 bases long, 17 to 22 bases long, 17 to 21 bases long, 17 to 20 bases long, 17 to 19 bases long, 17 to 18 bases long, 18 to 30 bases long, 18 to 29 bases long, 18 to 28 bases long, 18 to 27 bases long, 18 to 26 bases long, 18 to 25 bases long, 18 to 24 bases long, 18 to 23 bases long, 18 to 22 bases long, 18 to 21 bases long, 18 to 20 bases long, 18 to 19 bases long, 19 to 30 bases long, 19 to 29 bases long, 19 to 28 bases long, 19 to 27 bases long, 19 to 26 bases long, 19 to 25 bases long, 19 to 24 bases long, 19 to 23 bases long, 19 to 22 bases long, 19 to 21 bases long, 19 to 20 bases long, 20 to 30 bases long, 20 to 29 bases long, 20 to 28 bases long, 20 to 27 bases long, 20 to 26 bases long, 20 to 25 bases long, 20 to 24 bases long, 20 to 23 bases long, 20 to 22 bases long, 20 to 21 bases long, 5 to 25 bases long, 5 to 24 bases long, 5 to 23 bases long, 5 to 22 bases long, 5 to 21 bases long, 5 to 20 bases long, 5 to 19 bases long, 5 to 18 bases long, 5 to 17 bases long, 5 to 16 bases long, 5 to 15 bases long, 5 to 14 bases long, 5 to 13 bases long, 5 to 12 bases long, 7 to 25 bases long, 7 to 24 bases long, 7 to 23 bases long, 7 to 22 bases long, 7 to 21 bases long, 7 to 20 bases long, 7 to 19 bases long, 7 to 18 bases long, 7 to 17 bases long, 7 to 16 bases long, 7 to 15 bases long, 7 to 14 bases long, 7 to 13 bases long, 7 to 12 bases long, 9 to 25 bases long, 9 to 24 bases long, 9 to 23 bases long, 9 to 22 bases long, 9 to 21 bases long, 9 to 20 bases long, 9 to 19 bases long, 9 to 18 bases long, 9 to 17 bases long, 9 to 16 bases long, 9 to 15 bases long, 9 to 14 bases long, 9 to 13 bases long, 9 to 12 bases long, 10 to 25 bases long, 10 to 24 bases long, 10 to 23 bases long, 10 to 22 bases long, 10 to 21 bases long, 10 to 20 bases long, 10 to 19 bases long, 10 to 18 bases long, 10 to 17 bases long, 10 to 16 bases long, 10 to 15 bases long, 10 to 14 bases long, 10 to 13 bases long, 10 to 12 bases long, 60 bases long, 59 bases long, 58 bases long, 57 bases long, 56 bases long, 55 bases long, 54 bases long, 53 bases long, 52 bases long, 51 bases long, 50 bases long, 49 bases long, 48 bases long, 47 bases long, 46 bases long, 45 bases long, 44 bases long, 43 bases long, 42 bases long, 41 bases long, 40 bases long, 39 bases long, 38 bases long, 37 bases long, 36 bases long, 35 bases long, 34 bases long, 33 bases long, 32 bases long, 31 bases long, 30 bases long, 29 bases long, 28 bases long, 27 bases long, 26 bases long, 25 bases long, 24 bases long, 23 bases long, 22 bases long, 21 bases long, 20 bases long, 19 bases long, 18 bases long, 17 bases long, 16 bases long, 15 bases long, 14 bases long, 13 bases long, 12 bases long, 11 bases long, 10 bases long, 9 bases long, 8 bases long, 7 bases long, 6 bases long, or 5 bases long, but not limited thereto. These lengths may be increased or decreased by 1, 2, or 3 bases.

The antisense oligomer of the present invention has an activity to cause simultaneous skipping of any two or more numerically consecutive exons selected from the group consisting of the 45th exon to the 55th exon in human dystrophin pre-mRNA. As used herein, such skipping of two or more numerically consecutive exons from pre-mRNA of interest is referred to as “multi-exon skipping” or “multi-skipping”, and this activity is referred to as “multi-exon skipping activity” or “multi-skipping activity”.

Herein, the any two or more numerically consecutive exons selected from the group consisting of the 45th exon to the 55th exon mean a plurality of exons that increase one by one in exon number among 11 exons from the 45th exon to the 55th exon included in pre-mRNA. The exon number means a number assigned to exons in order from the 5′ end to the 3′ end with an exon at the most upstream position of pre-mRNA defined as the first exon, followed by the second, the third, . . . , and the 79th exons among 79 exons included in human dystrophin pre-mRNA. An intron is numbered as the same number as that of an exon positioned on the 5′ side thereof. Specifically, the 45th intron is flanked by the 45th exon positioned on the 5′ side thereof and the 46th exon positioned on the 3′ side thereof. As used herein, the “nth” exon or intron means the nth exon or intron counted from the 5′ end toward the 3′ end in pre-mRNA.

Table 4 shows combinations of exons included in the any two or more numerically consecutive exons selected from the group consisting of the 45th exon to the 55th exon.

TABLE 4

| Combination | Exons included |
|---|---|
| Combination 1 | 45, 46 |
| Combination 2 | 45-47 |
| Combination 3 | 45-48 |
| Combination 4 | 45-49 |
| Combination 5 | 45-50 |
| Combination 6 | 45-51 |
| Combination 7 | 45-52 |
| Combination 8 | 45-53 |
| Combination 9 | 45-54 |
| Combination 10 | 45-55 |
| Combination 11 | 46, 47 |
| Combination 12 | 46-48 |
| Combination 13 | 46-49 |
| Combination 14 | 46-50 |
| Combination 15 | 46-51 |
| Combination 16 | 46-52 |
| Combination 17 | 46-53 |
| Combination 18 | 46-54 |
| Combination 19 | 46-55 |
| Combination 20 | 47, 48 |
| Combination 21 | 47-49 |
| Combination 22 | 47-50 |
| Combination 23 | 47-51 |
| Combination 24 | 47-52 |
| Combination 25 | 47-53 |
| Combination 26 | 47-54 |
| Combination 27 | 47-55 |
| Combination 28 | 48, 49 |
| Combination 29 | 48-50 |
| Combination 30 | 48-51 |
| Combination 31 | 48-52 |
| Combination 32 | 48-53 |
| Combination 33 | 48-54 |
| Combination 34 | 48-55 |
| Combination 35 | 49, 50 |
| Combination 36 | 49-51 |
| Combination 37 | 49-52 |
| Combination 38 | 49-53 |
| Combination 39 | 49-54 |
| Combination 40 | 49-55 |
| Combination 41 | 50, 51 |
| Combination 42 | 50-52 |
| Combination 43 | 50-53 |
| Combination 44 | 50-54 |
| Combination 45 | 50-55 |
| Combination 46 | 51, 52 |
| Combination 47 | 51-53 |
| Combination 48 | 51-54 |
| Combination 49 | 51-55 |
| Combination 50 | 52, 53 |
| Combination 51 | 52-54 |
| Combination 52 | 52-55 |
| Combination 53 | 53, 54 |
| Combination 54 | 53-55 |
| Combination 55 | 54, 55 |

Among the combinations of exons described in Table 4, for example, the combination 1, 2, 3, 4, 6, 8, 10, 18, 20, 21, 23, 25, 27, 28, 30, 32, 34, 36, 38, 40, 41, 43, 45, 46, 50, 52, or 55 is a skipping pattern expected to exert higher therapeutic effects on DMD. Multi-exon skipping in such a combination is expected to exert therapeutic effects on more patients with DMD.

The any two or more numerically consecutive exons selected from the group consisting of the 45th exon to the 55th exon may include a plurality of groups of consecutive exons and may be, for example, but not limited to, (example 1) exons 45 and 46 (first exon group) and exons 48 to 53 (second exon group), or (example 2) exons 46 and 47 (first exon group), exons 49 and 50 (second exon group), and exons 52 to 54 (third exon group).

In the present invention, the term “activity to cause skipping” (i.e., multi-skipping activity) means, when human dystrophin pre-mRNA is taken as an example, an activity to produce human dystrophin mRNA having deletion of any two or more numerically consecutive exons selected from the group consisting of the 45th exon to the 55th exon in the human dystrophin pre-mRNA.

In other words, this activity means that by binding of the antisense oligomer of the present invention to a target site in human dystrophin pre-mRNA, the 5′-terminal nucleotide of an exon immediately downstream of the exons to be deleted is linked to the 3′-terminal nucleotide of an exon immediately upstream of the exons to be deleted when the pre-mRNA undergoes splicing, thus resulting in formation of mature mRNA which is free of codon frame shift (i.e., mature mRNA having deletion of the exons without frame shift).

The antisense oligomer of the present invention exhibits a multi-skipping activity under physiological conditions. The term “under physiological conditions” refers to conditions set to mimic the in vivo environment in terms of pH, salt composition and temperature. The conditions are, for example, 25 to 40° C., preferably 37° C., pH 5 to 8, preferably pH 7.4 and 150 mM of sodium chloride concentration. When multi-skipping is induced, one or more, for example, 2, 3, 4, 5, 6, 7, 8, 9 or 10 antisense oligomers, can be used in combination as the antisense oligomer of the present invention.

Whether multi-skipping is caused or not can be confirmed by introducing the antisense oligomer of the present invention into a dystrophin expression cell (e.g., human rhabdomyosarcoma cells), amplifying the region surrounding exons 45 to 55 of mRNA of the human dystrophin gene from the total RNA of the dystrophin expression cell by RT-PCR and performing nested PCR or sequence analysis on the PCR amplified product. The multi-skipping efficiency can be determined as follows. The mRNA for the human dystrophin gene is collected from test cells; in the mRNA, the polynucleotide level “A” of the band where any two or more numerically consecutive exons among exons 45 to 55 are skipped, the polynucleotide level “B” of the band where any one exon among exons 45 to 55 is skipped, and the polynucleotide level “C” of the band where no skipping is caused are measured. Using these measurement values of “A”, “B”, and “C”, the efficiency is calculated by the following equation.

Skipping efficiency (%)=A/(A+B+C)×100

For example, the multi-skipping efficiency of exons 45 to 55 can be determined by using a forward primer for exon 44 and a reverse primer for exon 56 to measure the polynucleotide level “A” of the band where exons 45 to 55 are multi-skipped, using the forward primer for exon 44 and a reverse primer for exon 46 to measure the polynucleotide level “B” of the band where exon 45 is single-skipped, and using the forward primer for exon 44 and the reverse primer for exon 46 to measure the polynucleotide level “C” of the band where no skipping is caused, followed by calculation by the equation using these measurement values of “A”, “B”, and “C”.

The number of exons to be deleted in human dystrophin mRNA by the antisense oligomer of the present invention is 2, 3, 4, 5, 6, 7, 8, 9, 10, or 11. This is referred to as a deletion pattern, and various deletion patterns may exist in admixture in results obtained in one skipping experiment or skipping treatment. For example, mRNA admixture having deletion of 2, 3, 4, 5, 6, 7, 8, 9, 10, or 11 exons is obtained by introducing the antisense oligomer of the present invention to cells expressing human dystrophin pre-mRNA, and collecting its mRNA.

In a certain embodiment, the term “activity to cause skipping” can be defined as (C1) to (C10) below.

(C1) Any two numerically consecutive exons selected from the group consisting of the 45th exon to the 55th exon in human dystrophin pre-mRNA are skipped with the efficiency of 5% or higher, 10% or higher, 15% or higher, 20% or higher, 25% or higher, 30% or higher, 35% or higher, 40% or higher, 45% or higher, 50% or higher, 55% or higher, 60% or higher, 65% or higher, 70% or higher, 75% or higher, 80% or higher, 85% or higher, 90% or higher, or 95% or higher.

Herein, the two numerically consecutive exons may be the 45th and the 46th exons, the 46th and the 47th exons, the 47th and the 48th exons, the 48th and the 49th exons, the 49th and the 50th exons, the 50th and the 51st exons, the 51st and the 52nd exons, the 52nd and the 53rd exons, the 53rd and the 54th exons, or the 54th and the 55th exons.

(C2) Any three numerically consecutive exons selected from the group consisting of the 45th exon to the 55th exon in human dystrophin pre-mRNA are skipped with the efficiency of 5% or higher, 10% or higher, 15% or higher, 20% or higher, 25% or higher, 30% or higher, 35% or higher, 40% or higher, 45% or higher, 50% or higher, 55% or higher, 60% or higher, 65% or higher, 70% or higher, 75% or higher, 80% or higher, 85% or higher, 90% or higher, or 95% or higher.

Herein, the three numerically consecutive exons may be the 45th to the 47th exons, the 46th to the 48th exons, the 47th to the 49th exons, the 48th to the 50th exons, the 49th to the 51st exons, the 50th to the 52nd exons, the 51st to the 53rd exons, the 52nd to the 54th exons, or the 53rd to the 55th exons.

(C3) Any four numerically consecutive exons selected from the group consisting of the 45th exon to the 55th exon in human dystrophin pre-mRNA are skipped with the efficiency of 5% or higher, 10% or higher, 15% or higher, 20% or higher, 25% or higher, 30% or higher, 35% or higher, 40% or higher, 45% or higher, 50% or higher, 55% or higher, 60% or higher, 65% or higher, 70% or higher, 75% or higher, 80% or higher, 85% or higher, 90% or higher, or 95% or higher.

Herein, the four numerically consecutive exons may be the 45th to the 48th exons, the 46th to the 49th exons, the 47th to the 50th exons, the 48th to the 51st exons, the 49th to the 52nd exons, the 50th to the 53rd exons, the 51st to the 54th exons, or the 52nd to the 55th exons.

(C4) Any five numerically consecutive exons selected from the group consisting of the 45th exon to the 55th exon in human dystrophin pre-mRNA are skipped with the efficiency of 5% or higher, 10% or higher, 15% or higher, 20% or higher, 25% or higher, 30% or higher, 35% or higher, 40% or higher, 45% or higher, 50% or higher, 55% or higher, 60% or higher, 65% or higher, 70% or higher, 75% or higher, 80% or higher, 85% or higher, 90% or higher, or 95% or higher.

Herein, the five numerically consecutive exons may be the 45th to the 49th exons, the 46th to the 50th exons, the 47th to the 51st exons, the 48th to the 52nd exons, the 49th to the 53rd exons, the 50th to the 54th exons, or the 51st to the 55th exons.

(C5) Any six numerically consecutive exons selected from the group consisting of the 45th exon to the 55th exon in human dystrophin pre-mRNA are skipped with the efficiency of 5% or higher, 10% or higher, 15% or higher, 20% or higher, 25% or higher, 30% or higher, 35% or higher, 40% or higher, 45% or higher, 50% or higher, 55% or higher, 60% or higher, 65% or higher, 70% or higher, 75% or higher, 80% or higher, 85% or higher, 90% or higher, or 95% or higher.

Herein, the six numerically consecutive exons may be the 45th to the 50th exons, the 46th to the 51st exons, the 47th to the 52nd exons, the 48th to the 53rd exons, the 49th to the 54th exons, or the 50th to the 55th exons.

(C6) Any seven numerically consecutive exons selected from the group consisting of the 45th exon to the 55th exon in human dystrophin pre-mRNA are skipped with the efficiency of 5% or higher, 10% or higher, 15% or higher, 20% or higher, 25% or higher, 30% or higher, 35% or higher, 40% or higher, 45% or higher, 50% or higher, 55% or higher, 60% or higher, 65% or higher, 70% or higher, 75% or higher, 80% or higher, 85% or higher, 90% or higher, or 95% or higher.

Herein, the seven numerically consecutive exons may be the 45th to the 51st exons, the 46th to the 52nd exons, the 47th to the 53rd exons, the 48th to the 54th exons, or the 49th to the 55th exons.

(C7) Any eight numerically consecutive exons selected from the group consisting of the 45th exon to the 55th exon in human dystrophin pre-mRNA are skipped with the efficiency of 5% or higher, 10% or higher, 15% or higher, 20% or higher, 25% or higher, 30% or higher, 35% or higher, 40% or higher, 45% or higher, 50% or higher, 55% or higher, 60% or higher, 65% or higher, 70% or higher, 75% or higher, 80% or higher, 85% or higher, 90% or higher, or 95% or higher.

Herein, the eight numerically consecutive exons may be the 45th to the 52nd exons, the 46th to the 53rd exons, the 47th to the 54th exons, or the 48th to the 55th exons.

(C8) Any nine numerically consecutive exons selected from the group consisting of the 45th exon to the 55th exon in human dystrophin pre-mRNA are skipped with the efficiency of 5% or higher, 10% or higher, 15% or higher, 20% or higher, 25% or higher, 30% or higher, 35% or higher, 40% or higher, 45% or higher, 50% or higher, 55% or higher, 60% or higher, 65% or higher, 70% or higher, 75% or higher, 80% or higher, 85% or higher, 90% or higher, or 95% or higher.

Herein, the nine numerically consecutive exons may be the 45th to the 53rd exons, the 46th to the 54th exons, or the 47th to the 55th exons.

(C9) Any ten numerically consecutive exons selected from the group consisting of the 45th exon to the 55th exon in human dystrophin pre-mRNA are skipped with the efficiency of 5% or higher, 10% or higher, 15% or higher, 20% or higher, 25% or higher, 30% or higher, 35% or higher, 40% or higher, 45% or higher, 50% or higher, 55% or higher, 60% or higher, 65% or higher, 70% or higher, 75% or higher, 80% or higher, 85% or higher, 90% or higher, or 95% or higher.

Herein, the ten numerically consecutive exons may be the 45th to the 54th exons, or the 46th to the 55th exons.

(C10) Eleven numerically consecutive exons selected from the group consisting of the 45th exon to the 55th exon in human dystrophin pre-mRNA are skipped with the efficiency of 5% or higher, 10% or higher, 15% or higher, 20% or higher, 25% or higher, 30% or higher, 35% or higher, 40% or higher, 45% or higher, 50% or higher, 55% or higher, 60% or higher, 65% or higher, 70% or higher, 75% or higher, 80% or higher, 85% or higher, 90% or higher, or 95% or higher.

Herein, the eleven numerically consecutive exons may be the 45th to the 55th exons.

The antisense oligomer of the present invention may be 10 to 60 bases long, 10 to 55 bases long, 10 to 50 bases long, 10 to 45 bases long, 10 to 40 bases long, 10 to 35 bases long, 10 to 30 bases long, 10 to 25 bases long, 15 to 60 bases long, 15 to 55 bases long, 15 to 50 bases long, 15 to 45 bases long, 15 to 40 bases long, 15 to 35 bases long, 15 to 30 bases long, 15 to 25 bases long, 16 to 60 bases long, 16 to 55 bases long, 16 to 50 bases long, 16 to 45 bases long, 16 to 40 bases long, 16 to 35 bases long, 16 to 30 bases long, 16 to 25 bases long, 17 to 60 bases long, 17 to 55 bases long, 17 to 50 bases long, 17 to 45 bases long, 17 to 40 bases long, 17 to 35 bases long, 17 to 30 bases long, 17 to 25 bases long, 18 to 60 bases long, 18 to 55 bases long, 18 to 50 bases long, 18 to 45 bases long, 18 to 40 bases long, 18 to 35 bases long, 18 to 30 bases long, 18 to 25 bases long, 19 to 60 bases long, 19 to 55 bases long, 19 to 50 bases long, 19 to 45 bases long, 19 to 40 bases long, 19 to 35 bases long, 19 to 30 bases long, 19 to 25 bases long, 20 to 60 bases long, 20 to 55 bases long, 20 to 50 bases long, 20 to 45 bases long, 20 to 40 bases long, 20 to 35 bases long, 20 to 30 bases long, 20 to 25 bases long, 15 to 30 bases long, 15 to 29 bases long, 15 to 28 bases long, 15 to 27 bases long, 15 to 26 bases long, 15 to 25 bases long, 15 to 24 bases long, 15 to 23 bases long, 15 to 22 bases long, 15 to 21 bases long, 15 to 20 bases long, 15 to 19 bases long, 15 to 18 bases long, 16 to 30 bases long, 16 to 29 bases long, 16 to 28 bases long, 16 to 27 bases long, 16 to 26 bases long, 16 to 25 bases long, 16 to 24 bases long, 16 to 23 bases long, 16 to 22 bases long, 16 to 21 bases long, 16 to 20 bases long, 16 to 19 bases long, 16 to 18 bases long, 17 to 30 bases long, 17 to 29 bases long, 17 to 28 bases long, 17 to 27 bases long, 17 to 26 bases long, 17 to 25 bases long, 17 to 24 bases long, 17 to 23 bases long, 17 to 22 bases long, 17 to 21 bases long, 17 to 20 bases long, 17 to 19 bases long, 17 to 18 bases long, 18 to 30 bases long, 18 to 29 bases long, 18 to 28 bases long, 18 to 27 bases long, 18 to 26 bases long, 18 to 25 bases long, 18 to 24 bases long, 18 to 23 bases long, 18 to 22 bases long, 18 to 21 bases long, 18 to 20 bases long, 18 to 19 bases long, 19 to 30 bases long, 19 to 29 bases long, 19 to 28 bases long, 19 to 27 bases long, 19 to 26 bases long, 19 to 25 bases long, 19 to 24 bases long, 19 to 23 bases long, 19 to 22 bases long, 19 to 21 bases long, 19 to 20 bases long, 20 to 30 bases long, 20 to 29 bases long, 20 to 28 bases long, 20 to 27 bases long, 20 to 26 bases long, 20 to 25 bases long, 20 to 24 bases long, 20 to 23 bases long, 20 to 22 bases long, 20 to 21 bases long, 60 bases long, 59 bases long, 58 bases long, 57 bases long, 56 bases long, 55 bases long, 54 bases long, 53 bases long, 52 bases long, 51 bases long, 50 bases long, 49 bases long, 48 bases long, 47 bases long, 46 bases long, 45 bases long, 44 bases long, 43 bases long, 42 bases long, 41 bases long, 40 bases long, 39 bases long, 38 bases long, 37 bases long, 36 bases long, 35 bases long, 34 bases long, 33 bases long, 32 bases long, 31 bases long, 30 bases long, 29 bases long, 28 bases long, 27 bases long, 26 bases long, 25 bases long, 24 bases long, 23 bases long, 22 bases long, 21 bases long, 20 bases long, 19 bases long, 18 bases long, 17 bases long, 16 bases long, 15 bases long, 14 bases long, 13 bases long, 12 bases long, 11 bases long, or 10 bases long, but not limited thereto. These lengths may be increased or decreased by 1, 2, or 3 bases.

One embodiment of the antisense oligomer of the present invention is a linked-type antisense oligomer configured to comprise a plurality of unit oligomers linked to each other, a pharmaceutically acceptable salt thereof, or hydrate thereof (hereinafter, referred to as the “linked-type antisense oligomer of the present invention”). The unit oligomers mean respective oligomers constituting the linked-type antisense oligomer of the present invention. Specifically, the unit oligomers mean moieties (units) comprising base sequences that hybridize with target base sequences having consecutive base sequences when the linked-type antisense oligomer of the present invention binds to the target base sequences in human dystrophin pre-mRNA.

Specifically, the linked-type antisense oligomer of the present invention is an antisense oligomer comprising two or more unit oligomers linked to each other, or a pharmaceutically acceptable salt thereof, or hydrate thereof, wherein

each of the unit oligomers comprises a base sequence complementary to a base sequence of any one region selected from the group consisting of the regions R1 to R24 (which may include the wild-type regions represented by SEQ ID NOs: 233 to 256, 341 to 369, and 385 to 389 and their mutant types), or a partial base sequence thereof, and the respective base sequences of the unit oligomers are neither consecutive nor overlapped with each other.

The unit oligomers may be linked via a linker that does not contribute to hybridization, or may be linked directly without the mediation of a linker. When the unit oligomers are linked directly to each other, the 3′ end of the unit positioned on the 5′ side and the 5′ end of the unit positioned on the 3′ side form a phosphate bond or any one of the following groups.

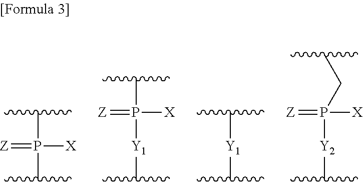

wherein X represents —OH, —CH₂R¹, —O—CH₂R¹, —S—CH₂R¹, —NR²R³ or F;

R¹ represents H or an alkyl;

R² and R³, which may be the same or different, each represents H, an alkyl, a cycloalkyl or an aryl;

Y₁ represents O, S, CH₂, or NR¹;

Y₂ represents O, S, or NR¹;

Z represents O or S.

In the linked-type antisense oligomer of the present invention, the unit oligomers may respectively target base sequences included in separate regions among the regions R1 to R24, or may respectively target base sequences included in the same region. In the linked-type antisense oligomer of the present invention, the respective base sequences of the unit oligomers are neither consecutive nor overlapped with each other. The respective base sequences of the unit oligomers that are not consecutive mean that the respective target base sequences of the unit oligomers constituting the linked-type antisense oligomer of the present invention are not consecutive as base sequences in human dystrophin pre-mRNA. Thus, when the respective target base sequences of the unit oligomers are compared to the base sequence of human dystrophin pre-mRNA, at least one base intervenes between the target base sequences as the base sequence of human dystrophin pre-mRNA. On the other hand, the respective base sequences of the unit oligomers that do not overlapped with each other mean that the respective target base sequences of the unit oligomers do not overlapped with each other as base sequences in human dystrophin pre-mRNA. The accidental presence of a match of about 1, 2, 3, 4, 5, 6, 7, 8, 9, or 10 bases between the respective base sequences of the unit oligomers is accepted.

The linked-type antisense oligomer of the present invention may be configured such that each of the unit oligomers comprises a base sequence complementary to

(D1) any one base sequence selected from the group consisting of SEQ ID NOs: 233 to 256, 341 to 369, and 385 to 389,
(D2) a base sequence that has at least 85% identity with any one base sequence selected from the group consisting of SEQ ID NOs: 233 to 256, 341 to 369, and 385 to 389, and has a length within ±15% of the length of the any one base sequence selected,
(D3) a base sequence that hybridizes under stringent conditions to a base sequence complementary to any one base sequence selected from the group consisting of SEQ ID NOs: 233 to 256, 341 to 369, and 385 to 389, or
(D4) a partial base sequence of any one base sequence selected from the group consisting of the base sequences (D1), (D2), and (D3), and the respective base sequences of the unit oligomers are neither consecutive nor overlapped with each other.

The linked-type antisense oligomer of the present invention may comprise, as described above, a base sequence complementary to any one base sequence selected from the group consisting of (D1), (D2), (D3), and (D4). The base sequence (D2) is a mutant of the base sequence (D1), and examples thereof also include

(D2-1) a base sequence that has at least 85% identity with any one base sequence selected from the group consisting of SEQ ID NOs: 233 to 256, 341 to 369, and 385 to 389, and has a length within ±15% of the length of the any one base sequence selected,
(D2-2) a base sequence that has at least 86% identity with any one base sequence selected from the group consisting of SEQ ID NOs: 233 to 256, 341 to 369, and 385 to 389, and has a length within ±14% of the length of the any one base sequence selected,
(D2-3) a base sequence that has at least 87% identity with any one base sequence selected from the group consisting of SEQ ID NOs: 233 to 256, 341 to 369, and 385 to 389, and has a length within ±13% of the length of the any one base sequence selected,
(D2-4) a base sequence that has at least 88% identity with any one base sequence selected from the group consisting of SEQ ID NOs: 233 to 256, 341 to 369, and 385 to 389, and has a length within ±12% of the length of the any one base sequence selected,
(D2-5) a base sequence that has at least 89% identity with any one base sequence selected from the group consisting of SEQ ID NOs: 233 to 256, 341 to 369, and 385 to 389, and has a length within ±11% of the length of the any one base sequence selected,
(D2-6) a base sequence that has at least 90% identity with any one base sequence selected from the group consisting of SEQ ID NOs: 233 to 256, 341 to 369, and 385 to 389, and has a length within ±10% of the length of the any one base sequence selected,
(D2-7) a base sequence that has at least 91% identity with any one base sequence selected from the group consisting of SEQ ID NOs: 233 to 256, 341 to 369, and 385 to 389, and has a length within ±9% of the length of the any one base sequence selected,
(D2-8) a base sequence that has at least 92% identity with any one base sequence selected from the group consisting of SEQ ID NOs: 233 to 256, 341 to 369, and 385 to 389, and has a length within ±8% of the length of the any one base sequence selected,
(D2-9) a base sequence that has at least 93% identity with any one base sequence selected from the group consisting of SEQ ID NOs: 233 to 256, 341 to 369, and 385 to 389, and has a length within ±7% of the length of the any one base sequence selected,
(D2-10) a base sequence that has at least 94% identity with any one base sequence selected from the group consisting of SEQ ID NOs: 233 to 256, 341 to 369, and 385 to 389, and has a length within ±6% of the length of the any one base sequence selected,
(D2-11) a base sequence that has at least 95% identity with any one base sequence selected from the group consisting of SEQ ID NOs: 233 to 256, 341 to 369, and 385 to 389, and has a length within ±5% of the length of the any one base sequence selected,
(D2-12) a base sequence that has at least 96% identity with any one base sequence selected from the group consisting of SEQ ID NOs: 233 to 256, 341 to 369, and 385 to 389, and has a length within ±4% of the length of the any one base sequence selected,
(D2-13) a base sequence that has at least 97% identity with any one base sequence selected from the group consisting of SEQ ID NOs: 233 to 256, 341 to 369, and 385 to 389, and has a length within ±3% of the length of the any one base sequence selected,
(D2-14) a base sequence that has at least 98% identity with any one base sequence selected from the group consisting of SEQ ID NOs: 233 to 256, 341 to 369, and 385 to 389, and has a length within ±2% of the length of the any one base sequence selected,
(D2-15) a base sequence that has at least 99% identity with any one base sequence selected from the group consisting of SEQ ID NOs: 233 to 256, 341 to 369, and 385 to 389, and has a length within ±1% of the length of the any one base sequence selected, and
(D2-16) a base sequence that has at least 99.5% identity consisting of SEQ ID NOs: 233 to 256, 341 to 369, and 385 to 389, and has a length within ±0.5% of the length of the any one base sequence selected.

Thus, each of the unit oligomers constituting the linked-type antisense oligomer of the present invention may comprise a base sequence complementary to any one base sequence selected from the group consisting of the base sequences (D1), (D2-1) to (D2-16), (D3), and (D4).

The linked-type antisense oligomer of the present invention may comprise, as described above, a base sequence complementary to any one base sequence selected from the group consisting of (D1), (D2), (D3), and (D4). The base sequence (D4) relates to a partial base sequence of any one base sequence selected from the group consisting of the base sequences (D1), (D2), and (D3). In relation to (D4), the term “partial” means a partial region of consecutive bases, except for the full length, of (D1), (D2), or (D3). The partial region may be 5 to 25 bases long, 5 to 24 bases long, 5 to 23 bases long, 5 to 22 bases long, 5 to 21 bases long, 5 to 20 bases long, 5 to 19 bases long, 5 to 18 bases long, 5 to 17 bases long, 5 to 16 bases long, 5 to 15 bases long, 5 to 14 bases long, 5 to 13 bases long, 5 to 12 bases long, 7 to 25 bases long, 7 to 24 bases long, 7 to 23 bases long, 7 to 22 bases long, 7 to 21 bases long, 7 to 20 bases long, 7 to 19 bases long, 7 to 18 bases long, 7 to 17 bases long, 7 to 16 bases long, 7 to 15 bases long, 7 to 14 bases long, 7 to 13 bases long, 7 to 12 bases long, 9 to 25 bases long, 9 to 24 bases long, 9 to 23 bases long, 9 to 22 bases long, 9 to 21 bases long, 9 to 20 bases long, 9 to 19 bases long, 9 to 18 bases long, 9 to 17 bases long, 9 to 16 bases long, 9 to 15 bases long, 9 to 14 bases long, 9 to 13 bases long, 9 to 12 bases long, 10 to 25 bases long, 10 to 24 bases long, 10 to 23 bases long, 10 to 22 bases long, 10 to 21 bases long, 10 to 20 bases long, 10 to 19 bases long, 10 to 18 bases long, 10 to 17 bases long, 10 to 16 bases long, 10 to 15 bases long, 10 to 14 bases long, 10 to 13 bases long, 10 to 12 bases long, 25 bases long, 24 bases long, 23 bases long, 22 bases long, 21 bases long, 20 bases long, 19 bases long, 18 bases long, 17 bases long, 16 bases long, 15 bases long, 14 bases long, 13 bases long, 12 bases long, 11 bases long, 10 bases long, 9 bases long, 8 bases long, 7 bases long, 6 bases long, or 5 bases long, but not limited thereto. These lengths may be increased or decreased by 1, 2, or 3 bases.

The size of each unit oligomer may be 5 to 30 bases long, 5 to 29 bases long, 5 to 28 bases long, 5 to 27 bases long, 5 to 26 bases long, 5 to 25 bases long, 5 to 24 bases long, 5 to 23 bases long, 5 to 22 bases long, 5 to 21 bases long, 5 to 20 bases long, 5 to 19 bases long, 5 to 18 bases long, 5 to 17 bases long, 5 to 16 bases long, 5 to 15 bases long, 5 to 14 bases long, 5 to 13 bases long, 5 to 12 bases long, 7 to 30 bases long, 7 to 29 bases long, 7 to 28 bases long, 7 to 27 bases long, 7 to 26 bases long, 7 to 25 bases long, 7 to 24 bases long, 7 to 23 bases long, 7 to 22 bases long, 7 to 21 bases long, 7 to 20 bases long, 7 to 19 bases long, 7 to 18 bases long, 7 to 17 bases long, 7 to 16 bases long, 7 to 15 bases long, 7 to 14 bases long, 7 to 13 bases long, 7 to 12 bases long, 9 to 30 bases long, 9 to 29 bases long, 9 to 28 bases long, 9 to 27 bases long, 9 to 26 bases long, 9 to 25 bases long, 9 to 24 bases long, 9 to 23 bases long, 9 to 22 bases long, 9 to 21 bases long, 9 to 20 bases long, 9 to 19 bases long, 9 to 18 bases long, 9 to 17 bases long, 9 to 16 bases long, 9 to 15 bases long, 9 to 14 bases long, 9 to 13 bases long, 9 to 12 bases long, 10 to 30 bases long, 10 to 29 bases long, 10 to 28 bases long, 10 to 27 bases long, 10 to 26 bases long, 10 to 25 bases long, 10 to 24 bases long, 10 to 23 bases long, 10 to 22 bases long, 10 to 21 bases long, 10 to 20 bases long, 10 to 19 bases long, 10 to 18 bases long, 10 to 17 bases long, 10 to 16 bases long, 10 to 15 bases long, 10 to 14 bases long, 10 to 13 bases long, 10 to 12 bases long, 30 bases long, 29 bases long, 28 bases long, 27 bases long, 26 bases long, 25 bases long, 24 bases long, 23 bases long, 22 bases long, 21 bases long, 20 bases long, 19 bases long, 18 bases long, 17 bases long, 16 bases long, 15 bases long, 14 bases long, 13 bases long, 12 bases long, 11 bases long, 10 bases long, 9 bases long, 8 bases long, 7 bases long, 6 bases long, 5 bases long, but not limited thereto. These lengths may be increased or decreased by 1, 2, or 3 bases. The unit oligomers may have the same size or different sizes.

In a certain embodiment, the linked-type antisense oligomer of the present invention may be an antisense oligomer consisting of

(E1) any one base sequence selected from the group consisting of SEQ ID NOs: 18 to 21, 80 to 111, and 225 to 232, or
(E2) a base sequence that has at least 85% identity with any one base sequence selected from the group consisting of SEQ ID NOs: 18 to 21, 80 to 111, and 225 to 232, and has a length within ±15% of the length of the any one base sequence selected, or a pharmaceutically acceptable salt thereof, or hydrate thereof.

Herein, the base sequence (E2) is a mutant type of the base sequence (E1), and examples of such a mutant type also include

(E2-1) a base sequence that has at least 85% identity with any one base sequence selected from the group consisting of SEQ ID NOs: 18 to 21, 80 to 111, and 225 to 232, and has a length within ±15% of the length of the any one base sequence selected,
(E2-2) a base sequence that has at least 86% identity with any one base sequence selected from the group consisting of SEQ ID NOs: 18 to 21, 80 to 111, and 225 to 232, and has a length within ±14% of the length of the any one base sequence selected,
(E2-3) a base sequence that has at least 87% identity with any one base sequence selected from the group consisting of SEQ ID NOs: 18 to 21, 80 to 111, and 225 to 232, and has a length within ±13% of the length of the any one base sequence selected,
(E2-4) a base sequence that has at least 88% identity with any one base sequence selected from the group consisting of SEQ ID NOs: 18 to 21, 80 to 111, and 225 to 232, and has a length within ±12% of the length of the any one base sequence selected,
(E2-5) a base sequence that has at least 89% identity with any one base sequence selected from the group consisting of SEQ ID NOs: 18 to 21, 80 to 111, and 225 to 232, and has a length within ±11% of the length of the any one base sequence selected,
(E2-6) a base sequence that has at least 90% identity with any one base sequence selected from the group consisting of SEQ ID NOs: 18 to 21, 80 to 111, and 225 to 232, and has a length within ±10% of the length of the any one base sequence selected,
(E2-7) a base sequence that has at least 91% identity with any one base sequence selected from the group consisting of SEQ ID NOs: 18 to 21, 80 to 111, and 225 to 232, and has a length within ±9% of the length of the any one base sequence selected,
(E2-8) a base sequence that has at least 92% identity with any one base sequence selected from the group consisting of SEQ ID NOs: 18 to 21, 80 to 111, and 225 to 232, and has a length within ±8% of the length of the any one base sequence selected,
(E2-9) a base sequence that has at least 93% identity with any one base sequence selected from the group consisting of SEQ ID NOs: 18 to 21, 80 to 111, and 225 to 232, and has a length within ±7% of the length of the any one base sequence selected,
(E2-10) a base sequence that has at least 94% identity with any one base sequence selected from the group consisting of SEQ ID NOs: 18 to 21, 80 to 111, and 225 to 232, and has a length within ±6% of the length of the any one base sequence selected,
(E2-11) a base sequence that has at least 95% identity with any one base sequence selected from the group consisting of SEQ ID NOs: 18 to 21, 80 to 111, and 225 to 232, and has a length within ±5% of the length of the any one base sequence selected,
(E2-12) a base sequence that has at least 96% identity consisting of SEQ ID NOs: 18 to 21, 80 to 111, and 225 to 232, and has a length within ±4% of the length of the any one base sequence selected,
(E2-13) a base sequence that has at least 97% identity with any one base sequence selected from the group consisting of SEQ ID NOs: 18 to 21, 80 to 111, and 225 to 232, and has a length within ±3% of the length of the any one base sequence selected,
(E2-14) a base sequence that has at least 98% identity with any one base sequence selected from the group consisting of SEQ ID NOs: 18 to 21, 80 to 111, and 225 to 232, and has a length within ±2% of the length of the any one base sequence selected,
(E2-15) a base sequence that has at least 99% identity with any one base sequence selected from the group consisting of SEQ ID NOs: 18 to 21, 80 to 111, and 225 to 232, and has a length within ±1% of the length of the any one base sequence selected, and
(E2-16) a base sequence that has at least 99.5% identity with any one base sequence selected from the group consisting of SEQ ID NOs: 18 to 21, 80 to 111, and 225 to 232, and has a length within ±0.5% of the length of the any one, base sequence selected.

Thus, the linked-type antisense oligomer of the present invention may consist of any one base sequence selected from the group consisting of the base sequences (E1) and (E2-1) to (E2-16).

Preferably, the linked-type antisense oligomer of the present invention comprises any one base sequence selected from the group consisting of SEQ ID NOs: 18 to 21, 80 to 111, and 225 to 232. In a certain embodiment, the linked-type antisense oligomer of the present invention consists of any one base sequence selected from the group consisting of SEQ ID NOs: 18 to 21, 80 to 111, and 225 to 232.

Further, the antisense oligomer of the present invention may be an antisense oligomer consisting of (E′1) any one base sequence selected from the group consisting of SEQ ID NOs: 1 to 111, 113 to 177, 179, 180, 182, 183, 185 to 193, 195 to 198, 200 to 223, and 225 to 232, or

(E′2) a base sequence that has at least 85% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±15% of the length of the any one base sequence selected, or
a pharmaceutically acceptable salt thereof, or hydrate thereof.

Herein, the base sequence (E′2) is a mutant type of the base sequence (E′1), and examples of such a mutant type also include

(E′2-1) a base sequence that has at least 85% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±15% of the length of the any one base sequence selected,
(E′2-2) a base sequence that has at least 86% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±14% of the length of the any one base sequence selected,
(E′2-3) a base sequence that has at least 87% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±13% of the length of the any one base sequence selected,
(E′2-4) a base sequence that has at least 88% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±12% of the length of the any one base sequence selected,
(E′2-5) a base sequence that has at least 89% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±11% of the length of the any one base sequence selected,
(E′2-6) a base sequence that has at least 90% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±10% of the length of the any one base sequence selected,
(E′2-7) a base sequence that has at least 91% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±9% of the length of the any one base sequence selected,
(E′2-8) a base sequence that has at least 92% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±8% of the length of the any one base sequence selected,
(E′2-9) a base sequence that has at least 93% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±7% of the length of the any one base sequence selected,
(E′2-10) a base sequence that has at least 94% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±6% of the length of the any one base sequence selected,
(E′2-11) a base sequence that has at least 95% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±5% of the length of the any one base sequence selected,
(E′2-12) a base sequence that has at least 96% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±4% of the length of the any one base sequence selected,
(E′2-13) a base sequence that has at least 97% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±3% of the length of the any one base sequence selected,
(E′2-14) a base sequence that has at least 98% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±2% of the length of the any one base sequence selected,
(E′2-15) a base sequence that has at least 99% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±1% of the length of the any one base sequence selected, and
(E′2-16) a base sequence that has at least 99.5% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±0.5% of the length of the any one base sequence selected.

Thus, the antisense oligomer of the present invention may consist of any one base sequence selected from the group consisting of the base sequences (E′1) and (E′2-1) to (E′2-16).

Preferably, the antisense oligomer of the present invention comprises any one base sequence selected from the group consisting of SEQ ID NOs: 1 to 111, 113 to 177, 179, 180, 182, 183, 185 to 193, 195 to 198, 200 to 223, and 225 to 232. In a certain embodiment, the antisense oligomer of the present invention consists of any one base sequence selected from the group consisting of SEQ ID NOs: 1 to 111, 113 to 177, 179, 180, 182, 183, 185 to 193, 195 to 198, 200 to 223, and 225 to 232.

In a certain embodiment, the antisense oligomer of the present invention may be an antisense oligomer consisting of

(E″1) any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, or
(E″2) a base sequence that has at least 85% identity with any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±15% of the length of the any one base sequence selected,
or a pharmaceutically acceptable salt thereof, or hydrate thereof.

Herein, the base sequence (E″2) is a mutant type of the base sequence (E″1), and examples of such a mutant type also include

(E″2-1) a base sequence that has at least 85% identity with any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±15% of the length of the any one base sequence selected, (E″2-2) a base sequence that has at least 86% identity with any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±14% of the length of the any one base sequence selected,
(E″2-3) a base sequence that has at least 87% identity with any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±13% of the length of the any one base sequence selected,
(E″2-4) a base sequence that has at least 88% identity with any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±12% of the length of the any one base sequence selected,
(E″2-5) a base sequence that has at least 89% identity consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±11% of the length of the any one base sequence selected,
(E″2-6) a base sequence that has at least 90% identity with any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±10% of the length of the any one base sequence selected,
(E″2-7) a base sequence that has at least 91% identity with any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±9% of the length of the any one base sequence selected,
(E″2-8) a base sequence that has at least 92% identity with any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±8% of the length of the any one base sequence selected,
(E″2-9) a base sequence that has at least 93% identity with any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±7% of the length of the any one base sequence selected,
(E″2-10) a base sequence that has at least 94% identity with any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±6% of the length of the any one base sequence selected,
(E″2-11) a base sequence that has at least 95% identity with any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±5% of the length of the any one base sequence selected,
(E″2-12) a base sequence that has at least 96% identity with any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±4% of the length of the any one base sequence selected,
(E″2-13) a base sequence that has at least 97% identity with any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±3% of the length of the any one base sequence selected,
(E″2-14) a base sequence that has at least 98% identity with any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±2% of the length of the any one base sequence selected,
(E″2-15) a base sequence that has at least 99% identity with any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±1% of the length of the any one base sequence selected, and
(E″2-16) a base sequence that has at least 99.5% identity with any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and has a length within ±0.5% of the length of the any one base sequence selected.

Thus, the antisense oligomer of the present invention may consist of any one base sequence selected from the group consisting of the base sequences (E″1) and (E″2-1) to (E″2-16).

In a certain embodiment, the antisense oligomer of the present invention may be an antisense oligomer consisting of

(E′″1) any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, or
(E′″2) a base sequence that has at least 85% identity with any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±15% of the length of the any one base sequence selected,
or a pharmaceutically acceptable salt thereof, or hydrate thereof.

Herein, the base sequence (E2) is a mutant type of the base sequence (E1), and examples of such a mutant type also include

(E′″2-1) a base sequence that has at least 85% identity consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±15% of the length of the any one base sequence selected,
(E′″2-2) a base sequence that has at least 86% identity with any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±14% of the length of the any one base sequence selected,
(E′″2-3) a base sequence that has at least 87% identity with any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±13% of the length of the any one base sequence selected,
(E′″2-4) a base sequence that has at least 88% identity with any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±12% of the length of the any one base sequence selected,
(E′″2-5) a base sequence that has at least 89% identity with any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±11% of the length of the any one base sequence selected,
(E′″2-6) a base sequence that has at least 90% identity with any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±10% of the length of the any one base sequence selected,
(E′″2-7) a base sequence that has at least 91% identity with any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±9% of the length of the any one base sequence selected,
(E′″2-8) a base sequence that has at least 92% identity with any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±8% of the length of the any one base sequence selected,
(E′″2-9) a base sequence that has at least 93% identity with any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±7% of the length of the any one base sequence selected,
(E′″2-10) a base sequence that has at least 94% identity with any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±6% of the length of the any one base sequence selected,
(E′″2-11) a base sequence that has at least 95% identity with any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±5% of the length of the any one base sequence selected,
(E′″2-12) a base sequence that has at least 96% identity with any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±4% of the length of the any one base sequence selected,
(E′″2-13) a base sequence that has at least 97% identity with any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±3% of the length of the any one base sequence selected,
(E′″2-14) a base sequence that has at least 98% identity with any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±2% of the length of the any one base sequence selected,
(E′″2-15) a base sequence that has at least 99% identity with any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±1% of the length of the any one base sequence selected, and
(E′″2-16) a base sequence that has at least 99.5% identity with any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, and has a length within ±0.5% of the length of the any one base sequence selected.

Thus, the antisense oligomer of the present invention may consist of any one base sequence selected from the group consisting of the base sequences (E′″1) and (E′″2′2-1) to (E′″2-16).

Preferably, the antisense oligomer of the present invention is an antisense oligomer comprising any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, or a pharmaceutically acceptable salt thereof, or hydrate thereof. In a certain embodiment, the antisense oligomer of the present invention is an antisense oligomer consisting of any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, or a pharmaceutically acceptable salt thereof, or hydrate thereof.

More preferably, the antisense oligomer of the present invention is an antisense oligomer comprising any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, or a pharmaceutically acceptable salt thereof, or hydrate thereof. In a certain embodiment, the antisense oligomer of the present invention is an antisense oligomer consisting of any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228, or a pharmaceutically acceptable salt thereof, or hydrate thereof.

In a certain embodiment, the linked-type antisense oligomer of the present invention is an antisense oligomer comprising any one base sequence selected from the group consisting of SEQ ID NOs: 80, 82, 86, 92, 97, 98, 100, 102, 225, 228, 231, and 232, or a pharmaceutically acceptable salt thereof, or hydrate thereof. In a certain embodiment, the linked-type antisense oligomer of the present invention consists of any one base sequence selected from the group consisting of SEQ ID NOs: 80, 82, 86, 92, 97, 98, 100, 102, 225, 228, 231, and 232. Further preferably, the linked-type antisense oligomer of the present invention comprises any one base sequence selected from the group consisting of SEQ ID NOs: 102, 225, and 228. In a certain embodiment, the linked-type antisense oligomer of the present invention is an antisense oligomer consisting of any one base sequence selected from the group consisting of SEQ ID NOs: 102, 225, and 228, or a pharmaceutically acceptable salt thereof, or hydrate thereof.

The antisense oligomer of the present invention comprises a base sequence complementary to a base sequence of at least any one region selected from the group consisting of the regions R1 to R24, or a partial base sequence thereof. Any base sequence selected from the group consisting of (E1) and (E2-1) to (E2-16), the group consisting of (E′1) and (E′2-1) to (E′2-16), the group consisting of (E″1) and (E″2-1) to (E″2-16), or the group consisting of (E′″1) and (E′″2-1) to (E′″2-16) may be selected as the base sequence complementary to the base sequence of each region included in the regions R1 to R24, or a partial base sequence thereof. For example, as the base sequences complementary to the base sequences of the regions R1 to R24, or partial base sequences thereof, a base sequence complementary to the base sequence of each region, or a partial base sequence thereof may be used as to the regions R4 to R24, and a base sequence that is selected from the group consisting of (E1) and (E2-1) to (E2-16) and is included in each of the regions R1 to R3 may be used as to the regions R1 to R3.

The antisense oligomer of the present invention (including the linked-type antisense oligomer of the present invention) may be an oligonucleotide, morpholino oligomer or peptide nucleic acid (PNA) oligomer (hereinafter, also referred to as the “antisense oligonucleotide of the present invention”, the “antisense morpholino oligomer of the present invention”, or the “antisense peptide nucleic acid oligomer of the present invention”).

The antisense oligonucleotide of the present invention is an antisense oligomer composed of nucleotides as constituent units. Such nucleotides may be any of ribonucleotides, deoxyribonucleotides and modified nucleotides.

The modified nucleotide refers to one having fully or partly modified nucleobases, sugar moieties and/or phosphate bond moieties, which constitute the ribonucleotide or deoxyribonucleotide.

The nucleobase includes, for example, adenine, guanine, hypoxanthine, cytosine, thymine, uracil, and modified bases thereof. Examples of such modified bases include, but not limited to, pseudouracil, 3-methyluracil, dihydrouracil, 5-alkylcytosines (e.g., 5-methylcytosine), 5-alkyluracils (e.g., 5-ethyluracil), 5-halouracils (e.g., 5-bromouracil), 6-azapyrimidine, 6-alkylpyrimidines (e.g., 6-methyluracil), 2-thiouracil, 4-thiouracil, 4-acetylcytosine, 5-(carboxyhydroxymethyl) uracil, 5′-carboxymethylaminomethyl-2-thiouracil, 5-carboxymethylaminomethyluracil, 1-methyladenine, 1-methylhypoxanthine, 2,2-dimethylguanine, 3-methylcytosine, 2-methyladenine, 2-methylguanine, N6-methyladenine, 7-methylguanine, 5-methoxyaminomethyl-2-thiouracil, 5-methylaminomethyluracil, 5-methylcarbonylmethyluracil, 5-methyloxyuracil, 5-methyl-2-thiouracil, 2-methylthio-N6-isopentenyladenine, uracil-5-oxyacetic acid, 2-thiocytosine, purine, 2,6-diaminopurine, 2-aminopurine, isoguanine, indole, imidazole, xanthine, etc.

Modification of the sugar moiety may include, for example, modifications at the 2′-position of ribose and modifications of the other positions of the sugar. The modification at the 2′-position of ribose includes a modification of replacing the 2′-OH of ribose with —OR, —R, —R′OR, —SH, —SR, —NH₂, —NHR, —NR₂, —N₃, —CN, —F, —Cl, —Br or —I, wherein R represents an alkyl or an aryl and R′ represents an alkylene.

The modification for the other positions of the sugar includes, for example, replacement of O at the 4′ position of ribose or deoxyribose with S, bridging between 2′ and 4′ positions of the sugar, e.g., LNA (locked nucleic acid) or ENA (2′-0,4′-C-ethylene-bridged nucleic acids), but is not limited thereto.

A modification of the phosphate bond moiety includes, for example, a modification of replacing phosphodiester bond with phosphorothioate bond, phosphorodithioate bond, alkyl phosphonate bond, phosphoramidate bond or boranophosphate bond (cf., e.g., Enya et al: Bioorganic & Medicinal Chemistry, 2008, 18, 9154-9160) (cf., e.g., Japan Domestic Re-Publications of PCT Application Nos. 2006/129594 and 2006/038608).

As used herein, the alkyl is preferably a straight or branched alkyl having 1 to 6 carbon atoms. Specific examples include methyl, ethyl, n-propyl, isopropyl, n-butyl, isobutyl, sec-butyl, tert-butyl, n-pentyl, isopentyl, neopentyl, tert-pentyl, n-hexyl and isohexyl. The alkyl may optionally be substituted. Examples of such substituents are a halogen, an alkoxy, cyano and nitro. The alkyl may be substituted with 1 to 3 substituents.

As used herein, the cycloalkyl is preferably a cycloalkyl having 3 to 12 carbon atoms. Specific examples include cyclopropyl, cyclobutyl, cyclopentyl, cyclohexyl, cycloheptyl, cyclooctyl, cyclodecyl and cyclododecyl.

As used herein, the halogen includes fluorine, chlorine, bromine and iodine.

As used herein, the alkoxy is a straight or branched alkoxy having 1 to 6 carbon atoms such as methoxy, ethoxy, n-propoxy, isopropoxy, n-butoxy, isobutoxy, sec-butoxy, tert-butoxy, n-pentyloxy, isopentyloxy, n-hexyloxy, isohexyloxy, etc. Among others, an alkoxy having 1 to 3 carbon atoms is preferred.

As used herein, the aryl is preferably an aryl having 6 to 10 carbon atoms. Specific examples include phenyl, α-naphthyl and β-naphthyl. Among others, phenyl is preferred. The aryl may optionally be substituted. Examples of such substituents are an alkyl, a halogen, an alkoxy, cyano and nitro. The aryl may be substituted with one to three of such substituents.

As used herein, the alkylene is preferably a straight or branched alkylene having 1 to 6 carbon atoms. Specific examples include methylene, ethylene, trimethylene, tetramethylene, pentamethylene, hexamethylene, 2-(ethyl) trimethylene and 1-(methyl) tetramethylene.

As used herein, the acyl includes a straight or branched alkanoyl or aroyl. Examples of the alkanoyl include formyl, acetyl, 2-methylacetyl, 2,2-dimethylacetyl, propionyl, butyryl, isobutyryl, pentanoyl, 2,2-dimethylpropionyl, hexanoyl, etc. Examples of the aroyl include benzoyl, toluoyl and naphthoyl. The aroyl may optionally be substituted at substitutable positions and may be substituted with an alkyl(s).

Preferably, the antisense oligonucleotide of the present invention is the antisense oligomer of the present invention having a group represented by general formula below as a constituent unit wherein the —OH group at position 2′ of ribose is substituted with methoxy and the phosphate bond moiety is a phosphorothioate bond:

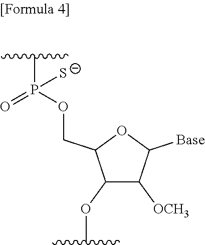

wherein Base represents a nucleobase.

The antisense oligonucleotide of the present invention may be easily synthesized using various automated synthesizer (e.g., AKTA oligopilot plus 10/100 (GE Healthcare)). Alternatively, the synthesis may also be entrusted to a third-party organization (e.g., Promega Corp. or Takara Co.), etc.

The antisense morpholino oligomer of the present invention is an antisense oligomer comprising the constituent unit represented by general formula below:

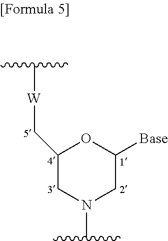

wherein Base has the same significance as defined above, and,
W represents a group shown by any one of the following groups:

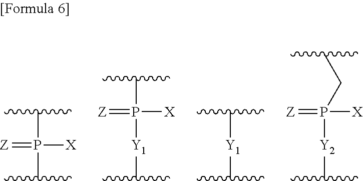

wherein X represents —CH₂R1, —O—CH₂R1, —S—CH₂R1, —NR²R3, or F;

R¹ represents H or an alkyl;

R² and R³, which may be the same or different, each represents H, an alkyl, a cycloalkyl, or an aryl;

Y₁ represents O, S, CH₂, or NR¹;

Y₂ represents O, S, or NR¹;

Z represents O or S.

Examples of morpholino monomer compounds that are used in synthesis of the antisense morpholino oligomer of the present invention include, but not limited to, the following morpholino monomer compound (A), morpholino monomer compound (C), morpholino monomer compound (T), and morpholino monomer compound (G) shown in Table 5.

TABLE 5

| Morpholino monomer | Morpholino monomer |
|---|---|
| compound | compound |
| (A) | (C) |
| 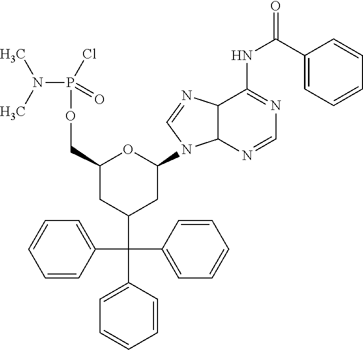 [SMILES: CN(C)P(=O)(Cl)OC[C@@H]1CC(C(c2ccccc2)(c2ccccc2)c2ccccc2)C[C@H](N2C=NC3C(NC(=O)c4ccccc4)=NC=NC32)O1] | 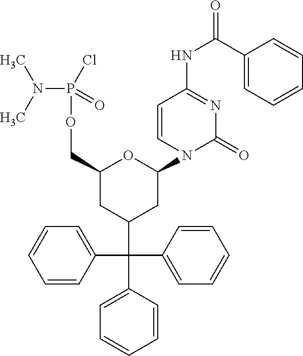 [SMILES: CN(C)P(=O)(Cl)OC[C@@H]1CC(C(c2ccccc2)(c2ccccc2)c2ccccc2)C[C@H](n2ccc(NC(=O)c3ccccc3)nc2=O)O1] |
| Morpholino monomer | Morpholino monomer |
| compound | compound |
| (T) | (G) |
| 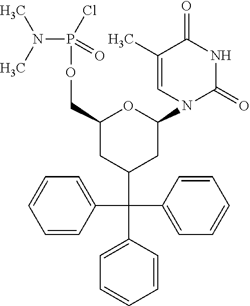 [SMILES: Cc1cn([C@H]2CC(C(c3ccccc3)(c3ccccc3)c3ccccc3)C[C@@H](COP(=O)(Cl)N(C)C)O2)c(=O)[nH]c1=O] | 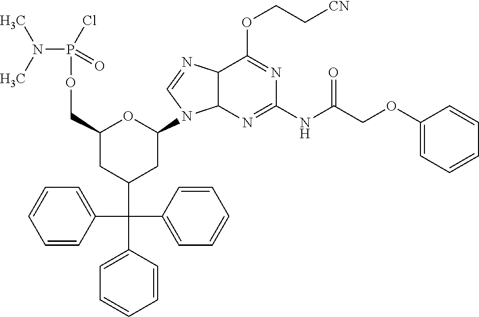 [SMILES: CN(C)P(=O)(Cl)OC[C@@H]1CC(C(c2ccccc2)(c2ccccc2)c2ccccc2)C[C@H](N2C=NC3C(OCCC#N)=NC(NC(=O)COc4ccccc4)=NC32)O1] |

In the present invention, preferably, the morpholino oligomer is an oligomer having a group represented by general formula below as a constituent unit (phosphorodiamidate morpholino oligomer (hereinafter referred to as “PMO”)).

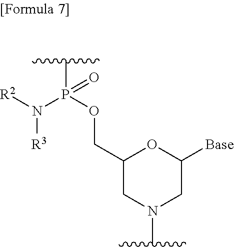

wherein Base, R² and R³ have the same significance as defined above.

The morpholino oligomer may be produced by the procedure described in, e.g., WO 1991/009033 or WO 2009/064471. In particular, PMO can be produced by the procedure described in WO 2009/064471 or WO2013/100190.

The antisense peptide nucleic acid oligomer of the present invention is an antisense oligomer having a group represented by general formula below as a constituent unit:

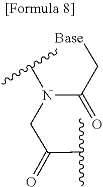

wherein Base has the same significance as defined above.

The peptide nucleic acid oligomer can be produced in accordance with, e.g., the following literatures:
1) P. E. Nielsen, M. Egholm, R. H. Berg, O. Buchardt, Science, 254, 1497 (1991)
2) M. Egholm, O. Buchardt, P. E. Nielsen, R. H. Berg, JACS, 114, 1895 (1992)
3) K. L. Dueholm, M. Egholm, C. Behrens, L. Christensen, H. F. Hansen, T. Vulpius, K. H. Petersen, R. H. Berg, P. E. Nielsen, O. Buchardt, J. Org. Chem., 59, 5767 (1994)
4) L. Christensen, R. Fitzpatrick, B. Gildea, K. H. Petersen, H. F. Hansen, T. Koch, M. Egholm, O. Buchardt, P. E. Nielsen, J. Coull, R. H. Berg, J. Pept. Sci., 1, 175 (1995)
5) T. Koch, H. F. Hansen, P. Andersen, T. Larsen, H. G. Batz, K. Otteson, H. Orum, J. Pept. Res., 49, 80 (1997)

The antisense oligomer of the present invention (including the linked-type antisense oligomer of the present invention) may be in the form of a pharmaceutically acceptable salt thereof, in the form of a hydrate thereof, or in the form of a hydrate of the pharmaceutically acceptable salt.

Examples of the pharmaceutically acceptable salt of the antisense oligomer of the present invention are alkali metal salts such as salts of sodium, potassium and lithium; alkaline earth metal salts such as salts of calcium and magnesium; metal salts such as salts of aluminum, iron, zinc, copper, nickel, cobalt, etc.; ammonium salts; organic amine salts such as salts of t-octylamine, dibenzylamine, morpholine, glucosamine, phenylglycine alkyl ester, ethylenediamine, N-methylglucamine, guanidine, diethylamine, triethylamine, dicyclohexylamine, N,N′-dibenzylethylenediamine, chloroprocaine, procaine, diethanolamine, N-benzyl-phenethylamine, piperazine, tetramethylammonium, tris(hydroxymethyl)aminomethane; hydrohalide salts such as salts of hydrofluorides, hydrochlorides, hydrobromides and hydroiodides; inorganic acid salts such as nitrates, perchlorates, sulfates, phosphates, etc.; lower alkane sulfonates such as methanesulfonates, trifluoromethanesulfonates and ethanesulfonates; arylsulfonates such as benzenesulfonates and p-toluenesulfonates; organic acid salts such as acetates, malates, fumarates, succinates, citrates, tartarates, oxalates, maleates, etc.; and, amino acid salts such as salts of glycine, lysine, arginine, ornithine, glutamic acid and aspartic acid. These salts may be produced by known methods. Alternatively, the antisense oligomer of the present invention may be in the form of a hydrate thereof.

### 2. Suppressor Antisense Oligomer

Another embodiment of the present invention provides a suppressor antisense oligomer which suppresses single exon skipping (hereinafter, referred to as “single skipping”). The suppressor antisense oligomer can suppress single skipping and thereby enhance an effect of multi-exon skipping by an antisense oligomer. The single skipping relates to skipping of only one exon, not simultaneous skipping of a plurality of exons.

Specifically, the present invention provides a suppressor antisense oligomer or a pharmaceutically acceptable salt thereof, or hydrate thereof which suppresses single skipping of any one exon selected from the group consisting of the 45th exon to the 55th exon in human dystrophin pre-mRNA. Hereinafter, the suppressor antisense oligomer and the pharmaceutically acceptable salt thereof and the hydrate of the antisense oligomer or the salt are also collectively referred to as the “suppressor antisense oligomer of the present invention”. The suppressor antisense oligomer of the present invention may refer to each of the suppressor antisense oligomer or the pharmaceutically acceptable salt thereof, or hydrate thereof.

The suppressor antisense oligomer of the present invention suppresses single skipping by targeting the site of a splicing silencer sequence, a branch site sequence, or a splice site sequence in human dystrophin pre-mRNA and inhibiting splicing. The suppressor antisense oligomer of the present invention reduces the efficiency of single skipping of an intended exon as compared to a control.

For example, the suppressor antisense oligomer that suppresses single skipping of any one exon selected from the group consisting of the 45th exon to the 55th exon targets a splicing silencer sequence of any one of the 44th to 56th exons, or a splicing silencer sequence, a branch site sequence, or a splice site sequence of any one of the 44th to 55th introns.

As used herein, the splicing silencer sequence is a base sequence element that functions to suppress recognition of an exon in pre-mRNA. Examples of the splicing silencer sequence include recognition sequences of proteins or protein complexes, such as heterogeneous nuclear ribonucleoprotein A1 (hnRNP A1), hnRNP A2/B1, DAZAP1, hnRNP I, Fox-1, Fox-2, hnRNP H1, hnRNP H2, hnRNP H3, hnRNP L, Sam68, and SRp40.

As used herein, the splice site is the boundary between an exon and an intron. The splice site can be predicted from the 5′ end of the intron starting at GU, AU, or the like, and the 3′ end of the intron ending at AG, AC, or the like.

As used herein, the branch site is a sequence within an intron that initiates an attack on a 5′ splice site during splicing reaction and forms a covalent bond. The branch site sequence refers to a base sequence in an intron containing the branch site.

Examples of the splicing silencer sequence, the branch site sequence, or the splice site sequence which is the target (hereinafter, referred to as a “suppressor sequence”) of the suppressor antisense oligomer of the present invention include base sequences given below (SEQ ID NOs: 370 to 384 and 390). The positions of the suppressor sequence, the branch site sequence, and the splice site sequence can be detected using software such as SpliceAid (Francesco Piva et al., Bioinformatics, 25 (9), 1211-1213, 2009), SpliceAid-2 (Francesco Piva et al., Human Mutation, 33 (1), 81-85, 2012), SpliceAid-F (Matteo Giulietti et al., Nucleic Acids Res., 41, D125-D131, 2012), ATtRACT-a (Girolamo Giudice et al., Database (Oxford), baw035, 2016), SROOGLE (Schraga Schwartz et al., Nucleic Acids Res., 37, W189-W192, 2009), Reg RNA (Hsi-Yuan Huang et al., Nucleic Acids Res., 34, W429-W434, 2006), Reg RNA 2.0 (Tzu-Hao Chang et al., BMC bioinformatics, 14 (Suppl 2), S4, 2013), Human Splicing Finder (Francois-Olivier Desmet et al., Nucleic Acids Res., 37, 9, e67, 2009), or SVM-BPfinder (Andre Corvelo et al., PLoS Comput Biol., 6, 11, e1001016, 2010).

TABLE 6

| SEQ ID | Name of protein or protein complex | Base |
|---|---|---|
| NO | or sequence site to be bound | sequence |
| 370 | hnRNP A1 | UAGGA |
| 371 | | UAGGAU |
| 372 | | UAGGAA |
| 373 | | UAGGCA |
| 374 | | UAGGCU |
| 375 | | UAGGGA |
| 376 | | UAGGGC |
| 377 | | UAGGGU |
| 378 | | GGUAGGGC |
| 379 | | AGAAC |
| 380 | Fox-1 | AGCAUG |
| 381 | | UGACUG |
| 382 | | UGCAUG |
| 383 | Branch site | yUnAy |
| 384 | Splice site | GU |
| 390 | | AG |

y: A or U,
n: A, G, C, U, or T

Herein, specific examples of the splicing silencer sequence include SEQ ID NOs: 370 to 382. Examples of mutants of SEQ ID NOs: 370 to 382 also include

(F1) a base sequence that has at least 85% identity with any one base sequence selected from the group consisting of SEQ ID NOs: 370 to 382, and has a length within ±15% of the length of the any one base sequence selected,
(F2) a base sequence that has at least 86% identity with any one base sequence selected from the group consisting of SEQ ID NOs: 370 to 382, and has a length within ±14% of the length of the any one base sequence selected,
(F3) a base sequence that has at least 87% identity with any one base sequence selected from the group consisting of SEQ ID NOs: 370 to 382, and has a length within ±13% of the length of the any one base sequence selected,
(F4) a base sequence that has at least 88% identity with any one base sequence selected from the group consisting of SEQ ID NOs: 370 to 382, and has a length within ±12% of the length of the any one base sequence selected,
(F5) a base sequence that has at least 89% identity with any one base sequence selected from the group consisting of SEQ ID NOs: 370 to 382, and has a length within ±11% of the length of the any one base sequence selected,
(F6) a base sequence that has at least 90% identity with any one base sequence selected from the group consisting of SEQ ID NOs: 370 to 382, and has a length within ±10% of the length of the any one base sequence selected,
(F7) a base sequence that has at least 91% identity with any one base sequence selected from the group consisting of SEQ ID NOs: 370 to 382, and has a length within ±9% of the length of the any one base sequence selected,
(F8) a base sequence that has at least 92% identity with any one base sequence selected from the group consisting of SEQ ID NOs: 370 to 382, and has a length within ±8% of the length of the any one base sequence selected,
(F9) a base sequence that has at least 93% identity with any one base sequence selected from the group consisting of SEQ ID NOs: 370 to 382, and has a length within ±7% of the length of the any one base sequence selected,
(F10) a base sequence that has at least 94% identity with any one base sequence selected from the group consisting of SEQ ID NOs: 370 to 382, and has a length within ±6% of the length of the any one base sequence selected,
(F11) a base sequence that has at least 95% identity with any one base sequence selected from the group consisting of SEQ ID NOs: 370 to 382, and has a length within ±5% of the length of the any one base sequence selected,
(F12) a base sequence that has at least 96% identity with any one base sequence selected from the group consisting of SEQ ID NOs: 370 to 382, and has a length within ±4% of the length of the any one base sequence selected,
(F13) a base sequence that has at least 97% identity with any one base sequence selected from the group consisting of SEQ ID NOs: 370 to 382, and has a length within ±3% of the length of the any one base sequence selected,
(F14) a base sequence that has at least 98% identity with any one base sequence selected from the group consisting of SEQ ID NOs: 370 to 382, and has a length within ±2% of the length of the any one base sequence selected,
(F15) a base sequence that has at least 99% identity with any one base sequence selected from the group consisting of SEQ ID NOs: 370 to 382, and has a length within ±1% of the length of the any one base sequence selected, and
(F16) a base sequence that has at least 99.5% identity with any one base sequence selected from the group consisting of SEQ ID NOs: 370 to 382, and has a length within ±0.5% of the length of the any one base sequence selected.

In a certain embodiment of the present invention, the splicing silencer sequence may be a recognition sequence of heterogeneous nuclear ribonucleoprotein A1 (hnRNP A1). Examples of the recognition sequence of human hnRNP A1 are shown in SEQ ID NOs: 370 to 379.

Herein, specific examples of the splice site sequence include SEQ ID NOs: 384 and 390. Examples of mutants of SEQ ID NOs: 384 and 390 also include

(G1) a base sequence that has at least 80% identity with the base sequence of SEQ ID NO: 384 or 390, and has a length within ±20% of the length of the any one base sequence selected.

Herein, specific examples of the branch site sequence include SEQ ID NO: 383. Examples of a mutant of SEQ ID NO: 383 also include

(H1) a base sequence that has at least 80% identity with the base sequence of SEQ ID NO: 383, and has a length within ±20% of the length of the any one base sequence selected.

As for a mutant type of the suppressor sequence, the following target regions are also included in the target base sequence of the suppressor antisense oligomer of the present invention:

(I1) a base sequence that has at least 85% identity with any one base sequence selected from the group consisting of SEQ ID NOs: 370 to 384 and 390, and has a length within ±15% of the length of the any one base sequence selected,
(I2) a base sequence that has at least 86% identity with any one base sequence selected from the group consisting of SEQ ID NOs: 370 to 384 and 390, and has a length within ±14% of the length of the any one base sequence selected,
(I3) a base sequence that has at least 87% identity with any one base sequence selected from the group consisting of SEQ ID NOs: 370 to 384 and 390, and has a length within ±13% of the length of the any one base sequence selected,
(I4) a base sequence that has at least 88% identity with any one base sequence selected from the group consisting of SEQ ID NOs: 370 to 384 and 390, and has a length within ±12% of the length of the any one base sequence selected,
(I5) a base sequence that has at least 89% identity with any one base sequence selected from the group consisting of SEQ ID NOs: 370 to 384 and 390, and has a length within ±11% of the length of the any one base sequence selected,
(I6) a base sequence that has at least 90% identity with any one base sequence selected from the group consisting of SEQ ID NOs: 370 to 384 and 390, and has a length within ±10% of the length of the any one base sequence selected,
(I7) a base sequence that has at least 91% identity with any one base sequence selected from the group consisting of SEQ ID NOs: 370 to 384 and 390, and has a length within ±9% of the length of the any one base sequence selected,
(I8) a base sequence that has at least 92% identity with any one base sequence selected from the group consisting of SEQ ID NOs: 370 to 384 and 390, and has a length within ±8% of the length of the any one base sequence selected,
(I9) a base sequence that has at least 93% identity with any one base sequence selected from the group consisting of SEQ ID NOs: 370 to 384 and 390, and has a length within ±7% of the length of the any one base sequence selected,
(I10) a base sequence that has at least 94% identity with any one base sequence selected from the group consisting of SEQ ID NOs: 370 to 384 and 390, and has a length within ±6% of the length of the any one base sequence selected,
(I11) a base sequence that has at least 95% identity with any one base sequence selected from the group consisting of SEQ ID NOs: 370 to 384 and 390, and has a length within ±5% of the length of the any one base sequence selected,
(I12) a base sequence that has at least 96% identity with any one base sequence selected from the group consisting of SEQ ID NOs: 370 to 384 and 390, and has a length within ±4% of the length of the any one base sequence selected,
(I13) a base sequence that has at least 97% identity with any one base sequence selected from the group consisting of SEQ ID NOs: 370 to 384 and 390, and has a length within ±3% of the length of the any one base sequence selected,
(I14) a base sequence that has at least 98% identity with any one base sequence selected from the group consisting of SEQ ID NOs: 370 to 384 and 390, and has a length within ±2% of the length of the any one base sequence selected,
(I15) a base sequence that has at least 99% identity with any one base sequence selected from the group consisting of SEQ ID NOs: 370 to 384 and 390, and has a length within ±1% of the length of the any one base sequence selected, and
(I16) a base sequence that has at least 99.5% identity with any one base sequence selected from the group consisting of SEQ ID NOs: 370 to 384 and 390, and has a length within ±0.5% of the length of the any one base sequence selected.

In one embodiment, the suppressor antisense oligomer of the present invention comprises a base sequence complementary to any one base sequence of a base sequence selected from the group consisting of the base sequences represented by SEQ ID NOs: 370 to 384 and 390 and the mutant type base sequences shown in (I1) to (I16) above, or a partial base sequence thereof. In another embodiment, the suppressor antisense oligomer of the present invention consists of a base sequence complementary to any one base sequence of a base sequence selected from the group consisting of the base sequences represented by SEQ ID NOs: 370 to 384 and 390, or a partial base sequence thereof.

Herein, the term “partial” is as defined in the section about the antisense oligomer of the present invention.

As a further embodiment, the suppressor antisense oligomer of the present invention is a suppressor antisense oligomer which consists of

(J1) any one base sequence selected from the group consisting of SEQ ID NOs: 257 to 275, or
(J2) a base sequence that has at least 85% identity with any one base sequence selected from the group consisting of SEQ ID NOs: 257 to 275, and has a length within ±15% of the length of the any one base sequence selected, or a pharmaceutically acceptable salt thereof, or hydrate thereof.

Herein, the base sequence (J2) is a mutant type of the base sequence (J1). Such a mutant type also includes

(J2-1) a base sequence that has at least 85% identity with any one base sequence selected from the group consisting of SEQ ID NOs: 257 to 275, and has a length within ±15% of the length of the any one base sequence selected,
(J2-2) a base sequence that has at least 86% identity consisting of SEQ ID NOs: 257 to 275, and has a length within ±14% of the length of the any one base sequence selected,
(J2-3) a base sequence that has at least 87% identity with any one base sequence selected from the group consisting of SEQ ID NOs: 257 to 275, and has a length within ±13% of the length of the any one base sequence selected,
(J2-4) a base sequence that has at least 88% identity with any one base sequence selected from the group consisting of SEQ ID NOs: 257 to 275, and has a length within ±12% of the length of the any one base sequence selected,
(J2-5) a base sequence that has at least 89% identity with any one base sequence selected from the group consisting of SEQ ID NOs: 257 to 275, and has a length within ±11% of the length of the any one base sequence selected,
(J2-6) a base sequence that has at least 90% identity with any one base sequence selected from the group consisting of SEQ ID NOs: 257 to 275, and has a length within ±10% of the length of the any one base sequence selected,
(J2-7) a base sequence that has at least 91% identity with any one base sequence selected from the group consisting of SEQ ID NOs: 257 to 275, and has a length within ±9% of the length of the any one base sequence selected,
(J2-8) a base sequence that has at least 92% identity with any one base sequence selected from the group consisting of SEQ ID NOs: 257 to 275, and has a length within ±8% of the length of the any one base sequence selected,
(J2-9) a base sequence that has at least 93% identity with any one base sequence selected from the group consisting of SEQ ID NOs: 257 to 275, and has a length within ±7% of the length of the any one base sequence selected,
(J2-10) a base sequence that has at least 94% identity with any one base sequence selected from the group consisting of SEQ ID NOs: 257 to 275, and has a length within ±6% of the length of the any one base sequence selected,
(J2-11) a base sequence that has at least 95% identity with any one base sequence selected from the group consisting of SEQ ID NOs: 257 to 275, and has a length within ±5% of the length of the any one base sequence selected,
(J2-12) a base sequence that has at least 96% identity with any one base sequence selected from the group consisting of SEQ ID NOs: 257 to 275, and has a length within ±4% of the length of the any one base sequence selected,
(J2-13) a base sequence that has at least 97% identity with any one base sequence selected from the group consisting of SEQ ID NOs: 257 to 275, and has a length within ±3% of the length of the any one base sequence selected,
(J2-14) a base sequence that has at least 98% identity with any one base sequence selected from the group consisting of SEQ ID NOs: 257 to 275, and has a length within ±2% of the length of the any one base sequence selected,
(J2-15) a base sequence that has at least 99% identity with any one base sequence selected from the group consisting of SEQ ID NOs: 257 to 275, and has a length within ±1% of the length of the any one base sequence selected, and
(J2-16) a base sequence that has at least 99.5% identity with any one base sequence selected from the group consisting of SEQ ID NOs: 257 to 275, and has a length within ±0.5% of the length of the any one base sequence selected.

Thus, a further embodiment of the suppressor antisense oligomer of the present invention is a suppressor antisense oligomer consisting of any one base sequence selected from the group consisting of (J1) and

(J2-1) to (J2-16) or a pharmaceutically acceptable salt thereof, or hydrate thereof.

A further embodiment of the suppressor antisense oligomer of the present invention is a suppressor antisense oligomer consisting of

(J′1) any one base sequence selected from the group consisting of SEQ ID NO: 260, 261, and 263, or
(J′2) a base sequence that has at least 85% identity with any one base sequence selected from the group consisting of SEQ ID NOs: 260, 261, and 263, and has a length within ±15% of the length of the any one base sequence selected, or
a pharmaceutically acceptable salt thereof, or hydrate thereof.

Herein, the base sequence (J′2) is a mutant type of the base sequence (J′1). Such a mutant type also includes

(J′2-1) a base sequence that has at least 85% identity with any one base sequence selected from the group consisting of SEQ ID NOs: 260, 261, and 263, and has a length within ±15% of the length of the any one base sequence selected,
(J′2-2) a base sequence that has at least 86% identity with any one base sequence selected from the group consisting of SEQ ID NOs: 260, 261, and 263, and has a length within ±14% of the length of the any one base sequence selected,
(J′2-3) a base sequence that has at least 87% identity with any one base sequence selected from the group consisting of SEQ ID NOs: 260, 261, and 263, and has a length within ±13% of the length of the any one base sequence selected,
(J′2-4) a base sequence that has at least 88% identity with any one base sequence selected from the group consisting of SEQ ID NOs: 260, 261, and 263, and has a length within ±12% of the length of the any one base sequence selected,
(J′2-5) a base sequence that has at least 89% identity with any one base sequence selected from the group consisting of SEQ ID NOs: 260, 261, and 263, and has a length within ±11% of the length of the any one base sequence selected,
(J′2-6) a base sequence that has at least 90% identity with any one base sequence selected from the group consisting of SEQ ID NOs: 260, 261, and 263, and has a length within ±10% of the length of the any one base sequence selected,
(J′2-7) a base sequence that has at least 91% identity with any one base sequence selected from the group consisting of SEQ ID NOs: 260, 261, and 263, and has a length within ±9% of the length of the any one base sequence selected,
(J′2-8) a base sequence that has at least 92% identity consisting of SEQ ID NOs: 260, 261, and 263, and has a length within ±8% of the length of the any one base sequence selected,
(J′2-9) a base sequence that has at least 93% identity with any one base sequence selected from the group consisting of SEQ ID NOs: 260, 261, and 263, and has a length within ±7% of the length of the any one base sequence selected,
(J′2-10) a base sequence that has at least 94% identity with any one base sequence selected from the group consisting of SEQ ID NOs: 260, 261, and 263, and has a length within ±6% of the length of the any one base sequence selected,
(J′2-11) a base sequence that has at least 95% identity with any one base sequence selected from the group consisting of SEQ ID NOs: 260, 261, and 263, and has a length within ±5% of the length of the any one base sequence selected,
(J′2-12) a base sequence that has at least 96% identity with any one base sequence selected from the group consisting of SEQ ID NOs: 260, 261, and 263, and has a length within ±4% of the length of the any one base sequence selected,
(J′2-13) a base sequence that has at least 97% identity with any one base sequence selected from the group consisting of SEQ ID NOs: 260, 261, and 263, and has a length within ±3% of the length of the any one base sequence selected,
(J′2-14) a base sequence that has at least 98% identity with any one base sequence selected from the group consisting of SEQ ID NOs: 260, 261, and 263, and has a length within ±2% of the length of the any one base sequence selected,
(J′2-15) a base sequence that has at least 99% identity with any one base sequence selected from the group consisting of SEQ ID NOs: 260, 261, and 263, and has a length within ±1% of the length of the any one base sequence selected, and
(J′2-16) a base sequence that has at least 99.5% identity with any one base sequence selected from the group consisting of SEQ ID NOs: 260, 261, and 263, and has a length within ±0.5% of the length of the any one base sequence selected.

Thus, a further embodiment of the suppressor antisense oligomer of the present invention is a suppressor antisense oligomer consisting of any one base sequence selected from the group consisting of (J′1) and (J′2-1) to (J′2-16) or a pharmaceutically acceptable salt thereof, or hydrate thereof.

The suppressor antisense oligomer of the present invention enhances a multi-skipping effect under physiological conditions. The term “under physiological conditions” refers to conditions set to mimic the in vivo environment in terms of pH, salt composition and temperature. The conditions are, for example, 25 to 40° C., preferably 37° C., pH 5 to 8, preferably pH 7.4 and 150 mM of sodium chloride concentration.

Whether the suppressor antisense oligomer of the present invention enhances a multi-exon skipping effect or not can be confirmed by providing (i) an experimental system for multi-exon skipping using only the antisense oligomer of the present invention alone and (ii) an experimental system for multi-exon skipping using the antisense oligomer of the present invention and the suppressor antisense oligomer of the present invention such that the other conditions are the same therebetween, and observing the difference between a multi-exon skipping effect obtained in the experimental system (ii) and a multi-exon skipping effect obtained in the experimental system (i).

The multi-skipping effect is measured as mentioned in the section “1. Antisense oligomer” about the antisense oligomer of the present invention.

The suppressor antisense oligomer of the present invention is 10 to 60 bases long and may be 10 to 55 bases long, 10 to 50 bases long, 10 to 45 bases long, 10 to 40 bases long, 10 to 35 bases long, 10 to 30 bases long, 10 to 25 bases long, 15 to 60 bases long, 15 to 55 bases long, 15 to 50 bases long, 15 to 45 bases long, 15 to 40 bases long, 15 to 35 bases long, 15 to 30 bases long, 15 to 25 bases long, 16 to 60 bases long, 16 to 55 bases long, 16 to 50 bases long, 16 to 45 bases long, 16 to 40 bases long, 16 to 35 bases long, 16 to 30 bases long, 16 to 25 bases long, 17 to 60 bases long, 17 to 55 bases long, 17 to 50 bases long, 17 to 45 bases long, 17 to 40 bases long, 17 to 35 bases long, 17 to 30 bases long, 17 to 25 bases long, 18 to 60 bases long, 18 to 55 bases long, 18 to 50 bases long, 18 to 45 bases long, 18 to 40 bases long, 18 to 35 bases long, 18 to 30 bases long, 18 to 25 bases long, 19 to 60 bases long, 19 to 55 bases long, 19 to 50 bases long, 19 to 45 bases long, 19 to 40 bases long, 19 to 35 bases long, 19 to 30 bases long, 19 to 25 bases long, 20 to 60 bases long, 20 to 55 bases long, 20 to 50 bases long, 20 to 45 bases long, 20 to 40 bases long, 20 to 35 bases long, 20 to 30 bases long, 20 to 25 bases long, 15 to 30 bases long, 15 to 29 bases long, 15 to 28 bases long, 15 to 27 bases long, 15 to 26 bases long, 15 to 25 bases long, 15 to 24 bases long, 15 to 23 bases long, 15 to 22 bases long, 15 to 21 bases long, 15 to 20 bases long, 15 to 19 bases long, 15 to 18 bases long, 16 to 30 bases long, 16 to 29 bases long, 16 to 28 bases long, 16 to 27 bases long, 16 to 26 bases long, 16 to 25 bases long, 16 to 24 bases long, 16 to 23 bases long, 16 to 22 bases long, 16 to 21 bases long, 16 to 20 bases long, 16 to 19 bases long, 16 to 18 bases long, 17 to 30 bases long, 17 to 29 bases long, 17 to 28 bases long, 17 to 27 bases long, 17 to 26 bases long, 17 to 25 bases long, 17 to 24 bases long, 17 to 23 bases long, 17 to 22 bases long, 17 to 21 bases long, 17 to 20 bases long, 17 to 19 bases long, 17 to 18 bases long, 18 to 30 bases long, 18 to 29 bases long, 18 to 28 bases long, 18 to 27 bases long, 18 to 26 bases long, 18 to 25 bases long, 18 to 24 bases long, 18 to 23 bases long, 18 to 22 bases long, 18 to 21 bases long, 18 to 20 bases long, 18 to 19 bases long, 19 to 30 bases long, 19 to 29 bases long, 19 to 28 bases long, 19 to 27 bases long, 19 to 26 bases long, 19 to 25 bases long, 19 to 24 bases long, 19 to 23 bases long, 19 to 22 bases long, 19 to 21 bases long, 19 to 20 bases long, 20 to 30 bases long, 20 to 29 bases long, 20 to 28 bases long, 20 to 27 bases long, 20 to 26 bases long, 20 to 25 bases long, 20 to 24 bases long, 20 to 23 bases long, 20 to 22 bases long, 20 to 21 bases long, 60 bases long, 59 bases long, 58 bases long, 57 bases long, 56 bases long, 55 bases long, 54 bases long, 53 bases long, 52 bases long, 51 bases long, 50 bases long, 49 bases long, 48 bases long, 47 bases long, 46 bases long, 45 bases long, 44 bases long, 43 bases long, 42 bases long, 41 bases long, 40 bases long, 39 bases long, 38 bases long, 37 bases long, 36 bases long, 35 bases long, 34 bases long, 33 bases long, 32 bases long, 31 bases long, 30 bases long, 29 bases long, 28 bases long, 27 bases long, 26 bases long, 25 bases long, 24 bases long, 23 bases long, 22 bases long, 21 bases long, 20 bases long, 19 bases long, 18 bases long, 17 bases long, 16 bases long, 15 bases long, 14 bases long, 13 bases long, 12 bases long, 11 bases long, or 10 bases long, but not limited thereto. These lengths may be increased or decreased by 1, 2, or 3 bases.

The suppressor antisense oligomer of the present invention may be a linked-type antisense oligomer configured to comprise a plurality of unit oligomers linked to each other, or a pharmaceutically acceptable salt thereof, or hydrate thereof. The unit oligomers may be linked via a linker that does not contribute to hybridization, or may be linked directly without the mediation of a linker, as mentioned above. When the suppressor antisense oligomer of the present invention is a linked type, the respective base sequences of the unit oligomers are neither consecutive nor overlapped with each other.

The suppressor antisense oligomer of the present invention may be an oligonucleotide, morpholino oligomer, or peptide nucleic acid (PNA) oligomer.

When the suppressor antisense oligomer of the present invention is an oligonucleotide (hereinafter, referred to as the “suppressor antisense oligonucleotide of the present invention”), the suppressor antisense oligonucleotide of the present invention is a suppressor antisense oligomer composed of nucleotides as constituent units. Such nucleotides may be any of ribonucleotides, deoxyribonucleotides and modified nucleotides.

The modified nucleotide is as mentioned above.

Preferably, the suppressor antisense oligonucleotide of the present invention is the suppressor antisense oligomer of the present invention having a group represented by general formula below as a constituent unit wherein the —OH group at position 2′ of ribose is substituted with methoxy and the phosphate bond moiety is a phosphorothioate bond:

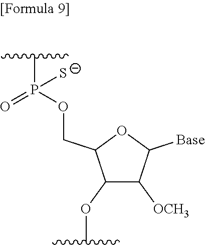

wherein Base has the same significance as defined above.

The suppressor antisense oligonucleotide of the present invention may be easily synthesized using various automated synthesizer (e.g., AKTA oligopilot plus 10/100 (GE Healthcare)). Alternatively, the synthesis may also be entrusted to a third-party organization (e.g., Promega Corp. or Takara Co.), etc.

When the suppressor antisense oligomer of the present invention is a morpholino oligomer (hereinafter, referred to as the “suppressor antisense morpholino oligomer of the present invention”), the suppressor antisense morpholino oligomer of the present invention is a suppressor antisense oligomer having a group represented by general formula below as a constituent unit:

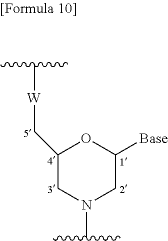

wherein Base and W have the same significance as defined above.

Examples of morpholino monomer compounds that are used in synthesis of the suppressor antisense morpholino oligomer of the present invention include, but not limited to, the following morpholino monomer compound (A), morpholino monomer compound (C), morpholino monomer compound (T), and morpholino monomer compound (G) shown in Table 7.

TABLE 7

| Morpholino monomer | Morpholino monomer |
|---|---|
| compound | compound |
| (A) | (C) |
| 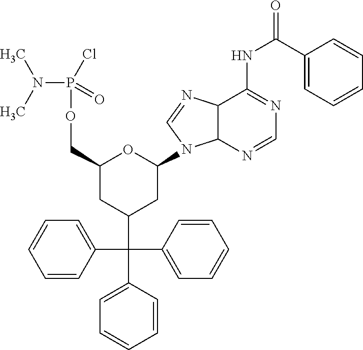 [SMILES: CN(C)P(=O)(Cl)OC[C@@H]1CC(C(c2ccccc2)(c2ccccc2)c2ccccc2)C[C@H](N2C=NC3C(NC(=O)c4ccccc4)=NC=NC32)O1] | 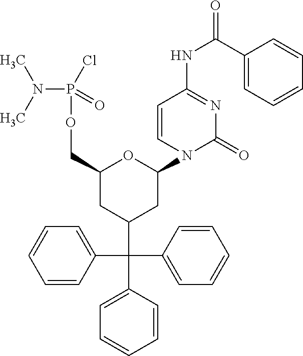 [SMILES: CN(C)P(=O)(Cl)OC[C@@H]1CC(C(c2ccccc2)(c2ccccc2)c2ccccc2)C[C@H](n2ccc(NC(=O)c3ccccc3)nc2=O)O1] |
| Morpholino monomer | Morpholino monomer |
| compound | compound |
| (T) | (G) |
| 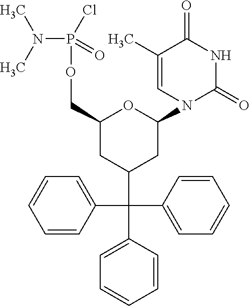 [SMILES: Cc1cn([C@H]2CC(C(c3ccccc3)(c3ccccc3)c3ccccc3)C[C@@H](COP(=O)(Cl)N(C)C)O2)c(=O)[nH]c1=O] | 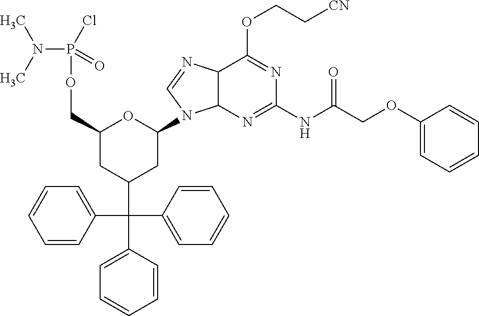 [SMILES: CN(C)P(=O)(Cl)OC[C@@H]1CC(C(c2ccccc2)(c2ccccc2)c2ccccc2)C[C@H](N2C=NC3C(OCCC#N)=NC(NC(=O)COc4ccccc4)=NC32)O1] |

In the suppressor antisense morpholino oligomer of the present invention, preferably the morpholino oligomer is PMO.

The morpholino oligomer may be produced by the procedure described in, e.g., WO 1991/009033 or WO 2009/064471. In particular, PMO can be produced by the procedure described in WO 2009/064471 or WO2013/100190.

When the suppressor antisense oligomer of the present invention is a peptide nucleic acid oligomer (hereinafter, referred to as the “suppressor antisense peptide nucleic acid oligomer of the present invention”), the suppressor antisense peptide nucleic acid oligomer of the present invention is a suppressor antisense oligomer having a group represented by general formula (I) below as a constituent unit:

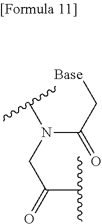

wherein Base has the same significance as defined above.

The peptide nucleic acid oligomer can be produced as mentioned above.

The suppressor antisense oligomer of the present invention may be in the form of a pharmaceutically acceptable salt thereof, in the form of a hydrate thereof, or in the form of a hydrate of the pharmaceutically acceptable salt.

Examples of the pharmaceutically acceptable salt of the suppressor antisense oligomer of the present invention are alkali metal salts such as salts of sodium, potassium and lithium; alkaline earth metal salts such as salts of calcium and magnesium; metal salts such as salts of aluminum, iron, zinc, copper, nickel, cobalt, etc.; ammonium salts; organic amine salts such as salts of t-octylamine, dibenzylamine, morpholine, glucosamine, phenylglycine alkyl ester, ethylenediamine, N-methylglucamine, guanidine, diethylamine, triethylamine, dicyclohexylamine, N,N′-dibenzylethylenediamine, chloroprocaine, procaine, diethanolamine, N-benzyl-phenethylamine, piperazine, tetramethylammonium, tris(hydroxymethyl)aminomethane; hydrohalide salts such as salts of hydrofluorides, hydrochlorides, hydrobromides and hydroiodides; inorganic acid salts such as nitrates, perchlorates, sulfates, phosphates, etc.; lower alkane sulfonates such as methanesulfonates, trifluoromethanesulfonates and ethanesulfonates; arylsulfonates such as benzenesulfonates and p-toluenesulfonates; organic acid salts such as acetates, malates, fumarates, succinates, citrates, tartarates, oxalates, maleates, etc.; and, amino acid salts such as salts of glycine, lysine, arginine, ornithine, glutamic acid and aspartic acid. These salts may be produced by known methods. Alternatively, the suppressor antisense oligomer of the present invention may be in the form of a hydrate thereof.

### [Method for Producing PMO]

Each of the antisense oligomer of the present invention and the suppressor antisense oligomer of the present invention may be PMO. An embodiment of PMO is, for example, the compound represented by general formula (I) below (hereinafter, referred to as PMO (I)).

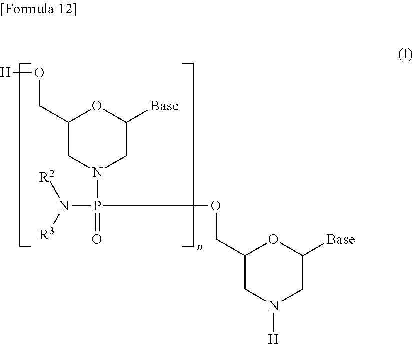

wherein Base, R² and R³ have the same significance as defined above; and,

n is a given integer of 1 to 99, preferably a given integer of 18 to 28.

PMO (I) can be produced in accordance with a known method (cf., e.g., WO2009/064471 or WO2013/100190).

In the antisense oligomer of the present invention or the suppressor antisense oligomer of the present invention, the 5′ end may be a group represented by any of chemical structures (1) to (3) below, and preferably is (3) —OH.

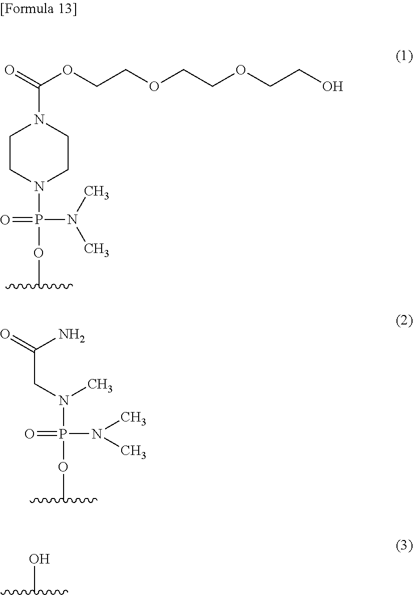

Hereinafter, the groups shown by (1), (2) and (3) above are referred to as “Group (1),” “Group (2)” and “Group (3),” respectively.

### 3. Medical Application

The present invention provides a pharmaceutical composition comprising the antisense oligomer of the present invention (also including the pharmaceutically acceptable salt thereof, or hydrate thereof) (hereinafter, referred to as the “pharmaceutical composition of the present invention”). The pharmaceutical composition of the present invention may further comprise the suppressor antisense oligomer of the present invention (also including the pharmaceutically acceptable salt thereof, or hydrate thereof) and/or a pharmaceutically acceptable carrier.

The present invention also provides a pharmaceutical composition comprising the antisense oligomer of the present invention (also including the pharmaceutically acceptable salt thereof, or hydrate thereof) and the suppressor antisense oligomer of the present invention (also including the pharmaceutically acceptable salt thereof, or hydrate thereof) (hereinafter, referred to as the “pharmaceutical combination of the present invention”).

When the pharmaceutical composition of the present invention comprises the antisense oligomer of the present invention and the suppressor antisense oligomer of the present invention, any combination of these oligomers is included. In the pharmaceutical combination of the present invention, any combination of the antisense oligomer of the present invention and the suppressor antisense oligomer of the present invention is included. The antisense oligomer contained in the pharmaceutical composition of the present invention or the pharmaceutical combination of the present invention can be any antisense oligomer of the present invention and is, but not particularly limited to, preferably an antisense oligomer consisting of any one base sequence selected from the group consisting of SEQ ID NOs: 1 to 111, 113 to 177, 179, 180, 182, 183, 185 to 193, 195 to 198, 200 to 223, and 225 to 232, more preferably an antisense oligomer consisting of any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232, and further preferably an antisense oligomer consisting of any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228.

The suppressor antisense oligomer contained in the pharmaceutical composition of the present invention or the pharmaceutical combination of the present invention can be any suppressor antisense oligomer of the present invention and is, but not particularly limited to, preferably a suppressor antisense oligomer consisting of any one base sequence selected from the group consisting of SEQ ID NOs: 257 to 275, more preferably SEQ ID NO: 1, 5, 6, 7, 8, 10, 11, 14, 26, or 27, and further preferably a suppressor antisense oligomer consisting of any one base sequence selected from the group consisting of SEQ ID NOs: 260, 261, and 263.

When the pharmaceutical composition of the present invention comprises the antisense oligomer of the present invention and the suppressor antisense oligomer of the present invention, preferably, the pharmaceutical composition comprises a combination of the antisense oligomer of the present invention and the suppressor antisense oligomer of the present invention described above. Preferably, the pharmaceutical combination of the present invention comprises a combination of the antisense oligomer of the present invention and the suppressor antisense oligomer of the present invention described above.

In the pharmaceutical combination of the present invention, examples of the combination of the antisense oligomer of the present invention and the suppressor antisense oligomer of the present invention include

(K1) a combination in which the antisense oligomer is an oligomer consisting of SEQ ID NO: 75 and the suppressor antisense oligomer is an oligomer consisting of SEQ ID NO: 260,
(K2) a combination in which the antisense oligomer is an oligomer consisting of SEQ ID NO: 75 and the suppressor antisense oligomer is an oligomer consisting of SEQ ID NO: 261,
(K3) a combination in which the antisense oligomer is an oligomer consisting of SEQ ID NO: 75 and the suppressor antisense oligomer is an oligomer consisting of SEQ ID NO: 263,
(K4) a combination in which the antisense oligomer is an oligomer consisting of SEQ ID NO: 65 and the suppressor antisense oligomer is an oligomer consisting of SEQ ID NO: 260,
(K5) a combination in which the antisense oligomer is an oligomer consisting of SEQ ID NO: 65 and the suppressor antisense oligomer is an oligomer consisting of SEQ ID NO: 261,
(K6) a combination in which the antisense oligomer is an oligomer consisting of SEQ ID NO: 65 and the suppressor antisense oligomer is an oligomer consisting of SEQ ID NO: 263,
(K7) a combination in which the antisense oligomer is a combination of oligomers consisting of SEQ ID NOs: 55 and 59 and the suppressor antisense oligomer is an oligomer consisting of SEQ ID NO: 260,
(K8) a combination in which the antisense oligomer is a combination of oligomers consisting of SEQ ID NOs: 55 and 59 and the suppressor antisense oligomer is an oligomer consisting of SEQ ID NO: 261, and
(K9) a combination in which the antisense oligomer is a combination of oligomers consisting of SEQ ID NOs: 55 and 59 and the suppressor antisense oligomer is an oligomer consisting of SEQ ID NO: 263.

The pharmaceutical composition of the present invention may comprise the antisense oligomer of the present invention and the suppressor antisense oligomer of the present invention in these combinations.

The pharmaceutical composition of the present invention and the pharmaceutical combination of the present invention can each be used for the treatment of, for example, Duchenne muscular dystrophy, Becker muscular dystrophy, limb-girdle muscular dystrophy (LGMD), congenital muscular dystrophy, Emery-Dreifuss muscular dystrophy, facioscapulohumeral muscular dystrophy, oculopharyngeal muscular dystrophy, cerebral autosomal dominant arteriopathy with subcortical infarct and leukoencephalopathy (CADASIL), and Alport's syndrome. The pharmaceutical combination of the present invention and the pharmaceutical composition of the present invention can each be administered to a human patient and in particular, a human patient with muscular dystrophy. The patient to receive the pharmaceutical combination of the present invention or the pharmaceutical composition of the present invention may be a human patient having a mutation that is amenable to skipping of two or more exons selected from the group consisting of exons 45 to 55 in the dystrophin gene.

One embodiment of the present invention provides a method for treatment of muscular dystrophy, which comprises administering to a patient with muscular dystrophy the antisense oligomer of the present invention or a combination of the antisense oligomer of the present invention and the suppressor antisense oligomer of the present invention.

Another embodiment of the present invention provides a method for treatment of muscular dystrophy, which comprises administering to a patient with muscular dystrophy the pharmaceutical composition of the present invention or the pharmaceutical combination of the present invention.

The method for treatment may involve performing skipping of any two or more numerically consecutive exons selected from the group consisting of the 45th exon to the 55th exon in human dystrophin pre-mRNA. In the method for treatment, the patient with muscular dystrophy may be a patient having a mutation that is amenable to exon 45 to 55 skipping in the dystrophin gene. The patient may be a human and may be a human patient having a mutation that is amenable to exon 45 to 55 skipping in the dystrophin gene.

The present invention further provides use of the antisense oligomer of the present invention or a combination of the antisense oligomer of the present invention and the suppressor antisense oligomer of the present invention, or the pharmaceutical composition of the present invention or the pharmaceutical combination of the present invention in manufacturing of a medicament for the treatment of muscular dystrophy.

The present invention further provides the antisense oligomer of the present invention or a combination of the antisense oligomer of the present invention and the suppressor antisense oligomer of the present invention, or the pharmaceutical composition of the present invention or the pharmaceutical combination of the present invention for use in the treatment of muscular dystrophy. The treatment may involve performing skipping of any two or more numerically consecutive exons selected from the group consisting of the 45th exon to the 55th exon in human dystrophin pre-mRNA. In the treatment, the patient with muscular dystrophy may be a patient having a mutation that is amenable to exon 45 to 55 skipping in the dystrophin gene. The patient may be a human and may be a human patient having a mutation that is amenable to exon 45 to 55 skipping in the dystrophin gene.

Administration route for the antisense oligomer of the present invention or the combination of the antisense oligomer of the present invention and the suppressor antisense oligomer of the present invention, or the pharmaceutical composition of the present invention or the pharmaceutical combination of the present invention is not particularly limited so long as it is pharmaceutically acceptable route for administration, and can be chosen depending upon method of treatment. In view of easiness in delivery to muscle tissues, preferred are intravenous administration, intraarterial administration, intramuscular administration, subcutaneous administration, oral administration, tissue administration, transdermal administration, etc. Also, dosage forms which are available for the composition of the present invention are not particularly limited, and include, for example, various injections, oral agents, drips, inhalations, ointments, lotions, etc.

In administration of the antisense oligomer of the present invention and/or the suppressor antisense oligomer of the present invention to patients with muscular dystrophy, preferably, the composition of the present invention contains a carrier to promote delivery of the oligomer to muscle tissues. Such a carrier is not particularly limited as far as it is pharmaceutically acceptable, and examples include cationic carriers such as cationic liposomes, cationic polymers, etc., or carriers using viral envelope. The cationic liposomes are, for example, liposomes composed of 2-O-(2-diethylaminoethyl)carbamoyl-1,3-O-dioleoylglycerol and phospholipids as the essential constituents (hereinafter referred to as “liposome A”), Oligofectamine (registered trademark) (manufactured by Invitrogen Corp.), Lipofectin (registered trademark) (manufactured by Invitrogen Corp.), Lipofectamine (registered trademark) (manufactured by Invitrogen Corp.), Lipofectamine 2000 (registered trademark) (manufactured by Invitrogen Corp.), DMRIE-C(registered trademark) (manufactured by Invitrogen Corp.), GeneSilencer (registered trademark) (manufactured by Gene Therapy Systems), TransMessenger (registered trademark) (manufactured by QIAGEN, Inc.), TransIT TKO (registered trademark) (manufactured by Mirus) and Nucleofector II (Lonza). Among others, liposome A is preferred. Examples of cationic polymers are JetSI (registered trademark) (manufactured by Qbiogene, Inc.) and Jet-PEI (registered trademark) (polyethylenimine, manufactured by Qbiogene, Inc.). An example of carriers using viral envelop is GenomeOne (registered trademark) (HVJ-E liposome, manufactured by Ishihara Sangyo). Alternatively, the medical devices described in Japanese Patent Nos. 2924179 and the cationic carriers described in Japanese Domestic Re-Publication PCT Nos. 2006/129594 and 2008/096690 may be used as well.

A concentration of the antisense oligomer of the present invention contained in the pharmaceutical composition of the present invention and/or the pharmaceutical combination of the present invention may vary depending on kind of the carrier, etc., and is appropriately in a range of 0.1 nM to 100 μM, preferably in a range of 1 nM to 10 μM, and more preferably in a range of 10 nM to 1 μM. A weight ratio of the antisense oligomer of the present invention contained in the composition of the present invention and the carrier (carrier/antisense oligomer of the present invention) may vary depending on property of the oligomer, type of the carrier, etc., and is appropriately in a range of 0.1 to 100, preferably in a range of 1 to 50, and more preferably in a range of 10 to 20.

A concentration of the suppressor antisense oligomer of the present invention contained in the pharmaceutical composition of the present invention and/or the pharmaceutical combination of the present invention may vary depending on kind of the carrier, etc., and is appropriately in a range of 0.1 nM to 100 μM, preferably in a range of 1 nM to 10 μM, and more preferably in a range of 10 nM to 1 μM. A weight ratio of the suppressor antisense oligomer of the present invention contained in the composition of the present invention and the carrier (carrier/suppressor antisense oligomer of the present invention) may vary depending on property of the oligomer, type of the carrier, etc., and is appropriately in a range of 0.1 to 100, preferably in a range of 1 to 50, and more preferably in a range of 10 to 20.

The pharmaceutical composition of the present invention and/or the pharmaceutical combination of the present invention may be in the form of an aqueous solution. In this case, the pharmaceutical composition of the present invention and/or the pharmaceutical combination of the present invention may comprise the antisense oligomer of the present invention in a concentration of 2.5 to 500 mg/mL, 5 to 450 mg/mL, 10 to 400 mg/mL, 15 to 350 mg/mL, 20 to 300 mg/mL, 20 to 250 mg/mL, 20 to 200 mg/mL, 20 to 150 mg/mL, 20 to 100 mg/mL, 20 to 50 mg/mL, 20 to 40 mg/mL, 20 to 30 mg/mL, 23 to 27 mg/mL, 24 to 26 mg/mL, or 25 mg/mL. Alternatively, the pharmaceutical composition of the present invention and/or the pharmaceutical combination of the present invention may comprise the antisense oligomer of the present invention in a concentration of 10 to 100 mg/mL, 15 to 95 mg/mL, 20 to 80 mg/mL, 25 to 75 mg/mL, 30 to 70 mg/mL, 35 to 65 mg/mL, 40 to 60 mg/mL, 45 to 55 mg/mL, 47 to 53 mg/mL, 48 to 52 mg/mL, 49 to 51 mg/mL, or 50 mg/mL.

The pharmaceutical composition of the present invention and/or the pharmaceutical combination of the present invention in the form of an aqueous solution may comprise the suppressor oligomer of the present invention in a concentration of 2.5 to 500 mg/mL, 5 to 450 mg/mL, 10 to 400 mg/mL, 15 to 350 mg/mL, 20 to 300 mg/mL, 20 to 250 mg/mL, 20 to 200 mg/mL, 20 to 150 mg/mL, 20 to 100 mg/mL, 20 to 50 mg/mL, 20 to 40 mg/mL, 20 to 30 mg/mL, 23 to 27 mg/mL, 24 to 26 mg/mL, or 25 mg/mL. Alternatively, the pharmaceutical composition of the present invention and/or the pharmaceutical combination of the present invention may comprise the suppressor oligomer of the present invention in a concentration of 10 to 100 mg/mL, 15 to 95 mg/mL, 20 to 80 mg/mL, 25 to 75 mg/mL, 30 to 70 mg/mL, 35 to 65 mg/mL, 40 to 60 mg/mL, 45 to 55 mg/mL, 47 to 53 mg/mL, 48 to 52 mg/mL, 49 to 51 mg/mL, or 50 mg/mL.

The pharmaceutical composition of the present invention and/or the pharmaceutical combination of the present invention may be in a dry form. In this case, in order to prepare the pharmaceutical composition of the present invention and/or the pharmaceutical combination of the present invention in an aqueous solution form, for example, 125 mg or 250 mg of the antisense oligomer of the present invention in a dry form may be mixed with 0.5 mL to 100 mL of water (which corresponds to the antisense oligomer of the present invention in a concentration of 1.25 mg/mL to 250 mg/mL or 2.5 mg/mL to 500 mg/mL), preferably with 1 mL to 50 mL of water (which corresponds to the antisense oligomer of the present invention in a concentration of 2.5 mg/mL to 125 mg/mL or 5 mg/mL to 250 mg/mL), more preferably with 5 mL to 10 mL of water (which correspond to the antisense oligomer of the present invention in a concentration of 12.5 mg/mL to 25 mg/mL or 25 mg/mL to 50 mg/mL) and used.

When the pharmaceutical composition of the present invention and/or the pharmaceutical combination of the present invention is in a dry form, in order to prepare the pharmaceutical composition of the present invention and/or the pharmaceutical combination of the present invention in an aqueous solution form, for example, 125 mg or 250 mg of the suppressor oligomer of the present invention in a dry form may be mixed with 0.5 mL to 100 mL of water (which corresponds to the suppressor oligomer of the present invention in a concentration of 1.25 mg/mL to 250 mg/mL or 2.5 mg/mL to 500 mg/mL), preferably with 1 mL to 50 mL of water (which corresponds to the suppressor oligomer of the present invention in a concentration of 2.5 mg/mL to 125 mg/mL or 5 mg/mL to 250 mg/mL), more preferably with 5 mL to 10 mL of water (which correspond to the suppressor oligomer of the present invention in a concentration of 12.5 mg/mL to 25 mg/mL or 25 mg/mL to 50 mg/mL) and used.

The concentrations of the antisense oligomer of the present invention and the suppressor oligomer of the present invention contained in the pharmaceutical composition of the present invention and/or the pharmaceutical combination of the present invention may be the respective concentrations of the antisense oligomer of the present invention and the suppressor oligomer of the present invention or may be the total concentration of the antisense oligomer of the present invention and the suppressor oligomer of the present invention.

In addition to the antisense oligomer of the present invention and/or the suppressor antisense oligomer of the present invention and the carrier described above, pharmaceutically acceptable additives may also be optionally formulated in the pharmaceutical composition of the present invention and/or the pharmaceutical combination of the present invention. Examples of such additives are emulsification aids (e.g., fatty acids having 6 to 22 carbon atoms and their pharmaceutically acceptable salts, albumin and dextran), stabilizers (e.g., cholesterol, phosphatidic acid, mannitol, and sorbitol), isotonizing agents (e.g., sodium chloride, glucose, maltose, lactose, sucrose, and trehalose), and pH controlling agents (e.g., hydrochloric acid, sulfuric acid, phosphoric acid, acetic acid, sodium hydroxide, potassium hydroxide and triethanolamine). One or more of these additives can be used. The content of the additive in the composition of the present invention is appropriately 90 wt % or less, preferably 70 wt % or less and more preferably, 50 wt % or less.

The pharmaceutical composition of the present invention and/or the pharmaceutical combination of the present invention can be prepared by adding the antisense oligomer of the present invention and/or the suppressor antisense oligomer of the present invention to a carrier dispersion and adequately stirring the mixture. Additives may be added at an appropriate step either before or after addition of the antisense oligomer of the present invention and/or the suppressor antisense oligomer of the present invention. An aqueous solvent that can be used in adding the antisense oligomer of the present invention and/or the suppressor antisense oligomer of the present invention is not particularly limited as far as it is pharmaceutically acceptable, and examples are injectable water or injectable distilled water, electrolyte fluid such as physiological saline, etc., and sugar fluid such as glucose fluid, maltose fluid, etc. A person skilled in the art can appropriately choose conditions for pH and temperature for such matter.

The pharmaceutical composition of the present invention and/or the pharmaceutical combination of the present invention may be prepared into, e.g., a liquid form and its lyophilized preparation. The lyophilized preparation can be prepared by lyophilizing the composition of the present invention in a liquid form in a conventional manner. The lyophilization can be performed, for example, by appropriately sterilizing the composition of the present invention in a liquid form, dispensing an aliquot into a vial container, performing preliminary freezing for 2 hours at conditions in a range of about −40° C. to −20° C., performing a primary drying in a range of about 0° C. to 10° C. under reduced pressure, and then performing a secondary drying in a range of about 15° C. to 25° C. under reduced pressure. In general, the lyophilized preparation of the composition of the present invention can be obtained by replacing the content of the vial with nitrogen gas and capping.

The lyophilized preparation of the pharmaceutical composition of the present invention and/or the pharmaceutical combination of the present invention can be used in general upon reconstitution by adding an optional suitable solution (reconstitution liquid) and redissolving the preparation. Such a reconstitution liquid includes injectable water, physiological saline and other infusion fluids. A volume of the reconstitution liquid may vary depending on the intended use, etc., is not particularly limited, and is suitably 0.5-fold to 2-fold greater than the volume prior to lyophilization or no more than 500 mL.

It is desired to control a dose of the pharmaceutical composition of the present invention and/or the pharmaceutical combination of the present invention to be administered, by taking the following factors into account: the type and dosage form of the antisense oligomer of the present invention and/or the suppressor antisense oligomer of the present invention contained; patients' conditions including age, body weight, etc.; administration route; and the characteristics and extent of the disease. A single dose calculated as the amount of the antisense oligomer of the present invention and/or the suppressor antisense oligomer of the present invention can be 0.1 mg to 1 g per kg body weight, preferably 1 mg to 100 mg per kg body weight, more preferably 1 mg to 90 mg per kg body weight, and further preferably 1 mg to 80 mg per kg body weight. The frequency of administration may be once per 1 to 3 days, once per week, or once per 2 to 3 weeks. This numerical range may vary occasionally depending on type of the target disease, administration route and target molecule. Therefore, a dose or frequency of administration lower than the range may be sufficient in some occasion and conversely, a dose or frequency of administration higher than the range may be required occasionally.

In still another embodiment of the pharmaceutical composition of the present invention and/or the pharmaceutical combination of the present invention, there is provided a pharmaceutical composition comprising a vector capable of expressing the antisense oligomer of the present invention and/or the suppressor antisense oligomer of the present invention and the carrier described above. Such an expression vector may be a vector capable of expressing a plurality of the antisense oligomers of the present invention and/or the suppressor antisense oligomers of the present invention. The composition may be formulated with pharmaceutically acceptable additives as in the case with the composition of the present invention containing the antisense oligomer of the present invention and/or the suppressor antisense oligomer of the present invention. A concentration of the expression vector contained in the composition may vary depending upon type of the career, etc., and is appropriately in a range of 0.1 nM to 100 μM, preferably in a range of 1 nM to 10 μM, and more preferably in a range of 10 nM to 1 μM. A weight ratio of the expression vector contained in the composition and the carrier (carrier/expression vector) may vary depending on property of the expression vector, type of the carrier, etc., and is appropriately in a range of 0.1 to 100, preferably in a range of 1 to 50, and more preferably in a range of 10 to 20. The content of the carrier contained in the composition is the same as in the case with the composition of the present invention containing the antisense oligomer of the present invention and/or the suppressor antisense oligomer of the present invention, and a method for producing the same is also the same as in the case with the composition of the present invention.

4. Pre-mRNA of Interest Other than Human Dystrophin Pre-mRNA

As used herein, the case where the pre-mRNA of interest is human dystrophin pre-mRNA is specifically described, but is not limited thereto. As for pre-mRNA of interest other than human dystrophin pre-mRNA, methods for designing, producing, and using an antisense oligomer and a suppressor antisense oligomer can be carried out in accordance with techniques and methods disclosed herein or known in the art. Those skilled in the art can also design, produce and use an antisense oligomer or a suppressor antisense oligomer for pre-mRNA of interest other than human dystrophin pre-mRNA on the basis of techniques and methods disclosed herein or known in the art. Likewise, those skilled in the art can perform multi-exon skipping of the pre-mRNA of interest using the antisense oligomer and enhance the efficiency of multi-exon skipping using the suppressor antisense oligomer on the basis of techniques and methods disclosed herein or known in the art.

As used herein, examples of the pre-mRNA of interest include human γ-sarcoglycan (SGCG) pre-mRNA, human Notch3 pre-mRNA, and human dystrophin pre-mRNA. In a certain embodiment of the present invention, the pre-mRNA of interest may be human dystrophin pre-mRNA. In another embodiment of the present invention, the pre-mRNA of interest may be SGCG or human Notch3 pre-mRNA.

The base sequences of target regions of the antisense oligomer for use in multi-exon skipping in the pre-mRNA of interest of human SGCG or human Notch3 are as given below.

In the present invention, the target regions in human SGCG pre-mRNA are shown as SEQ ID NOs: 283 and 284 below.

TABLE 8

| | | SEQ |
|---|---|---|
| Target region | Base sequence of target region | ID NO |
| Region in vicinity of | ATTACAAAAATATGGAAATAGAGAGTAGCAGGAAAAAGTATATGGCATT | 283 |
| acceptor of intron 3 | TCTATTACCTAGAACCCCACTGTTGTAGGIGTCTTTCCTTGACATAGCT | |
| (region indicated by | GTGACCA7ATAGCTCTGTTAAATTGCATATTTGTCTAAATAATTCATAT | |
| range of −400 bases | TTTTTTCTATCCCTATCTTGCGTTTGGAGCTCATTTTATGATTGTTATT | |
| to +50 bases with 3′ | CTTTTATCTTTCAATAAATACTCAAAATGTTAAAAAAATATGATTCAGG | |
| end of intron 3 defined | AATTTTTAATTGTCTTAGCCACAAATTTAIAGGATTTCCAGGATCTGTA | |
| as basing point 0) | ACAATGGATAAATAATTTTATAAAAATCCTAAATTTACACAGAATTATA | |
| | AAGATATAATCATTTTAAACAGCACCTATTTTGCAAATTTTATAAATCT | |
| | CTTTCTAGGACTCATCTCTGCTTCTACAATCAACCCAGAATGTGACTGT | |
| | AAATGCGCG | |
| Region in vicinity of | CACAGGCAGGTTAAAAGTCGGTGAGTCCAGCTTCATCATGGTGCTTTGC | 284 |
| donor of intron 4 | ATGCATGTTGTCCATGAATAGTGCTAAATGAATGCATTGTTTTTTCTTC | |
| (region indicated by | TAAAGAAATCAAAGCTACTTATGAACAAAATATGAATTTTCTAAATATC | |
| range of −20 bases to | ATGCTGTGTTGACCACAGACTAGCACCACAGAGTGGGGTGGGGGGTGAG | |
| +400 bases with 5′ | GGGACAGCCTGAAGTGCTTTGATTAGAGTTATTTTTGTTCCAAGAAATA | |
| end of intron 4 defined | GAGGACAATTAAAGCTAGTACAAGAAACAAAATGTTTA7TGTGGGTTAC | |
| as basing point 0) | CAAAGACTCTTGCAGCAACCAAAGGGAAGGAACCCAAGIAGCTGGCCTC | |
| | AAAGAGCACAGAGACCAGTGGCTGAGAGCCACGAATACATTCAGTTCCC | |
| | TCCCCGTTCTTTCTTCTCTTTTCTCTCC | |

In the present invention, the target regions in human Notch3 pre-mRNA are shown below.

TABLE 9

| | | SEQ |
|---|---|---|
| Target region | Base sequence of target region | ID NO |
| Region in vicinity of | AAGTGATCTGCCCGCCGCAGCCTCCCAAAGTTCTGGGATTACAGGCGTG | 285 |
| acceptor of intron 3 | AGCCACTGCTCCCAGCAAATGTGTGTTTGCTGCTCTGTTTCCCTGCGTG | |
| (region indicated by | TTTCTTGCCTGTCTTGTGTGTATCTTTGTGTCTGGGGCCATCCTGCCCT | |
| range of −400 bases | GTGCTGCCCAACCAAGCCATCTCTGCCCACAGGTGCCCGCCTGGCTGGG | |
| to +50 bases with 3′ | TGGGTGAGCGGTGTCAGCTGGAGGACCCCTGTCACTCAGGCCCCTGTGC | |
| end of intron 3 defined | TGGCCGTGGTGTCTGCCAGAGTTCAGTGGTGGCTGGCACCGCCCGATTC | |
| as basing point 0) | TCATGCCGGTGCCCCCGTGGCTTCCGAGGTGAGAGGGGAAGAGTCTGGA | |
| | GGGGAGGTAGTCGGGGGTGTGGTCAGTCCTAAACTCACCCTGTCCTGGT | |
| | CCCTCCAGGCCCTGACTGCTCCCTGCCAGATCCCTGCCTCAGCAGCCCT | |
| | TGTGCCCAC | |
| Region in vicinity of | CGACTGTGCCTGTCTTCCTGGTGAGTGAGCCCTACTCAGGAGAGTCAGA | 286 |
| donor of intron 4 | GGGGTGGGCGTGGGGACAGCAGGCCAGCCCGGCGGTGACCATCCTTGCC | |
| (region indicated by | CCCTTCCCTGCTAGGGTTTGAGGGTCAGAATTGTGAAGTGAACGTGGAC | |
| range of −20 bases to | GACTGTCCAGGACACCGATGTCTCAATGGGGGGACATGCGTGGATGGCG | |
| +400 bases with 5′ end | TCAACACCTATAACTGCCAGTGCCCTCCTGAGTGGACAGGTGGGCACTG | |
| of intron 4 defined as | CGGCCAGAGGGAGCGGGGAGGCAGGCCTCGGGTGGACATGCGCCAGGTG | |
| basing point 0) | GCTGGACTGCTGCATCTGTGTGCCACAGGCCAGTTCTGCACGGAGGACG | |
| | TGGATGAGTGTCAGCTGCAGCCCAACGCCTGCCACAATGGGGGTACCTG | |
| | CTTCAACACGCTGGGTGGCCACAGCTGC | |

The antisense oligomer for human SGCG that induces multi-exon skipping from human SGCG pre-mRNA targets, for example, the range of −400 to +50 bases with the 3′ end of intron 3 defined as a basing point, and as a specific example, is an antisense oligomer for human SGCG consisting of

(L1) any one base sequence selected from the group consisting of SEQ ID NOs: 287 to 308, or
(L2) a base sequence that has at least 85% identity with any one base sequence selected from the group consisting of SEQ ID NOs: 287 to 308, and has a length within ±15% of the length of the any one base sequence selected, or a pharmaceutically acceptable salt thereof, or hydrate thereof.

Herein, the base sequence (L2) is a mutant type of the base sequence (L1). Such a mutant type also includes (L2-1) a base sequence that has at least 85% identity with any one base sequence selected from the group consisting of SEQ ID NOs: 287 to 308, and has a length within ±15% of the length of the any one base sequence selected,

(L2-2) a base sequence that has at least 86% identity with any one base sequence selected from the group consisting of SEQ ID NOs: 287 to 308, and has a length within ±14a of the length of the any one base sequence selected,
(L2-3) a base sequence that has at least 87% identity with any one base sequence selected from the group consisting of SEQ ID NOs: 287 to 308, and has a length within ±13% of the length of the any one base sequence selected,
(L2-4) a base sequence that has at least 88% identity with any one base sequence selected from the group consisting of SEQ ID NOs: 287 to 308, and has a length within ±12% of the length of the any one base sequence selected,
(L2-5) a base sequence that has at least 89% identity with any one base sequence selected from the group consisting of SEQ ID NOs: 287 to 308, and has a length within ±11% of the length of the any one base sequence selected,
(L2-6) a base sequence that has at least 90% identity with any one base sequence selected from the group consisting of SEQ ID NOs: 287 to 308, and has a length within ±10% of the length of the any one base sequence selected,
(L2-7) a base sequence that has at least 91% identity with any one base sequence selected from the group consisting of SEQ ID NOs: 287 to 308, and has a length within ±9% of the length of the any one base sequence selected,
(L2-8) a base sequence that has at least 92% identity with any one base sequence selected from the group consisting of SEQ ID NOs: 287 to 308, and has a length within ±8% of the length of the any one base sequence selected,
(L2-9) a base sequence that has at least 93% identity with any one base sequence selected from the group consisting of SEQ ID NOs: 287 to 308, and has a length within ±7% of the length of the any one base sequence selected,
(L2-10) a base sequence that has at least 94% identity with any one base sequence selected from the group consisting of SEQ ID NOs: 287 to 308, and has a length within ±6% of the length of the any one base sequence selected,
(L2-11) a base sequence that has at least 95% identity with any one base sequence selected from the group consisting of SEQ ID NOs: 287 to 308, and has a length within ±5% of the length of the any one base sequence selected,
(L2-12) a base sequence that has at least 96% identity with any one base sequence selected from the group consisting of SEQ ID NOs: 287 to 308, and has a length within ±4% of the length of the any one base sequence selected,
(L2-13) a base sequence that has at least 97% identity with any one base sequence selected from the group consisting of SEQ ID NOs: 287 to 308, and has a length within ±3% of the length of the any one base sequence selected,
(L2-14) a base sequence that has at least 98% identity with any one base sequence selected from the group consisting of SEQ ID NOs: 287 to 308, and has a length within ±2% of the length of the any one base sequence selected,
(L2-15) a base sequence that has at least 99% identity with any one base sequence selected from the group consisting of SEQ ID NOs: 287 to 308, and has a length within ±1% of the length of the any one base sequence selected, and
(L2-16) a base sequence that has at least 99.5% identity consisting of SEQ ID NOs: 287 to 308, and has a length within ±0.5% of the length of the any one base sequence selected.

Thus, a further embodiment of the antisense oligomer for human SGCG is an antisense oligomer for human SGCG consisting of any one base sequence selected from the group consisting of (L1) and (L2-1) to (L2-16), or a pharmaceutically acceptable salt thereof, or hydrate thereof. The antisense oligomer for human SGCG is included in the present invention.

The suppressor antisense oligomer that suppresses single skipping from human SGCG pre-mRNA (suppressor antisense oligomer for human SGCG) targets, for example, a splicing silencer sequence of intron 4 or a splice site of single skipping, and as a specific example, is a suppressor antisense oligomer for human SGCG consisting of

(M1) any one base sequence selected from the group consisting of SEQ ID NOs: 331 to 335, or
(M2) a base sequence that has at least 85% identity with any one base sequence selected from the group consisting of SEQ ID NOs: 331 to 335, and has a length within ±15% of the length of the any one base sequence selected, or a pharmaceutically acceptable salt thereof, or hydrate thereof.

Herein, the base sequence (M2) is a mutant type of the base sequence (M1). Such a mutant type also includes

(M2-1) a base sequence that has at least 85% identity with any one base sequence selected from the group consisting of SEQ ID NOs: 331 to 335, and has a length within ±15% of the length of the any one base sequence selected,
(M2-2) a base sequence that has at least 86% identity with any one base sequence selected from the group consisting of SEQ ID NOs: 331 to 335, and has a length within ±14% of the length of the any one base sequence selected,
(M2-3) a base sequence that has at least 87% identity with any one base sequence selected from the group consisting of SEQ ID NOs: 331 to 335, and has a length within ±13% of the length of the any one base sequence selected,
(M2-4) a base sequence that has at least 88% identity with any one base sequence selected from the group consisting of SEQ ID NOs: 331 to 335, and has a length within ±12% of the length of the any one base sequence selected,
(M2-5) a base sequence that has at least 89% identity with any one base sequence selected from the group consisting of SEQ ID NOs: 331 to 335, and has a length within ±11% of the length of the any one base sequence selected,
(M2-6) a base sequence that has at least 90% identity with any one base sequence selected from the group consisting of SEQ ID NOs: 331 to 335, and has a length within ±10% of the length of the any one base sequence selected,
(M2-7) a base sequence that has at least 91% identity with any one base sequence selected from the group consisting of SEQ ID NOs: 331 to 335, and has a length within ±9% of the length of the any one base sequence selected,
(M2-8) a base sequence that has at least 92% identity with any one base sequence selected from the group consisting of SEQ ID NOs: 331 to 335, and has a length within ±8% of the length of the any one base sequence selected,
(M2-9) a base sequence that has at least 93% identity with any one base sequence selected from the group consisting of SEQ ID NOs: 331 to 335, and has a length within ±7% of the length of the any one base sequence selected,
(M2-10) a base sequence that has at least 94% identity with any one base sequence selected from the group consisting of SEQ ID NOs: 331 to 335, and has a length within ±6% of the length of the any one base sequence selected,
(M2-11) a base sequence that has at least 95% identity with any one base sequence selected from the group consisting of SEQ ID NOs: 331 to 335, and has a length within ±5% of the length of the any one base sequence selected,
(M2-12) a base sequence that has at least 96% identity with any one base sequence selected from the group consisting of SEQ ID NOs: 331 to 335, and has a length within ±4% of the length of the any one base sequence selected,
(M2-13) a base sequence that has at least 97% identity with any one base sequence selected from the group consisting of SEQ ID NOs: 331 to 335, and has a length within ±3% of the length of the any one base sequence selected,
(M2-14) a base sequence that has at least 98% identity with any one base sequence selected from the group consisting of SEQ ID NOs: 331 to 335, and has a length within ±2% of the length of the any one base sequence selected,
(M2-15) a base sequence that has at least 99% identity with any one base sequence selected from the group consisting of SEQ ID NOs: 331 to 335, and has a length within ±1% of the length of the any one base sequence selected, and
(M2-16) a base sequence that has at least 99.5% identity consisting of SEQ ID NOs: 331 to 335, and has a length within ±0.5% of the length of the any one base sequence selected.

Thus, a further embodiment of the suppressor antisense oligomer for human SGCG is a suppressor antisense oligomer for human SGCG consisting of any one base sequence selected from the group consisting of (M1) and (M2-1) to (M2-16), or a pharmaceutically acceptable salt thereof, or hydrate thereof. The suppressor antisense oligomer for human SGCG is included in the present invention. The suppressor antisense oligomer for human SGCG is included in the present invention. The suppressor antisense oligomer for human SGCG is capable of enhancing the efficiency of multi-skipping from SGCG pre-mRNA.

The antisense oligomer for human Notch3 that induces multi-exon skipping from human Notch3 pre-mRNA targets, for example, a region in the vicinity of an acceptor of intron 3, i.e., the range of −400 to +50 bases with the 3′ end of intron 3 defined as a basing point, and is, for example, an antisense oligomer for human Notch3 consisting of

(N1) any one base sequence selected from the group consisting of SEQ ID NOs: 309 to 330, or
(N2) a base sequence that has at least 85% identity with any one base sequence selected from the group consisting of SEQ ID NOs: 309 to 330, and has a length within ±15% of the length of the any one base sequence selected, or a pharmaceutically acceptable salt thereof, or hydrate thereof.

Herein, the base sequence (N2) is a mutant type of the base sequence (N1). Such a mutant type also includes

(N2-1) a base sequence that has at least 85% identity with any one base sequence selected from the group consisting of SEQ ID NOs: 309 to 330, and has a length within ±15% of the length of the any one base sequence selected,
(N2-2) a base sequence that has at least 86% identity with any one base sequence selected from the group consisting of SEQ ID NOs: 309 to 330, and has a length within ±14% of the length of the any one base sequence selected,
(N2-3) a base sequence that has at least 87% identity with any one base sequence selected from the group consisting of SEQ ID NOs: 309 to 330, and has a length within ±13% of the length of the any one base sequence selected,
(N2-4) a base sequence that has at least 88% identity with any one base sequence selected from the group consisting of SEQ ID NOs: 309 to 330, and has a length within ±12% of the length of the any one base sequence selected,
(N2-5) a base sequence that has at least 89% identity with any one base sequence selected from the group consisting of SEQ ID NOs: 309 to 330, and has a length within ±11% of the length of the any one base sequence selected,
(N2-6) a base sequence that has at least 90% identity with any one base sequence selected from the group consisting of SEQ ID NOs: 309 to 330, and has a length within ±10% of the length of the any one base sequence selected,
(N2-7) a base sequence that has at least 91% identity with any one base sequence selected from the group consisting of SEQ ID NOs: 309 to 330, and has a length within ±9% of the length of the any one base sequence selected,
(N2-8) a base sequence that has at least 92% identity with any one base sequence selected from the group consisting of SEQ ID NOs: 309 to 330, and has a length within ±8% of the length of the any one base sequence selected,
(N2-9) a base sequence that has at least 93% identity with any one base sequence selected from the group consisting of SEQ ID NOs: 309 to 330, and has a length within ±7% of the length of the any one base sequence selected,
(N2-10) a base sequence that has at least 94% identity with any one base sequence selected from the group consisting of SEQ ID NOs: 309 to 330, and has a length within ±6% of the length of the any one base sequence selected,
(N2-11) a base sequence that has at least 95% identity with any one base sequence selected from the group consisting of SEQ ID NOs: 309 to 330, and has a length within ±5% of the length of the any one base sequence selected,
(N2-12) a base sequence that has at least 96% identity with any one base sequence selected from the group consisting of SEQ ID NOs: 309 to 330, and has a length within ±4% of the length of the any one base sequence selected,
(N2-13) a base sequence that has at least 97% identity with any one base sequence selected from the group consisting of SEQ ID NOs: 309 to 330, and has a length within ±3% of the length of the any one base sequence selected,
(N2-14) a base sequence that has at least 98% identity with any one base sequence selected from the group consisting of SEQ ID NOs: 309 to 330, and has a length within ±2% of the length of the any one base sequence selected,
(N2-15) a base sequence that has at least 99% identity consisting of SEQ ID NOs: 309 to 330, and has a length within ±1% of the length of the any one base sequence selected, and
(N2-16) a base sequence that has at least 99.5% identity with any one base sequence selected from the group consisting of SEQ ID NOs: 309 to 330, and has a length within ±0.5% of the length of the any one base sequence selected.

Thus, a further embodiment of the antisense oligomer for human Notch3 is an antisense oligomer for human Notch3 consisting of any one base sequence selected from the group consisting of (N1) and (N2-1) to (N2-16), or a pharmaceutically acceptable salt thereof, or hydrate thereof. The antisense oligomer for human Notch3 is included in the present invention.

The suppressor antisense oligomer that suppresses single skipping from human Notch3 pre-mRNA (suppressor antisense oligomer for human Notch3) targets, for example, a splicing silencer sequence of intron 4 or a splice site of single skipping, and as a specific example, is a suppressor antisense oligomer for human Notch3 consisting of

(O1) any one base sequence selected from the group consisting of SEQ ID NOs: 336 to 340, or
(O2) a base sequence that has at least 85% identity with any one base sequence selected from the group consisting of SEQ ID NOs: 336 to 340, and has a length within ±15% of the length of the any one base sequence selected, or a pharmaceutically acceptable salt thereof, or hydrate thereof.

Herein, the base sequence (02) is a mutant type of the base sequence (01). Such a mutant type also includes

(O2-1) a base sequence that has at least 85% identity with any one base sequence selected from the group consisting of SEQ ID NOs: 336 to 340, and has a length within ±15% of the length of the any one base sequence selected,
(O2-2) a base sequence that has at least 86% identity with any one base sequence selected from the group consisting of SEQ ID NOs: 336 to 340, and has a length within ±14% of the length of the any one base sequence selected,
(O2-3) a base sequence that has at least 87% identity with any one base sequence selected from the group consisting of SEQ ID NOs: 336 to 340, and has a length within ±13% of the length of the any one base sequence selected,
(O2-4) a base sequence that has at least 88% identity with any one base sequence selected from the group consisting of SEQ ID NOs: 336 to 340, and has a length within ±12% of the length of the any one base sequence selected,
(O2-5) a base sequence that has at least 89% identity with any one base sequence selected from the group consisting of SEQ ID NOs: 336 to 340, and has a length within ±11% of the length of the any one base sequence selected,
(O2-6) a base sequence that has at least 90% identity with any one base sequence selected from the group consisting of SEQ ID NOs: 336 to 340, and has a length within ±10% of the length of the any one base sequence selected,
(O2-7) a base sequence that has at least 91% identity with any one base sequence selected from the group consisting of SEQ ID NOs: 336 to 340, and has a length within ±9% of the length of the any one base sequence selected,
(O2-8) a base sequence that has at least 92% identity with any one base sequence selected from the group consisting of SEQ ID NOs: 336 to 340, and has a length within ±8% of the length of the any one base sequence selected,
(O2-9) a base sequence that has at least 93% identity with any one base sequence selected from the group consisting of SEQ ID NOs: 336 to 340, and has a length within ±7% of the length of the any one base sequence selected,
(O2-10) a base sequence that has at least 94% identity with any one base sequence selected from the group consisting of SEQ ID NOs: 336 to 340, and has a length within ±6% of the length of the any one base sequence selected,
(O2-11) a base sequence that has at least 95% identity with any one base sequence selected from the group consisting of SEQ ID NOs: 336 to 340, and has a length within ±5% of the length of the any one base sequence selected,
(O2-12) a base sequence that has at least 96% identity with any one base sequence selected from the group consisting of SEQ ID NOs: 336 to 340, and has a length within ±4% of the length of the any one base sequence selected,
(O2-13) a base sequence that has at least 97% identity with any one base sequence selected from the group consisting of SEQ ID NOs: 336 to 340, and has a length within ±3% of the length of the any one base sequence selected,
(O2-14) a base sequence that has at least 98% identity with any one base sequence selected from the group consisting of SEQ ID NOs: 336 to 340, and has a length within ±2% of the length of the any one base sequence selected,
(O2-15) a base sequence that has at least 99% identity consisting of SEQ ID NOs: 336 to 340, and has a length within ±1% of the length of the any one base sequence selected, and
(O2-16) a base sequence that has at least 99.5% identity with any one base sequence selected from the group consisting of SEQ ID NOs: 336 to 340, and has a length within ±0.5% of the length of the any one base sequence selected.

Thus, a further embodiment of the suppressor antisense oligomer for human Notch3 is a suppressor antisense oligomer for human Notch3 consisting of any one base sequence selected from the group consisting of (01) and (02-1) to (02-16), or a pharmaceutically acceptable salt thereof, or hydrate thereof. The suppressor antisense oligomer for human Notch3 is included in the present invention. The suppressor antisense oligomer for human Notch3 is capable of enhancing the efficiency of multi-skipping from Notch3 pre-mRNA.

The base sequences of SEQ ID NOs: 287 to 340 will be shown below.

| SEQ | | |
|---|---|---|
| ID NO | Target sequence | Base sequence |
| 287 | SCGCex4_(−400)-(−371) | TGCTACTCTCTATTTCCATATTTTTGTAAT |
| 288 | SCGCex4_(−380)-(−351) | AAATGCCATATACTTTTTCCTGCTACTCTC |
| 289 | SCGCex4_(−360)-(−331) | AGTGGGGTTCTAGGTAATAGAAATGCCATA |
| 290 | SCGCex4_(−340)-(−311) | CAAGGAAAGACACCTACAACAGTGGGGTTC |
| 291 | SCGCex4_(−320)-(−291) | CTATATGGTCACAGCTATGTCAAGGAAAGA |
| 292 | SCGCex4_(−300)-(−271) | AAATATGCAATTTAACAGAGCTATATGGTC |
| 293 | SCGCex4_(−280)-(−251) | AAAATATGAATTATTTAGACAAATATGCAA |
| 294 | SCGCex4_(−260)-(−231) | ACGCAAGATAGGGATAGAAAAAAATATGAA |
| 295 | SCGCex4_(−240)-(−211) | AATCATAAAATGAGCTCCAAACGCAAGATA |
| 296 | SCGCex4_(−220)-(−191) | TTGAAAGATAAAAGAATAACAATCATAAAA |
| 297 | SCGCex4_(−200)-(−171) | TTTAACATTTTGAGTATTTATTGAAAGATA |
| 298 | SCGCex4_(−180)-(−151) | AAATTCCTGAATCATATTTTTTTAACATTT |
| 299 | SCGCex4_(−160)-(−131) | TTTGTGGCTAAGACAATTAAAAATTCCTGA |
| 300 | SCGCex4_(−140)-(−111) | GATCCTGGAAATCCTATAAATTTGTGGCTA |
| 301 | SCGCex4_(−120)-(−91) | ATTATTTATCCATTGTTACAGATCCTGGAA |
| 302 | SCGCex4_(−100)-(−71) | AAATTTAGGATTTTTATAAAATTATTTATC |
| 303 | SCGCex4_(−80)-(−51) | ATATCTTTATAATTCTGTGTAAATTTAGGA |
| 304 | SCGCex4_(−60)-(−31) | AGGTGCTGTTTAAAATGATTATATCTTTAT |
| 305 | SCGCex4_(−40)-(−11) | ATTTATAAAATTTGCAAAATAGGTGCTGTT |
| 306 | SCGCex4_(−20)-10 | GAGATGAGTCCTAGAAAGAGATTTATAAAA |
| 307 | SCGCex4_l-30 | CTGGGTTGATTGTAGAAGCAGAGATGAGTC |
| 308 | SCGCex4_21-50 | CGCGCATTTACAGTCACATTCTGGGTTGAT |
| 309 | Notch3ex4_(−400)-(−371) | CTTTGGGAGGCTGCGGCGGGCAGATCACTT |
| 310 | Notch3ex4_(−380)-(−351) | TCACGCCTGTAATCCCAGAACTTTGGGAGG |
| 311 | Notch3ex4_(−360)-(−331) | CATTTGCTGGGAGCAGTGGCTCACGCCTGT |
| 312 | Notch3ex4_(−340)-(−311) | GAAACAGAGCAGCAAACACACATTTGCTGG |
| 313 | Notch3ex4_(−320)-(−291) | ACAGGCAAGAAACACGCAGGGAAACAGAGC |
| 314 | Notch3ex4_(−300)-(−271) | GACACAAAGATACACACAAGACAGGCAAGA |
| 315 | Notch3ex4_(−280)-(−251) | CACAGGGCAGGATGGCCCCAGACACAAAGA |
| 316 | Notch3ex4_(−260)-(−231) | GAGATGGCTTGGTTGGGCAGCACAGGGCAG |
| 317 | Notch3ex4_(−240)-(−211) | CAGGCGGGCACCTGTGGGCAGAGATGGCTT |
| 318 | NoLch3ex4_(−220)-(−191) | ACACCGCTCACCCACCCAGCCAGGCGGGCA |
| 319 | Notch3ex4_(−200)-(−171) | TGACAGGGGTCCTCCAGCTGACACCGCTCA |
| 320 | Notch3ex4_(−180)-(−151) | GGCCAGCACAGGGGCCTGAGTGACAGGGGT |
| 321 | Notch3ex4_(−160)-(−131) | TGAACTCTGGCAGACACCACGGCCAGCACA |
| 322 | Notch3ex4_(−140)-(−111) | CGGGCGGTGCCAGCCACCACTGAACTCTGG |
| 323 | Notch3ex4_(−120)-(−91) | GGGGGCACCGGCATGAGAATCGGGCGGTGC |
| 324 | Notch3ex4_(−100)-(−71) | CCTCTCACCTCGGAAGCCACGGGGGCACCG |
| 325 | Notch3ex4_(−80)-(−51) | CCTCCCCTCCAGACTCTTCCCCTCTCACCT |
| 326 | Notch3ex4_(−60)-(−31) | ACTGACCACACCCCCGACTACCTCCCCTCC |
| 327 | Notch3ex4_(−40)-(−11) | CAGGACAGGGTGAGTTTAGGACTGACCACA |
| 328 | Notch3ex4_(−20)-10 | CAGTCAGGGCCTGGAGGGACCAGGACAGGG |
| 329 | Notch3ex4_1-30 | GGCAGGGATCTGGCAGGGAGCAGTCAGGGC |
| 330 | Notch3ex4_21-50 | GTGGGCACAAGGGCTGCTGAGGCAGGGATC |
| 331 | SGCGex4_216-246 | TGTGGTGCTAGTCTGTGGTCAACACAGCAT |
| 332 | SGCGex4_226-256 | CACCCCACTCTGTGGTGCTAGTCTGTGGTC |
| 333 | SGCGex4_236-266 | CCTCACCCCCCACCCCACTCTGTGGTGCTA |
| 334 | SGCGex4_246-276 | CACGCTGTCCCCTCACCCCCCACCCCACTC |
| 335 | SGCGex4_256-286 | CAAAGCACTTCAGGCTGTCCCCTCACCCCC |
| 336 | Notch3ex4_397-426 | AGGGAAGGGGGCAAGGATGGTCACCGCCGG |
| 337 | Notch3ex4_407-436 | CAAACCCTAGCAGGGAAGGGGGCAAGGATG |
| 338 | Notch3ex4_417-446 | TCTGACCCTCAAACCCTAGCAGGGAAGGGG |
| 339 | Notch3ex4_427-456 | ACTTCACAATTCTGACCCTCAAACCCTAGC |
| 340 | Notch3ex4_437-466 | GTCCACGTTCACTTCACAATTCTGACCCTC |

### 5. Method for Enhancing Efficiency of Multi-Exon Skipping

The present invention provides a method for enhancing the efficiency of skipping of two or more numerically consecutive exons, which comprises

inhibiting a splicing silencer sequence, a splice site sequence, or a branch site sequence of pre-mRNA of interest when the two or more numerically consecutive exons are skipped from the pre-mRNA of interest (hereinafter, referred to as the “enhancement method of the present invention”).

In the enhancement method of the present invention, the skipping of two or more numerically consecutive exons in the pre-mRNA of interest can be performed using an antisense oligomer that induces multi-exon skipping, for example, the antisense oligomer of the present invention or the pharmaceutically acceptable salt thereof, or hydrate thereof.

The enhancement method of the present invention can enhance the efficiency of multi-exon skipping by inhibiting a splicing silencer sequence, a splice site sequence, or a branch site sequence in pre-mRNA of interest, and thereby suppressing single skipping. The enhancement method of the present invention comprises inhibiting a function of the splicing silencer sequence, the splice site sequence, or the branch site sequence in the pre-mRNA of interest, and more specifically, comprises suppressing single exon skipping by targeting these sequences using a suppressor antisense oligomer.

In the enhancement method of the present invention, the inhibition of the splicing silencer sequence, the splice site, or the branch site sequence can be performed using a suppressor antisense oligomer such as the suppressor antisense oligomer of the present invention, or a pharmaceutically acceptable salt thereof, or hydrate thereof.

In the enhancement method of the present invention, an oligomer comprising a base sequence complementary to the splicing silencer sequence, the branch site sequence, or the splice site sequence in the pre-mRNA of interest can be used as the suppressor antisense oligomer in the inhibition of the splicing silencer sequence, the branch site sequence, or the splice site sequence.

The splicing silencer sequence, the branch site sequence, and the splice site sequence are as mentioned above. In a certain embodiment of the enhancement method of the present invention, the splicing silencer sequence may be a recognition sequence of heterogeneous nuclear ribonucleoprotein A1 (hnRNP A1). Examples of the recognition sequence of human hnRNP A1 are shown in SEQ ID NOs: 370 to 379.

The term “using” or “used” in relation to the antisense oligomer and the suppressor antisense oligomer in the enhancement method of the present invention means that cells expressing the pre-mRNA of interest are allowed to incorporate the antisense oligomer and the suppressor antisense oligomer so that exon skipping is caused in the pre-mRNA of interest. Examples of the method for allowing the cells to incorporate the antisense oligomer and the suppressor antisense oligomer include introduction methods using cationic carriers such as cationic liposomes, cationic polymers, etc., or carriers using viral envelope. The cationic liposomes are, for example, liposomes composed of 2-O-(2-diethylaminoethyl)carbamoyl-1,3-O-dioleoylglycerol and phospholipids as the essential constituents (hereinafter referred to as “liposome A”), Oligofectamine (registered trademark) (manufactured by Invitrogen Corp.), Lipofectin (registered trademark) (manufactured by Invitrogen Corp.), Lipofectamine (registered trademark) (manufactured by Invitrogen Corp.), Lipofectamine 2000 (registered trademark) (manufactured by Invitrogen Corp.), DMRIE-C(registered trademark) (manufactured by Invitrogen Corp.), GeneSilencer (registered trademark) (manufactured by Gene Therapy Systems), TransMessenger (registered trademark) (manufactured by QIAGEN, Inc.), TransIT TKO (registered trademark) (manufactured by Mirus) and Nucleofector II (Lonza). Among others, liposome A is preferred. Examples of cationic polymers are JetSI (registered trademark) (manufactured by Qbiogene, Inc.) and Jet-PEI (registered trademark) (polyethylenimine, manufactured by Qbiogene, Inc.). An example of carriers using viral envelop is GenomeOne (registered trademark) (HVJ-E liposome, manufactured by Ishihara Sangyo). Alternatively, the medical devices described in Japanese Patent Nos. 2924179 and the cationic carriers described in Japanese Domestic Re-Publication PCT Nos. 2006/129594 and 2008/096690 may be used as well.

For the method for allowing cells expressing pre-mRNA of interest in the body of a patient to incorporate the antisense oligomer and the suppressor antisense oligomer, the administration method described in the section “3. Medical application” can be referred.

In the enhancement method of the present invention, the inhibition of the splicing silencer sequence, the splice site sequence, or the branch site sequence in the pre-mRNA of interest enhances the efficiency of multi-exon skipping by 2% or higher, 4% or higher, 6% or higher, 8% or higher, 10% or higher, 12% or higher, 14% or higher, 16% or higher, 18% or higher, 20% or higher, 22% or higher, 24% or higher, 26% or higher, 28% or higher, 30% or higher, 32% or higher, 34% or higher, 36% or higher, 38% or higher, 40% or higher, 42% or higher, 44% or higher, 46% or higher, 48% or higher, 50% or higher, 52% or higher, 54% or higher, 56% or higher, 58% or higher, 60% or higher, 62% or higher, 64% or higher, 66% or higher, 68% or higher, 70% or higher, 72% or higher, 74% or higher, 76% or higher, 78% or higher, 80% or higher, 82% or higher, 84% or higher, 86% or higher, 88% or higher, 90% or higher, 92% or higher, 94% or higher, 96% or higher, 98% or higher, or 100% or higher when the efficiency obtained without the inhibition is defined as 100.

In the enhancement method of the present invention, examples of the pre-mRNA of interest include human γ-sarcoglycan (SGCG) pre-mRNA, human Notch3 pre-mRNA, and human dystrophin pre-mRNA. In a certain embodiment of the present invention, the pre-mRNA of interest is human dystrophin pre-mRNA. When the pre-mRNA of interest is human dystrophin pre-mRNA, the two or more numerically consecutive exons are exons selected from the group consisting of the 45th exon to the 55th exon in the human dystrophin pre-mRNA.

### EXAMPLES

Hereinafter, the present invention will be described in more detail with reference to Examples and Test Examples below, but is not deemed to be limited thereto.

### Example 1: Production of Antisense Oligomer

### Production of 4-{[(2S,6R)-6-(4-benzamido-2-oxopyrimidin-1-yl)-4-tritylmorpholin-2-yl]methoxy}-4-oxobutanoic acid supported on amino polystyrene resin (Compound 1)

### Step 1: Production of 4-{[(2S,6R)-6-(4-benzamido-2-oxopyrimidin-1(2H)-yl)-4-tritylmorpholin-2-yl]methoxy}-4-oxobutanoic acid

Under argon atmosphere, 3.44 g of N-{1-[(2R,6S)-6-(hydroxymethyl)-4-tritylmorpholin-2-yl]-2-oxo-1,2-dihydropyrimidin-4-yl}benzamide and 1.1 g of 4-dimethylaminopyridine (4-DMAP) were suspended in 50 mL of dichloromethane, and 0.90 g of succinic anhydride was added to the suspension, followed by stirring at room temperature for 3 hours. To the reaction mixture, 10 mL of methanol was added, and the mixture was concentrated under reduced pressure. The residue was extracted using ethyl acetate and 0.5 M aqueous potassium dihydrogenphosphate solution. The resulting organic layer was washed sequentially with 0.5 M aqueous potassium dihydrogenphosphate solution, water and brine in the order mentioned. The resulting organic layer was dried over sodium sulfate and concentrated under reduced pressure to give 4.0 g of the product.

### Step 2; Production of 4-{[(2S,6R)-6-(4-benzamido-2-oxopyrimidin-1-yl)-4-tritylmorpholin-2-yl]methoxy}-4-oxobutanoic acid supported on amino polystyrene resin

After 4.0 g of 4-{[(2S,6R)-6-(4-benzamido-2-oxopyrimidin-1(2H)-yl)-4-tritylmorpholin-2-yl]methoxy}-4-oxobutanoic acid was dissolved in 200 mL of pyridine (dehydrated), 0.73 g of 4-DMAP and 11.5 g of 1-ethyl-3-(3-dimethylaminopropyl)carbodiimide hydrochloride were added to the solution. Then, 25.0 g of amino polystyrene resin Primer support 200 amino (manufactured by GE Healthcare Japan Co., Ltd., 17-5214-97) and 8.5 mL of triethylamine were added to the mixture, followed by shaking at room temperature for 4 days. After completion of the reaction, the resin was taken out by filtration. The resulting resin was washed sequentially with pyridine, methanol and dichloromethane in the order mentioned, and dried under reduced pressure. To the resulting resin, 200 mL of tetrahydrofuran (dehydrate), 15 mL of acetic anhydride and 15 mL of 2,6-lutidine were added, and the mixture was shaken at room temperature for 2 hours. The resin was taken out by filtration, washed sequentially with pyridine, methanol and dichloromethane in the order mentioned and dried under reduced pressure to give 26.7 g of the product of interest.

The loading amount of the product was determined from the molar amount of the trityl per g resin by measuring UV absorbance at 409 nm using a known method. The loading amount of the resin was 129.2 μmol/g.

### Conditions of UV Measurement

Apparatus: U-2910 (Hitachi, Ltd.)

Solvent: methanesulfonic acid

Wavelength: 409 nm

ε value: 45000

### Production of 4-{[(2S,6R)-6-(5-methyl-2,4-dioxopyrimidin-1-yl)-4-tritylmorpholin-2-yl]methoxy}-4-oxobutanoic acid supported on amino polystyrene resin (Compound 2)

The title compound was produced in the same manner as in Compound 1 except that 1-[(2R,6S)-6-(hydroxymethyl)-4-tritylmorpholin-2-yl]-5-methylpyrimidine-2,4(1H,3H)-dione was used in this step instead of N-{1-[(2R,6S)-6-(hydroxymethyl)-4-tritylmorpholin-2-yl]-2-oxo-1,2-dihydropyrimidin-4-yl}benzamide used in Step 1 in the production of Compound 1.

The loading amount of the product was determined from the molar amount of the trityl per g resin by measuring UV absorbance at 409 nm using a known method. The loading amount of the resin was 164.0 μmol/g.

### Production of 4-{[(2S,6R)-6-(6-benzamidopurin-9-yl)-4-tritylmorpholin-2-yl]methoxy}-4-oxobutanoic acid supported on amino polystyrene resin (Compound 3)

The title compound was produced in the same manner as in Compound 1 except that N-{9-[(2R,6S)-6-(hydroxymethyl)-4-tritylmorpholin-2-yl]purin-6-yl}benzamide was used in this step instead of N-{1-[(2R,6S)-6-(hydroxymethyl)-4-tritylmorpholin-2-yl]-2-oxo-1,2-dihydropyrimidin-4-yl}benzamide used in Step 1 in the production of Compound 1.

The loading amount of the product was determined from the molar amount of the trityl per g resin by measuring UV absorbance at 409 nm using a known method. The loading amount of the resin was 185.7 μmol/g.

### Production of 4-{{(2S,6R)-6-{6-(2-cyanoethoxy)-2-[(2-phenoxyacetyl)amino]purin-9-yl}-4-tritylmorpholin-2-yl}methoxy}-4-oxobutanoic acid supported on amino polystyrene resin (Compound 4)

The title compound was produced in the same manner as in Compound 1 except that N-{6-(2-cyanoethoxy)-9-[(2R,6S)-6-(hydroxymethyl)-4-tritylmorpholin-2-yl]purin-2-yl}-2-phenoxyacetamide was used in this step instead of N-{1-[(2R,6S)-6-(hydroxymethyl)-4-tritylmorpholin-2-yl]-2-oxo-1,2-dihydropyrimidin-4-yl}benzamide used in Step 1 in the production of Compound 1.

The loading amount of the product was determined from the molar amount of the trityl per g resin by measuring UV absorbance at 409 nm using a known method. The loading amount of the resin was 164.8 μmol/g.

### Production of Antisense PMO Targeting Region in Vicinity of Donor or Acceptor of Intron in Human Dystrophin Gene

According to the description given below, PMOs having the base sequences of PMO Nos. 1 to 232 (SEQ ID NOs: 1 to 232) shown in Table 11 which targeted regions in the vicinity of donors or acceptors of introns 44 to 55 in human dystrophin pre-mRNA shown in Table 10 were synthesized. The 5′ end of each PMO is Group (3) below. The synthesized PMO was dissolved in water for injection (manufactured by Otsuka Pharmaceutical Factory, Inc.).

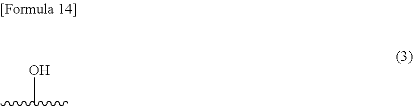 [SMILES: *O]

The target base sequence of the antisense oligomer of the present invention or the suppressor antisense oligomer of the present invention was described as “Ha_{1_}b₁-c₁”, “Ha_{2_}b₂-c_{2_}Ha_{3_}b₃-c₃”, or “Ha_{4_}b₄-c_{4_}Ha_{5_}b₅-c₅_Ha_{6_}b₆-c₆”.

“Ha_{1_}b₁-c₁” is the target base sequence of the antisense oligomer of the first embodiment used herein. “Ha₁” represents the ath exon of the human dystrophin gene, “b₁” represents the 5′-terminal base of the target base sequence, and “c₁” represents the 3′-terminal base of the target base sequence. “Ha_{1_}_b-c₁” may have one additional base at its 3′ end, and such a target base sequence is represented by “Ha_{1_}b₁-c_{1_}N” (“N” represents a given base).

When “b₁” and “c₁” are positive integers, “b₁” and “c₁” each represent a base number in the downstream direction when the 5′-terminal base of the ath exon is counted as the 1st base. On the other hand, when “b₁” and “c₁” are negative numbers, “b₁” and “c₁” each represent a base number in the upstream direction when the 3′-terminal base of the (a-1)th intron is counted as the 1st base.

For example, “H45_(−10)-15” means a base sequence in which the 5′ end of the target base sequence is the 10th base in the upstream direction from the 3′ end of the 44th intron and the 3′ end of the target base sequence is the 15th base in the downstream direction from the 5′ end of the 45th exon.

“Ha_{2_}b₂-c_{2_}Ha_{3_}b₃-c₃” is the target base sequence of the antisense oligomer of the second embodiment used herein. “Ha₂_b₂-c₂” which is the first part of “Ha_{2_}b₂-c_{2_}Ha_{3_}b₃-c₃” means the target base sequence of a 3′ unit oligomer constituting the antisense oligomer, and the second part “Ha_{3_}b₃-c₃” means the target base sequence of a 5′ unit oligomer constituting the antisense oligomer. “Ha_{3_}b₃-c₃” may have one additional base at its 3′ end, and such a target base sequence is represented by “Ha_{2_}b₂-c_{2_}Ha_{3_}b₃-c_{3_}N” (“N” represents a given base). Each of “Ha_{2_}b₂-c₂” and “Ha_{3_}b_{3_}-c₃” abides by the same notation as that of “Ha_{1_}b₁-c₁”.

When “Ha₂” and “Ha₃” are the same, the “_Ha₃” part may be omitted.

For example, “H45_(−5)-5_25-35” or “H45_(−5)-5_H45_25-35” means a base sequence in which the target base sequence of the 3′ unit oligomer constituting the antisense oligomer is “H45_(−5)-5” and the target base sequence of the 5′ unit oligomer constituting the antisense oligomer is “H45_25-35”.

“Ha_{4_}b₄-c_{4_}Ha_{5_}b₅-c_{5_}Ha_{6_}b₆-c₆” is the target base sequence of the antisense oligomer of the third embodiment used herein. “Ha_{4_}b₄-c₄” which is the first part of “Ha_{4_}b₄-c_{4_}Ha_{5_}b₅-c_{5_}Ha_{6_}b₆-c₆” means the target base sequence of a 3′ unit oligomer constituting the antisense oligomer, the second part “Ha_{5_}b₅-c₅” means the target base sequence of an intermediate unit oligomer constituting the antisense oligomer, and the third part “Ha_{6_}b₆-c₆” means the target base sequence of a 5′ unit oligomer constituting the antisense oligomer. “Ha_{6_}b₆-c₆” may have one additional base at its 3′ end, and such a target base sequence is represented by “Ha_{4_}b₄-c_{4_}Ha_{5_}b₅-c_{5_}Ha_{6_}b₆-c_{6_}N” (“N” represents a given base). Each of “Ha_{4_}b₄-c₄”, “Ha_{5_}b_{5_}-c₅”, and “Ha_{6_}b_{6_}-c₆” abides by the same notation as that of “Ha_{1_}b₁-c₁”.

When “Ha₄” is the same with “Ha₅” and “Ha₆”, the “_Ha₅” and “_Ha₆” parts may be omitted.

For example, “H45_(−5)-5_25-35_60-70” or “H45_(−5)-5_H45_25-35_H45_60-70” means a base sequence in which the target base sequence of the 3′ unit oligomer constituting the antisense oligomer is “H45_(−5)-5”, the target base sequence of the intermediate unit oligomer is “H45_25-35”, and the target base sequence of the 5′ unit oligomer is “H45_60-70”.

TABLE 10
Target region in human dystrophin pre-mRNA

| | | SEQ |
|---|---|---|
| Target region | Range of target region | ID NO |
| (R1) Region in vicinity of | Region indicated by range of −20 | 233 |
| donor of intron 44 | bases to +400 bases with 5′ end of | |
| | intron 44 defined as basing point 0 | |
| (R2) Region in vicinity of | Region indicated by range of −600 | 234 |
| acceptor of intron 44 | bases to +50 bases with 3′ end of | |
| | intron 44 defined as basing point 0 | |
| (R3) Region in vicinity of | Region indicated by range of −20 | 235 |
| donor of intron 45 | bases to +400 bases with 5′ end of | |
| | intron 45 defined as basing point 0 | |
| (R4) Region in vicinity of | Region indicated by range of −400 | 236 |
| acceptor of intron 45 | bases to +50 bases with 3′ end of | |
| | intron 45 defined as basing point 0 | |
| (R5) Region in vicinity of | Region indicated by range of −20 | 237 |
| donor of intron 46 | bases to +400 bases with 5′ end of | |
| | intron 46 defined as basing point 0 | |
| (R6) Region in vicinity of | Region indicated by range of −400 | 238 |
| acceptor of intron 46 | bases to +50 bases with 3′ end of | |
| | intron 46 defined as basing point 0 | |
| (R7) Region in vicinity of | Region indicated by range of −20 | 239 |
| donor of intron 47 | bases to +400 bases with 5′ end of | |
| | intron 47 defined as basing point 0 | |
| (R8) Region in vicinity of | Region indicated by range of −400 | 240 |
| acceptor of intron 47 | bases to +50 bases with 3′ end of | |
| | intron 47 defined as basing point 0 | |
| (R9) Region in vicinity of | Region indicated by range of −20 | 241 |
| donor of intron 48 | bases to +400 bases with 5′ end of | |
| | intron 48 defined as basing point 0 | |
| (R10) Region in vicinity of | Region indicated by range of −400 | 242 |
| acceptor of intron 48 | bases to +50 bases with 3′ end of | |
| | intron 48 defined as basing point 0 | |
| (R11) Region in vicinity of | Region indicated by range of −20 | 243 |
| donor of intron 49 | bases to +400 bases with 5′ end of | |
| | intron 49 defined as basing point 0 | |
| (R12) Region in vicinity of | Region indicated by range of −400 | 244 |
| acceptor of intron 49 | bases to +50 bases with 3′ end of | |
| | intron 49 defined as basing point 0 | |
| (R13) Region in vicinity of | Region indicated by range of −20 | 245 |
| donor of intron 50 | bases to +400 bases with 5′ end of | |
| | intron 50 defined as basing point 0 | |
| (R14) Region in vicinity of | Region indicated by range of −400 | 246 |
| acceptor of intron 50 | bases to +50 bases with 3′ end of | |
| | intron 50 defined as basing point 0 | |
| (R15) Region in vicinity of | Region indicated by range of −20 | 247 |
| donor of intron 51 | bases to +400 bases with 5′ end of | |
| | intron 51 defined as basing point 0 | |
| (R16) Region in vicinity of | Region indicated by range of −400 | 248 |
| acceptor of intron 51 | bases to +50 bases with 3′ end of | |
| | intron 51 defined as basing point 0 | |
| (R17) Region in vicinity of | Region indicated by range of −20 | 249 |
| donor of intron 52 | bases to +400 bases with 5′ end of | |
| | intron 52 defined as basing point 0 | |
| (R18) Region in vicinity of | Region indicated by range of −400 | 250 |
| acceptor of intron 52 | bases to +50 bases with 3′ end of | |
| | intron 52 defined as basing point 0 | |
| (R19) Region in vicinity of | Region indicated by range of −20 | 251 |
| donor of intron 53 | bases to +400 bases with 5′ end of | |
| | intron 53 defined as basing point 0 | |
| (R20) Region in vicinity of | Region indicated by range of −400 | 252 |
| acceptor of intron 53 | bases to +50 bases with 3′ end of | |
| | intron 53 defined as basing point 0 | |
| (R21) Region in vicinity of | Region indicated by range of −20 | 253 |
| donor of intron 54 | bases to +400 bases with 5′ end of | |
| | intron 54 defined as basing point 0 | |
| (R22) Region in vicinity of | Region indicated by range of −400 | 254 |
| acceptor of intron 54 | bases to +50 bases with 3′ end of | |
| | intron 54 defined as basing point 0 | |
| (R23) Region in vicinity of | Region indicated by range of −20 | 255 |
| donor of intron 55 | bases to +400 bases with 5′ end of | |
| | intron 55 defined as basing point 0 | |
| (R24) Region in vicinity of | Region indicated by range of −400 | 256 |
| acceptor of intron 55 | bases to +50 bases with 3′ end of | |
| | intron 55 defined as basing point 0 | |

TABLE 11

| PMO | Target | | | SEQ |
|---|---|---|---|---|
| No. | region | Target base sequence | Base sequence of PMO | ID NO |
| 1 | R1 | H44150-175 | TTTCGAAAAAACAAATCAAAGACTTA | 1 |
| 2 | | H44_157-196 | ATGTGCTGAAGATAAATACAATTTCGAAAA | 2 |
| | | | AACAAATCAA | |
| 3 | | H44_l59-193 | TGCTGAAGATAAATACAATTTCGAAAAAAC | 3 |
| | | | AAATC | |
| 4 | | H44_170-199 | CAGATGTGCTGAAGATAAATACAATTTCGA | 4 |
| 5 | | H44_l79-206 | AAGAGTCCAGATGTGCTGAAGATAAATA | 5 |
| 6 | | H44_209-236 | ACCCTTCAGAACCTGATCTTTAAGAAGT | 6 |
| 7 | | H44_239-266 | TGACAACAACAGTCAAAAGTAATTTCCA | 7 |
| 8 | | H44_269-296 | ATGATAATTTTCTTTCTAGTAATATAAT | 8 |
| 9 | | H44_299-326 | TCCATAGCACCGTGCTCTAATATTATCA | 9 |
| 10 | | H44_330-355 | GGCAAACTCTCTCATCCTGACACAAA | 10 |
| 11 | | H44_354-381 | TTTATCAGATAAACCAGCTCCGTCCAGG | 11 |
| 12 | | H44_389-416 | CTTCCCTCTGTCACAGATTCAATTATAT | 12 |
| 13 | | H44_413-438 | AAAACACCTTGCTGTTACGATGCTTC | 13 |
| 14 | | H44_423-452 | TGCCCCAAAGCCACAAAACACCTTGCTGTT | 14 |
| 15 | | H44_455-480 | GGTTCCAACATAAAGCCGAAATACAC | 15 |
| 16 | | H44_479-506 | TATGCCACAAGTTCTCCTTCTGGAAAGG | 16 |
| 17 | | H44_509-536 | GATATTTCTAGCAACTTCATTTTAGCTA | 17 |
| 18 | | H44_150-164_179-193 | TGCTGAAGATAAATACAAATCAAAGACTTA | 18 |
| 19 | | H44_150-164_192-206 | AAGAGTCCAGATGTGCAAATCAAAGACTTA | 19 |
| 20 | | H44_161-175_179-193 | TGCTGAAGATAAATATTTCGAAAAAACAAA | 20 |
| 21 | | H44_161-175_192-206 | AAGAGTCCAGATGTGTTTCGAAAAAACAAA | 21 |
| 22 | R2 | H45_(−598)-(−571) | TTAATTGCTTTCAGGAGCATCCCATCAA | 22 |
| 23 | | H45_(−568)-(−541) | TTTGCATTAGAAGCCACAAAAAACTGAG | 23 |
| 24 | | H45_(−538)-(−511) | TCATTTCAAATTCTGTCTGCGTCAATGT | 24 |
| 25 | | H45_(−518)-(−491) | TTGCTATATTAGAAGAAAATTCATTTCA | 25 |
| 26 | | H45_(−515)-(−488) | TAATTGCTATATTAGAAGAAAATTCATT | 26 |
| 27 | | H45_(−508)-(−479) | AAATAAAATTAATTGCTATATTAGAAGAAA | 27 |
| 28 | | H45_(−478)-(−449) | CAGTATTAAAAAAAAACTCTAGAGATATTT | 28 |
| 29 | | H45_(−470)-(−443) | TAGTCACAGTATTAAAAAAAAACTCTAG | 29 |
| 30 | | H45_(−448)-(−421) | GTGAAAAAGAACAAACATAGGTTAGTCA | 30 |
| 31 | | H45_(−439)-(−412) | ATACGAGAGGTGAAAAAGAACAAACATA | 31 |
| 32 | | H45_(−436)-(−409) | TGGATACGAGAGGTGAAAAAGAACAAAC | 32 |
| 33 | | H45_(−422)-(−395) | TTTCTTAGTGATCGTGGATACGAGAGGT | 33 |
| 34 | | H45_(−418)-(−394) | GTTTCTTAGTGATCGTGGATACGAG | 34 |
| 35 | | H45_(−418)-(−391) | TGGGTTTCTTAGTGATCGTGGATACGAG | 35 |
| 36 | | H45_(−415)-(−391) | TGGGTTTCTTAGTGATCGTGGATAC | 36 |
| 37 | | H45_(−413)-(−386) | GTATTTGGGTTTCTTAGTGATCGTGGAT | 37 |
| 38 | | H45_(−405)-(−378) | TGAACAAAGTATTTGGGTTTCTTAGTGA | 38 |
| 39 | | H45_(−389)-(−362) | TTGTAAAATTTAAACATGAACAAAGTAT | 39 |
| 40 | | H45_(−349)-(−322) | TCCCCACAAGGATGTTCCATGTTTAATA | 40 |
| 41 | | H45_(−320)-(−293) | ACCTTTTCAAGAGCAAATTCGATTTCTT | 41 |
| 42 | | H45_(−305)-(−280) | CAATTAGTTGGAAACCTTTTCAAGAG | 42 |
| 43 | | H45_(−290)-(−263) | TATAATGTCCTACAAATCAATTAGTTGG | 43 |
| 44 | | H45_(−275)-(−250) | AGCTAGAGGATGTTATAATGTCCTAC | 44 |
| 45 | | H45_(−260)-(−233) | ATTTTTGTAAGCTTGTCAGCTAGAGGAT | 45 |
| 46 | | H45_(−230)-(−203) | AAAGCACCCTCTCGGTTAGCTCCAGTTT | 46 |
| 47 | | H45_(−200)-(−173) | CAGAAAGACACCTTTTATGTGTCAGGGA | 47 |
| 48 | | H45_(−190)-(−163) | GGATACAAGACAGAAAGACACCTTTTAT | 48 |
| 49 | | 145_(−185)-(−160) | AAAGGATACAAGACAGAAAGACACCT | 49 |
| 50 | | H45_(−l7l)-(−144) | TGACATGCCCATATCCAAAGGATACAAG | 50 |
| 51 | | H45_(−168)-(−141) | AACTGACATGCCCATATCCAAAGGATAC | 51 |
| 52 | | H45_(−142)-(−115) | AGCTCCATGTGAAAATTTCCCTATGAAA | 52 |
| 53 | | H45_(−110)-(−83) | TGCAGTTGTACTGGCAAAGAAAGAAATA | 53 |
| 54 | | H45_(−80)-(−53) | TGAGAAAAGATTAAACAGTGTGCTACCA | 54 |
| 55 | | H45_(−78)-(−51) | TTTGACAAAAGATTAAACAGTGTGCTAC | 55 |
| 56 | | H45_(−70)-(−43) | TCTTTTTATTTGAGAAAAGATTAAACAG | 56 |
| 57 | | H45_(−60)-(−33) | AAGCCCCATGTCTTTTTATTTGAGAAAA | 57 |
| 58 | | H45_(−50)-(−23) | AACAAAAATGAAGCCCCATGTCTTTTTA | 58 |
| 59 | | H45_(−44)-(−17) | AGGCAAAACAAAAATGAAGCCCCATGTC | 59 |
| 60 | | H45_(−25)-3 | TTCCTGTAAGATACCAAAAAGGCAAAAC | 60 |
| 61 | | H45_(−23)-5 | AGTTCCTGTAAGATACCAAAAAGGCAAA | 61 |
| 62 | | H45_(−22)-3 | TTCCTGTAAGATACCAAAAAGGCAA | 62 |
| 63 | | H45_(−21)-7 | GGAGTTCCTGTAAGATACCAAAAAGGCA | 63 |
| 64 | | H45_(−20)-5 | AGTTCCTGTAAGATACCAAAAAGGC | 64 |
| 65 | | H45_(−20)-8 | TGGAGTTCCTGTAAGATACCAAAAAGGC | 65 |
| 66 | | H45_(−18)-10 | CCTGGAGTTCCTGTAAGATACCAAAAAG | 66 |
| 67 | | H45_(−17)-8 | TGGAGTTCCTGTAAGATACCAAAAA | 67 |
| 68 | | H45_(−16)-12 | ATCCTGGAGTTCCTGTAAGATACCAAAA | 68 |
| 69 | | H45_(−15)-10 | CCTGGAGTTCCTGTAAGATACCAAA | 69 |
| 70 | | H45_(−l4)-14 | CCATCCTGGAGTTCCTGTAAGATACCAA | 70 |
| 71 | | H45_(−10)-16 | TGCCATCCTGGAGTTCCTGTAAGATA | 71 |
| 72 | | H45_(−5)-20 | CCAATCCCATCCTGGAGTTCCTGTA | 72 |
| 73 | | H45_l-25 | GCTGCCCAATGCCATCCTGGAGTTC | 73 |
| 74 | | H45_6-30 | TTGCCGCTGCCCAATGCCATCCTGG | 74 |
| 75 | | H45_11-35 | ACAGTTTGCCGCTGCCCAATGCCAT | 75 |
| 76 | | H45_l1-40 | TGACAACAGTTTGCCGCTGCCCAATGCCAT | 76 |
| 77 | | H45_16-35 | ACAGTTTGCCGCTGCCCAAT | 77 |
| 78 | | H45_l6-40 | TGACAACAGTTTGCCGCTGCCCAAT | 78 |
| 79 | | H45_21-45 | TGTTCTGACAACAGTTTGCCGCTGC | 79 |
| 80 | | H45_(−423)-(−412)_16- | AGTTTGCCGCTGCCCAATATACGAGAGGTG | 80 |
| | | 33 | | |
| 81 | | H45_(−416)-(−402)_16- | TTCCCGCTGCCCAATGTGATCGTGGATACG | 81 |
| | | 30 | | |
| 82 | | H45_(−411)-(−400)_16- | AGTTTGCCCCTGCCCAATTAGTGATCGTGG | 82 |
| | | 33 | | |
| 83 | | H45_(−405)-(−391)_16- | TTGCCGCTGCCCAATTGGGTTTCTTAGTGA | 83 |
| | | 30 | | |
| 84 | | H45_(−402)-(−391)_16- | AGTTTGCCGCTGCCCAATTGGGTTTCTTAG | 84 |
| | | 33 | | |
| 85 | | H45_(−156)-(−143)- | AATTTCCCTATGAACTGACATGCCCATA | 85 |
| | | (−141)-(−128) | | |
| 86 | | H45_(−78)-(−65)_(−44)- | TGAAGCCCCATGTCAAACAGTGTGCTAC | 86 |
| | | (−31) | | |
| 87 | | H45_(−78)-(−65)_(−34)- | AAAACAAAAATGAAGAACAGTGTGCTAC | 87 |
| | | (−21) | | |
| 88 | | H45_(−78)-(−65)_16-29 | TGCCGCTGCCCAATAAACAGTGTGCTAC | 88 |
| 89 | | B45_(−78)-(−65)_21-34 | CAGTTTGCCGCTGCAAACAGTGTGCTAC | 89 |
| 90 | | H45_(−78)-(−65)_27-40 | TGACAACAGTTTGCAAACAGTGTGCTAC | 90 |
| 91 | | H45_(−74)-(−62)_(−40)- | AAATGAAGCCCCAATTAAACAGTGTG | 91 |
| | | (−28) | | |
| 92 | | H45_(−72)-(−58)_(−38)- | AAAAATGAAGCCCAAAGATTAAACAGTG | 92 |
| | | (−26) | | |
| 93 | | H45_(−71)-(−58)_(−38)- | CAAAAATGAAGCCCAAAGATTAAACAGT | 93 |
| | | (−25) | | |
| 94 | | H45_(−71)-(−58)_16-29 | TGCCCCTGCCCAATAAAGATTAAACAGT | 94 |
| 95 | | H45_(−71)-(−58)_21-34 | CAGTTTGCCGCTGCAAAGATTAAACAGT | 95 |
| 96 | | H45_(−71)-(−58)_27-40 | TGACAACAGTTTGCAAAGATTAAACAGT | 96 |
| 97 | | H45_(−68)-(−60)_(−36)- | GCCGCTGCCC_AAATGAAGCAGATTAAAC | 97 |
| | | (−28)_19-28 | | |
| 98 | | H45_(−64)-(−51)_(−44)- | TGAAGCCCCATGTCTTTGAGAAAAGATT | 98 |
| | | (−31) | | |
| 99 | | H45_(−64)-(−51)_(−35)- | AAACAAAAATGAAGTTTGAGAAAAGATT | 99 |
| | | (−22) | | |
| 100 | | H45_(−64)-(−51)-16-29 | TGCCGCTGCCCAATTTTGAGAAAAGATT | 100 |
| 101 | | H45_(−64)-(−51)_21-34 | CAGTTTGCCGCTGCTTTGAGAAAAGATT | 101 |
| 102 | | H45_(−64)-(−51)-27-40 | TGACAACAGTTTGCTTTGAGAAAAGATT | 102 |
| 103 | | H45_(−44)-(−3l)_l6-29 | TGCCGCTGCCCAATTGAAGCCCCATGTC | 103 |
| 104 | | H45_(−44)-(−31)_27-40 | TGACAACAGTTTGCTGAAGCCCCATGTC | 104 |
| 105 | | H45_(−44)-(−31)_21- | TAGTTTGCCGCTGCTGAAGCCCCATGTC | 105 |
| | | 33_T | | |
| 106 | | H45_(−38)-(−25)_16-29 | TGCCGCTGCCCAATCAAAAATGAAGCCC | 106 |
| 107 | | H45_(−38)-(−25)_21-34 | CAGTTTGCCGCTGCCAAAAATGAAGCCC | 107 |
| 108 | | H45_(−38)-(−25)_27-40 | TGACAACAGTTTGCCAAAAATGAAGCCC | 108 |
| 109 | | H45_(−34)-(−21)_16-29 | TGCCGCTGCCCAATAAAACAAAAATGAA | 109 |
| 110 | | H45_(−34)-(−21)_21-34 | CAGTTTGCCGCTGCAAAACAAAAATGAA | 110 |
| 111 | | H45_(−34)-(−21)_27-40 | TGACAACAGTTTGCAAAACAAAAATGAA | 111 |
| 112 | | H45-1-21 | CCCAATGCCATCCTGGAGTTC | 112 |
| 144 | R3 | H45_167-194 | TTAGATCTGTCGCCCTACCTCTTTTTTC | 144 |
| 145 | | H45_227-254 | TCTCATGAAATATTCTTCTAAAGAAAGC | 145 |
| 146 | | H45_257-284 | ATGTTAGTGCCTTTCACCCTGCTTATAA | 146 |
| 147 | | H45_287-314 | GCTGTTGATTAATGGTTGATAGGTTCTT | 147 |
| 148 | | H45_317-344 | TGAAAAAAAGAAATAAAAAATTTCTTTA | 148 |
| 149 | | H45_347-374 | TAACTAGCCACAAGTATATATTTTAGTA | 149 |
| 150 | R4 | H46_(−200)-(−173) | TCAAGAATCTCTAAATGATAAGAGATTA | 150 |
| 151 | | H46_(−171)-(−144) | TTCACTTTGAACAAAGTAATTTCAATAT | 151 |
| 152 | | H46_(−146)-(−119) | ACCATACATAATTTAAGAAAATTCATTC | 152 |
| 153 | | H46_(−141)-(−114) | TGTTAACCATACATAATTTAAGAAAATT | 153 |
| 154 | | H46_(−136)-(−109) | AAAGATGTTAACCATACATAATTTAAGA | 154 |
| 155 | | H46_(−126)-(−99) | AAGCAATTTAAAAGATGTTAACCATACA | 155 |
| 156 | | H46_(−116)-(−89) | ATTTAAAAATAAGCAATTTAAAAGATGT | 156 |
| 157 | | H46_(−111)-(−84) | TGGCAATTTAAAAATAAGCAATTTAAAA | 157 |
| 158 | | H46_(−106)-(−79) | AAACATGGCAATTTAAAAATAAGCAATT | 158 |
| 159 | | H46_(−81)-(−54) | TATTTGTTAATGCAAACTGGGACACAAA | 159 |
| 160 | | H46_(−51)-(−24) | TTATTTTTTTTTCCAACATAGTTCTCAA | 160 |
| 161 | | H46_(−18)-6 | TCTAGCCTGGAGAAAGAAGAATAA | 161 |
| 162 | | H46_(−15)-9 | TCTTCTAGCCTGGAGAAAGAAGAA | 162 |
| 163 | | H46_(−9)-15 | TTTTGTTCTTCTAGCCTGGAGAAA | 163 |
| 164 | R5 | H46_139-166 | TTGAGAAAATAAAATTACCTTGACTTGC | 164 |
| 165 | | H46_l69-196 | ACTTCTTTATGCAAGCAGGCCCTGGGGG | 165 |
| 166 | | H46_199-226 | CAATGATTGAATTAAAAAATAGATTCAT | 166 |
| 167 | | H46_229-256 | GAACTATGAATAACCTAATGGGCAGAAA | 167 |
| 168 | | H46_259-286 | ATAAAGTTGTGAGAAAAACACTTTAGCA | 168 |
| 169 | | H46_289-316 | ACTGGTTCAGAACTGCAGGGTTAAGAAG | 169 |
| 170 | | H46_319-346 | ACACACATATATACATATGTTCTTATGT | 170 |
| 171 | R6 | H47_(−200)-(−173) | CTCACCCCCTCAGTAGATAAATCTCTGT | 171 |
| 172 | | H47_(−171)-(−144) | TCTGAGCACAGAGCTGATTGACTGAAAC | 172 |
| 173 | | H47_(−141)-(−114) | AGTGGTACCTCAAATACCAACAGTTTTC | 173 |
| 174 | | H47_(−111)-(−84) | ATCAAAATGAAGCGACTTGACCGAGGGC | 174 |
| 175 | | H47_(−81)-(−54) | TACCTTGTCTTTGCTTCTATTGATTAGT | 175 |
| 176 | | H47_(−51)-(−24) | GTTTAAAATGAATTACAGCACAATTCCA | 176 |
| 177 | | H47_(−16)-12 | TTCCACCAGTAACTGAAACAGACAAATG | 177 |
| 178 | | H47_l-29 | TGGCGCAGGGGCAACTCTTCCACCAGTAA | 178 |
| 179 | R7 | H47_135-162 | TTAATGTCTAACCTTTATCCACTGGAGA | 179 |
| 180 | R8 | H48_(−21)-7 | TGGAAACCTGAAAGGAAAATACATTTTA | 180 |
| 181 | | H48_1-28 | CTTGTTTCTCAGGTAAAGCTCTGGAAAC | 181 |
| 182 | R9 | H48_177-204 | AAGCAAAAAGTTCCCTACCTGAACGTCA | 182 |
| 183 | R10 | H49_(−21)-7 | CAGTTTCCTGGGGAAAAGAACCCATATA | 183 |
| 184 | | H49_22-47 | ATCTCTTCCACATCCGGTTGTTTAGC | 184 |
| 185 | R11 | H49_93-l20 | TAGAGGTTGCTTCATTACCTTCACTGGC | 185 |
| 186 | R12 | H50_(−200)-(−173) | ACCCTACAAATATTTATCAATTGCTCCA | 186 |
| 187 | | H50_(−171)-(−144) | TAAAGGAATTATAATTATTTTAGCCAAC | 187 |
| 188 | | H50_(-141)-(−114) | TAAATTAACTTTAGTGGGTAGAATTTCT | 188 |
| 189 | | H50_(−111)-(−84) | TATTATTGGATTTCTATTATATTTTACT | 189 |
| 190 | | H50_(−81)-(−54) | TCATGAACATCTTAATCCATTTGGTGAA | 190 |
| 191 | | H50_(−51)-(−24) | TACTTATTCGATTAACACTTTGAAGATA | 191 |
| 192 | | H50_(−21)-7 | ACTTCCTCTTTAACAGAAAAGCATACAC | 192 |
| 193 | R13 | H50_l00-127 | GGGATCCAGTATACTTACAGGCTCCAAT | 193 |
| 194 | | H50_90-114 | CTTACAGGCTCCAATAGTGGTCAGT | 194 |
| 195 | R14 | H51_(−181-10 | TGAGTAGGAGCTAAAATATTTTGGGTTT | 195 |
| 196 | R15 | H51_224-251 | TATCATTTTTTCTCATACCTTCTGCTTG | 196 |
| 197 | R16 | H52_(−47)-(−20) | TTAGTATCAGGGTTCTTCAGCGTTGTGT | 197 |
| 198 | | H52_(−18)-10 | GCATTGTTGCCTGTAAGAACAAATATCC | 198 |
| 199 | | H52_9-38 | AACTGGGGACGCCTCTGTTCCAAATCCTGC | 199 |
| 200 | R17 | H52_109-136 | GCTTGTTAAAAAACTTACTTCGATCCGT | 200 |
| 201 | R18 | H53_(−197)-(−170) | AATATTAGTTTCTGTTAAATTATTTTCC | 201 |
| 202 | | H53_(−167)-(−140) | ACAACAGGATTCTTTGCTTTTTTGATGG | 202 |
| 203 | | H53_(−137)-(−110) | TTATTCATTGTGTTATGGCTAGGATGAT | 203 |
| 204 | | H53_(−107)-(−80) | ATCTCACATTTATGTTGCTTATTTAAAA | 204 |
| 205 | | H53_(−77)-(−50) | GAGACATTTTAAATGTAACTTCCAAACG | 205 |
| 206 | | H53_(−47)-(−20) | AAATATATAGTAGTAAATGCTAGTCTGG | 206 |
| 207 | | H53_(−18)-10 | ATTCTTTCAACTAGAATAAAAGGAAAAA | 207 |
| 208 | R19 | H53_203-230 | GGTATCTTTGATACTAACCTTGGTTTCT | 208 |
| 209 | R20 | H54_(−18)-10 | TGGCCAACTGCTATAGATTTTTATGAGA | 209 |
| 210 | R21 | H54_146-173 | AAATAATGTAATTCATACCTTTTATGAA | 210 |
| 211 | | H54_176-203 | CCCCATTATTACAGCCAACAGTAGTTTT | 211 |
| 212 | | H54_206-233 | CAAATCCTCATGGTCCATCCAGTTTCAC | 212 |
| 213 | | H54_236-263 | CCAAGCTCCAGTTTAGCTGGATTGGAAA | 213 |
| 214 | | H54_266-293 | TTTAGTTGGTATTTATCGTCTTGAACCC | 214 |
| 215 | | H54_296-323 | TAAAATAGAAGTCTGAGCCAAGTCCGTG | 215 |
| 216 | | H54_326-353 | GGGCAAATGAGATCTTATGTTCCTCGTT | 216 |
| 217 | R22 | H55_(−197)-(−170) | TTTACTTTCATTTTTATTTAACTTAAAG | 217 |
| 218 | | H55_(−167)-(−140) | TAAGCAACAACTATAATATTGTGCAGTC | 218 |
| 219 | | H55_(−137)-(−110) | TGGGGTGAGTTGTTGCTACAGCTCTTCC | 219 |
| 220 | | H55_(−107)-(−80) | TGGAGGAACTAAATTGTAATATACCAAC | 220 |
| 221 | | H55_(−77)-(−50) | CACCTAGTGAACTCCATAAAAAGAGAAA | 221 |
| 222 | | H55_(−47)-(−20) | CAGATGCAATTATTAAATATCAGAATGG | 222 |
| 223 | | H55_(−18)-10 | CTCACTCACCCTGCAAAGGACCAAATGT | 223 |
| 224 | | H55_12-34 | AGTTTCTTCCAAAGCAGCCTCTC | 224 |
| 113 | R23 | H55_177-204 | AATGCCTGACTTACTTGCCATTGTTTCA | 113 |
| 114 | | H55_191-218 | AGTGCTAAAGCGGAAATGCCTGACTTAC | 114 |
| 115 | | H55_221-248 | TGCTGAGAATTGTTCAATTGCATCCACA | 115 |
| 116 | | H55_251-278 | TGTCCCTGGCTTGTCAGTTACAAGTACA | 116 |
| 117 | | H55_281-308 | TCAGGCTGTATAAAAGCAACTATTTTGI | 117 |
| 118 | | H55_311-338 | CTCTCCTCCTTGTCCAAATACCGAAATA | 118 |
| 119 | | H55_334-361 | GATGTTTCCTTCTCCCTCTGCCTCTCTC | 119 |
| 120 | | H55_341-368 | TATAAATGATGTTTCCTTCTCCCTCTGC | 120 |
| 121 | | H55_371-398 | TTTTTCTAAGACGAGGGTGTTAAGTGGA | 121 |
| 122 | | H55_401-428 | TGCAAATGTTTTCCTGGTCAGAGCATGT | 122 |
| 123 | | H55_431-458 | TTTCTCCTTGACCGAAGCTCTGGTTTTA | 123 |
| 124 | | H55_461-488 | CCAATCCCTAAGTTATTTCTCTGAGCAA | 124 |
| 125 | | H55_491-518 | CAAAAATGTCAACTTTTAAAATTTAATA | 125 |
| 126 | | H55_521-548 | GCTTTCCCTGTAAAATATTAAATAAACA | 126 |
| 127 | | H55_548-575 | TAAATAAAACAGACAATTCATACAGATG | 127 |
| 128 | R24 | H56_(−400)-(−373) | AATAATTTCATTACTATATGGATCAAGT | 128 |
| 129 | | H56_(−380)-(−355) | AAAGTGTACCCCAGTGCCAATAATTT | 129 |
| 130 | | H56_(−345)-(−318) | TATTTTATGGAAGTGATAGGCAATAAAA | 130 |
| 131 | | H56_(−315)-(−288) | TATATCTTCTAGTCTATGGACAAAATGT | 131 |
| 132 | | H56_(−285)-(−258) | TGTATTTATAACTTTATAAAGTTCACAA | 132 |
| 133 | | H56_(−255)-(−228) | TATTCCTTGCCATTATGAGTTGGAAAGT | 133 |
| 134 | | H56_(−225)-(−198) | AAATGGGCATCTTATTAGTTGTAATAGA | 134 |
| 135 | | H56_(−195)-(−168) | ATTGCCTTCTCCTGTTATTATGTAGATT | 135 |
| 136 | | H56_(−165)-(−138) | AGATACATCTCAAATCCCTTTTCTTGGC | 136 |
| 137 | | H56_(−135)-(−108) | AGACATTTCAATCAGGCTAAACTAACAA | 137 |
| 138 | | H56_(−105)-(−78) | TTGCAAAATATAAATAATTATTAGTTCA | 138 |
| 139 | | H56_(−76)-(−51) | ACAAGCGATGAATGTGAATTTGGAGA | 139 |
| 140 | | H56_(−59)-(−34) | ATTACCAAACAAAAGAAACAAGCGAT | 140 |
| 141 | | H56_(−39)-(−14) | GGAAGAAGAATATGTGCAGAATTACC | 141 |
| 142 | | H56_(−31)-(−6) | GGACAGCAGGAAGAAGAATATGTGCA | 142 |
| 143 | | H56_(−14)-13 | TCACCTTGGAGGTCCTACAGGACAGCAG | 143 |
| 225 | R1 + | H44_188-2D2_H56_ | ACATCTCAAATCCCTGTCCAGATGTGCTGA | 225 |
| | R24 | (−156)-(−142) | | |
| 226 | | H44_188-202_H56_ | ACATTTCAATCAGGCGTCCAGATGTGCTGA | 226 |
| | | (−124)-(−110) | | |
| 227 | | H44_188-202_H56_(−85)- | TGGAGAATTGCAAAAGTCCAGATGTGCTGA | 227 |
| | | (−71) | | |
| 228 | | H56_(−156)- | GTCCAGATGTGCTGAACATCTCAAATCCCT | 228 |
| | | (−142)_H44_188-202 | | |
| 229 | | H56_(−124)- | GTCCAGATGTGCTGAACATTTCAATCAGGC | 229 |
| | | (−110)_H44_188-202 | | |
| 230 | | H56_(−85)- | GTCCAGATGTGCTGATGGAGAATTGCAAAA | 230 |
| | | (−71)_H44_188-202 | | |
| 231 | R2 + | H45_16-35_H46_(−134)- | GTTAACCATACATAATTTAAACAGTTTGCC | 231 |
| | R4 | (−115) | GCTGCCCAAT | |
| 232 | | H45_16-35_H46_(−103)- | TGGCAATTTAAAAATAAGCAACAGTTTGCC | 232 |
| | | (−84) | GCTGCCCAAT | |

In the PMO of Table 11,

PMO for the target region R2 includes PMO Nos. 22 to 71, 91 to 93, 98 and 99 in another embodiment, and PMO Nos. 22 to 59, 91 to 93, 98 and 99 in still another embodiment;
PMO for the target region R4 includes PMO Nos. 150 to 163 in another embodiment, and PMO Nos. 150 to 160 in still another embodiment;
PMO for the target region R6 includes PMO Nos. 171 to 177 in another embodiment, and PMO Nos. 171 to 176 in still another embodiment;
PMO for the target region R8 includes PMO No. 180 in another embodiment; PMO for the target region R10 includes PMO No. 183 in another embodiment; PMO for the target region R12 includes PMO Nos. 186 to 191 in another embodiment PMO for the target region R16 includes PMO Nos. 197 and 198 in another embodiment, and PMO No. 197 in still another embodiment;
PMO for the target region R18 includes PMO Nos. 201 to 206 in another embodiment;
PMO for the target region R22 includes PMO Nos. 217 to 223 in another embodiment, and PMO Nos. 217 to 222 in still another embodiment; and
PMO for the target region R24 includes PMO Nos. 128 to 142 in another embodiment.

0.2 g of 4-{[(2S,6R)-6-(4-benzamide-2-oxopyrimidin-1-yl)-4-tritylmorpholin-2-yl]methoxy}-4-oxobutanoic acid supported on an aminopolystyrene resin (Compound 1), 4-{[(2S,6R)-6-(5-methyl-2,4-dioxopyrimidin-1-yl)-4-tritylmorpholin-2-yl]methoxy}-4-oxobutanoic acid supported on amino polystyrene resin (Compound 2), 4-{[(2S,6R)-6-(6-benzamidopurin-9-yl)-4-tritylmorpholin-2-yl]methoxy}-4-oxobutanoic acid supported on amino polystyrene resin (Compound 3), or 4-{{(2S,6R)-6-{6-(2-cyanoethoxy)-2-[(2-phenoxyacetyl)amino]purin-9-yl}-4-tritylmorpholin-2-yl}methoxy}-4-oxobutanoic acid supported on amino polystyrene resin (Compound 4) appropriate for the 5′-terminal base was filled in a column with a filter tip. Then, the synthetic cycle shown in Table 12 was started using a nucleic acid synthesizing machine (AKTA Oligopilot 10 plus). The desired morpholino monomer compound was added in each coupling cycle to give the base sequence of each PMO described in Table 11 (see Table 12).

TABLE 12

| Step | Reagent | Volume (mL) | Time (min) |
|---|---|---|---|
| 1 | deblocking solution | 18-32 | 1.8-3.2 |
| 2 | neutralizing and washing | 30 | 1.5 |
| | solution | | |
| 3 | coupling solution B | 5 | 0.5 |
| 4 | coupling solution A | 1.3 | 0.25 |
| 5 | coupling reaction by the | | 120-300 |
| | reagents added in the steps | | |
| | 3 and 4 | | |
| 6 | acetonitrile | 20 | 1.0 |
| 7 | capping solution | 9 | 2.0 |
| 8 | acetonitrile | 30 | 2.0 |

Note that only for 3′-terminal acetylation, only the steps 1, 2, 7, and 8 were carried out again after the final cycle.

The deblocking solution used was dichloromethane solution containing 3% (w/v) trifluoroacetic acid. The neutralizing and washing solution used was a solution obtained by dissolving N,N-diisopropylethylamine to be 10% (v/v) and tetrahydrofuran to be 5% (v/v) in dichloromethane containing 35% (v/v) acetonitrile. The coupling solution A used was a solution obtained by dissolving the morpholino monomer compound in tetrahydrofuran to be 0.10 M. The coupling solution B used was a solution obtained by dissolving N,N-diisopropylethylamine to be 20% (v/v) and tetrahydrofuran to be 10% (v/v) in acetonitrile. The capping solution used was a solution obtained by dissolving 20% (v/v) acetic anhydride and 30% (v/v) 2,6-lutidine in acetonitrile.

The morpholino monomer compound (A), the morpholino monomer compound (C), the morpholino monomer compound (T), and the morpholino monomer compound (G) described in Table 13 were used as the morpholino monomer compound.

TABLE 13

| Morpholino monomer | Morpholino monomer |
|---|---|
| compound | compound |
| (A) | (C) |
| 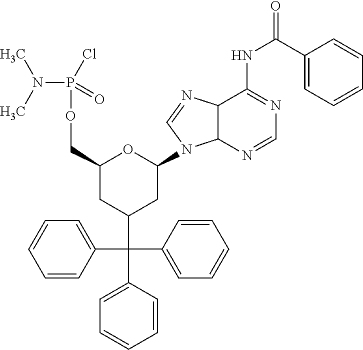 [SMILES: CN(C)P(=O)(Cl)OC[C@@H]1CC(C(c2ccccc2)(c2ccccc2)c2ccccc2)C[C@H](N2C=NC3C(NC(=O)c4ccccc4)=NC=NC32)O1] | 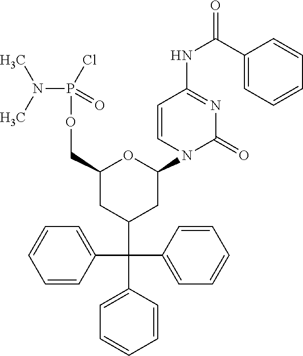 [SMILES: CN(C)P(=O)(Cl)OC[C@@H]1CC(C(c2ccccc2)(c2ccccc2)c2ccccc2)C[C@H](n2ccc(NC(=O)c3ccccc3)nc2=O)O1] |
| Morpholino monomer | Morpholino monomer |
| compound | compound |
| (T) | (G) |
| 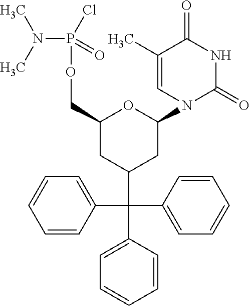 [SMILES: Cc1cn([C@H]2CC(C(c3ccccc3)(c3ccccc3)c3ccccc3)C[C@@H](COP(=O)(Cl)N(C)C)O2)c(=O)[nH]c1=O] | 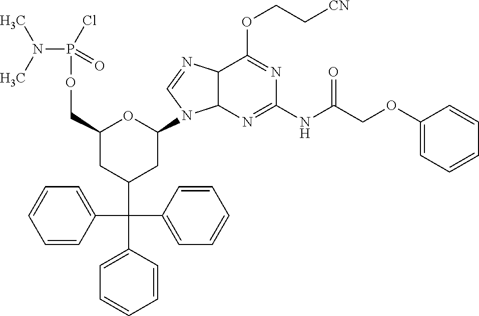 [SMILES: CN(C)P(=O)(Cl)OC[C@@H]1CC(C(c2ccccc2)(c2ccccc2)c2ccccc2)C[C@H](N2C=NC3C(OCCC#N)=NC(NC(=O)COc4ccccc4)=NC32)O1] |

The aminopolystyrene resin loaded with the PMO synthesized above was recovered from the reaction vessel and dried at room temperature for at least 2 hours under reduced pressure. The dried PMO loaded onto aminopolystyrene resin was charged in a reaction vessel, and 5 mL of 28% ammonia water-ethanol (1/4) was added thereto. The mixture was stirred at 55° C. for 15 hours. The aminopolystyrene resin was separated by filtration and washed with 1 mL of water-ethanol (1/4). The resulting filtrate was concentrated under reduced pressure. The resulting residue was dissolved in 10 mL of a solvent mixture of 20 mM of acetic acid-triethylamine buffer (TEAA buffer) and acetonitrile (4/1) and filtered through a membrane filter. The filtrate obtained was purified by reversed phase HPLC. The conditions used are shown in Table 14.

TABLE 14

| | |
|---|---|
| Column | XBridge 5 μm C18 (Waters, ϕ19 × 50 mm, |
| | 1 CV = 14 mL) |
| Flow rate | 10 mL/min |
| Column | room temperature |
| temperature | |
| Solution A | 20 mM TEAA buffer |
| Solution B | acetonitrile (CH₃CN) |
| Gradient | Solution B: (10% → 70%)/15 CV |

CV: column volume

Each fraction was analyzed, and the product of interest was recovered and concentrated under reduced pressure. To the concentrated residue, 0.5 mL of 2 M phosphoric acid aqueous solution was added, and the mixture was stirred for 15 minutes. Furthermore, 2 mL of 2 M sodium hydroxide aqueous solution was added to make the mixture alkaline, followed by filtration through a membrane filter (0.45 μm).

The resulting aqueous solution containing the product of interest was purified by an anionic exchange resin column. The conditions used are shown in Table 15.

TABLE 15

| | |
|---|---|
| Column | Source 15Q (GE Healthcare, ϕ10 × 108 mm, |
| | 1 CV = 8.5 mL) |
| Flow rate | 8.5 mL/min |
| Column | room temperature |
| temperature | |
| Solution A | 10 mM sodium hydroxide aqueous solution |
| Solution B | 10 mM sodium hydroxide aqueous solution, |
| | 1M sodium chloride aqueous solution |
| Gradient | Solution B: (1% → 50%)/40 CV |

Each fraction was analyzed (on HPLC) and the product of interest was obtained as an aqueous solution. To the resulting aqueous solution was added 0.1 M phosphate buffer (pH 6.0) for neutralization. Next, the mixture obtained was demineralized by reversed phase HPLC under the conditions described in Table 16.

TABLE 16

| | |
|---|---|
| Column | XBridge 5 μm C18 (Waters, ϕ10 × 50 mm, |
| | 1 CV = 4 mL) |
| Flow rate | 4 mL/min |
| Column | 60° C. |
| temperature | |
| Solution A | water |
| Solution B | CH₃CN |
| Gradient | Solution B: (0% → 50%)/20 CV |

The product of interest was recovered and the mixture was concentrated under reduced pressure. The resulting residue was dissolved in water. The aqueous solution obtained was freeze-dried to give each PMO as the compound of interest as a white cotton-like solid. The calculated and measured values of ESI-TOF-MS are shown in Table 17 below.

TABLE 17

| PMO No. | Target base sequence | Calculated | Measured |
|---|---|---|---|
| 1 | H44_150-175 | 8610.02 | 8610.48 |
| 2 | H44_157-196 | 13354.66 | 13354.45 |
| 3 | H44_159-193 | 11652.07 | 11652.09 |
| 4 | H44_170-199 | 10012.48 | 10013.39 |
| 5 | H44_179-206 | 9392.27 | 9392.54 |
| 6 | H44_209-236 | 9230.21 | 9230.92 |
| 7 | H44_239-266 | 9256.24 | 9256.17 |
| 8 | H44_269-296 | 9264.2 | 9263.99 |
| 9 | H44_299-326 | 9181.19 | 9181.46 |
| 10 | H44_330-355 | 8514.98 | 8514.36 |
| 11 | H44_354-381 | 9231.21 | 9231.24 |
| 12 | H44_389-416 | 9147.17 | 9147.4 |
| 13 | H44_413-438 | 8551.97 | 8552.71 |
| 14 | H44_423-452 | 9830.43 | 9830.2 |
| 15 | H44_455-480 | 8588.01 | 8587.53 |
| 16 | H44_479-506 | 9262.21 | 9262.15 |
| 17 | H44_509-536 | 9226.19 | 9225.7 |
| 18 | H44_150-164_l79-193 | 9989.49 | 9989.23 |
| 19 | H44_150-164_l92-206 | 10006.49 | 10006.81 |
| 20 | H44_161-175_179-193 | 10013.5 | 10013.95 |
| 21 | H44_161-175_192-206 | 10030.5 | 10031.48 |
| 22 | H45_(−598)-(−571) | 9221.2 | 9221.27 |
| 23 | H45_(−568)-(−541) | 9312.25 | 9311.93 |
| 24 | H45_(−538)-(−511) | 9218.18 | 9218.33 |
| 25 | H45_(−518)-(−491) | 9283.22 | 9283.26 |
| 26 | H45_(−515)-(−488) | 9307.23 | 9307.34 |
| 27 | H45_(−508)-(−479) | 10027.5 | 10027.98 |
| 28 | H45_(−478)-(−449) | 9979.48 | 9980.37 |
| 29 | H45_(−470)-(−443) | 9295.25 | 9296.02 |
| 30 | H45_(−448)-(−421) | 9385.28 | 9385.45 |
| 31 | H45_(−439)-(−412) | 9403.3 | 9403.98 |
| 32 | H45_(−436)-(−409) | 9435.3 | 9435.78 |
| 33 | H45_(−422)-(−395) | 9388.23 | 9388.34 |
| 34 | H45_(−418)-(−394) | 8363.88 | 8363.44 |
| 35 | H45_(−418)-(−391) | 9404.23 | 9404.6 |
| 36 | H45_(−415)-(−391) | 8354.87 | 8354.48 |
| 37 | H45_(−413)-(−386) | 9385.21 | 9385.07 |
| 38 | H45_(−405)-(−378) | 9371.23 | 9371.33 |
| 39 | H45_(−389)-(−362) | 9325.25 | 9325.6 |
| 40 | H45_(−349)-(−322) | 9221.2 | 9221.14 |
| 41 | H45_(−320)-(−293) | 9211.19 | 9211.14 |
| 42 | H45_(−305)-(−280) | 8640 | 8640.11 |
| 43 | H45_(−290)-(−263) | 9284.22 | 9284.05 |
| 44 | H45_(−275)-(−250) | 8656 | 8656.36 |
| 45 | H45_(−260)-(−233) | 9347.22 | 9347 |
| 46 | H45_(−230)-(−203) | 9198.19 | 9198.43 |
| 47 | H45_(−200)-(−173) | 9335.24 | 9335.21 |
| 48 | H45_(−190)-(−163) | 9312.25 | 9312.03 |
| 49 | H45_(−185)-(−160) | 8670.05 | 8670.14 |
| 50 | H45_(−171)-(−144) | 9273.24 | 9273.53 |
| 51 | H45_(−188)-(−141) | 9233.23 | 9233.36 |
| 52 | H45_(−142)-(−115) | 9254.22 | 9254.39 |
| 53 | H45_(−110)-(−83) | 9392.27 | 9392.55 |
| 54 | H45_(−80)-(−53) | 9352.26 | 9352.54 |
| 55 | H45_(−78)-(−51) | 9358.25 | 9358.63 |
| 56 | H45_(−70)-(−43) | 9323.23 | 9323.51 |
| 57 | H45_(−60)-(−33) | 9260.21 | 9260.39 |
| 58 | H45_(−50)-(−23) | 9238.22 | 9238.79 |
| 59 | H45_(−44)-(−17) | 9291.26 | 9291.37 |
| 60 | H45_(−25)-3 | 9281.25 | 9281.54 |
| 61 | H45_(−23)-5 | 9321.26 | 9321.92 |
| 62 | H45_(−22)-3 | 8287.9 | 8287.54 |
| 63 | H45_(−21)-7 | 9353.26 | 9353.35 |
| 64 | E45_(−20)-5 | 8303.9 | 8303.52 |
| 65 | H45_(−20)-8 | 9344.25 | 9344.57 |
| 66 | H45_(−18)-10 | 9304.24 | 9304.44 |
| 67 | H45_(−17)-8 | 8318.9 | 8318.93 |
| 68 | H45_(−16)-12 | 9279.23 | 9278.95 |
| 69 | H45_(−15)-10 | 8270.88 | 8270.96 |
| 70 | H45_(−14)-14 | 9231.21 | 9231.28 |
| 71 | H45_(−10)-16 | 8607.98 | 8607.59 |
| 72 | H45_(−5)-20 | 8213.85 | 8213.45 |
| 73 | H45_l-25 | 8214.85 | 8214.52 |
| 74 | H45_6-30 | 8190.84 | 8190.04 |
| 75 | H45_11-35 | 8198.85 | 8198.45 |
| 76 | H45_11-40 | 9877.43 | 9877.12 |
| 77 | H45_16-35 | 6544.28 | 6544.07 |
| 78 | H45_16-40 | 8222.86 | 8222.85 |
| 79 | H45_21-45 | 8244.85 | 8245.01 |
| 80 | H45_(−423)-(−412)_16-33 | 9997.46 | 9997.49 |
| 81 | H45_(−416)-(−402)_16-30 | 9964.44 | 9964.38 |
| 82 | H45_(−411)-(−400)_16-33 | 9979.44 | 9979.18 |
| 83 | H45_(−405)-(−391)_16-30 | 9945.42 | 9945.04 |
| 84 | H45_(−402)-(−391)_16-33 | 9945.42 | 9945.07 |
| 85 | H45_(−156)-(−143)-(−141)-(−128) | 9190.2 | 9189.84 |
| 86 | H45_(−78)-(−65)_(−44)-(−31) | 9256.22 | 9256.49 |
| 87 | H45_(−78)-(−65)_(−34)-(−21) | 9354.28 | 9354.26 |
| 88 | H45_(−78)-(−65)-16-29 | 9232.21 | 9233.24 |
| 89 | H45_(−78)-(−65)_21-34 | 9263.21 | 9263.82 |
| 90 | H45_(−78)-(−65)_27-40 | 9295.23 | 9295.5 |
| 91 | H45_(−74)-(−62)_(−40)-(−28) | 8643.02 | 8643.41 |
| 92 | H45_(−72)-(−58)_(−38)-(−26) | 9354.28 | 9354.28 |
| 93 | H45_(−71)-(−58)_(−38)-(−25) | 9314.27 | 9314.5 |
| 94 | H45_(−71)-(−58)_16-29 | 9264.23 | 9262.81 |
| 95 | H45_(−71)-(−58)_21-34 | 9295.23 | 9295.83 |
| 96 | H45_(−71)-(−58)_27-40 | 9327.25 | 9327.61 |
| 97 | H45_(−68)-(−60)_(−36)-(−28)_19-28 | 9274.24 | 9274.8 |
| 98 | H45_(−64)-(−51)_(−44)-(−31) | 9310.23 | 9310.1 |
| 99 | 145_(−64)-(−51)_(−35)-(−22) | 9408.29 | 9408.3 |
| 100 | H45_(−64)-(−51)_16-29 | 9286.22 | 9286.47 |
| 101 | H45_(−64)-(−51)_21-34 | 9317.22 | 9317.12 |
| 102 | H45_(−64)-(−51)_27-40 | 9349.24 | 9349.37 |
| 103 | H45_(−44)-(−31)_16-29 | 9184.19 | 9183.98 |
| 104 | H45_(−44)-(−31)_27-40 | 9247.21 | 9247.14 |
| 105 | H45_(−44)-(−31)_21-33_T | 9230.19 | 9230.7 |
| 106 | H45_(−38)-(−25)_16-29 | 9210.22 | 9210.11 |
| 107 | H45_(−38)-(−25)_21-34 | 9241.22 | 9241.87 |
| 108 | H45_(−38)-(−25)_27-40 | 9273.24 | 9273.24 |
| 109 | H45_(−34)-(−21)_16-29 | 9266.25 | 9266.72 |
| 110 | H45_(−34)-(−21)_21-34 | 9297.25 | 9297.85 |
| 111 | H45_(−34)-(−21)_27-40 | 9329.27 | 9329.78 |
| 112 | H45_1-21 | 6859.387 | 6859.02 |
| 144 | H45_167-194 | 9121.14 | 9121.27 |
| 145 | H45_227-254 | 9253.22 | 9253.57 |
| 146 | H45_257-284 | 9203.18 | 9203.09 |
| 147 | H45_287-314 | 9369.21 | 9369.79 |
| 148 | H45_317-344 | 9342.27 | 9342.18 |
| 149 | H45_347-374 | 9268.22 | 9268.72 |
| 150 | H46_(−200)-(−173) | 9326.25 | 9325.78 |
| 151 | H46_(−171)-(−144) | 9243.21 | 9243.17 |
| 152 | H46_(−146)-(−119) | 9221.22 | 9221.07 |
| 153 | H46_(−141)-(−114) | 9276.23 | 9275.87 |
| 154 | H46_(−136)-(−109) | 9310.25 | 9310.07 |
| 155 | H46_(−126)-(−99) | 9295.25 | 9295.56 |
| 156 | H46_(−116)-(−89) | 9349.26 | 9349.16 |
| 157 | H46_(−111)-(−84) | 9334.26 | 9333.8 |
| 158 | H46_(−106)-(−79) | 9319.26 | 9319.07 |
| 159 | H46_(−81)-(−54) | 9318.24 | 9317.86 |
| 160 | H46_(−51)-(−24) | 9167.16 | 9167.63 |
| 161 | H46_(−18)-6 | 8037.81 | 8037.34 |
| 162 | H46_(−15)-9 | 8004.79 | 8004.24 |
| 163 | H46_(−9)-15 | 7943.74 | 7944.19 |
| 164 | H46_139-l66 | 9293.23 | 9292.5 |
| 165 | H46_169-196 | 9319.22 | 9319.52 |
| 166 | H46_199-226 | 9325.25 | 9325.91 |
| 167 | H46_229-256 | 9361.27 | 9361.58 |
| 168 | H46_259-286 | 9351.26 | 9351.88 |
| 169 | H46_289-316 | 9400.26 | 9400.38 |
| 170 | H46_319-346 | 9219.2 | 9219.51 |
| 171 | H47_(−200)-(−173) | 9142.18 | 9142.19 |
| 172 | H47_(−171)-(−144) | 9320.24 | 9320.43 |
| 173 | H47_(−141)-(−114} | 9230.21 | 9230.7 |
| 174 | H47_(−111)-(−84) | 9354.26 | 9354.05 |
| 175 | H47_(−81)-(−54) | 9215.16 | 9215.57 |
| 176 | H47_(−51)-(−24) | 9262.23 | 9262.28 |
| 177 | H47_(−16)-12 | 9257.24 | 9257.23 |
| 178 | H47_l-29 | 9612.33 | 9613.79 |
| 179 | H47_135-162 | 9236.2 | 9236.32 |
| 180 | H48_(−21)-7 | 9351.26 | 9350.96 |
| 181 | H48_l-28 | 9277.21 | 9277.18 |
| 182 | H48_177-204 | 9233.23 | 9232.72 |
| 183 | H49_(−21)-7 | 9304.24 | 9304.25 |
| 184 | H49_22-47 | 8509.94 | 8509.66 |
| 185 | H49_93-120 | 9244.19 | 9244.51 |
| 186 | H50_(−200)-(−173) | 9149.19 | 9148.87 |
| 187 | H50_(−171)-(−144) | 9292.23 | 9291.93 |
| 188 | H50_(−141)-(−114) | 9330.22 | 9330.33 |
| 189 | H50_(−111)-(−84) | 9237.17 | 9236.8 |
| 190 | H50_(−81)-(−54) | 9260.21 | 9259.85 |
| 191 | H50_(−51)-(−24) | 9259.21 | 9258.85 |
| 192 | H50_(−21)-7 | 9183.21 | 9183.31 |
| 193 | H50_l00-127 | 9271.22 | 9271.25 |
| 194 | H50_90-114 | 8277.87 | 8278.28 |
| 195 | H51_(−18)-10 | 9411.24 | 9411.6 |
| 196 | H51_224-251 | 9135.14 | 9135.51 |
| 197 | H52_(−47)-(−20) | 9330.2 | 9330.03 |
| 198 | H52_(−18)-10 | 9270.22 | 9270.37 |
| 199 | H52_9-38 | 9893.42 | 9893.2 |
| 200 | H52_109-136 | 9236.2 | 9235.9 |
| 201 | H53_(−197)-(−170) | 9231.18 | 9231.18 |
| 202 | H53_(−167)-(−140) | 9298.2 | 9298.58 |
| 203 | H53_(−137)-(−110) | 9353.21 | 9353.6 |
| 204 | H53_(−107)-(−80) | 9225.19 | 9224.55 |
| 205 | H53_(−77)-(−50) | 9278.23 | 9278.35 |
| 206 | H53_(−47)-(−20) | 9373.25 | 9373.48 |
| 207 | H53_(−18)-10 | 9319.26 | 9319 |
| 208 | H53_203-230 | 9249.18 | 9249.06 |
| 209 | H54_(−18)-10 | 9316.22 | 9316.33 |
| 210 | H54_146-173 | 9267.22 | 9267.45 |
| 211 | H54_176-203 | 9181.19 | 9181.18 |
| 212 | H54_206-233 | 9142.18 | 9142.41 |
| 213 | H54_236-263 | 9311.23 | 9311.57 |
| 214 | H54_266-293 | 9249.18 | 9248.66 |
| 215 | H54_296-323 | 9344.25 | 9344.57 |
| 216 | H54_326-353 | 9308.21 | 9307.9 |
| 217 | H55_(−197)-(−170) | 9206.17 | 9206.95 |
| 218 | H55_(−167)-(−140) | 9278.23 | 9278.65 |
| 219 | H55_(−137)-(−110) | 9316.2 | 9316.05 |
| 220 | H55_(−107)-(−80) | 9327.25 | 9326.87 |
| 221 | H55_(−77)-(−50) | 9290.26 | 9290.43 |
| 222 | II55_(−47)-(−20) | 9342.25 | 9341.86 |
| 223 | H55_(−18)-10 | 9185.21 | 9185.78 |
| 224 | H55_12-34 | 6858.387 | 6858.97 |
| 113 | H55_177-204 | 9203.18 | 9203.35 |
| 114 | H55_191-218 | 9320.24 | 9320.46 |
| 115 | H55_221-248 | 9301.22 | 9301.7 |
| 116 | H55_251-278 | 9253.2 | 9253.12 |
| 117 | H55_281-308 | 9300.22 | 9300.57 |
| 118 | H55_311-338 | 9126.18 | 9126.01 |
| 119 | H55_334-361 | 9058.12 | 9057.77 |
| 120 | H55_341-368 | 9154.16 | 9154.22 |
| 121 | H55_371-398 | 9412.24 | 9412.07 |
| 122 | H55_401-428 | 9308.21 | 9308.06 |
| 123 | H55_431-458 | 9210.17 | 9209.71 |
| 124 | H55_461-488 | 9181.19 | 9181.31 |
| 125 | H55_491-518 | 9260.23 | 9260.72 |
| 126 | H55_521-548 | 9237.22 | 9235.93 |
| 127 | H55_548-575 | 9304.26 | 9304.44 |
| 128 | H56_(−400)-(−373) | 9283.22 | 9282.84 |
| 129 | H56_(−380)-(−355) | 8584.99 | 8585.11 |
| 130 | H56_(−345)-(−318) | 9397.26 | 9397.16 |
| 131 | H56_(−315)-(−288) | 9275.21 | 9275.43 |
| 132 | H56_(−285)-(−258) | 9258.21 | 9257.72 |
| 133 | H56_(−255)-(−228) | 9307.21 | 9307.98 |
| 134 | H56_(−225)-(−198) | 9364.24 | 9364.79 |
| 135 | H56_(−195)-(−168) | 9224.17 | 9224.08 |
| 136 | H56_(−165)-(−138) | 9172.18 | 9172.28 |
| 137 | H56_(−135)-(−108) | 9256.24 | 9256.9 |
| 138 | H56_(−105)-(−78) | 9291.23 | 9291.37 |
| 139 | H56_(−76)-(−51) | 8754.04 | 8754.09 |
| 140 | H56_(−59)-(−34) | 8629.04 | 8629.59 |
| 141 | H56_(−39)-(−14) | 8723.04 | 8723.84 |
| 142 | H56_(−31)-(−6) | 8773.06 | 8773.68 |
| 143 | H56_(−14)-13 | 9297.23 | 9296.6 |
| 225 | H44_188-202_H56_(−156)-(−142) | 9876.43 | 9876.87 |
| 226 | H44_188-202_H56_(−124)-(−110) | 9956.45 | 9956.24 |
| 227 | H44_188-202_H56_(−85)-(−71) | 10069.49 | 10070.12 |
| 228 | H56_(−156)-(−142)_H44_188-202 | 9876.43 | 9876.63 |
| 229 | H56_(−124)-(−110)_H44_188-202 | 9956.45 | 9956.72 |
| 230 | H56_(−85)-(−71)_H44_188-202 | 10069.49 | 10069.93 |
| 231 | H45_16-35_H46_(−134)-(−115) | 13207.58 | 13207.56 |
| 232 | H45_16-35_H46_(−103)-(−84) | 13281.61 | 13281.25 |

### Production of Antisense PMO Targeting Splicing Silencer in Intron of Human Dystrophin Gene or Splice Site Involved in Single Skipping of Human Dystrophin Gene

(i) PMO having the base sequence of PMO No. 233 (SEQ ID NO: 257) targeting a splicing silencer sequence of intron 44 or a splice site involved in single skipping, (ii) PMOs having the base sequences of PMO Nos. 234 to 244 (SEQ ID NOs: 258 to 268) targeting a splicing silencer sequence of intron 45 or a splice site involved in single skipping, (iii) PMOs (linked type) having the base sequences of PMO Nos. 245 to 249 (SEQ ID NOs: 269 to 273) targeting a region (R2) in the vicinity of an acceptor of intron 44 and a splicing silencer sequence of intron 45 or a splice site involved in single skipping, and (iv) PMOs having the base sequences of PMO Nos. 250 to 251 (SEQ ID NOs: 274 to 275) targeting a splicing silencer sequence of exon 45 in the human dystrophin gene were synthesized in the same manner as in the PMO targeting a region in the vicinity of a donor or an acceptor of an intron. The 5′ end of each PMO is Group (3), as in the PMO targeting a region in the vicinity of a donor or an acceptor of an intron. Each PMO is shown in Table 18, and the calculated (Clcd) and measured (Found) values of ESI-TOF-MS are shown in Table 19.

TABLE 18

| PMO | | | | SEQ |
|---|---|---|---|---|
| No. | Target | Target base sequence | Base sequence of PMO | ID NO |
| 233 | (i) | H45_(−93)-(−70) | GTGTGCTACCACATGCAGTTGTAC | 257 |
| 234 | (ii) | H45_166-189 | TCTGTCGCCCTACCTCTTTTTTCT | 258 |
| 235 | | H45_178-201 | ATTCCTATTAGATCTGTCGCCCTA | 259 |
| 236 | | H45_183-206 | TTTTCATTCCTATTAGATCTGTCG | 260 |
| 237 | | H45_188-211 | AAATGTTTTCATTCCTATTAGATC | 261 |
| 238 | | H45_191-214 | CTAAAATGTTTTCATTCCTATTAG | 262 |
| 239 | | H45_197-220 | AGTCTGCTAAAATGTTTTCATTCC | 263 |
| 240 | | H46_(−35)-(−12) | AGAATAAAATTGTTATTTTTTTTT | 264 |
| 241 | | H46_(−22)_2 | GCCTGGAGAAAGAAGAATAAAATT | 265 |
| 242 | | H46_(−12)_12 | TGTTCTTCTAGCCTGGAGAAAGAA | 266 |
| 243 | | H46_(−2)_22 | GATATTCTTTTGTTCTTCTAGCCT | 267 |
| 244 | | H45_20236-20259 | ATCCATGCATTCTTCCATGCACAG | 268 |
| 245 | (iii) | H45_16-30_191-205 | TTTCATTCCTATTAGTTGCCGCTGCCCAAT | 269 |
| 246 | | H45_16-30_197-211 | AAATGTTTTCATTCCTTGCCGCTGCCCAAT | 270 |
| 247 | | H45_16-33_197-208 | TGTTTTCATTCCAGTTTGCCGCTGCCCAAT | 271 |
| 248 | | H45_16-35_183-192 | AGATCTGTCGACAGTTTGCCGCTGCCCAAT | 272 |
| 249 | | H45 18-35_191-202 | CATTCCTATTAGACAGTTTGCCGCTGCCCA | 273 |
| 250 | (iv) | H45_31-54 | TGCATTCAATGTTCTGACAACAGT | 274 |
| 251 | | H45_131-154 | GTTTGCAGACCTCCTGCCACCGCA | 275 |

A base sequence presumed to correspond to the splicing silencer sequence in the intron or the splice site involved in single skipping is underlined.

A base sequence presumed to correspond to the splicing silencer sequence in the intron or the splice site involved in single skipping is underlined.

TABLE 19

| PMO No. | Target base sequence | Calculated | Measured |
|---|---|---|---|
| 233 | H45_(−93)-(−70) | 7938.75 | 7938.95 |
| 234 | H45_166-189 | 7757.67 | 7757.89 |
| 235 | H45_178-201 | 7848.72 | 7848.99 |
| 236 | H45_183-206 | 7869.71 | 7869.94 |
| 237 | H45_188-211 | 7886.73 | 7886.24 |
| 238 | H45_191-214 | 7886.73 | 7886.37 |
| 239 | H45_197-220 | 7887.73 | 7887.49 |
| 240 | H46_(−35)-(−12) | 7955.74 | 7955.61 |
| 241 | H46_(−22)_2 | 8061.82 | 8062.19 |
| 242 | H46_(−12)_12 | 7986.77 | 7986.42 |
| 243 | H46_(−2)_22 | 7860.7 | 7860.83 |
| 244 | H45_20236-20259 | 7842.73 | 7842.9 |
| 245 | H45_16-30_l91-205 | 9824.39 | 9824.66 |
| 246 | H45_16-30_l97-211 | 9833.4 | 9833.32 |
| 247 | H45_16-33_197-208 | 9840.39 | 9840.1 |
| 248 | H45_16-35_183-192 | 9908.43 | 9908.14 |
| 249 | H45_18-35_l91-202 | 9843.41 | 9843.08 |
| 250 | H45_31-54 | 7848.72 | 7848.99 |
| 251 | H45_131-154 | 7844.73 | 7844.85 |

### Example 2: Test on Multi-Exon Skipping Activity of Antisense Oligomer

### Test Example 1

### In Vitro Assay of Multi-Exon Skipping in Human Dystrophin Gene—(1): Induction of Multi-Exon Skipping

### Procedures

Each antisense oligomer (PMO Nos. 55, 59, 78, 65, 75, 77, 102, 35, 52, 47, 50, 46, 43, 40, 139, 119, 142, 113, 51, 11, 45, 54, 41, 74, 66, 64, 63, 68, 76, 28, 27, 225, 228, 1, 5, 132, 131, 128, 163, 162, and 161 (SEQ ID NO: 55, 59, 78, 65, 75, 77, 102, 35, 52, 47, 50, 46, 43, 40, 139, 119, 142, 113, 51, 11, 45, 54, 41, 74, 66, 64, 63, 68, 76, 28, 27, 225, 228, 1, 5, 132, 131, 128, 163, 162, and 161)) of Table 11 was transfected in a concentration of 25 to 50 μM either singly or in combination of two or three oligomers with 3.5×10⁵ of RD cells (human rhabdomyosarcoma cell line, CCL-136, purchased from ATCC). The reagent used for the transfection was an Amaxa Cell Line Nucleofector Kit L on Nucleofector II (Lonza). The Program T-030 was used.

After transfection, the cells were cultured for three nights in 2 mL of Eagle's minimal essential medium (EMEM) (manufactured by Sigma, hereinafter the same) containing 10% fetal bovine serum (FBS) (manufactured by Invitrogen) under conditions of 37° C. and 5% CO₂.

The RD cells were washed once with PBS (manufactured by Nissui, hereinafter the same) and 350 μL of Buffer RLT (manufactured by Qiagen) containing 1% 2-mercaptoethanol (manufactured by Nacalai Tesque, Inc.) was added to the cells. After the cells were allowed to stand at room temperature for a few minutes to lyse the cells, the lysate was collected into a QIAshredder homogenizer (manufactured by Qiagen). A homogenate was produced by centrifugation at 15,000 rpm for 2 minutes. The total RNA was extracted according to the protocol attached to RNeasy Mini Kit (manufactured by Qiagen). The concentration of the total RNA extracted was determined using a NanoDrop ND-1000 (manufactured by LMS Co., Ltd.).

One-Step RT-PCR was performed with 800 ng of the extracted total RNA using a QIAGEN One Step RT-PCR Kit (manufactured by Qiagen). A reaction solution was prepared in accordance with the protocol attached to the kit. TaKaRa PCR Thermal Cycler Dice Touch (manufactured by Takara Bio Inc.) was used as the thermal cycler. The RT-PCR program used is as follows.

50° C., 30 mins: reverse transcription reaction

95° C., 15 mins: polymerase activation, reverse transcriptase inactivation, cDNA thermal denaturation

[94° C., 10 seconds; 57° C., 30 seconds; 72° C., 1 minute]×33 cycles: PCR amplification

72° C., 10 mins: final extension

The base sequences of the forward primer and reverse primer used for RT-PCR are given below.

Forward primer:
(SEQ ID NO: 276)
5′-ATTTGACAGATCTGTTGAGAAATGG-3′
Reverse primer 1:
(SEQ ID NO: 277)
5′-GGCTGTTTTCATCCAGGTTGTG-3′
Reverse primer 2:
(SEQ ID NO: 278)
5′-AGTTGCTGCTCTTTTCCAGGT-3′

Transcripts having multi-exon skipping of exons 45 to 55 can be detected by a combination of the forward primer and the reverse primer 2. Transcripts having no skipping and transcripts having single exon skipping of exon 45 can be detected by a combination of the forward primer and the reverse primer 1.

The reaction product of the PCR above was analyzed using a Bioanalyzer (manufactured by Agilent Technologies, Inc.) and MultiNA (manufactured by Shimadzu Corp.).

The polynucleotide level “A” of the band with skipping of any two or more numerically consecutive exons among exons 45 to 55, the polynucleotide level “B” of the band with skipping of any one exon among exons 45 to 55, and the polynucleotide level “C” of the band without skipping were measured. Based on these measurement values of “A”, “B”, and “C”, the skipping efficiency of multi-exon skipping was determined by the following equation.

Skipping efficiency (%)=A/(A+B+C)×100

### Results

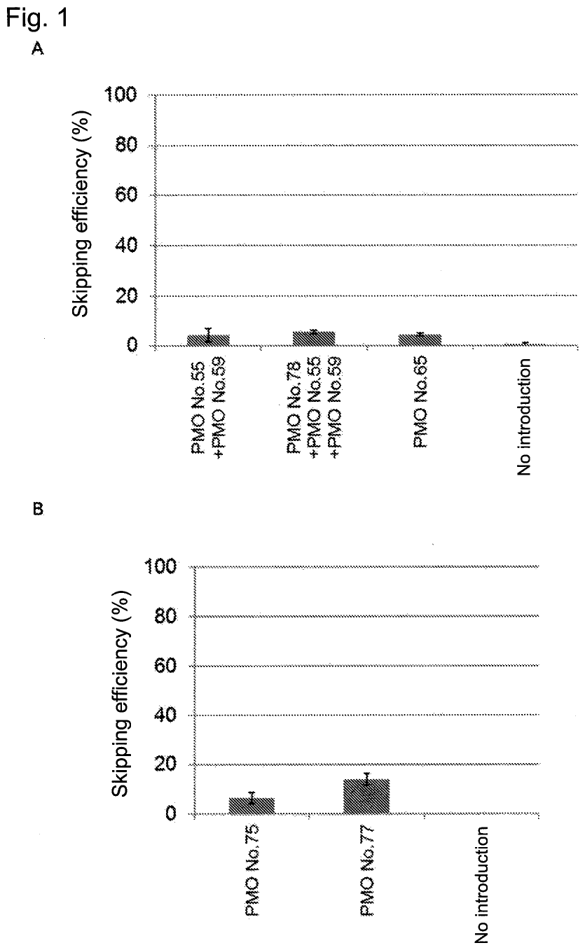

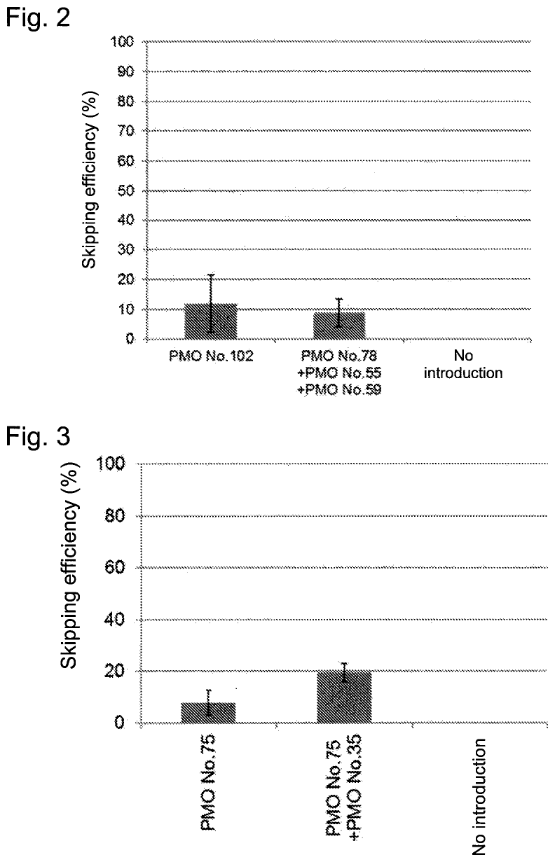

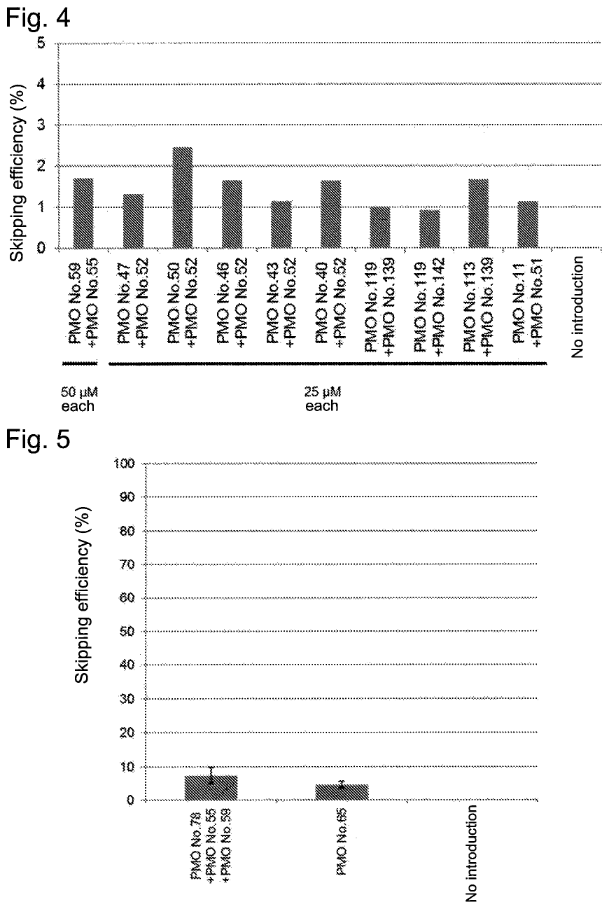

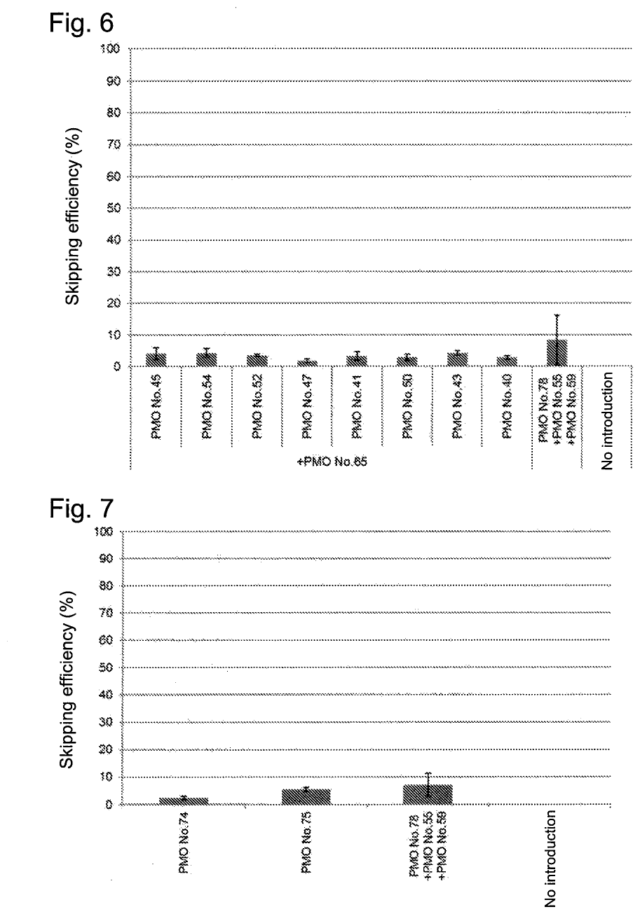

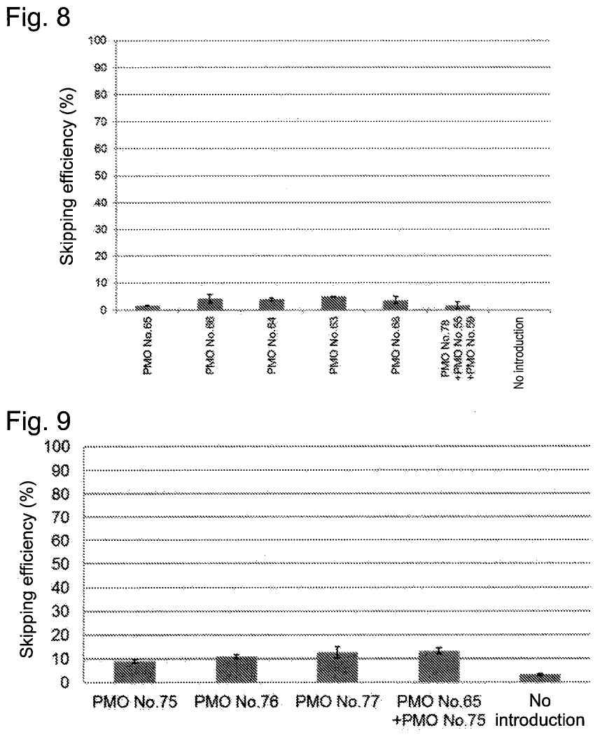

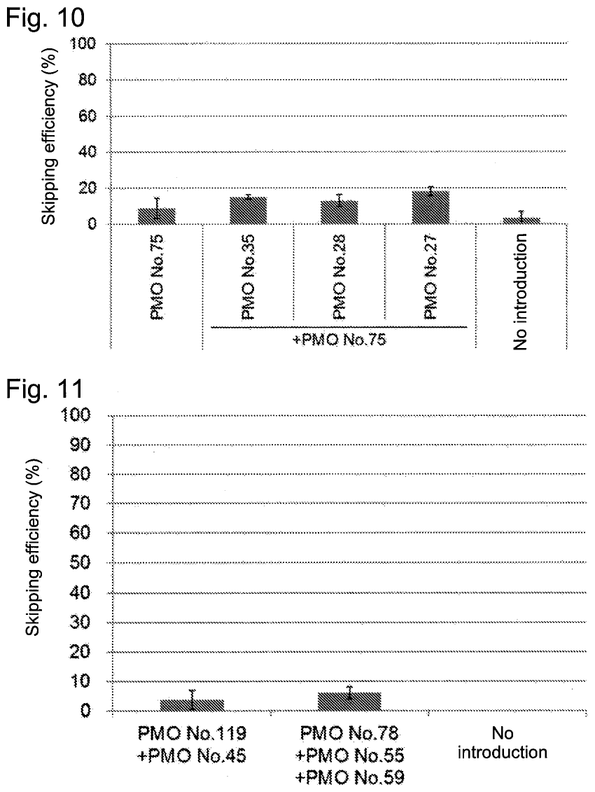

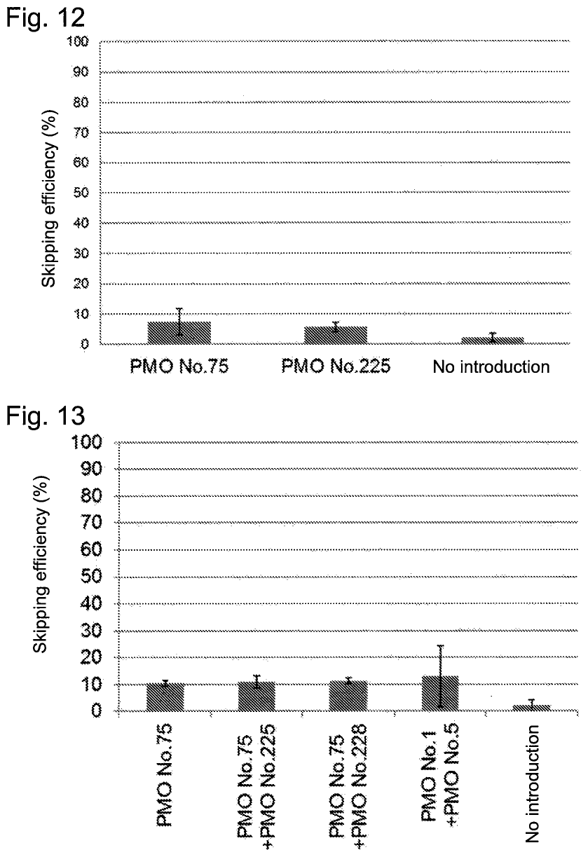

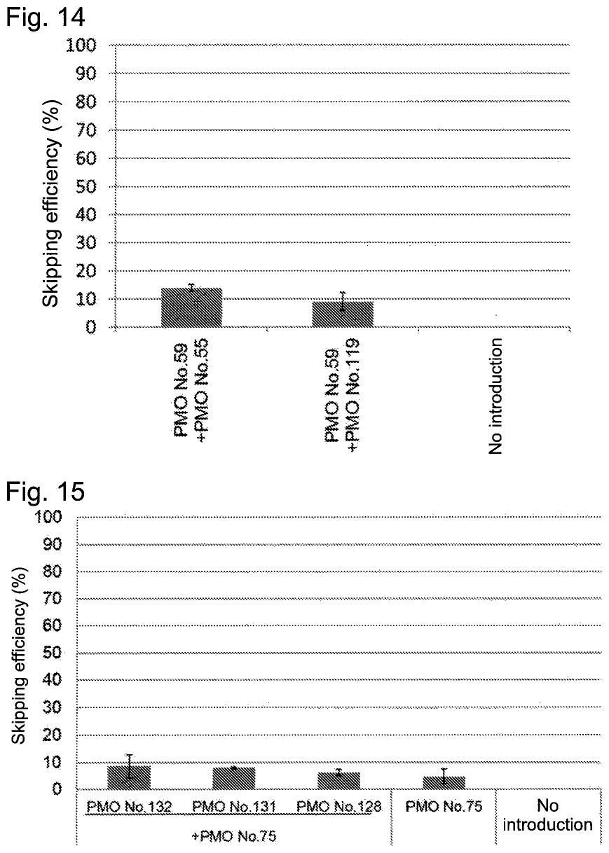

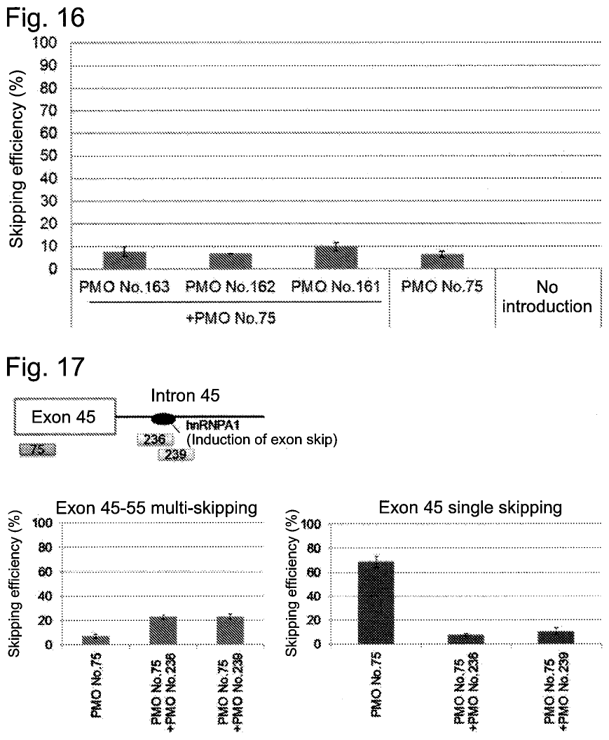

The results are shown in FIGS. 1 to 16. The introduction of PMO Nos. 55, 59, 78, 65, 75, 77, 102, 35, 52, 47, 50, 46, 43, 40, 139, 119, 142, 113, 51, 11, 45, 54, 41, 74, 66, 64, 63, 68, 76, 28, 27, 225, 228, 1, 5, 132, 131, 128, 163, 162, and 161 (SEQ ID NO: 55, 59, 78, 65, 75, 77, 102, 35, 52, 47, 50, 46, 43, 40, 139, 119, 142, 113, 51, 11, 45, 54, 41, 74, 66, 64, 63, 68, 76, 28, 27, 225, 228, 1, 5, 132, 131, 128, 163, 162, and 161) singly or in combination induced multi-exon skipping of exons 45 to 55.

These results revealed that the antisense oligomers of the present invention described in Table 11 effectively induce or would effectively induce multi-exon skipping when used singly or in combination.

It was also confirmed that the introduction of PMO Nos. 6, 7, 8, 10, 14, 26, 29, 38, 39, 53, 58, 67, 80, 82, 86, 92, 97, 98, 100, 121, 122, 124, 125, 126, 130, 144, 146, 147, 150, 151, 152, 153, 154, 155, 156, 157, 158, 159, 160, 231, 232 (SEQ ID NO: 6, 7, 8, 10, 14, 26, 29, 38, 39, 53, 58, 67, 80, 82, 86, 92, 97, 98, 100, 121, 122, 124, 125, 126, 130, 144, 146, 147, 150, 151, 152, 153, 154, 155, 156, 157, 158, 159, 160, 231, 232) also induce multi-exon skipping of exons 45 to 55.

### Test Example 2

### In Vitro Assay of Multi-Exon Skipping in Human Dystrophin Gene—(2): Promotion of Multi-Exon Skipping

### Procedures

The antisense oligomer (PMO No. 75) of Table 11 was added in a concentration of 25 to 50 μM to 3.5×10⁵ of RD cells (human rhabdomyosarcoma cell line, CCL-136, purchased from ATCC), and each antisense oligomer (PMO No. 236, 237, or 239) of Table 18 was introduced in combination therewith in a concentration of 25 to 50 μM. The assay was conducted by the same procedures as in Test Example 1.

### Results

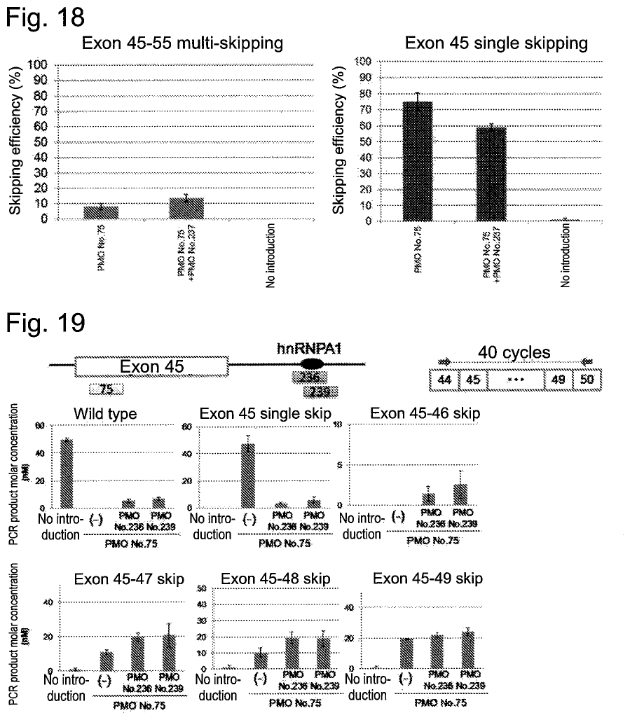

The results are shown in FIGS. 17 and 18. The introduction of PMO No. 75 (SEQ ID NO: 75) in combination with PMO No. 236, 237, or 239 (SEQ ID NO: 260, 261, or 263) enhanced the skipping efficiency of multi-exon skipping of exons 45 to 55 as compared to the case of PMO No. 75 singly. On the other hand, this approach decreased the skipping efficiency of single exon skipping of exon 45.

These results revealed that the antisense oligomer of the present invention described in Table 11 promote or would promote multi-exon skipping when used in combination with the suppressor antisense oligomer of the present invention described in Table 18.

### Test Example 3

### In Vitro Assay of Multi-Exon Skipping in Human Dystrophin Gene—(3): Promotion of Overall Multi-Exon Skipping

### Procedures

Test Example 3 was conducted by the same procedures as in Test Example 2 except that One-Step RT-PCR was performed as follows.

One-Step RT-PCR was performed with 800 ng of the extracted total RNA using a QIAGEN One Step RT-PCR Kit (manufactured by Qiagen). A reaction solution was prepared in accordance with the protocol attached to the kit. TaKaRa PCR Thermal Cycler Dice Touch (manufactured by Takara Bio Inc.) was used as the thermal cycler. The RT-PCR program used is as follows.

50° C., 30 mins: reverse transcription reaction

95° C., 15 mins: polymerase activation, reverse transcriptase inactivation, cDNA thermal denaturation

[94° C., 10 seconds; 60° C., 30 seconds; 72° C., 2 min]×40 cycles: PCR amplification

72° C., 10 mins: final extension

The base sequences of the forward primer and reverse primer used for RT-PCR are given below.

Forward primer 2:
(SEQ ID NO: 279)
5′-ATTTGACAGATCTGTTGAGAAATGG-3′
Reverse primer 3:
(SEQ ID NO: 280)
5′-GGCTCCAATAGTGGTCAGTCC-3′
Forward primer 3:
(SEQ ID NO: 281)
5′-CCTGAGAATTGGGAACATGC-3′
Reverse primer 4:
(SEQ ID NO: 282)
5′-CCTCCTTCCATGACTCAAGC-3′

Transcripts having multi-exon skipping of exons 45 and 46, exons 45 to 47, exons 45 to 48, or exons 45 to 49 in the human dystrophin gene can be detected by a combination of the forward primer 2 and the reverse primer 3. On the other hand, transcripts having multi-exon skipping of exons 45 and 46, exons 45 to 47, exons 45 to 48, exons 45 to 49, exons 45 to 50, exons 45 to 51, or exons 45 to 52 are detected by a combination of the forward primer 3 and the reverse primer 4.

The reaction product of the PCR above was analyzed using a Bioanalyzer (manufactured by Agilent Technologies, Inc.) and MultiNA (manufactured by Shimadzu Corp.) to quantify the amount of the PCR product of the transcript having each exon skipping.

### Results

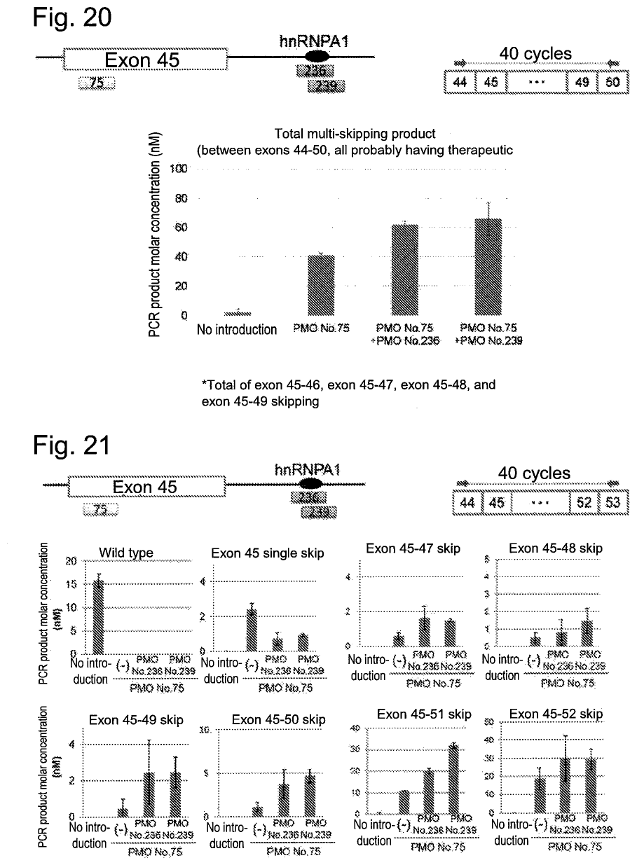

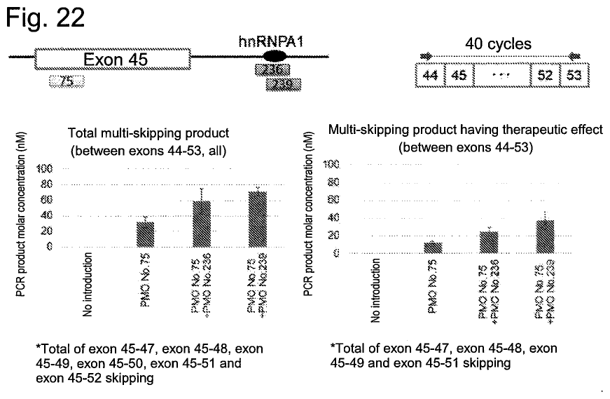

The results are shown in FIGS. 19 to 22. The introduction of PMO No. 75 (SEQ ID NO: 75) in combination with PMO No. 236 or 239 (SEQ ID NO: 260 or 263) enhanced the skipping efficiency of multi-exon skipping of exons 45 to 55 (Test Example 2) as well as multi-exon skipping of exons 45 and 46, exons 45 to 47, exons 45 to 48, exons 45 to 49, exons 45 to 50, exons 45 to 51, and exons 45 to 52 as compared to the case of PMO No. 75 singly.

These results revealed that the antisense oligomer of the present invention described in Table 11 promote or would promote overall multi-exon skipping when used in combination with the suppressor antisense oligomer of the present invention described in Table 18.

### INDUSTRIAL APPLICABILITY

The present invention provides an antisense oligomer and a suppressor antisense oligomer that cause multi-exon skipping in human dystrophin. Use of the antisense oligomer and the suppressor antisense oligomer of the present invention provides a novel therapeutic agent for DMD and method for treatment of DMD.

1. An antisense oligomer or a pharmaceutically acceptable salt thereof, or hydrate thereof which causes simultaneous skipping of any two or more numerically consecutive exons selected from the group consisting of the 45th exon to the 55th exon in human dystrophin pre-mRNA,
the antisense oligomer comprising a base sequence complementary to a base sequence of at least one region selected from the group consisting of regions R1 to R24 represented by
region Rn (wherein n is an odd number of 1 to 23) which consists of a base sequence of NX bases in the upstream direction from the 3′ end of the NAth exon and a base sequence of NY bases in the downstream direction from the 5′ end of the NBth intron in the human dystrophin pre-mRNA, and
region Rn (wherein n is an even number of 2 to 24) which consists of a base sequence of NX bases in the upstream direction from the 3′ end of the NAth intron and a base sequence of NY bases in the downstream direction from the 5′ end of the NBth exon in the human dystrophin pre-mRNA,
or a partial base sequence thereof, wherein
when n is 1, NA=44, NB=44, NX=20, and NY=400,
when n is 2, NA=44, NB=45, NX=600, and NY=50,
when n is 3, NA=45, NB=45, NX=20, and NY=400,
when n is 4, NA=45, NB=46, NX=400, and NY=50,
when n is 5, NA=46, NB=46, NX=20, and NY=400,
when n is 6, NA=46, NB=47, NX=400, and NY=50,
when n is 7, NA=47, NB=47, NX=20, and NY=400,
when n is 8, NA=47, NB=48, NX=400, and NY=50,
when n is 9, NA=48, NB=48, NX=20, and NY=400,
when n is 10, NA=48, NB=49, NX=400, and NY=50,
when n is 11, NA=49, NB=49, NX=20, and NY=400,
when n is 12, NA=49, NB=50, NX=400, and NY=50,
when n is 13, NA=50, NB=50, NX=20, and NY=400,
when n is 14, NA=50, NB=51, NX=400, and NY=50,
when n is 15, NA=51, NB=51, NX=20, and NY=400,
when n is 16, NA=51, NB=52, NX=400, and NY=50,
when n is 17, NA=52, NB=52, NX=20, and NY=400,
when n is 18, NA=52, NB=53, NX=400, and NY=50,
when n is 19, NA=53, NB=53, NX=20, and NY=400,
when n is 20, NA=53, NB=54, NX=400, and NY=50,
when n is 21, NA=54, NB=54, NX=20, and NY=400,
when n is 22, NA=54, NB=55, NX=400, and NY=50,
when n is 23, NA=55, NB=55, NX=20, and NY=400, or
when n is 24, NA=55, NB=56, NX=400, and NY=50.

2. The antisense oligomer or the pharmaceutically acceptable salt thereof, or hydrate thereof according to claim 1, wherein
the region R1 is a region that consists of a base sequence of 20 bases in the upstream direction from the 3′ end of the 44th exon and a base sequence of 400 bases in the downstream direction from the 5′ end of the 44th intron in the human dystrophin pre-mRNA,
the region R2 is a region that consists of a base sequence of 600 bases in the upstream direction from the 3′ end of the 44th intron and a base sequence of 50 bases in the downstream direction from the 5′ end of the 45th exon in the human dystrophin pre-mRNA,
the region R3 is a region that consists of a base sequence of 20 bases in the upstream direction from the 3′ end of the 45th exon and a base sequence of 400 bases in the downstream direction from the 5′ end of the 45th intron in the human dystrophin pre-mRNA,
the region R4 is a region that consists of a base sequence of 400 bases in the upstream direction from the 3′ end of the 45th intron and a base sequence of 50 bases in the downstream direction from the 5′ end of the 46th exon in the human dystrophin pre-mRNA,
the region R5 is a region that consists of a base sequence of 20 bases in the upstream direction from the 3′ end of the 46th exon and a base sequence of 400 bases in the downstream direction from the 5′ end of the 46th intron in the human dystrophin pre-mRNA,
the region R6 is a region that consists of a base sequence of 400 bases in the upstream direction from the 3′ end of the 46th intron and a base sequence of 50 bases in the downstream direction from the 5′ end of the 47th exon in the human dystrophin pre-mRNA,
the region R7 is a region that consists of a base sequence of 20 bases in the upstream direction from the 3′ end of the 47th exon and a base sequence of 400 bases in the downstream direction from the 5′ end of the 47th intron in the human dystrophin pre-mRNA,
the region R8 is a region that consists of a base sequence of 400 bases in the upstream direction from the 3′ end of the 47th intron and a base sequence of 50 bases in the downstream direction from the 5′ end of the 48th exon in the human dystrophin pre-mRNA,
the region R9 is a region that consists of a base sequence of 20 bases in the upstream direction from the 3′ end of the 48th exon and a base sequence of 400 bases in the downstream direction from the 5′ end of the 48th intron in the human dystrophin pre-mRNA,
the region R10 is a region that consists of a base sequence of 400 bases in the upstream direction from the 3′ end of the 48th intron and a base sequence of 50 bases in the downstream direction from the 5′ end of the 49th exon in the human dystrophin pre-mRNA,
the region R11 is a region that consists of a base sequence of 20 bases in the upstream direction from the 3′ end of the 49th exon and a base sequence of 400 bases in the downstream direction from the 5′ end of the 49th intron in the human dystrophin pre-mRNA,
the region R12 is a region that consists of a base sequence of 400 bases in the upstream direction from the 3′ end of the 49th intron and a base sequence of 50 bases in the downstream direction from the 5′ end of the 50th exon in the human dystrophin pre-mRNA,
the region R13 is a region that consists of a base sequence of 20 bases in the upstream direction from the 3′ end of the 50th exon and a base sequence of 400 bases in the downstream direction from the 5′ end of the 50th intron in the human dystrophin pre-mRNA,
the region R14 is a region that consists of a base sequence of 400 bases in the upstream direction from the 3′ end of the 50th intron and a base sequence of 50 bases in the downstream direction from the 5′ end of the 51st exon in the human dystrophin pre-mRNA,
the region R15 is a region that consists of a base sequence of 20 bases in the upstream direction from the 3′ end of the 51st exon and a base sequence of 400 bases in the downstream direction from the 5′ end of the 51st intron in the human dystrophin pre-mRNA,
the region R16 is a region that consists of a base sequence of 400 bases in the upstream direction from the 3′ end of the 51st intron and a base sequence of 50 bases in the downstream direction from the 5′ end of the 52nd exon in the human dystrophin pre-mRNA,
the region R17 is a region that consists of a base sequence of 20 bases in the upstream direction from the 3′ end of the 52nd exon and a base sequence of 400 bases in the downstream direction from the 5′ end of the 52nd intron in the human dystrophin pre-mRNA,
the region R18 is a region that consists of a base sequence of 400 bases in the upstream direction from the 3′ end of the 52nd intron and a base sequence of 50 bases in the downstream direction from the 5′ end of the 53rd exon in the human dystrophin pre-mRNA,
the region R19 is a region that consists of a base sequence of 20 bases in the upstream direction from the 3′ end of the 53rd exon and a base sequence of 400 bases in the downstream direction from the 5′ end of the 53rd intron in the human dystrophin pre-mRNA,
the region R20 is a region that consists of a base sequence of 400 bases in the upstream direction from the 3′ end of the 53rd intron and a base sequence of 50 bases in the downstream direction from the 5′ end of the 54th exon in the human dystrophin pre-mRNA,
the region R21 is a region that consists of a base sequence of 20 bases in the upstream direction from the 3′ end of the 54th exon and a base sequence of 400 bases in the downstream direction from the 5′ end of the 54th intron in the human dystrophin pre-mRNA,
the region R22 is a region that consists of a base sequence of 400 bases in the upstream direction from the 3′ end of the 54th intron and a base sequence of 50 bases in the downstream direction from the 5′ end of the 55th exon in the human dystrophin pre-mRNA,
the region R23 is a region that consists of a base sequence of 20 bases in the upstream direction from the 3′ end of the 55th exon and a base sequence of 400 bases in the downstream direction from the 5′ end of the 55th intron in the human dystrophin pre-mRNA, or
the region R24 is a region that consists of a base sequence of 400 bases in the upstream direction from the 3′ end of the 55th intron and a base sequence of 50 bases in the downstream direction from the 5′ end of the 56th exon in the human dystrophin pre-mRNA.

3. The antisense oligomer or the pharmaceutically acceptable salt thereof, or hydrate thereof according to claim 1, wherein
the antisense oligomer comprises a base sequence complementary to
(a) any one base sequence selected from the group consisting of SEQ ID NOs: 233 to 256, 341 to 369, and 385 to 389,
(b) a base sequence that hybridizes under stringent conditions to a base sequence complementary to any one base sequence selected from the group consisting of SEQ ID NOs: 233 to 256, 341 to 369, and 385 to 389,
(c) a base sequence that has at least 85% identity with any one base sequence selected from the group consisting of SEQ ID NOs: 233 to 256, 341 to 369, and 385 to 389, and has a length within ±15% of the length of the any one base sequence selected, or
(d) a partial base sequence of any one base sequence selected from the group consisting of the base sequences (a), (b), and (c).

4. The antisense oligomer or the pharmaceutically acceptable salt thereof, or hydrate thereof according to claim 1, wherein
the antisense oligomer is an antisense oligomer comprising two or more unit oligomers linked to each other, wherein
each of the unit oligomers comprises a base sequence complementary to a base sequence of any one region selected from the group consisting of the regions R1 to R24, or a partial base sequence thereof, and the respective base sequences of the unit oligomers are neither consecutive nor overlapped with each other.

5. The antisense oligomer or the pharmaceutically acceptable salt thereof, or hydrate thereof according to claim 1, wherein
the antisense oligomer is an antisense oligomer comprising two or more unit oligomers linked to each other, wherein
each of the unit oligomers comprises a base sequence complementary to
(a) any one base sequence selected from the group consisting of SEQ ID NOs: 233 to 256, 341 to 369, and 385 to 389,
(b) a base sequence that hybridizes under stringent conditions to a base sequence complementary to any one base sequence selected from the group consisting of SEQ ID NOs: 233 to 256, 341 to 369, and 385 to 389,
(c) a base sequence that has at least 85% identity with any one base sequence selected from the group consisting of SEQ ID NOs: 233 to 256, 341 to 369, and 385 to 389, and has a length within ±15% of the length of the any one base sequence selected, or
(d) a partial base sequence of any one base sequence selected from the group consisting of the base sequences (a), (b), and (c),
and the respective base sequences of the unit oligomers are neither consecutive nor overlapped with each other.

6. The antisense oligomer or the pharmaceutically acceptable salt thereof, or hydrate thereof according to claim 4, wherein each of the unit oligomers comprises a base sequence complementary to a consecutive base sequence of 5- to 20-base length in the region.

7. The antisense oligomer or the pharmaceutically acceptable salt thereof, or hydrate thereof according to claim 1, wherein the antisense oligomer consists of
(1) any one base sequence selected from the group consisting of SEQ ID NOs: 1 to 111, 113 to 177, 179, 180, 182, 183, 185 to 193, 195 to 198, 200 to 223, and 225 to 232, or
(2) a base sequence that has at least 85% identity with any one base sequence selected from the group consisting of SEQ ID NOs: 1 to 111, 113 to 177, 179, 180, 182, 183, 185 to 193, 195 to 198, 200 to 223, and 225 to 232, and has a length within ±15% of the length of the any one base sequence selected.

8. The antisense oligomer or the pharmaceutically acceptable salt thereof, or hydrate thereof according to claim 1, wherein the antisense oligomer consists of any one base sequence selected from the group consisting of SEQ ID NOs: 1, 5, 6, 7, 8, 10, 11, 14, 26, 27, 28, 29, 35, 38, 39, 40, 41, 43, 45, 46, 47, 50, 51, 52, 53, 54, 55, 58, 59, 63, 64, 65, 66, 67, 68, 74, 75, 76, 77, 78, 80, 82, 86, 92, 97, 98, 100, 102, 113, 119, 121, 122, 124, 125, 126, 128, 130, 131, 132, 139, 142, 144, 146, 147, 150, 151, 152, 153, 154, 155, 156, 157, 158, 159, 160, 161, 162, 163, 225, 228, 231, and 232.

9. The antisense oligomer or the pharmaceutically acceptable salt thereof, or hydrate thereof according to claim 1, wherein the antisense oligomer consists of any one base sequence selected from the group consisting of SEQ ID NOs: 1, 5, 11, 27, 28, 35, 40, 41, 43, 45, 46, 47, 50, 51, 52, 54, 55, 59, 63, 64, 65, 66, 68, 74, 75, 76, 77, 78, 102, 113, 119, 128, 131, 132, 139, 142, 161, 162, 163, 225, and 228.

10. The antisense oligomer or the pharmaceutically acceptable salt thereof, or hydrate thereof according to claim 1, wherein the antisense oligomer is an oligonucleotide.

11. The antisense oligomer or the pharmaceutically acceptable salt thereof, or hydrate thereof according to claim 10, wherein the sugar moiety and/or the phosphate bond moiety of at least one nucleotide constituting the oligonucleotide is modified.

12. The antisense oligomer or the pharmaceutically acceptable salt thereof, or hydrate thereof according to claim 10, wherein the sugar moiety of at least one nucleotide constituting the oligonucleotide is a ribose in which the 2′-OH group is replaced by any one selected from the group consisting of —OR, —R, —R′OR, —SH, —SR, —NH₂, —NHR, —NR₂, —N₃, —CN, —F, —Cl, —Br, and —I (wherein R is an alkyl or an aryl and R′ is an alkylene).

13. The antisense oligomer or the pharmaceutically acceptable salt thereof, or hydrate thereof according to claim 10, wherein the phosphate bond moiety of at least one nucleotide constituting the oligonucleotide is any one selected from the group consisting of a phosphorothioate bond, a phosphorodithioate bond, an alkylphosphonate bond, a phosphoramidate bond and a boranophosphate bond.

14. The antisense oligomer or the pharmaceutically acceptable salt thereof, or hydrate thereof according to claim 1, wherein the antisense oligomer is a morpholino oligomer.

15. The antisense oligomer or the pharmaceutically acceptable salt thereof, or hydrate thereof according to claim 14, wherein the antisense oligomer is a phosphorodiamidate morpholino oligomer.

16. The antisense oligomer or the pharmaceutically acceptable salt thereof, or hydrate thereof according to claim 14, wherein the 5′ end is any one of chemical formulae (1) to (3) below:

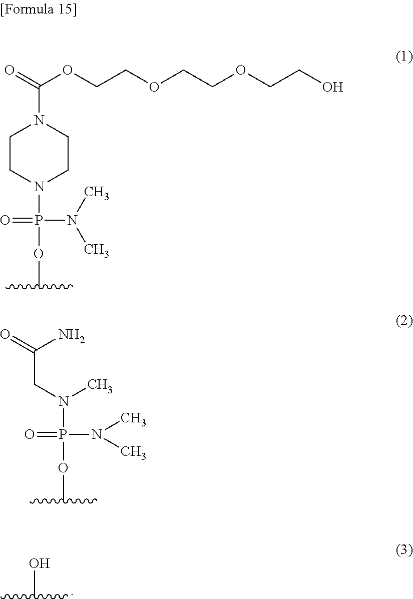

17. A suppressor antisense oligomer or a pharmaceutically acceptable salt thereof, or hydrate thereof which suppresses single skipping of any one exon selected from the group consisting of the 45th exon to the 55th exon in human dystrophin pre-mRNA,
the suppressor antisense oligomer comprising a base sequence complementary to
(a) any one base sequence of a base sequence selected from the group consisting of SEQ ID NOs: 370 to 384,
(b) a base sequence that has at least 85% identity with any one base sequence of a base sequence selected from the group consisting of SEQ ID NOs: 370 to 384, and has a length within ±15% of the length of the any one base sequence selected, or
(c) a partial base sequence of the base sequence (a) or (b).

18. The suppressor antisense oligomer or the pharmaceutically acceptable salt thereof, or hydrate thereof according to claim 17, wherein the suppressor antisense oligomer consists of
(1) any one base sequence selected from the group consisting of SEQ ID NOs: 257 to 275, or (2) a base sequence that has at least 85% identity with any one base sequence selected from the group consisting of SEQ ID NOs: 257 to 275, and has a length within ±15% of the length of the any one base sequence selected.

19. The suppressor antisense oligomer or the pharmaceutically acceptable salt thereof, or hydrate thereof according to claim 17, wherein the suppressor antisense oligomer consists of any one base sequence selected from the group consisting of SEQ ID NOs: 260, 261, and 263.

20. The suppressor antisense oligomer or the pharmaceutically acceptable salt thereof, or hydrate thereof according to claim 17, wherein the suppressor antisense oligomer is an oligonucleotide.

21. The suppressor antisense oligomer or the pharmaceutically acceptable salt thereof, or hydrate thereof according to claim 20, wherein the sugar moiety and/or the phosphate bond moiety of at least one nucleotide constituting the oligonucleotide is modified.

22. The suppressor antisense oligomer or the pharmaceutically acceptable salt thereof, or hydrate thereof according to claim 20, wherein the sugar moiety of at least one nucleotide constituting the oligonucleotide is a ribose in which the 2′-OH group is replaced by any one selected from the group consisting of —OR, —R, —R′OR, —SH, —SR, —NH₂, —NHR, —NR₂, —N₃, —CN, —F, —Cl, —Br, and —I (wherein R is an alkyl or an aryl and R′ is an alkylene).

23. The suppressor antisense oligomer or the pharmaceutically acceptable salt thereof, or hydrate thereof according to claim 20, wherein the phosphate bond moiety of at least one nucleotide constituting the oligonucleotide is any one selected from the group consisting of a phosphorothioate bond, a phosphorodithioate bond, an alkylphosphonate bond, a phosphoramidate bond and a boranophosphate bond.

24. The suppressor antisense oligomer or the pharmaceutically acceptable salt thereof, or hydrate thereof according to claim 17, wherein the suppressor antisense oligomer is a morpholino oligomer.

25. The suppressor antisense oligomer or the pharmaceutically acceptable salt thereof, or hydrate thereof according to claim 24, wherein the suppressor antisense oligomer is a phosphorodiamidate morpholino oligomer.

26. The suppressor antisense oligomer or the pharmaceutically acceptable salt thereof, or hydrate thereof according to claim 24, wherein the 5′ end is any one of chemical formulae (1) to (3) below:

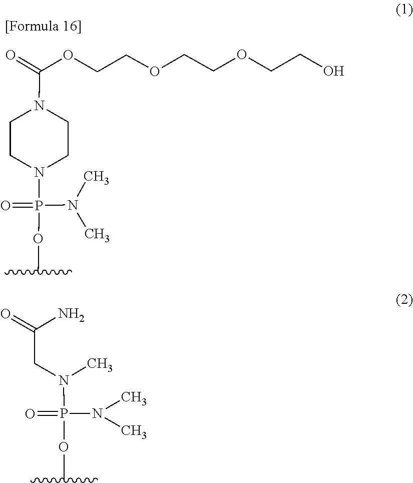 [SMILES: *OP(=O)(N(C)C)N1CCN(C(=O)OCCOCCOCCO)CC1 ; *OP(=O)(N(C)C)N(C)CC(N)=O]

27. A pharmaceutical composition comprising the antisense oligomer or the pharmaceutically acceptable salt thereof, or hydrate thereof according to claim 1.

28. The pharmaceutical composition according to claim 27, further comprising a suppressor antisense oligomer or a pharmaceutically acceptable salt thereof, or hydrate thereof,
wherein the suppressor antisense oligomer suppresses single skipping of any one exon selected from the group consisting of the 45th exon to the 55th exon in human dystrophin pre-mRNA, and
wherein the suppressor antisense oligomer comprises a base sequence complementary to
(a) any one base sequence of a base sequence selected from the group consisting of SEQ ID NOs: 370 to 384,
(b) a base sequence that has at least 85% identity with any one base sequence of a base sequence selected from the group consisting of SEQ ID NOs: 370 to 384, and has a length within ±15% of the length of the any one base sequence selected, or
(c) a partial base sequence of the base sequence (a) or (b).

29. (canceled)

30. The pharmaceutical composition according to claim 28, wherein
(1) the antisense oligomer is an oligomer consisting of SEQ ID NO: 75, and the suppressor antisense oligomer is an oligomer consisting of SEQ ID NO: 260,
(2) the antisense oligomer is an oligomer consisting of SEQ ID NO: 75, and the suppressor antisense oligomer is an oligomer consisting of SEQ ID NO: 261, or
(3) the antisense oligomer is an oligomer consisting of SEQ ID NO: 75, and the suppressor antisense oligomer is an oligomer consisting of SEQ ID NO: 263.

31. The pharmaceutical composition according to claim 27, further comprising a pharmaceutically acceptable carrier.

32-33. (canceled)

34. A method for treatment of muscular dystrophy, which comprises administering to a patient with muscular dystrophy the antisense oligomer or the pharmaceutically acceptable salt thereof, or hydrate thereof according to claim 1.

35. The method for treatment according to claim 34, wherein the patient with muscular dystrophy is a patient with a mutation that is amenable to exon 45 to 55 skipping in the dystrophin gene.

36. The method for treatment according to claim 34, wherein the patient is a human.

37-38. (canceled)

39. The method for treatment according to claim 34, wherein the treatment involves performing skipping of any two or more numerically consecutive exons selected from the group consisting of the 45th exon to the 55th exon in human dystrophin pre-mRNA.

40. (canceled)

41. A method for enhancing the efficiency of skipping of two or more numerically consecutive exons, which comprises
inhibiting a splicing silencer sequence, a splice site sequence, or a branch site sequence of pre-mRNA of interest when the two or more numerically consecutive exons are skipped from the pre-mRNA of interest.

42. The method according to claim 41, wherein the splicing silencer sequence is a recognition sequence of heterogeneous nuclear ribonucleoprotein A1 (hnRNPA1).

43. The method according to claim 41, wherein the pre-mRNA of interest is human dystrophin pre-mRNA.

44. The method according to claim 41, wherein the two or more numerically consecutive exons are selected from the group consisting of the 45th exon to the 55th exon in human dystrophin pre-mRNA.

45. The method according to claim 44, wherein the skipping of the two or more numerically consecutive exons of the pre-mRNA of interest is performed using an antisense oligomer or a pharmaceutically acceptable salt thereof, or hydrate thereof,
wherein the antisense oligomer causes simultaneous skipping of any two or more numerically consecutive exons selected from the group consisting of the 45th exon to the 55th exon in human dystrophin pre-mRNA, and
wherein the antisense oligomer comprises a base sequence complementary to a base sequence of at least one region selected from the group consisting of regions R1 to R24 represented by
region Rn (wherein n is an odd number of 1 to 23) which consists of a base sequence of NX bases in the upstream direction from the 3′ end of the NAth exon and a base sequence of NY bases in the downstream direction from the 5′ end of the NBth intron in the human dystrophin pre-mRNA, and
region Rn (wherein n is an even number of 2 to 24) which consists of a base sequence of NX bases in the upstream direction from the 3′ end of the NAth intron and a base sequence of NY bases in the downstream direction from the 5′ end of the NBth exon in the human dystrophin pre-mRNA,
or a partial base sequence thereof, wherein
when n is 1, NA=44, NB=44, NX=20, and NY=400,
when n is 2, NA=44, NB=45, NX=600, and NY=50,
when n is 3, NA=45, NB=45, NX=20, and NY=400,
when n is 4, NA=45, NB=46, NX=400, and NY=50,
when n is 5, NA=46, NB=46, NX=20, and NY=400,
when n is 6, NA=46, NB=47, NX=400, and NY=50,
when n is 7, NA=47, NB=47, NX=20, and NY=400,
when n is 8, NA=47, NB=48, NX=400, and NY=50,
when n is 9, NA=48, NB=48, NX=20, and NY=400,
when n is 10, NA=48, NB=49, NX=400, and NY=50,
when n is 11, NA=49, NB=49, NX=20, and NY=400,
when n is 12, NA=49, NB=50, NX=400, and NY=50,
when n is 13, NA=50, NB=50, NX=20, and NY=400,
when n is 14, NA=50, NB=51, NX=400, and NY=50,
when n is 15, NA=51, NB=51, NX=20, and NY=400,
when n is 16, NA=51, NB=52, NX=400, and NY=50,
when n is 17, NA=52, NB=52, NX=20, and NY=400,
when n is 18, NA=52, NB=53, NX=400, and NY=50,
when n is 19, NA=53, NB=53, NX=20, and NY=400,
when n is 20, NA=53, NB=54, NX=400, and NY=50,
when n is 21, NA=54, NB=54, NX=20, and NY=400,
when n is 22, NA=54, NB=55, NX=400, and NY=50,
when n is 23, NA=55, NB=55, NX=20, and NY=400, or
when n is 24, NA=55, NB=56, NX=400, and NY=50.

46. The method according to claim 44, wherein
specific inhibition of the splicing silencer sequence, the splice site sequence, or the branch site sequence is performed using a suppressor antisense oligomer or a pharmaceutically acceptable salt thereof, or hydrate thereof,
wherein the suppressor antisense oligomer suppresses single skipping of any one exon selected from the group consisting of the 45th exon to the 55th exon in human dystrophin pre-mRNA, and
wherein the suppressor antisense oligomer comprises a base sequence complementary to
(a) any one base sequence of a base sequence selected from the group consisting of SEQ ID NOs: 370 to 384,
(b) a base sequence that has at least 85% identity with any one base sequence of a base sequence selected from the group consisting of SEQ ID NOs: 370 to 384, and has a length within ±15% of the length of the any one base sequence selected, or
(c) a partial base sequence of the base sequence (a) or (b).


SEQUENCE LISTING (390 sequences)

SEQ ID NO: 1 - DNA, 26 bp, Artificial
  Synthetic Nucleic Acid
tttcgaaaaaacaaatcaaagactta

SEQ ID NO: 2 - DNA, 40 bp, Artificial
  Synthetic Nucleic Acid
atgtgctgaagataaatacaatttcgaaaaaacaaatcaa

SEQ ID NO: 3 - DNA, 35 bp, Artificial
  Synthetic Nucleic Acid
tgctgaagataaatacaatttcgaaaaaacaaatc

SEQ ID NO: 4 - DNA, 30 bp, Artificial
  Synthetic Nucleic Acid
cagatgtgctgaagataaatacaatttcga

SEQ ID NO: 5 - DNA, 28 bp, Artificial
  Synthetic Nucleic Acid
aagagtccagatgtgctgaagataaata

SEQ ID NO: 6 - DNA, 28 bp, Artificial
  Synthetic Nucleic Acid
acccttcagaacctgatctttaagaagt

SEQ ID NO: 7 - DNA, 28 bp, Artificial
  Synthetic Nucleic Acid
tgacaacaacagtcaaaagtaatttcca

SEQ ID NO: 8 - DNA, 28 bp, Artificial
  Synthetic Nucleic Acid
atgataattttctttctagtaatataat

SEQ ID NO: 9 - DNA, 28 bp, Artificial
  Synthetic Nucleic Acid
tccatagcaccgtgctctaatattatca

SEQ ID NO: 10 - DNA, 26 bp, Artificial
  Synthetic Nucleic Acid
ggcaaactctctcatcctgacacaaa

SEQ ID NO: 11 - DNA, 28 bp, Artificial
  Synthetic Nucleic Acid
tttatcagataaaccagctccgtccagg

SEQ ID NO: 12 - DNA, 28 bp, Artificial
  Synthetic Nucleic Acid
cttccctctgtcacagattcaattatat

SEQ ID NO: 13 - DNA, 26 bp, Artificial
  Synthetic Nucleic Acid
aaaacaccttgctgttacgatgcttc

SEQ ID NO: 14 - DNA, 30 bp, Artificial
  Synthetic Nucleic Acid
tgccccaaagccacaaaacaccttgctgtt

SEQ ID NO: 15 - DNA, 26 bp, Artificial
  Synthetic Nucleic Acid
ggttccaacataaagccgaaatacac

SEQ ID NO: 16 - DNA, 28 bp, Artificial
  Synthetic Nucleic Acid
tatgccacaagttctccttctggaaagg

SEQ ID NO: 17 - DNA, 28 bp, Artificial
  Synthetic Nucleic Acid
gatatttctagcaacttcattttagcta

SEQ ID NO: 18 - DNA, 30 bp, Artificial
  Synthetic Nucleic Acid
tgctgaagataaatacaaatcaaagactta

SEQ ID NO: 19 - DNA, 30 bp, Artificial
  Synthetic Nucleic Acid
aagagtccagatgtgcaaatcaaagactta

SEQ ID NO: 20 - DNA, 30 bp, Artificial
  Synthetic Nucleic Acid
tgctgaagataaatatttcgaaaaaacaaa

SEQ ID NO: 21 - DNA, 30 bp, Artificial
  Synthetic Nucleic Acid
aagagtccagatgtgtttcgaaaaaacaaa

SEQ ID NO: 22 - DNA, 28 bp, Artificial
  Synthetic Nucleic Acid
ttaattgctttcaggagcatcccatcaa

SEQ ID NO: 23 - DNA, 28 bp, Artificial
  Synthetic Nucleic Acid
tttgcattagaagccacaaaaaactgag

SEQ ID NO: 24 - DNA, 28 bp, Artificial
  Synthetic Nucleic Acid
tcatttcaaattctgtctgcgtcaatgt

SEQ ID NO: 25 - DNA, 28 bp, Artificial
  Synthetic Nucleic Acid
ttgctatattagaagaaaattcatttca

SEQ ID NO: 26 - DNA, 28 bp, Artificial
  Synthetic Nucleic Acid
taattgctatattagaagaaaattcatt

SEQ ID NO: 27 - DNA, 30 bp, Artificial
  Synthetic Nucleic Acid
aaataaaattaattgctatattagaagaaa

SEQ ID NO: 28 - DNA, 30 bp, Artificial
  Synthetic Nucleic Acid
cagtattaaaaaaaaactctagagatattt

SEQ ID NO: 29 - DNA, 28 bp, Artificial
  Synthetic Nucleic Acid
tagtcacagtattaaaaaaaaactctag

SEQ ID NO: 30 - DNA, 28 bp, Artificial
  Synthetic Nucleic Acid
gtgaaaaagaacaaacataggttagtca

SEQ ID NO: 31 - DNA, 28 bp, Artificial
  Synthetic Nucleic Acid
atacgagaggtgaaaaagaacaaacata

SEQ ID NO: 32 - DNA, 28 bp, Artificial
  Synthetic Nucleic Acid
tggatacgagaggtgaaaaagaacaaac

SEQ ID NO: 33 - DNA, 28 bp, Artificial
  Synthetic Nucleic Acid
tttcttagtgatcgtggatacgagaggt

SEQ ID NO: 34 - DNA, 25 bp, Artificial
  Synthetic Nucleic Acid
gtttcttagtgatcgtggatacgag

SEQ ID NO: 35 - DNA, 28 bp, Artificial
  Synthetic Nucleic Acid
tgggtttcttagtgatcgtggatacgag

SEQ ID NO: 36 - DNA, 25 bp, Artificial
  Synthetic Nucleic Acid
tgggtttcttagtgatcgtggatac

SEQ ID NO: 37 - DNA, 28 bp, Artificial
  Synthetic Nucleic Acid
gtatttgggtttcttagtgatcgtggat

SEQ ID NO: 38 - DNA, 28 bp, Artificial
  Synthetic Nucleic Acid
tgaacaaagtatttgggtttcttagtga

SEQ ID NO: 39 - DNA, 28 bp, Artificial
  Synthetic Nucleic Acid
ttgtaaaatttaaacatgaacaaagtat

SEQ ID NO: 40 - DNA, 28 bp, Artificial
  Synthetic Nucleic Acid
tccccacaaggatgttccatgtttaata

SEQ ID NO: 41 - DNA, 28 bp, Artificial
  Synthetic Nucleic Acid
accttttcaagagcaaattcgatttctt

SEQ ID NO: 42 - DNA, 26 bp, Artificial
  Synthetic Nucleic Acid
caattagttggaaaccttttcaagag

SEQ ID NO: 43 - DNA, 28 bp, Artificial
  Synthetic Nucleic Acid
tataatgtcctacaaatcaattagttgg

SEQ ID NO: 44 - DNA, 26 bp, Artificial
  Synthetic Nucleic Acid
agctagaggatgttataatgtcctac

SEQ ID NO: 45 - DNA, 28 bp, Artificial
  Synthetic Nucleic Acid
atttttgtaagcttgtcagctagaggat

SEQ ID NO: 46 - DNA, 28 bp, Artificial
  Synthetic Nucleic Acid
aaagcaccctctcggttagctccagttt

SEQ ID NO: 47 - DNA, 28 bp, Artificial
  Synthetic Nucleic Acid
cagaaagacaccttttatgtgtcaggga

SEQ ID NO: 48 - DNA, 28 bp, Artificial
  Synthetic Nucleic Acid
ggatacaagacagaaagacaccttttat

SEQ ID NO: 49 - DNA, 26 bp, Artificial
  Synthetic Nucleic Acid
aaaggatacaagacagaaagacacct

SEQ ID NO: 50 - DNA, 28 bp, Artificial
  Synthetic Nucleic Acid
tgacatgcccatatccaaaggatacaag

SEQ ID NO: 51 - DNA, 28 bp, Artificial
  Synthetic Nucleic Acid
aactgacatgcccatatccaaaggatac

SEQ ID NO: 52 - DNA, 28 bp, Artificial
  Synthetic Nucleic Acid
agctccatgtgaaaatttccctatgaaa

SEQ ID NO: 53 - DNA, 28 bp, Artificial
  Synthetic Nucleic Acid
tgcagttgtactggcaaagaaagaaata

SEQ ID NO: 54 - DNA, 28 bp, Artificial
  Synthetic Nucleic Acid
tgagaaaagattaaacagtgtgctacca

SEQ ID NO: 55 - DNA, 28 bp, Artificial
  Synthetic Nucleic Acid
tttgagaaaagattaaacagtgtgctac

SEQ ID NO: 56 - DNA, 28 bp, Artificial
  Synthetic Nucleic Acid
tctttttatttgagaaaagattaaacag

SEQ ID NO: 57 - DNA, 28 bp, Artificial
  Synthetic Nucleic Acid
aagccccatgtctttttatttgagaaaa

SEQ ID NO: 58 - DNA, 28 bp, Artificial
  Synthetic Nucleic Acid
aacaaaaatgaagccccatgtcttttta

SEQ ID NO: 59 - DNA, 28 bp, Artificial
  Synthetic Nucleic Acid
aggcaaaacaaaaatgaagccccatgtc

SEQ ID NO: 60 - DNA, 28 bp, Artificial
  Synthetic Nucleic Acid
ttcctgtaagataccaaaaaggcaaaac

SEQ ID NO: 61 - DNA, 28 bp, Artificial
  Synthetic Nucleic Acid
agttcctgtaagataccaaaaaggcaaa

SEQ ID NO: 62 - DNA, 25 bp, Artificial
  Synthetic Nucleic Acid
ttcctgtaagataccaaaaaggcaa

SEQ ID NO: 63 - DNA, 28 bp, Artificial
  Synthetic Nucleic Acid
ggagttcctgtaagataccaaaaaggca

SEQ ID NO: 64 - DNA, 25 bp, Artificial
  Synthetic Nucleic Acid
agttcctgtaagataccaaaaaggc

SEQ ID NO: 65 - DNA, 28 bp, Artificial
  Synthetic Nucleic Acid
tggagttcctgtaagataccaaaaaggc

SEQ ID NO: 66 - DNA, 28 bp, Artificial
  Synthetic Nucleic Acid
cctggagttcctgtaagataccaaaaag

SEQ ID NO: 67 - DNA, 25 bp, Artificial
  Synthetic Nucleic Acid
tggagttcctgtaagataccaaaaa

SEQ ID NO: 68 - DNA, 28 bp, Artificial
  Synthetic Nucleic Acid
atcctggagttcctgtaagataccaaaa

SEQ ID NO: 69 - DNA, 25 bp, Artificial
  Synthetic Nucleic Acid
cctggagttcctgtaagataccaaa

SEQ ID NO: 70 - DNA, 28 bp, Artificial
  Synthetic Nucleic Acid
ccatcctggagttcctgtaagataccaa

SEQ ID NO: 71 - DNA, 26 bp, Artificial
  Synthetic Nucleic Acid
tgccatcctggagttcctgtaagata

SEQ ID NO: 72 - DNA, 25 bp, Artificial
  Synthetic Nucleic Acid
ccaatgccatcctggagttcctgta

SEQ ID NO: 73 - DNA, 25 bp, Artificial
  Synthetic Nucleic Acid
gctgcccaatgccatcctggagttc

SEQ ID NO: 74 - DNA, 25 bp, Artificial
  Synthetic Nucleic Acid
ttgccgctgcccaatgccatcctgg

SEQ ID NO: 75 - DNA, 25 bp, Artificial
  Synthetic Nucleic Acid
acagtttgccgctgcccaatgccat

SEQ ID NO: 76 - DNA, 30 bp, Artificial
  Synthetic Nucleic Acid
tgacaacagtttgccgctgcccaatgccat

SEQ ID NO: 77 - DNA, 20 bp, Artificial
  Synthetic Nucleic Acid
acagtttgccgctgcccaat

SEQ ID NO: 78 - DNA, 25 bp, Artificial
  Synthetic Nucleic Acid
tgacaacagtttgccgctgcccaat

SEQ ID NO: 79 - DNA, 25 bp, Artificial
  Synthetic Nucleic Acid
tgttctgacaacagtttgccgctgc

SEQ ID NO: 80 - DNA, 30 bp, Artificial
  Synthetic Nucleic Acid
agtttgccgctgcccaatatacgagaggtg

SEQ ID NO: 81 - DNA, 30 bp, Artificial
  Synthetic Nucleic Acid
ttgccgctgcccaatgtgatcgtggatacg

SEQ ID NO: 82 - DNA, 30 bp, Artificial
  Synthetic Nucleic Acid
agtttgccgctgcccaattagtgatcgtgg

SEQ ID NO: 83 - DNA, 30 bp, Artificial
  Synthetic Nucleic Acid
ttgccgctgcccaattgggtttcttagtga

SEQ ID NO: 84 - DNA, 30 bp, Artificial
  Synthetic Nucleic Acid
agtttgccgctgcccaattgggtttcttag

SEQ ID NO: 85 - DNA, 28 bp, Artificial
  Synthetic Nucleic Acid
aatttccctatgaactgacatgcccata

SEQ ID NO: 86 - DNA, 28 bp, Artificial
  Synthetic Nucleic Acid
tgaagccccatgtcaaacagtgtgctac

SEQ ID NO: 87 - DNA, 28 bp, Artificial
  Synthetic Nucleic Acid
aaaacaaaaatgaagaacagtgtgctac

SEQ ID NO: 88 - DNA, 28 bp, Artificial
  Synthetic Nucleic Acid
tgccgctgcccaataaacagtgtgctac

SEQ ID NO: 89 - DNA, 28 bp, Artificial
  Synthetic Nucleic Acid
cagtttgccgctgcaaacagtgtgctac

SEQ ID NO: 90 - DNA, 28 bp, Artificial
  Synthetic Nucleic Acid
tgacaacagtttgcaaacagtgtgctac

SEQ ID NO: 91 - DNA, 26 bp, Artificial
  Synthetic Nucleic Acid
aaatgaagccccaattaaacagtgtg

SEQ ID NO: 92 - DNA, 28 bp, Artificial
  Synthetic Nucleic Acid
aaaaatgaagcccaaagattaaacagtg

SEQ ID NO: 93 - DNA, 28 bp, Artificial
  Synthetic Nucleic Acid
caaaaatgaagcccaaagattaaacagt

SEQ ID NO: 94 - DNA, 28 bp, Artificial
  Synthetic Nucleic Acid
tgccgctgcccaataaagattaaacagt

SEQ ID NO: 95 - DNA, 28 bp, Artificial
  Synthetic Nucleic Acid
cagtttgccgctgcaaagattaaacagt

SEQ ID NO: 96 - DNA, 28 bp, Artificial
  Synthetic Nucleic Acid
tgacaacagtttgcaaagattaaacagt

SEQ ID NO: 97 - DNA, 28 bp, Artificial
  Synthetic Nucleic Acid
gccgctgcccaaatgaagcagattaaac

SEQ ID NO: 98 - DNA, 28 bp, Artificial
  Synthetic Nucleic Acid
tgaagccccatgtctttgagaaaagatt

SEQ ID NO: 99 - DNA, 28 bp, Artificial
  Synthetic Nucleic Acid
aaacaaaaatgaagtttgagaaaagatt

SEQ ID NO: 100 - DNA, 28 bp, Artificial
  Synthetic Nucleic Acid
tgccgctgcccaattttgagaaaagatt

SEQ ID NO: 101 - DNA, 28 bp, Artificial
  Synthetic Nucleic Acid
cagtttgccgctgctttgagaaaagatt

SEQ ID NO: 102 - DNA, 28 bp, Artificial
  Synthetic Nucleic Acid
tgacaacagtttgctttgagaaaagatt

SEQ ID NO: 103 - DNA, 28 bp, Artificial
  Synthetic Nucleic Acid
tgccgctgcccaattgaagccccatgtc

SEQ ID NO: 104 - DNA, 28 bp, Artificial
  Synthetic Nucleic Acid
tgacaacagtttgctgaagccccatgtc

SEQ ID NO: 105 - DNA, 28 bp, Artificial
  Synthetic Nucleic Acid
tagtttgccgctgctgaagccccatgtc

SEQ ID NO: 106 - DNA, 28 bp, Artificial
  Synthetic Nucleic Acid
tgccgctgcccaatcaaaaatgaagccc

SEQ ID NO: 107 - DNA, 28 bp, Artificial
  Synthetic Nucleic Acid
cagtttgccgctgccaaaaatgaagccc

SEQ ID NO: 108 - DNA, 28 bp, Artificial
  Synthetic Nucleic Acid
tgacaacagtttgccaaaaatgaagccc

SEQ ID NO: 109 - DNA, 28 bp, Artificial
  Synthetic Nucleic Acid
tgccgctgcccaataaaacaaaaatgaa

SEQ ID NO: 110 - DNA, 28 bp, Artificial
  Synthetic Nucleic Acid
cagtttgccgctgcaaaacaaaaatgaa

SEQ ID NO: 111 - DNA, 28 bp, Artificial
  Synthetic Nucleic Acid
tgacaacagtttgcaaaacaaaaatgaa

SEQ ID NO: 112 - DNA, 21 bp, Artificial
  Synthetic Nucleic Acid
cccaatgccatcctggagttc

SEQ ID NO: 113 - DNA, 28 bp, Artificial
  Synthetic Nucleic Acid
aatgcctgacttacttgccattgtttca

SEQ ID NO: 114 - DNA, 28 bp, Artificial
  Synthetic Nucleic Acid
agtgctaaagcggaaatgcctgacttac

SEQ ID NO: 115 - DNA, 28 bp, Artificial
  Synthetic Nucleic Acid
tgctgagaattgttcaattggatccaca

SEQ ID NO: 116 - DNA, 28 bp, Artificial
  Synthetic Nucleic Acid
tgtccctggcttgtcagttacaagtaca

SEQ ID NO: 117 - DNA, 28 bp, Artificial
  Synthetic Nucleic Acid
tcaggctgtataaaagcaactattttgt

SEQ ID NO: 118 - DNA, 28 bp, Artificial
  Synthetic Nucleic Acid
ctctcctccttgtccaaataccgaaata

SEQ ID NO: 119 - DNA, 28 bp, Artificial
  Synthetic Nucleic Acid
gatgtttccttctccctctgcctctctc

SEQ ID NO: 120 - DNA, 28 bp, Artificial
  Synthetic Nucleic Acid
tataaatgatgtttccttctccctctgc

SEQ ID NO: 121 - DNA, 28 bp, Artificial
  Synthetic Nucleic Acid
tttttctaagacgagggtgttaagtgga

SEQ ID NO: 122 - DNA, 28 bp, Artificial
  Synthetic Nucleic Acid
tgcaaatgttttcctggtcagagcatgt

SEQ ID NO: 123 - DNA, 28 bp, Artificial
  Synthetic Nucleic Acid
tttctccttgaccgaagctctggtttta

SEQ ID NO: 124 - DNA, 28 bp, Artificial
  Synthetic Nucleic Acid
ccaatccctaagttatttctctgagcaa

SEQ ID NO: 125 - DNA, 28 bp, Artificial
  Synthetic Nucleic Acid
caaaaatgtcaacttttaaaatttaata

SEQ ID NO: 126 - DNA, 28 bp, Artificial
  Synthetic Nucleic Acid
gctttccctgtaaaatattaaataaaca

SEQ ID NO: 127 - DNA, 28 bp, Artificial
  Synthetic Nucleic Acid
taaataaaacagacaattcatacagatg

SEQ ID NO: 128 - DNA, 28 bp, Artificial
  Synthetic Nucleic Acid
aataatttcattactatatggatcaagt

SEQ ID NO: 129 - DNA, 26 bp, Artificial
  Synthetic Nucleic Acid
aaagtgtaccccagtgccaataattt

SEQ ID NO: 130 - DNA, 28 bp, Artificial
  Synthetic Nucleic Acid
tattttatggaagtgataggcaataaaa

SEQ ID NO: 131 - DNA, 28 bp, Artificial
  Synthetic Nucleic Acid
tatatcttctagtctatggacaaaatgt

SEQ ID NO: 132 - DNA, 28 bp, Artificial
  Synthetic Nucleic Acid
tgtatttataactttataaagttcacaa

SEQ ID NO: 133 - DNA, 28 bp, Artificial
  Synthetic Nucleic Acid
tattccttgccattatgagttggaaagt

SEQ ID NO: 134 - DNA, 28 bp, Artificial
  Synthetic Nucleic Acid
aaatgggcatcttattagttgtaataga

SEQ ID NO: 135 - DNA, 28 bp, Artificial
  Synthetic Nucleic Acid
attgccttctcctgttattatgtagatt

SEQ ID NO: 136 - DNA, 28 bp, Artificial
  Synthetic Nucleic Acid
agatacatctcaaatcccttttcttggc

SEQ ID NO: 137 - DNA, 28 bp, Artificial
  Synthetic Nucleic Acid
agacatttcaatcaggctaaactaacaa

SEQ ID NO: 138 - DNA, 28 bp, Artificial
  Synthetic Nucleic Acid
ttgcaaaatataaataattattagttca

SEQ ID NO: 139 - DNA, 26 bp, Artificial
  Synthetic Nucleic Acid
acaagcgatgaatgtgaatttggaga

SEQ ID NO: 140 - DNA, 26 bp, Artificial
  Synthetic Nucleic Acid
attaccaaacaaaagaaacaagcgat

SEQ ID NO: 141 - DNA, 26 bp, Artificial
  Synthetic Nucleic Acid
ggaagaagaatatgtgcagaattacc

SEQ ID NO: 142 - DNA, 26 bp, Artificial
  Synthetic Nucleic Acid
ggacagcaggaagaagaatatgtgca

SEQ ID NO: 143 - DNA, 28 bp, Artificial
  Synthetic Nucleic Acid
tcaccttggaggtcctacaggacagcag

SEQ ID NO: 144 - DNA, 28 bp, Artificial
  Synthetic Nucleic Acid
ttagatctgtcgccctacctcttttttc

SEQ ID NO: 145 - DNA, 28 bp, Artificial
  Synthetic Nucleic Acid
tctcatgaaatattcttctaaagaaagc

SEQ ID NO: 146 - DNA, 28 bp, Artificial
  Synthetic Nucleic Acid
atgttagtgcctttcaccctgcttataa

SEQ ID NO: 147 - DNA, 28 bp, Artificial
  Synthetic Nucleic Acid
gctgttgattaatggttgataggttctt

SEQ ID NO: 148 - DNA, 28 bp, Artificial
  Synthetic Nucleic Acid
tgaaaaaaagaaataaaaaatttcttta

SEQ ID NO: 149 - DNA, 28 bp, Artificial
  Synthetic Nucleic Acid
taactagccacaagtatatattttagta

SEQ ID NO: 150 - DNA, 28 bp, Artificial
  Synthetic Nucleic Acid
tcaagaatctctaaatgataagagatta

SEQ ID NO: 151 - DNA, 28 bp, Artificial
  Synthetic Nucleic Acid
ttcactttgaacaaagtaatttcaatat

SEQ ID NO: 152 - DNA, 28 bp, Artificial
  Synthetic Nucleic Acid
accatacataatttaagaaaattcattc

SEQ ID NO: 153 - DNA, 28 bp, Artificial
  Synthetic Nucleic Acid
tgttaaccatacataatttaagaaaatt

SEQ ID NO: 154 - DNA, 28 bp, Artificial
  Synthetic Nucleic Acid
aaagatgttaaccatacataatttaaga

SEQ ID NO: 155 - DNA, 28 bp, Artificial
  Synthetic Nucleic Acid
aagcaatttaaaagatgttaaccataca

SEQ ID NO: 156 - DNA, 28 bp, Artificial
  Synthetic Nucleic Acid
atttaaaaataagcaatttaaaagatgt

SEQ ID NO: 157 - DNA, 28 bp, Artificial
  Synthetic Nucleic Acid
tggcaatttaaaaataagcaatttaaaa

SEQ ID NO: 158 - DNA, 28 bp, Artificial
  Synthetic Nucleic Acid
aaacatggcaatttaaaaataagcaatt

SEQ ID NO: 159 - DNA, 28 bp, Artificial
  Synthetic Nucleic Acid
tatttgttaatgcaaactgggacacaaa

SEQ ID NO: 160 - DNA, 28 bp, Artificial
  Synthetic Nucleic Acid
ttatttttttttccaacatagttctcaa

SEQ ID NO: 161 - DNA, 24 bp, Artificial
  Synthetic Nucleic Acid
tctagcctggagaaagaagaataa

SEQ ID NO: 162 - DNA, 24 bp, Artificial
  Synthetic Nucleic Acid
tcttctagcctggagaaagaagaa

SEQ ID NO: 163 - DNA, 24 bp, Artificial
  Synthetic Nucleic Acid
ttttgttcttctagcctggagaaa

SEQ ID NO: 164 - DNA, 28 bp, Artificial
  Synthetic Nucleic Acid
ttgagaaaataaaattaccttgacttgc

SEQ ID NO: 165 - DNA, 28 bp, Artificial
  Synthetic Nucleic Acid
acttctttatgcaagcaggccctggggg

SEQ ID NO: 166 - DNA, 28 bp, Artificial
  Synthetic Nucleic Acid
caatgattgaattaaaaaatagattcat

SEQ ID NO: 167 - DNA, 28 bp, Artificial
  Synthetic Nucleic Acid
gaactatgaataacctaatgggcagaaa

SEQ ID NO: 168 - DNA, 28 bp, Artificial
  Synthetic Nucleic Acid
ataaagttgtgagaaaaacactttagca

SEQ ID NO: 169 - DNA, 28 bp, Artificial
  Synthetic Nucleic Acid
actggttcagaactgcagggttaagaag

SEQ ID NO: 170 - DNA, 28 bp, Artificial
  Synthetic Nucleic Acid
acacacatatatacatatgttcttatgt

SEQ ID NO: 171 - DNA, 28 bp, Artificial
  Synthetic Nucleic Acid
ctcaccccctcagtagataaatctctgt

SEQ ID NO: 172 - DNA, 28 bp, Artificial
  Synthetic Nucleic Acid
tctgagcacagagctgattgactgaaac

SEQ ID NO: 173 - DNA, 28 bp, Artificial
  Synthetic Nucleic Acid
agtggtacctcaaataccaacagttttc

SEQ ID NO: 174 - DNA, 28 bp, Artificial
  Synthetic Nucleic Acid
atcaaaatgaagcgacttgaccgagggc

SEQ ID NO: 175 - DNA, 28 bp, Artificial
  Synthetic Nucleic Acid
taccttgtctttgcttctattgattagt

SEQ ID NO: 176 - DNA, 28 bp, Artificial
  Synthetic Nucleic Acid
gtttaaaatgaattacagcacaattcca

SEQ ID NO: 177 - DNA, 28 bp, Artificial
  Synthetic Nucleic Acid
ttccaccagtaactgaaacagacaaatg

SEQ ID NO: 178 - DNA, 29 bp, Artificial
  Synthetic Nucleic Acid
tggcgcaggggcaactcttccaccagtaa

SEQ ID NO: 179 - DNA, 28 bp, Artificial
  Synthetic Nucleic Acid
ttaatgtctaacctttatccactggaga

SEQ ID NO: 180 - DNA, 28 bp, Artificial
  Synthetic Nucleic Acid
tggaaacctgaaaggaaaatacatttta

SEQ ID NO: 181 - DNA, 28 bp, Artificial
  Synthetic Nucleic Acid
cttgtttctcaggtaaagctctggaaac

SEQ ID NO: 182 - DNA, 28 bp, Artificial
  Synthetic Nucleic Acid
aagcaaaaagttccctacctgaacgtca

SEQ ID NO: 183 - DNA, 28 bp, Artificial
  Synthetic Nucleic Acid
cagtttcctggggaaaagaacccatata

SEQ ID NO: 184 - DNA, 26 bp, Artificial
  Synthetic Nucleic Acid
atctcttccacatccggttgtttagc

SEQ ID NO: 185 - DNA, 28 bp, Artificial
  Synthetic Nucleic Acid
tagaggttgcttcattaccttcactggc

SEQ ID NO: 186 - DNA, 28 bp, Artificial
  Synthetic Nucleic Acid
accctacaaatatttatcaattgctcca

SEQ ID NO: 187 - DNA, 28 bp, Artificial
  Synthetic Nucleic Acid
taaaggaattataattattttagccaac

SEQ ID NO: 188 - DNA, 28 bp, Artificial
  Synthetic Nucleic Acid
taaattaactttagtgggtagaatttct

SEQ ID NO: 189 - DNA, 28 bp, Artificial
  Synthetic Nucleic Acid
tattattggatttctattatattttact

SEQ ID NO: 190 - DNA, 28 bp, Artificial
  Synthetic Nucleic Acid
tcatgaacatcttaatccatttggtgaa

SEQ ID NO: 191 - DNA, 28 bp, Artificial
  Synthetic Nucleic Acid
tacttattcgattaacactttgaagata

SEQ ID NO: 192 - DNA, 28 bp, Artificial
  Synthetic Nucleic Acid
acttcctctttaacagaaaagcatacac

SEQ ID NO: 193 - DNA, 28 bp, Artificial
  Synthetic Nucleic Acid
gggatccagtatacttacaggctccaat

SEQ ID NO: 194 - DNA, 25 bp, Artificial
  Synthetic Nucleic Acid
cttacaggctccaatagtggtcagt

SEQ ID NO: 195 - DNA, 28 bp, Artificial
  Synthetic Nucleic Acid
tgagtaggagctaaaatattttgggttt

SEQ ID NO: 196 - DNA, 28 bp, Artificial
  Synthetic Nucleic Acid
tatcattttttctcataccttctgcttg

SEQ ID NO: 197 - DNA, 28 bp, Artificial
  Synthetic Nucleic Acid
ttagtatcagggttcttcagcgttgtgt

SEQ ID NO: 198 - DNA, 28 bp, Artificial
  Synthetic Nucleic Acid
gcattgttgcctgtaagaacaaatatcc

SEQ ID NO: 199 - DNA, 30 bp, Artificial
  Synthetic Nucleic Acid
aactggggacgcctctgttccaaatcctgc

SEQ ID NO: 200 - DNA, 28 bp, Artificial
  Synthetic Nucleic Acid
gcttgttaaaaaacttacttcgatccgt

SEQ ID NO: 201 - DNA, 28 bp, Artificial
  Synthetic Nucleic Acid
aatattagtttctgttaaattattttcc

SEQ ID NO: 202 - DNA, 28 bp, Artificial
  Synthetic Nucleic Acid
acaacaggattctttgcttttttgatgg

SEQ ID NO: 203 - DNA, 28 bp, Artificial
  Synthetic Nucleic Acid
ttattcattgtgttatggctaggatgat

SEQ ID NO: 204 - DNA, 28 bp, Artificial
  Synthetic Nucleic Acid
atctcacatttatgttgcttatttaaaa

SEQ ID NO: 205 - DNA, 28 bp, Artificial
  Synthetic Nucleic Acid
gagacattttaaatgtaacttccaaacg

SEQ ID NO: 206 - DNA, 28 bp, Artificial
  Synthetic Nucleic Acid
aaatatatagtagtaaatgctagtctgg

SEQ ID NO: 207 - DNA, 28 bp, Artificial
  Synthetic Nucleic Acid
attctttcaactagaataaaaggaaaaa

SEQ ID NO: 208 - DNA, 28 bp, Artificial
  Synthetic Nucleic Acid
ggtatctttgatactaaccttggtttct

SEQ ID NO: 209 - DNA, 28 bp, Artificial
  Synthetic Nucleic Acid
tggccaactgctatagatttttatgaga

SEQ ID NO: 210 - DNA, 28 bp, Artificial
  Synthetic Nucleic Acid
aaataatgtaattcataccttttatgaa

SEQ ID NO: 211 - DNA, 28 bp, Artificial
  Synthetic Nucleic Acid
ccccattattacagccaacagtagtttt

SEQ ID NO: 212 - DNA, 28 bp, Artificial
  Synthetic Nucleic Acid
caaatcctcatggtccatccagtttcac

SEQ ID NO: 213 - DNA, 28 bp, Artificial
  Synthetic Nucleic Acid
ccaagctccagtttagctggattggaaa

SEQ ID NO: 214 - DNA, 28 bp, Artificial
  Synthetic Nucleic Acid
tttagttggtatttatcgtcttgaaccc

SEQ ID NO: 215 - DNA, 28 bp, Artificial
  Synthetic Nucleic Acid
taaaatagaagtctgagccaagtccgtg

SEQ ID NO: 216 - DNA, 28 bp, Artificial
  Synthetic Nucleic Acid
gggcaaatgagatcttatgttcctcgtt

SEQ ID NO: 217 - DNA, 28 bp, Artificial
  Synthetic Nucleic Acid
tttactttcatttttatttaacttaaag

SEQ ID NO: 218 - DNA, 28 bp, Artificial
  Synthetic Nucleic Acid
taagcaacaactataatattgtgcagtc

SEQ ID NO: 219 - DNA, 28 bp, Artificial
  Synthetic Nucleic Acid
tggggtgagttgttgctacagctcttcc

SEQ ID NO: 220 - DNA, 28 bp, Artificial
  Synthetic Nucleic Acid
tggaggaactaaattgtaatataccaac

SEQ ID NO: 221 - DNA, 28 bp, Artificial
  Synthetic Nucleic Acid
cacctagtgaactccataaaaagagaaa

SEQ ID NO: 222 - DNA, 28 bp, Artificial
  Synthetic Nucleic Acid
cagatgcaattattaaatatcagaatgg

SEQ ID NO: 223 - DNA, 28 bp, Artificial
  Synthetic Nucleic Acid
ctcactcaccctgcaaaggaccaaatgt

SEQ ID NO: 224 - DNA, 23 bp, Artificial
  Synthetic Nucleic Acid
agtttcttccaaagcagcctctc

SEQ ID NO: 225 - DNA, 30 bp, Artificial
  Synthetic Nucleic Acid
acatctcaaatccctgtccagatgtgctga

SEQ ID NO: 226 - DNA, 30 bp, Artificial
  Synthetic Nucleic Acid
acatttcaatcaggcgtccagatgtgctga

SEQ ID NO: 227 - DNA, 30 bp, Artificial
  Synthetic Nucleic Acid
tggagaattgcaaaagtccagatgtgctga

SEQ ID NO: 228 - DNA, 30 bp, Artificial
  Synthetic Nucleic Acid
gtccagatgtgctgaacatctcaaatccct

SEQ ID NO: 229 - DNA, 30 bp, Artificial
  Synthetic Nucleic Acid
gtccagatgtgctgaacatttcaatcaggc

SEQ ID NO: 230 - DNA, 30 bp, Artificial
  Synthetic Nucleic Acid
gtccagatgtgctgatggagaattgcaaaa

SEQ ID NO: 231 - DNA, 40 bp, Artificial
  Synthetic Nucleic Acid
gttaaccatacataatttaaacagtttgccgctgcccaat

SEQ ID NO: 232 - DNA, 40 bp, Artificial
  Synthetic Nucleic Acid
tggcaatttaaaaataagcaacagtttgccgctgcccaat

SEQ ID NO: 233 - DNA, 420 bp, Homo Sapiens
aatacaaatggtatcttaaggtaagtctttgatttgttttttcgaaattgtatttatcttcagcacatctggactctttaacttcttaaagatcaggttctgaagggtgatggaaattacttttgactgttgttgtcatcattatattactagaaagaaaattatcataatgataatattagagcacggtgctatggactttttgtgtcaggatgagagagtttgcctggacggagctggtttatctgataaactgcaaaatataattgaatctgtgacagagggaagcatcgtaacagcaaggtgttttgtggctttggggcagtgtgtatttcggctttatgttggaacctttccagaaggagaacttgtggcatacttagctaaaatgaagttgctagaaatatccatcatgataaa

SEQ ID NO: 234 - DNA, 650 bp, Homo Sapiens
tcttgatgggatgctcctgaaagcaattaattctcagttttttgtggcttctaatgcaaaatacattgacgcagacagaatttgaaatgaattttcttctaatatagcaattaattttatttaaatatctctagagtttttttttaatactgtgactaacctatgtttgttctttttcacctctcgtatccacgatcactaagaaacccaaatactttgttcatgtttaaattttacaacatttcatagactattaaacatggaacatccttgtggggacaagaaatcgaatttgctcttgaaaaggtttccaactaattgatttgtaggacattataacatcctctagctgacaagcttacaaaaataaaaactggagctaaccgagagggtgcttttttccctgacacataaaaggtgtctttctgtcttgtatcctttggatatgggcatgtcagtttcatagggaaattttcacatggagcttttgtatttctttctttgccagtacaactgcatgtggtagcacactgtttaatcttttctcaaataaaaagacatggggcttcatttttgttttgcctttttggtatcttacaggaactccaggatggcattgggcagcggcaaactgttgtcagaacattgaa

SEQ ID NO: 235 - DNA, 420 bp, Homo Sapiens
ctgtcagacagaaaaaagaggtagggcgacagatctaataggaatgaaaacattttagcagactttttaagctttctttagaagaatatttcatgagagattataagcagggtgaaaggcactaacattaaagaacctatcaaccattaatcaacagcagtaaagaaattttttatttctttttttcatatactaaaatatatacttgtggctagttagtggttttctgctattttaaacttgaagtttgctttaaaaatcacccatgattgcttaaaggtgaatatcttcaatatattttaacttcaacaagctgaatctcagttgtttttcaagaagattttagaaagcaattataaatgattgttttgtaggaaagacagatctttgcttagttttaaaaatagctatgaatatgac

SEQ ID NO: 236 - DNA, 450 bp, Homo Sapiens
attacaggcgcctgccaccaaacctggcaaatttttgtatttttagtgtagacggggtttcaccatatttgccaggctggtcgcaaactcctgacctcaagtgatccgcccacatcggcctccctaagcgctagggttacaggcatgagccactgcgcctggccaggaatttttgaatcagaatttttcttgttcgattttaatctcttatcatttagagattcttgaaatattgaaattactttgttcaaagtgaatgaattttcttaaattatgtatggttaacatcttttaaattgcttatttttaaattgccatgtttgtgtcccagtttgcattaacaaatagtttgagaactatgttggaaaaaaaaataacaattttattcttctttctccaggctagaagaacaaaagaatatcttgtcagaatttcaaagagatttaaatg

SEQ ID NO: 237 - DNA, 420 bp, Homo Sapiens
aaaagcttgagcaagtcaaggtaattttattttctcaaatcccccagggcctgcttgcataaagaagtatatgaatctattttttaattcaatcattggttttctgcccattaggttattcatagttccttgctaaagtgtttttctcacaactttatttcttcttaaccctgcagttctgaaccagtgcacataagaacatatgtatatatgtgtgtgtgtgtatttatatatacacacacacatattgcatctatacatctacacatatagatgtatagattcaatatgtctaaatgtatataattcacagtttttatctttgatttgaaattaattttagattttacttgagaacttcacaacttcatataattttaaaaactgaagaccagattgtggaatcataaaatctaaatc

SEQ ID NO: 238 - DNA, 450 bp, Homo Sapiens
ataatgcaatttctcagcagcaagctacggtatgctatggcatgctatgatacccaagaggctgatgaatttgttcacattgttctatttctgatagagagataggttttcagacactaactttatttggagtgttgctttaccatctcacatttttctcttaaaaaatttatgagggataatataatcgtttattttctacagagatttatctactgagggggtgagtgtttcagtcaatcagctctgtgctcagatagaaaactgttggtatttgaggtaccactgggccctcggtcaagtcgcttcattttgatagactaatcaatagaagcaaagacaaggtagttggaattgtgctgtaattcattttaaacgttgttgcatttgtctgtttcagttactggtggaagagttgcccctgcgccagggaattctcaaacaattaaa

SEQ ID NO: 239 - DNA, 420 bp, Homo Sapiens
caaatctccagtggataaaggttagacattaaccatctcttccgtcacatgtgttaaatgttgcaagtatttgtatgtattttgtttcctgggtgcttcattggtcggggaggaggctggtatgtggattgttgttttgttttgtttttttaacctgaccgtttgctttggctatatgttttgttgtggctagaaaaaatgatgatggtgaatggctttacattaatgaccaaatgccaaaatttataccacaattttttgcataaattattctgaagaatcagactgaagaaatggcgaagtatttaattcagtggccaggcatgtactgacagtatttaagctgaaaggacgtggtctggttctagttaaacaagtgtcataaatcaaaattaattattcacacctgtggtatggact

SEQ ID NO: 240 - DNA, 450 bp, Homo Sapiens
tattcatttttataactgcaaaggaagcgcgtatggcatataatacacaacacaccagtatatttagtaactgagtgaataaatgaaagatgtatttctttactttatcagttgcagttggctatgcctttgtgtaaggtgtgtgttttgaaattccaaaaaggtattagtttctttaaagcaaagaatttttgtagcaggttaatgaataattttgaatacattggttaaatcccaacatgtaatatatgtaaataatcaatattatgctgctaaaataacacaaatcagtaagattctgtaatatttcatgataaataacttttgaaaatatatttttaaacattttggcttatgccttgagaattatttacctttttaaaatgtattttcctttcaggtttccagagctttacctgagaaacaaggagaaattgaagctcaaataaa

SEQ ID NO: 241 - DNA, 420 bp, Homo Sapiens
aaggaccatttgacgttcaggtagggaactttttgctttaaatatttttgtcttttttaagaaaaatggcaatatcactgaattttctcatttggtatcattattaaagacaaaatattacttgttaaagtgtggtaaggaagactttattcaggataaccacaataggcacagggaccactgcaatggagtattacaggaggttggatagagagagattgggctcaactctaaatacagcacagtggaagtaggaatttatagccaaggagcagtgtaggagtcagtagatggaaaattattaagaggaaacatcaggggtaagtgggattctggctaaaccaacctcacaggattcttgctgaagataggccagggttatcttatcagacaacccttggggaatggtggagaatactg

SEQ ID NO: 242 - DNA, 450 bp, Homo Sapiens
tactaaacacagaattttgtaaaacaataagtgtataaagtaaaatgaacattaggattattgagattattgtagctaaaactagtgtttattcatataaattatgttaataaattgtattgtcattattgcattttacttttttgaaaagtagttaatgcctgtgtttctatatgagtattatataattcaagaagatattggatgaattttttttaagtttaatgtgtttcacatctctgtttcttttctctgcaccaaaagctacatttttgtgcccttatgtaccaggcagaaattgatctgcaatacatgtggagtctccaagggtatatttaaatttagtaattttattgctaactgtgaagttaatctgcactatatgggttcttttccccaggaaactgaaatagcagttcaagctaaacaaccggatgtggaagagatttt

SEQ ID NO: 243 - DNA, 420 bp, Homo Sapiens
cagccactcagccagtgaaggtaatgaagcaacctctagcaatatccattacctcataatgggttatgcttcccctgttgtacatttgccattgacgtggactatttataatcagtgaaataacttgtaaggaaatactggccatactgtaatagcagaggcaaagctgtctttttgatcagcatatcctatttatatattgtgatcttaaggctattaacgagtcattgctttaaaggactcatttctgtcctggtgtgctgccatcaatacaaaagtagtcccaccttcaaggtagattaaattctttggggctttattgctttgcttgccagccttgatgcttttcatattgtttggtttaattcaaatcaagctactgcatcatagtgtctgtctccaacagctgtaaagaatcac

SEQ ID NO: 244 - DNA, 450 bp, Homo Sapiens
atataattgactgggggtgagccagtacattaggattttcctaaagttatctggataattttagtatgcaaccacaatagatactcttcaagaattaagctagttgctgagagggaactgttttttgttggtttgttttcactaatgtttgcactctacttcctttaaataaaattatgcctggagaaagggtttttgtatggagcaattgataaatatttgtagggtggttggctaaaataattataattcctttaaaagaaattctacccactaaagttaatttagaagtaaaatataatagaaatccaataatatattcaccaaatggattaagatgttcatgaattatcttcaaagtgttaatcgaataagtaatgtgtatgcttttctgttaaagaggaagttagaagatctgagctctgagtggaaggcggtaaaccgtttact

SEQ ID NO: 245 - DNA, 420 bp, Homo Sapiens
actgaccactattggagcctgtaagtatactggatcccattctctttggctctagctatttgttcaaaagtgcaactatgaagtgatgactgggtgagagagaaaatttgtttcaattctaaagatagagataaacctttgtgttattgactgtgcaaaaagtcttagagtacattccttggaaattgactctgattcaaagtgttgcatgacaacgggatatggggagtgttctctggagatacacccacaaggaagagaagagcacaagggagattgtgggagagtctgaaatgtgatttgtctgcagcagaggcctaagccagtctcgcaggagccctacatctgggctggctgtgcagagctgtcctgaattgcaggcagtgggcctggcccttgtattcctgatccagccagcca

SEQ ID NO: 246 - DNA, 450 bp, Homo Sapiens
tcttgaataaaaaaaaaataagtaaaatttatttccctggcaaggtctgaaaacttttgttttctttaccacttccacaatgtatatgattgttactgagaaggcttatttaacttaagttacttgtccaggcatgagaatgagcaaaatcgttttttaaaaaattgttaaatgtatattaatgaaaaggttgaatcttttcattttctaccatgtattgctaaacaaagtatccacattgttagaaaaagatatataatgtcatgaataagagtttggctcaaattgttactcttcaattaaatttgacttattgttattgaaattggctctttagcttgtgtttctaatttttctttttcttcttttttcctttttgcaaaaacccaaaatattttagctcctactcagactgttactctggtgacacaacctgtggttactaaggaa

SEQ ID NO: 247 - DNA, 420 bp, Homo Sapiens
agatgatcatcaagcagaaggtatgagaaaaaatgataaaagttggcagaagtttttctttaaaatgaagattttccaccaatcactttactctcctagaccatttcccaccagttcttaggcaactgtttctctctcagcaaacacattactctcactattcagcctaagtataatcaaggatataaattaatgcaaataacaaaagtagccatacattaaaaaggaaatatacaaaaaaaaaaaaaaaaaaaagcagaaaccttacaagaatagttgtctcagttaaatttactaaacaacctggtattttaaaaatctattttataccaaataagtcactcaactgagctatttacatttaaactgtttgttttggcactacgcagcccaacatattgcagaatcaaatataatagt

SEQ ID NO: 248 - DNA, 450 bp, Homo Sapiens
tttaaaataaatataccttaattttgacgtcacacagaatgatattataagtataaatagttatctatcttttaaatacattgtcgtaattcagaataacatttcttactcaaggcattcagacagtggtttaagtaatccgaggtactccggaatgtctccatttgagcctttaaatgaagaaaatctatagtcaagattttcatttgaaatatttttgatatctaagaatgaaacatatttcctgttaaattgttttctataaacccttatacagtaacatcttttttatttctaaaagtgttttggctggtctcacaattgtactttactttgtattatgtaaaaggaatacacaacgctgaagaaccctgatactaagggatatttgttcttacaggcaacaatgcaggatttggaacagaggcgtccccagttggaagaactcat

SEQ ID NO: 249 - DNA, 420 bp, Homo Sapiens
aacaatcattacggatcgaagtaagttttttaacaagcatgggacacacaaagcaagatgcatgacaagtttcaataaaaacttaagttcatatatccccctcacatttataaaaataatgtgaaataattgtaaatgataacaattgtgctgagattttcagtccataatgttaccttttaataaatgaatgtaattccattgaatagaagaaatacatttttaaatcaattcagggcttatatagttgcaaagcatgcattgatgggtgtggtgaccacagtgtggcagaacatttgtggcagaacatttgttctttagttgtcatctgggctggcatccatggagatgccagtctctccctcatatccttggctgttggtccaagcaggcagtggcttcttcctgggccatctttca

SEQ ID NO: 250 - DNA, 450 bp, Homo Sapiens
caaactcctgtggcaacagaaagccttcaggcaatgaaatgctggcactgggaaatcaggctgatgggtgctgaagtggcaaggatgaggggatatggatattctgctgtagtgcttttctaacagatgattcatatttggttctagggatcaagaattgagttaaaattttatatatatgttgatgttctatgtcaccttcaggaaaataatttaacagaaactaatatttgccatcaaaaaagcaaagaatcctgttgttcatcatcctagccataacacaatgaataattttttaaataagcaacataaatgtgagataacgtttggaagttacatttaaaatgtctcctccagactagcatttactactatatatttatttttccttttattctagttgaaagaattcagaatcagtgggatgaagtacaagaacaccttcagaac

SEQ ID NO: 251 - DNA, 420 bp, Homo Sapiens
agaaaatcacagaaaccaaggttagtatcaaagatacctttttaaaataaaatactggttacatttgataaaattataccatagattgtaatttaatgatgtttaatgtaaagttattaacagaaaatcacgttaaagctgaaatgaacagtagactttgtatatttattttcttagagacagagtctcactgtcacccaggctaaagtgcagtggcacaatcatagctcactgagccttgaactctggggctcaagcagtcctcctgcctcagcctccctagtagctgggactactagccaggcgtgtaccaccacgcctggctaattttttaaaaatttttgttttctgtagagatgggttcttgaactcttggcctcaagcaattctccttccttggcctcccaaagcactaggatt

SEQ ID NO: 252 - DNA, 450 bp, Homo Sapiens
caacaagttaatgaagagggaaagaaatgtatgaggtttttttcgttcaaatgttgttatatgtcacatattcaacaattatatatgagcttatttttgtagtttttttctcttgtgataaaaacaattaagcccactttattgccaattaattgctactaagttgaaatacttgatactggttattgctcaagatgctgcatttgaaaagtttgtcctgaaaggtgggttaccttatactgtcatgattgactaaatcatatggtaggttaaaagcaatctaatatatgtattctgacctgaggattcagaagctgtttacgaagtattttaagacactccaactagagatttcataaaaaaaactgacattcattctctttctcataaaaatctatagcagttggccaaagacctccgccagtggcagacaaatgtagatgtggcaaa

SEQ ID NO: 253 - DNA, 420 bp, Homo Sapiens
tggagaagcattcataaaaggtatgaattacattatttctaaaactactgttggctgtaataatggggtggtgaaactggatggaccatgaggatttgtttttccaatccagctaaactggagcttgggagggttcaagacgataaataccaactaaactcacggacttggctcagacttctattttaaaaacgaggaacataagatctcatttgcccgctgtcacaaaagtagtgacataaccaagagattaaacaaaaagcaaaatactgatttatagctagaagagccatttatcagtctactttgataactctatccaaaggaatatctttctatctcatcatggcgcacactgccttacctgttatctgataaataagtcactttgggattcatgatagagttatagctgtacat

SEQ ID NO: 254 - DNA, 450 bp, Homo Sapiens
tctcaaatttggcagtatattaaaaataagctttcaaaattgaccaacaaaaactacaaaattgaaaaaaaggtactttgaactttcacatgttcaaatatatgtatatatatttcacatatatatatgaaacctcctctgtggagaggggtttatagaaatctgtaattgtcattcttgcatgccttcccccatacaaacgcctttaagttaaataaaaatgaaagtaaatagactgcacaatattatagttgttgcttaaaggaagagctgtagcaacaactcaccccattgttggtatattacaatttagttcctccatctttctctttttatggagttcactaggtgcaccattctgatatttaataattgcatctgaacatttggtcctttgcagggtgagtgagcgagaggctgctttggaagaaactcatagattactgcaac

SEQ ID NO: 255 - DNA, 420 bp, Homo Sapiens
agctgatgaaacaatggcaagtaagtcaggcatttccgctttagcactcttgtggatccaattgaacaattctcagcatttgtacttgtaactgacaagccagggacaaaacaaaatagttgcttttatacagcctgatgtatttcggtatttggacaaggaggagagaggcagagggagaaggaaacatcatttataattccacttaacaccctcgtcttagaaaaagtacatgctctgaccaggaaaacatttgcatataaaaccagagcttcggtcaaggagaaactttgctcagagaaataacttagggattggtttattaaattttaaaagttgacatttttgagtgtttatttaatattttacagggaaagcatctgtatgaattgtctgttttatttagcgttgctaactgaa

SEQ ID NO: 256 - DNA, 450 bp, Homo Sapiens
acttgatccatatagtaatgaaattattggcactggggtacactttatcatagaattttattgcctatcacttccataaaataatacattttgtccatagactagaagatataacttgtgaactttataaagttataaatacattactttccaactcataatggcaaggaataaatctattacaactaataagatgcccattttaaatctacataataacaggagaaggcaatacgccaagaaaagggatttgagatgtatcttcttgttagtttagcctgattgaaatgtcttttgaactaataattatttatattttgcaattctccaaattcacattcatcgcttgtttcttttgtttggtaattctgcacatattcttcttcctgctgtcctgtaggacctccaaggtgaaattgaagctcacacagatgtttatcacaacctgga

SEQ ID NO: 257 - DNA, 24 bp, Artificial
  Synthetic Nucleic Acid
gtgtgctaccacatgcagttgtac

SEQ ID NO: 258 - DNA, 24 bp, Artificial
  Synthetic Nucleic Acid
tctgtcgccctacctcttttttct

SEQ ID NO: 259 - DNA, 24 bp, Artificial
  Synthetic Nucleic Acid
attcctattagatctgtcgcccta

SEQ ID NO: 260 - DNA, 24 bp, Artificial
  Synthetic Nucleic Acid
ttttcattcctattagatctgtcg

SEQ ID NO: 261 - DNA, 24 bp, Artificial
  Synthetic Nucleic Acid
aaatgttttcattcctattagatc

SEQ ID NO: 262 - DNA, 24 bp, Artificial
  Synthetic Nucleic Acid
ctaaaatgttttcattcctattag

SEQ ID NO: 263 - DNA, 24 bp, Artificial
  Synthetic Nucleic Acid
agtctgctaaaatgttttcattcc

SEQ ID NO: 264 - DNA, 24 bp, Artificial
  Synthetic Nucleic Acid
agaataaaattgttattttttttt

SEQ ID NO: 265 - DNA, 24 bp, Artificial
  Synthetic Nucleic Acid
gcctggagaaagaagaataaaatt

SEQ ID NO: 266 - DNA, 24 bp, Artificial
  Synthetic Nucleic Acid
tgttcttctagcctggagaaagaa

SEQ ID NO: 267 - DNA, 24 bp, Artificial
  Synthetic Nucleic Acid
gatattcttttgttcttctagcct

SEQ ID NO: 268 - DNA, 24 bp, Artificial
  Synthetic Nucleic Acid
atccatgcattcttccatgcacag

SEQ ID NO: 269 - DNA, 30 bp, Artificial
  Synthetic Nucleic Acid
tttcattcctattagttgccgctgcccaat

SEQ ID NO: 270 - DNA, 30 bp, Artificial
  Synthetic Nucleic Acid
aaatgttttcattccttgccgctgcccaat

SEQ ID NO: 271 - DNA, 30 bp, Artificial
  Synthetic Nucleic Acid
tgttttcattccagtttgccgctgcccaat

SEQ ID NO: 272 - DNA, 30 bp, Artificial
  Synthetic Nucleic Acid
agatctgtcgacagtttgccgctgcccaat

SEQ ID NO: 273 - DNA, 30 bp, Artificial
  Synthetic Nucleic Acid
cattcctattagacagtttgccgctgccca

SEQ ID NO: 274 - DNA, 24 bp, Artificial
  Synthetic Nucleic Acid
tgcattcaatgttctgacaacagt

SEQ ID NO: 275 - DNA, 24 bp, Artificial
  Synthetic Nucleic Acid
gtttgcagacctcctgccaccgca

SEQ ID NO: 276 - DNA, 25 bp, Artificial
  Synthetic Nucleic Acid
atttgacagatctgttgagaaatgg

SEQ ID NO: 277 - DNA, 22 bp, Artificial
  Synthetic Nucleic Acid
ggctgttttcatccaggttgtg

SEQ ID NO: 278 - DNA, 21 bp, Artificial
  Synthetic Nucleic Acid
agttgctgctcttttccaggt

SEQ ID NO: 279 - DNA, 25 bp, Artificial
  Synthetic Nucleic Acid
atttgacagatctgttgagaaatgg

SEQ ID NO: 280 - DNA, 21 bp, Artificial
  Synthetic Nucleic Acid
ggctccaatagtggtcagtcc

SEQ ID NO: 281 - DNA, 20 bp, Artificial
  Synthetic Nucleic Acid
cctgagaattgggaacatgc

SEQ ID NO: 282 - DNA, 20 bp, Artificial
  Synthetic Nucleic Acid
cctccttccatgactcaagc

SEQ ID NO: 283 - DNA, 450 bp, Homo Sapiens
attacaaaaatatggaaatagagagtagcaggaaaaagtatatggcatttctattacctagaaccccactgttgtaggtgtctttccttgacatagctgtgaccatatagctctgttaaattgcatatttgtctaaataattcatatttttttctatccctatcttgcgtttggagctcattttatgattgttattcttttatctttcaataaatactcaaaatgttaaaaaaatatgattcaggaatttttaattgtcttagccacaaatttataggatttccaggatctgtaacaatggataaataattttataaaaatcctaaatttacacagaattataaagatataatcattttaaacagcacctattttgcaaattttataaatctctttctaggactcatctctgcttctacaatcaacccagaatgtgactgtaaatgcgcg

SEQ ID NO: 284 - DNA, 420 bp, Homo Sapiens
cacaggcaggttaaaagtcggtgagtccagcttcatcatggtgctttgcatgcatgttgtccatgaatagtgctaaatgaatgcattgttttttcttctaaagaaatcaaagctacttatgaacaaaatatgaattttctaaatatcatgctgtgttgaccacagactagcaccacagagtggggtggggggtgaggggacagcctgaagtgctttgattagagttatttttgttccaagaaatagaggacaattaaagctagtacaagaaacaaaatgtttattgtgggttaccaaagactcttgcagcaaccaaagggaaggaacccaagtagctggcctcaaagagcacagagaccagtggctgagagccacgaatacattcagttccctccccgttctttcttctcttttctctcc

SEQ ID NO: 285 - DNA, 450 bp, Homo Sapiens
aagtgatctgcccgccgcagcctcccaaagttctgggattacaggcgtgagccactgctcccagcaaatgtgtgtttgctgctctgtttccctgcgtgtttcttgcctgtcttgtgtgtatctttgtgtctggggccatcctgccctgtgctgcccaaccaagccatctctgcccacaggtgcccgcctggctgggtgggtgagcggtgtcagctggaggacccctgtcactcaggcccctgtgctggccgtggtgtctgccagagttcagtggtggctggcaccgcccgattctcatgccggtgcccccgtggcttccgaggtgagaggggaagagtctggaggggaggtagtcgggggtgtggtcagtcctaaactcaccctgtcctggtccctccaggccctgactgctccctgccagatccctgcctcagcagcccttgtgcccac

SEQ ID NO: 286 - DNA, 420 bp, Homo Sapiens
cgactgtgcctgtcttcctggtgagtgagccctactcaggagagtcagaggggtgggcgtggggacagcaggccagcccggcggtgaccatccttgcccccttccctgctagggtttgagggtcagaattgtgaagtgaacgtggacgactgtccaggacaccgatgtctcaatggggggacatgcgtggatggcgtcaacacctataactgccagtgccctcctgagtggacaggtgggcactgcggccagagggagcggggaggcaggcctcgggtggacatgcgccaggtggctggactgctgcatctgtgtgccacaggccagttctgcacggaggacgtggatgagtgtcagctgcagcccaacgcctgccacaatgggggtacctgcttcaacacgctgggtggccacagctgc

SEQ ID NO: 287 - DNA, 30 bp, Artificial
  Synthetic Nucleic Acid
tgctactctctatttccatatttttgtaat

SEQ ID NO: 288 - DNA, 30 bp, Artificial
  Synthetic Nucleic Acid
aaatgccatatactttttcctgctactctc

SEQ ID NO: 289 - DNA, 30 bp, Artificial
  Synthetic Nucleic Acid
agtggggttctaggtaatagaaatgccata

SEQ ID NO: 290 - DNA, 30 bp, Artificial
  Synthetic Nucleic Acid
caaggaaagacacctacaacagtggggttc

SEQ ID NO: 291 - DNA, 30 bp, Artificial
  Synthetic Nucleic Acid
ctatatggtcacagctatgtcaaggaaaga

SEQ ID NO: 292 - DNA, 30 bp, Artificial
  Synthetic Nucleic Acid
aaatatgcaatttaacagagctatatggtc

SEQ ID NO: 293 - DNA, 30 bp, Artificial
  Synthetic Nucleic Acid
aaaatatgaattatttagacaaatatgcaa

SEQ ID NO: 294 - DNA, 30 bp, Artificial
  Synthetic Nucleic Acid
acgcaagatagggatagaaaaaaatatgaa

SEQ ID NO: 295 - DNA, 30 bp, Artificial
  Synthetic Nucleic Acid
aatcataaaatgagctccaaacgcaagata

SEQ ID NO: 296 - DNA, 30 bp, Artificial
  Synthetic Nucleic Acid
ttgaaagataaaagaataacaatcataaaa

SEQ ID NO: 297 - DNA, 30 bp, Artificial
  Synthetic Nucleic Acid
tttaacattttgagtatttattgaaagata

SEQ ID NO: 298 - DNA, 30 bp, Artificial
  Synthetic Nucleic Acid
aaattcctgaatcatatttttttaacattt

SEQ ID NO: 299 - DNA, 30 bp, Artificial
  Synthetic Nucleic Acid
tttgtggctaagacaattaaaaattcctga

SEQ ID NO: 300 - DNA, 30 bp, Artificial
  Synthetic Nucleic Acid
gatcctggaaatcctataaatttgtggcta

SEQ ID NO: 301 - DNA, 30 bp, Artificial
  Synthetic Nucleic Acid
attatttatccattgttacagatcctggaa

SEQ ID NO: 302 - DNA, 30 bp, Artificial
  Synthetic Nucleic Acid
aaatttaggatttttataaaattatttatc

SEQ ID NO: 303 - DNA, 30 bp, Artificial
  Synthetic Nucleic Acid
atatctttataattctgtgtaaatttagga

SEQ ID NO: 304 - DNA, 30 bp, Artificial
  Synthetic Nucleic Acid
aggtgctgtttaaaatgattatatctttat

SEQ ID NO: 305 - DNA, 30 bp, Artificial
  Synthetic Nucleic Acid
atttataaaatttgcaaaataggtgctgtt

SEQ ID NO: 306 - DNA, 30 bp, Artificial
  Synthetic Nucleic Acid
gagatgagtcctagaaagagatttataaaa

SEQ ID NO: 307 - DNA, 30 bp, Artificial
  Synthetic Nucleic Acid
ctgggttgattgtagaagcagagatgagtc

SEQ ID NO: 308 - DNA, 30 bp, Artificial
  Synthetic Nucleic Acid
cgcgcatttacagtcacattctgggttgat

SEQ ID NO: 309 - DNA, 30 bp, Artificial
  Synthetic Nucleic Acid
ctttgggaggctgcggcgggcagatcactt

SEQ ID NO: 310 - DNA, 30 bp, Artificial
  Synthetic Nucleic Acid
tcacgcctgtaatcccagaactttgggagg

SEQ ID NO: 311 - DNA, 30 bp, Artificial
  Synthetic Nucleic Acid
catttgctgggagcagtggctcacgcctgt

SEQ ID NO: 312 - DNA, 30 bp, Artificial
  Synthetic Nucleic Acid
gaaacagagcagcaaacacacatttgctgg

SEQ ID NO: 313 - DNA, 30 bp, Artificial
  Synthetic Nucleic Acid
acaggcaagaaacacgcagggaaacagagc

SEQ ID NO: 314 - DNA, 30 bp, Artificial
  Synthetic Nucleic Acid
gacacaaagatacacacaagacaggcaaga

SEQ ID NO: 315 - DNA, 30 bp, Artificial
  Synthetic Nucleic Acid
cacagggcaggatggccccagacacaaaga

SEQ ID NO: 316 - DNA, 30 bp, Artificial
  Synthetic Nucleic Acid
gagatggcttggttgggcagcacagggcag

SEQ ID NO: 317 - DNA, 30 bp, Artificial
  Synthetic Nucleic Acid
caggcgggcacctgtgggcagagatggctt

SEQ ID NO: 318 - DNA, 30 bp, Artificial
  Synthetic Nucleic Acid
acaccgctcacccacccagccaggcgggca

SEQ ID NO: 319 - DNA, 30 bp, Artificial
  Synthetic Nucleic Acid
tgacaggggtcctccagctgacaccgctca

SEQ ID NO: 320 - DNA, 30 bp, Artificial
  Synthetic Nucleic Acid
ggccagcacaggggcctgagtgacaggggt

SEQ ID NO: 321 - DNA, 30 bp, Artificial
  Synthetic Nucleic Acid
tgaactctggcagacaccacggccagcaca

SEQ ID NO: 322 - DNA, 30 bp, Artificial
  Synthetic Nucleic Acid
cgggcggtgccagccaccactgaactctgg

SEQ ID NO: 323 - DNA, 30 bp, Artificial
  Synthetic Nucleic Acid
gggggcaccggcatgagaatcgggcggtgc

SEQ ID NO: 324 - DNA, 30 bp, Artificial
  Synthetic Nucleic Acid
cctctcacctcggaagccacgggggcaccg

SEQ ID NO: 325 - DNA, 30 bp, Artificial
  Synthetic Nucleic Acid
cctcccctccagactcttcccctctcacct

SEQ ID NO: 326 - DNA, 30 bp, Artificial
  Synthetic Nucleic Acid
actgaccacacccccgactacctcccctcc

SEQ ID NO: 327 - DNA, 30 bp, Artificial
  Synthetic Nucleic Acid
caggacagggtgagtttaggactgaccaca

SEQ ID NO: 328 - DNA, 30 bp, Artificial
  Synthetic Nucleic Acid
cagtcagggcctggagggaccaggacaggg

SEQ ID NO: 329 - DNA, 30 bp, Artificial
  Synthetic Nucleic Acid
ggcagggatctggcagggagcagtcagggc

SEQ ID NO: 330 - DNA, 30 bp, Artificial
  Synthetic Nucleic Acid
gtgggcacaagggctgctgaggcagggatc

SEQ ID NO: 331 - DNA, 30 bp, Artificial
  Synthetic Nucleic Acid
tgtggtgctagtctgtggtcaacacagcat

SEQ ID NO: 332 - DNA, 30 bp, Artificial
  Synthetic Nucleic Acid
caccccactctgtggtgctagtctgtggtc

SEQ ID NO: 333 - DNA, 30 bp, Artificial
  Synthetic Nucleic Acid
cctcaccccccaccccactctgtggtgcta

SEQ ID NO: 334 - DNA, 30 bp, Artificial
  Synthetic Nucleic Acid
caggctgtcccctcaccccccaccccactc

SEQ ID NO: 335 - DNA, 30 bp, Artificial
  Synthetic Nucleic Acid
caaagcacttcaggctgtcccctcaccccc

SEQ ID NO: 336 - DNA, 30 bp, Artificial
  Synthetic Nucleic Acid
agggaagggggcaaggatggtcaccgccgg

SEQ ID NO: 337 - DNA, 30 bp, Artificial
  Synthetic Nucleic Acid
caaaccctagcagggaagggggcaaggatg

SEQ ID NO: 338 - DNA, 30 bp, Artificial
  Synthetic Nucleic Acid
tctgaccctcaaaccctagcagggaagggg

SEQ ID NO: 339 - DNA, 30 bp, Artificial
  Synthetic Nucleic Acid
acttcacaattctgaccctcaaaccctagc

SEQ ID NO: 340 - DNA, 30 bp, Artificial
  Synthetic Nucleic Acid
gtccacgttcacttcacaattctgaccctc

SEQ ID NO: 341 - DNA, 619 bp, Homo Sapiens
tcttgatgggatgctcctgaaagcaattaattctcagttttttgtggcttctaatgcaaaatacattgacgcagacagaatttgaaatgaattttcttctaatatagcaattaattttatttaaatatctctagagtttttttttaatactgtgactaacctatgtttgttctttttcacctctcgtatccacgatcactaagaaacccaaatactttgttcatgtttaaattttacaacatttcatagactattaaacatggaacatccttgtggggacaagaaatcgaatttgctcttgaaaaggtttccaactaattgatttgtaggacattataacatcctctagctgacaagcttacaaaaataaaaactggagctaaccgagagggtgcttttttccctgacacataaaaggtgtctttctgtcttgtatcctttggatatgggcatgtcagtttcatagggaaattttcacatggagcttttgtatttctttctttgccagtacaactgcatgtggtagcacactgtttaatcttttctcaaataaaaagacatggggcttcatttttgttttgcctttttggtatcttacaggaactccaggatggcattg

SEQ ID NO: 342 - DNA, 425 bp, Homo Sapiens
tcttgaataaaaaaaaaataagtaaaatttatttccctggcaaggtctgaaaacttttgttttctttaccacttccacaatgtatatgattgttactgagaaggcttatttaacttaagttacttgtccaggcatgagaatgagcaaaatcgttttttaaaaaattgttaaatgtatattaatgaaaaggttgaatcttttcattttctaccatgtattgctaaacaaagtatccacattgttagaaaaagatatataatgtcatgaataagagtttggctcaaattgttactcttcaattaaatttgacttattgttattgaaattggctctttagcttgtgtttctaatttttctttttcttcttttttcctttttgcaaaaacccaaaatattttagctcctactcagactgttactctggt

SEQ ID NO: 343 - DNA, 424 bp, Homo Sapiens
tttaaaataaatataccttaattttgacgtcacacagaatgatattataagtataaatagttatctatcttttaaatacattgtcgtaattcagaataacatttcttactcaaggcattcagacagtggtttaagtaatccgaggtactccggaatgtctccatttgagcctttaaatgaagaaaatctatagtcaagattttcatttgaaatatttttgatatctaagaatgaaacatatttcctgttaaattgttttctataaacccttatacagtaacatcttttttatttctaaaagtgttttggctggtctcacaattgtactttactttgtattatgtaaaaggaatacacaacgctgaagaaccctgatactaagggatatttgttcttacaggcaacaatgcaggatttggaacag

SEQ ID NO: 344 - DNA, 434 bp, Homo Sapiens
caaactcctgtggcaacagaaagccttcaggcaatgaaatgctggcactgggaaatcaggctgatgggtgctgaagtggcaaggatgaggggatatggatattctgctgtagtgcttttctaacagatgattcatatttggttctagggatcaagaattgagttaaaattttatatatatgttgatgttctatgtcaccttcaggaaaataatttaacagaaactaatatttgccatcaaaaaagcaaagaatcctgttgttcatcatcctagccataacacaatgaataattttttaaataagcaacataaatgtgagataacgtttggaagttacatttaaaatgtctcctccagactagcatttactactatatatttatttttccttttattctagttgaaagaattcagaatcagtgggatgaagtaca

SEQ ID NO: 345 - DNA, 425 bp, Homo Sapiens
tctcaaatttggcagtatattaaaaataagctttcaaaattgaccaacaaaaactacaaaattgaaaaaaaggtactttgaactttcacatgttcaaatatatgtatatatatttcacatatatatatgaaacctcctctgtggagaggggtttatagaaatctgtaattgtcattcttgcatgccttcccccatacaaacgcctttaagttaaataaaaatgaaagtaaatagactgcacaatattatagttgttgcttaaaggaagagctgtagcaacaactcaccccattgttggtatattacaatttagttcctccatctttctctttttatggagttcactaggtgcaccattctgatatttaataattgcatctgaacatttggtcctttgcagggtgagtgagcgagaggctgctttg

SEQ ID NO: 346 - DNA, 400 bp, Homo Sapiens
gtaagtctttgatttgttttttcgaaattgtatttatcttcagcacatctggactctttaacttcttaaagatcaggttctgaagggtgatggaaattacttttgactgttgttgtcatcattatattactagaaagaaaattatcataatgataatattagagcacggtgctatggactttttgtgtcaggatgagagagtttgcctggacggagctggtttatctgataaactgcaaaatataattgaatctgtgacagagggaagcatcgtaacagcaaggtgttttgtggctttggggcagtgtgtatttcggctttatgttggaacctttccagaaggagaacttgtggcatacttagctaaaatgaagttgctagaaatatccatcatgataaa

SEQ ID NO: 347 - DNA, 600 bp, Homo Sapiens
tcttgatgggatgctcctgaaagcaattaattctcagttttttgtggcttctaatgcaaaatacattgacgcagacagaatttgaaatgaattttcttctaatatagcaattaattttatttaaatatctctagagtttttttttaatactgtgactaacctatgtttgttctttttcacctctcgtatccacgatcactaagaaacccaaatactttgttcatgtttaaattttacaacatttcatagactattaaacatggaacatccttgtggggacaagaaatcgaatttgctcttgaaaaggtttccaactaattgatttgtaggacattataacatcctctagctgacaagcttacaaaaataaaaactggagctaaccgagagggtgcttttttccctgacacataaaaggtgtctttctgtcttgtatcctttggatatgggcatgtcagtttcatagggaaattttcacatggagcttttgtatttctttctttgccagtacaactgcatgtggtagcacactgtttaatcttttctcaaataaaaagacatggggcttcatttttgttttgcctttttggtatcttacag

SEQ ID NO: 348 - DNA, 400 bp, Homo Sapiens
gtagggcgacagatctaataggaatgaaaacattttagcagactttttaagctttctttagaagaatatttcatgagagattataagcagggtgaaaggcactaacattaaagaacctatcaaccattaatcaacagcagtaaagaaattttttatttctttttttcatatactaaaatatatacttgtggctagttagtggttttctgctattttaaacttgaagtttgctttaaaaatcacccatgattgcttaaaggtgaatatcttcaatatattttaacttcaacaagctgaatctcagttgtttttcaagaagattttagaaagcaattataaatgattgttttgtaggaaagacagatctttgcttagttttaaaaatagctatgaatatgac

SEQ ID NO: 349 - DNA, 400 bp, Homo Sapiens
attacaggcgcctgccaccaaacctggcaaatttttgtatttttagtgtagacggggtttcaccatatttgccaggctggtcgcaaactcctgacctcaagtgatccgcccacatcggcctccctaagcgctagggttacaggcatgagccactgcgcctggccaggaatttttgaatcagaatttttcttgttcgattttaatctcttatcatttagagattcttgaaatattgaaattactttgttcaaagtgaatgaattttcttaaattatgtatggttaacatcttttaaattgcttatttttaaattgccatgtttgtgtcccagtttgcattaacaaatagtttgagaactatgttggaaaaaaaaataacaattttattcttctttctccag

SEQ ID NO: 350 - DNA, 400 bp, Homo Sapiens
gtaattttattttctcaaatcccccagggcctgcttgcataaagaagtatatgaatctattttttaattcaatcattggttttctgcccattaggttattcatagttccttgctaaagtgtttttctcacaactttatttcttcttaaccctgcagttctgaaccagtgcacataagaacatatgtatatatgtgtgtgtgtgtatttatatatacacacacacatattgcatctatacatctacacatatagatgtatagattcaatatgtctaaatgtatataattcacagtttttatctttgatttgaaattaattttagattttacttgagaacttcacaacttcatataattttaaaaactgaagaccagattgtggaatcataaaatctaaatc

SEQ ID NO: 351 - DNA, 400 bp, Homo Sapiens
ataatgcaatttctcagcagcaagctacggtatgctatggcatgctatgatacccaagaggctgatgaatttgttcacattgttctatttctgatagagagataggttttcagacactaactttatttggagtgttgctttaccatctcacatttttctcttaaaaaatttatgagggataatataatcgtttattttctacagagatttatctactgagggggtgagtgtttcagtcaatcagctctgtgctcagatagaaaactgttggtatttgaggtaccactgggccctcggtcaagtcgcttcattttgatagactaatcaatagaagcaaagacaaggtagttggaattgtgctgtaattcattttaaacgttgttgcatttgtctgtttcag

SEQ ID NO: 352 - DNA, 400 bp, Homo Sapiens
gttagacattaaccatctcttccgtcacatgtgttaaatgttgcaagtatttgtatgtattttgtttcctgggtgcttcattggtcggggaggaggctggtatgtggattgttgttttgttttgtttttttaacctgaccgtttgctttggctatatgttttgttgtggctagaaaaaatgatgatggtgaatggctttacattaatgaccaaatgccaaaatttataccacaattttttgcataaattattctgaagaatcagactgaagaaatggcgaagtatttaattcagtggccaggcatgtactgacagtatttaagctgaaaggacgtggtctggttctagttaaacaagtgtcataaatcaaaattaattattcacacctgtggtatggact

SEQ ID NO: 353 - DNA, 400 bp, Homo Sapiens
tattcatttttataactgcaaaggaagcgcgtatggcatataatacacaacacaccagtatatttagtaactgagtgaataaatgaaagatgtatttctttactttatcagttgcagttggctatgcctttgtgtaaggtgtgtgttttgaaattccaaaaaggtattagtttctttaaagcaaagaatttttgtagcaggttaatgaataattttgaatacattggttaaatcccaacatgtaatatatgtaaataatcaatattatgctgctaaaataacacaaatcagtaagattctgtaatatttcatgataaataacttttgaaaatatatttttaaacattttggcttatgccttgagaattatttacctttttaaaatgtattttcctttcag

SEQ ID NO: 354 - DNA, 400 bp, Homo Sapiens
gtagggaactttttgctttaaatatttttgtcttttttaagaaaaatggcaatatcactgaattttctcatttggtatcattattaaagacaaaatattacttgttaaagtgtggtaaggaagactttattcaggataaccacaataggcacagggaccactgcaatggagtattacaggaggttggatagagagagattgggctcaactctaaatacagcacagtggaagtaggaatttatagccaaggagcagtgtaggagtcagtagatggaaaattattaagaggaaacatcaggggtaagtgggattctggctaaaccaacctcacaggattcttgctgaagataggccagggttatcttatcagacaacccttggggaatggtggagaatactg

SEQ ID NO: 355 - DNA, 400 bp, Homo Sapiens
tactaaacacagaattttgtaaaacaataagtgtataaagtaaaatgaacattaggattattgagattattgtagctaaaactagtgtttattcatataaattatgttaataaattgtattgtcattattgcattttacttttttgaaaagtagttaatgcctgtgtttctatatgagtattatataattcaagaagatattggatgaattttttttaagtttaatgtgtttcacatctctgtttcttttctctgcaccaaaagctacatttttgtgcccttatgtaccaggcagaaattgatctgcaatacatgtggagtctccaagggtatatttaaatttagtaattttattgctaactgtgaagttaatctgcactatatgggttcttttccccag

SEQ ID NO: 356 - DNA, 400 bp, Homo Sapiens
gtaatgaagcaacctctagcaatatccattacctcataatgggttatgcttcccctgttgtacatttgccattgacgtggactatttataatcagtgaaataacttgtaaggaaatactggccatactgtaatagcagaggcaaagctgtctttttgatcagcatatcctatttatatattgtgatcttaaggctattaacgagtcattgctttaaaggactcatttctgtcctggtgtgctgccatcaatacaaaagtagtcccaccttcaaggtagattaaattctttggggctttattgctttgcttgccagccttgatgcttttcatattgtttggtttaattcaaatcaagctactgcatcatagtgtctgtctccaacagctgtaaagaatcac

SEQ ID NO: 357 - DNA, 400 bp, Homo Sapiens
atataattgactgggggtgagccagtacattaggattttcctaaagttatctggataattttagtatgcaaccacaatagatactcttcaagaattaagctagttgctgagagggaactgttttttgttggtttgttttcactaatgtttgcactctacttcctttaaataaaattatgcctggagaaagggtttttgtatggagcaattgataaatatttgtagggtggttggctaaaataattataattcctttaaaagaaattctacccactaaagttaatttagaagtaaaatataatagaaatccaataatatattcaccaaatggattaagatgttcatgaattatcttcaaagtgttaatcgaataagtaatgtgtatgcttttctgttaaag

SEQ ID NO: 358 - DNA, 400 bp, Homo Sapiens
gtaagtatactggatcccattctctttggctctagctatttgttcaaaagtgcaactatgaagtgatgactgggtgagagagaaaatttgtttcaattctaaagatagagataaacctttgtgttattgactgtgcaaaaagtcttagagtacattccttggaaattgactctgattcaaagtgttgcatgacaacgggatatggggagtgttctctggagatacacccacaaggaagagaagagcacaagggagattgtgggagagtctgaaatgtgatttgtctgcagcagaggcctaagccagtctcgcaggagccctacatctgggctggctgtgcagagctgtcctgaattgcaggcagtgggcctggcccttgtattcctgatccagccagcca

SEQ ID NO: 359 - DNA, 400 bp, Homo Sapiens
tcttgaataaaaaaaaaataagtaaaatttatttccctggcaaggtctgaaaacttttgttttctttaccacttccacaatgtatatgattgttactgagaaggcttatttaacttaagttacttgtccaggcatgagaatgagcaaaatcgttttttaaaaaattgttaaatgtatattaatgaaaaggttgaatcttttcattttctaccatgtattgctaaacaaagtatccacattgttagaaaaagatatataatgtcatgaataagagtttggctcaaattgttactcttcaattaaatttgacttattgttattgaaattggctctttagcttgtgtttctaatttttctttttcttcttttttcctttttgcaaaaacccaaaatattttag

SEQ ID NO: 360 - DNA, 400 bp, Homo Sapiens
gtatgagaaaaaatgataaaagttggcagaagtttttctttaaaatgaagattttccaccaatcactttactctcctagaccatttcccaccagttcttaggcaactgtttctctctcagcaaacacattactctcactattcagcctaagtataatcaaggatataaattaatgcaaataacaaaagtagccatacattaaaaaggaaatatacaaaaaaaaaaaaaaaaaaaagcagaaaccttacaagaatagttgtctcagttaaatttactaaacaacctggtattttaaaaatctattttataccaaataagtcactcaactgagctatttacatttaaactgtttgttttggcactacgcagcccaacatattgcagaatcaaatataatagt

SEQ ID NO: 361 - DNA, 400 bp, Homo Sapiens
tttaaaataaatataccttaattttgacgtcacacagaatgatattataagtataaatagttatctatcttttaaatacattgtcgtaattcagaataacatttcttactcaaggcattcagacagtggtttaagtaatccgaggtactccggaatgtctccatttgagcctttaaatgaagaaaatctatagtcaagattttcatttgaaatatttttgatatctaagaatgaaacatatttcctgttaaattgttttctataaacccttatacagtaacatcttttttatttctaaaagtgttttggctggtctcacaattgtactttactttgtattatgtaaaaggaatacacaacgctgaagaaccctgatactaagggatatttgttcttacag

SEQ ID NO: 362 - DNA, 400 bp, Homo Sapiens
gtaagttttttaacaagcatgggacacacaaagcaagatgcatgacaagtttcaataaaaacttaagttcatatatccccctcacatttataaaaataatgtgaaataattgtaaatgataacaattgtgctgagattttcagtccataatgttaccttttaataaatgaatgtaattccattgaatagaagaaatacatttttaaatcaattcagggcttatatagttgcaaagcatgcattgatgggtgtggtgaccacagtgtggcagaacatttgtggcagaacatttgttctttagttgtcatctgggctggcatccatggagatgccagtctctccctcatatccttggctgttggtccaagcaggcagtggcttcttcctgggccatctttca

SEQ ID NO: 363 - DNA, 400 bp, Homo Sapiens
caaactcctgtggcaacagaaagccttcaggcaatgaaatgctggcactgggaaatcaggctgatgggtgctgaagtggcaaggatgaggggatatggatattctgctgtagtgcttttctaacagatgattcatatttggttctagggatcaagaattgagttaaaattttatatatatgttgatgttctatgtcaccttcaggaaaataatttaacagaaactaatatttgccatcaaaaaagcaaagaatcctgttgttcatcatcctagccataacacaatgaataattttttaaataagcaacataaatgtgagataacgtttggaagttacatttaaaatgtctcctccagactagcatttactactatatatttatttttccttttattctag

SEQ ID NO: 364 - DNA, 400 bp, Homo Sapiens
gttagtatcaaagatacctttttaaaataaaatactggttacatttgataaaattataccatagattgtaatttaatgatgtttaatgtaaagttattaacagaaaatcacgttaaagctgaaatgaacagtagactttgtatatttattttcttagagacagagtctcactgtcacccaggctaaagtgcagtggcacaatcatagctcactgagccttgaactctggggctcaagcagtcctcctgcctcagcctccctagtagctgggactactagccaggcgtgtaccaccacgcctggctaattttttaaaaatttttgttttctgtagagatgggttcttgaactcttggcctcaagcaattctccttccttggcctcccaaagcactaggatt

SEQ ID NO: 365 - DNA, 400 bp, Homo Sapiens
caacaagttaatgaagagggaaagaaatgtatgaggtttttttcgttcaaatgttgttatatgtcacatattcaacaattatatatgagcttatttttgtagtttttttctcttgtgataaaaacaattaagcccactttattgccaattaattgctactaagttgaaatacttgatactggttattgctcaagatgctgcatttgaaaagtttgtcctgaaaggtgggttaccttatactgtcatgattgactaaatcatatggtaggttaaaagcaatctaatatatgtattctgacctgaggattcagaagctgtttacgaagtattttaagacactccaactagagatttcataaaaaaaactgacattcattctctttctcataaaaatctatag

SEQ ID NO: 366 - DNA, 400 bp, Homo Sapiens
gtatgaattacattatttctaaaactactgttggctgtaataatggggtggtgaaactggatggaccatgaggatttgtttttccaatccagctaaactggagcttgggagggttcaagacgataaataccaactaaactcacggacttggctcagacttctattttaaaaacgaggaacataagatctcatttgcccgctgtcacaaaagtagtgacataaccaagagattaaacaaaaagcaaaatactgatttatagctagaagagccatttatcagtctactttgataactctatccaaaggaatatctttctatctcatcatggcgcacactgccttacctgttatctgataaataagtcactttgggattcatgatagagttatagctgtacat

SEQ ID NO: 367 - DNA, 400 bp, Homo Sapiens
tctcaaatttggcagtatattaaaaataagctttcaaaattgaccaacaaaaactacaaaattgaaaaaaaggtactttgaactttcacatgttcaaatatatgtatatatatttcacatatatatatgaaacctcctctgtggagaggggtttatagaaatctgtaattgtcattcttgcatgccttcccccatacaaacgcctttaagttaaataaaaatgaaagtaaatagactgcacaatattatagttgttgcttaaaggaagagctgtagcaacaactcaccccattgttggtatattacaatttagttcctccatctttctctttttatggagttcactaggtgcaccattctgatatttaataattgcatctgaacatttggtcctttgcag

SEQ ID NO: 368 - DNA, 400 bp, Homo Sapiens
gtaagtcaggcatttccgctttagcactcttgtggatccaattgaacaattctcagcatttgtacttgtaactgacaagccagggacaaaacaaaatagttgcttttatacagcctgatgtatttcggtatttggacaaggaggagagaggcagagggagaaggaaacatcatttataattccacttaacaccctcgtcttagaaaaagtacatgctctgaccaggaaaacatttgcatataaaaccagagcttcggtcaaggagaaactttgctcagagaaataacttagggattggtttattaaattttaaaagttgacatttttgagtgtttatttaatattttacagggaaagcatctgtatgaattgtctgttttatttagcgttgctaactgaa

SEQ ID NO: 369 - DNA, 400 bp, Homo Sapiens
acttgatccatatagtaatgaaattattggcactggggtacactttatcatagaattttattgcctatcacttccataaaataatacattttgtccatagactagaagatataacttgtgaactttataaagttataaatacattactttccaactcataatggcaaggaataaatctattacaactaataagatgcccattttaaatctacataataacaggagaaggcaatacgccaagaaaagggatttgagatgtatcttcttgttagtttagcctgattgaaatgtcttttgaactaataattatttatattttgcaattctccaaattcacattcatcgcttgtttcttttgtttggtaattctgcacatattcttcttcctgctgtcctgtag

SEQ ID NO: 370 - RNA, 5 nt, Homo Sapiens
uagga

SEQ ID NO: 371 - RNA, 6 nt, Homo Sapiens
uaggau

SEQ ID NO: 372 - RNA, 6 nt, Homo Sapiens
uaggaa

SEQ ID NO: 373 - RNA, 6 nt, Homo Sapiens
uaggca

SEQ ID NO: 374 - RNA, 6 nt, Homo Sapiens
uaggcu

SEQ ID NO: 375 - RNA, 6 nt, Homo Sapiens
uaggga

SEQ ID NO: 376 - RNA, 6 nt, Homo Sapiens
uagggc

SEQ ID NO: 377 - RNA, 6 nt, Homo Sapiens
uagggu

SEQ ID NO: 378 - RNA, 8 nt, Homo Sapiens
gguagggc

SEQ ID NO: 379 - RNA, 5 nt, Homo Sapiens
agaac

SEQ ID NO: 380 - RNA, 6 nt, Homo Sapiens
agcaug

SEQ ID NO: 381 - RNA, 6 nt, Homo Sapiens
ugacug

SEQ ID NO: 382 - RNA, 6 nt, Homo Sapiens
ugcaug

SEQ ID NO: 383 - RNA, 5 nt, Homo Sapiens
  misc_feature (3)..(3): n is a, c, g, or u
yunay

SEQ ID NO: 384 - RNA, 2 nt, Homo Sapiens
gu

SEQ ID NO: 385 - DNA, 437 bp, Homo Sapiens
ataatgcaatttctcagcagcaagctacggtatgctatggcatgctatgatacccaagaggctgatgaatttgttcacattgttctatttctgatagagagataggttttcagacactaactttatttggagtgttgctttaccatctcacatttttctcttaaaaaatttatgagggataatataatcgtttattttctacagagatttatctactgagggggtgagtgtttcagtcaatcagctctgtgctcagatagaaaactgttggtatttgaggtaccactgggccctcggtcaagtcgcttcattttgatagactaatcaatagaagcaaagacaaggtagttggaattgtgctgtaattcattttaaacgttgttgcatttgtctgtttcagttactggtggaagagttgcccctgcgccagggaattc

SEQ ID NO: 386 - DNA, 419 bp, Homo Sapiens
tattcatttttataactgcaaaggaagcgcgtatggcatataatacacaacacaccagtatatttagtaactgagtgaataaatgaaagatgtatttctttactttatcagttgcagttggctatgcctttgtgtaaggtgtgtgttttgaaattccaaaaaggtattagtttctttaaagcaaagaatttttgtagcaggttaatgaataattttgaatacattggttaaatcccaacatgtaatatatgtaaataatcaatattatgctgctaaaataacacaaatcagtaagattctgtaatatttcatgataaataacttttgaaaatatatttttaaacattttggcttatgccttgagaattatttacctttttaaaatgtattttcctttcaggtttccagagctttacctg

SEQ ID NO: 387 - DNA, 442 bp, Homo Sapiens
tactaaacacagaattttgtaaaacaataagtgtataaagtaaaatgaacattaggattattgagattattgtagctaaaactagtgtttattcatataaattatgttaataaattgtattgtcattattgcattttacttttttgaaaagtagttaatgcctgtgtttctatatgagtattatataattcaagaagatattggatgaattttttttaagtttaatgtgtttcacatctctgtttcttttctctgcaccaaaagctacatttttgtgcccttatgtaccaggcagaaattgatctgcaatacatgtggagtctccaagggtatatttaaatttagtaattttattgctaactgtgaagttaatctgcactatatgggttcttttccccaggaaactgaaatagcagttcaagctaaacaaccggatgtggaa

SEQ ID NO: 388 - DNA, 444 bp, Homo Sapiens
atataattgactgggggtgagccagtacattaggattttcctaaagttatctggataattttagtatgcaaccacaatagatactcttcaagaattaagctagttgctgagagggaactgttttttgttggtttgttttcactaatgtttgcactctacttcctttaaataaaattatgcctggagaaagggtttttgtatggagcaattgataaatatttgtagggtggttggctaaaataattataattcctttaaaagaaattctacccactaaagttaatttagaagtaaaatataatagaaatccaataatatattcaccaaatggattaagatgttcatgaattatcttcaaagtgttaatcgaataagtaatgtgtatgcttttctgttaaagaggaagttagaagatctgagctctgagtggaaggcggtaaaccg

SEQ ID NO: 389 - DNA, 443 bp, Homo Sapiens
caacaagttaatgaagagggaaagaaatgtatgaggtttttttcgttcaaatgttgttatatgtcacatattcaacaattatatatgagcttatttttgtagtttttttctcttgtgataaaaacaattaagcccactttattgccaattaattgctactaagttgaaatacttgatactggttattgctcaagatgctgcatttgaaaagtttgtcctgaaaggtgggttaccttatactgtcatgattgactaaatcatatggtaggttaaaagcaatctaatatatgtattctgacctgaggattcagaagctgtttacgaagtattttaagacactccaactagagatttcataaaaaaaactgacattcattctctttctcataaaaatctatagcagttggccaaagacctccgccagtggcagacaaatgtagatg

SEQ ID NO: 390 - RNA, 2 nt, Homo Sapiens
ag